(19) United States
(12) Patent Application Publication
(10) Pub. No.: US 2010/0261711 A1
(43) Pub. Date: Oct. 14, 2010

(54) SELECTIVE ANTICONVULSANT AGENTS AND THEIR USES

(75) Inventors: James M. Cook, Whitefish Bay, WI (US); Rahul V. Edwankar, Milwaukee, WI (US); Chitra R. Edwankar, Milwaukee, WI (US); Shengming Huang, Milpitas, CA (US); Hiteshkumar D. Jain, Mumbai (IN); Jie Yang, Rensselaer, NY (US); Felix Rivas, Waukegan, IL (US); Hao Zhou, Paramus, NJ (US)

Correspondence Address: WISYS TECHNOLOGY FOUNDATION, INC.;c/o MIRICK, O'CONNELL, DEMALLIE & LOUGEE, LLP, 1700 WEST PARK DRIVE/ ROGER P. ZIMMERMAN, ESQ., WESTBOROUGH, MA, 01581-3941, US

(73) Assignee: WiSys Technology Foundation, Madison, WI (US)

(21) Appl. No.: 12/725,763

(22) Filed: Mar. 17, 2010

Related U.S. Application Data
(60) Provisional application No. 61/161,986, filed on Mar. 20, 2009.

(51) Int. Cl. A61K 31/5517; A61K 31/5513; A61P 25/00

(52) U.S. Cl. 514/220; 514/221

(57) ABSTRACT

In preferred embodiments, the present invention provides methods of treatment and pharmaceutical compositions for the suppression, alleviation and prevention of seizures. The preferred embodiments of the present invention further relate to methods of treatment and pharmaceutical compositions using benzodiazepine derivatives that provide suppression, alleviation and prevention of seizures with reduced sedative and ataxic side effects.

### RELATED APPLICATION

This application claims benefit of U.S. Provisional Patent Application 61/161,986, filed Mar. 20, 2009, the entire contents of which are incorporated by reference for all purposes.

### STATEMENT REGARDING FEDERALLY SPONSORED RESEARCH OR DEVELOPMENT

This invention was made with United States government support under NIMH grant number MH46851. The United States government has certain rights to this invention.

### BACKGROUND OF THE INVENTION

In certain aspects, the present invention relates to compositions including a class of benzodiazepine derivatives that are subunit-selective GABA_{A} receptor agonists.

Gamma-aminobutyric acid (GABA) is the major inhibitory neurotransmitter in the central nervous system. GABA receptors are heteromeric, and are divided into three main classes: (1) GABA_{A} receptors, which are members of the ligand-gated ion channel superfamily; (2) GABA_{B} receptors, which may be members of the G-protein linked receptor superfamily; and (3) GABA_{C} receptors, also members of the ligand-gated ion channel superfamily, but their distribution is confined to the retina. Benzodiazepine receptor ligands do not bind to GABA_{B} and GABA_{c} receptors. Since the first cDNAs encoding individual GABA_{A} receptor subunits were cloned the number of known members of the mammalian family has grown to 21 including α, β, and γ subunits (6α, 4β, 4γ, 1δ, 1ε, 1π, 1θ, and 3ρ).

A characteristic property of GABA_{A} receptors is the presence of a number of modulatory sites, one of which is the benzodiazepine (BZ) site. The benzodiazepine binding site is the most explored of the GABA_{A} receptor modulatory sites, and is the site through which benzodiazepine-based anxiolytic drugs exert their effect. Before the cloning of the GABA_{A} receptor gene family, the benzodiazepine binding site was historically subdivided into two subtypes, BENZODIAZEPINE1 and BENZODIAZEPINE2, on the basis of radioligand binding studies on synaptosomal rat membranes. The BENZODIAZEPINE1 subtype has been shown to be pharmacologically equivalent to a GABA_{A} receptor comprising the α1 subunit in combination with a β subunit and γ2. It has been indicated that an α subunit, a β subunit and a γ subunit constitute the minimum requirement for forming a functional GABA_{A} receptor.

Receptor subtype assemblies for BZ-sensitive GABA_{A} receptors include amongst others the subunit combinations α1β2γ2, α2β2,3γ2, α3β2,3γ2, α2βγ3, and α5β3γ2,3. Subtype assemblies containing an α1 subunit (α1β2γ2) are present in most areas of the brain and are thought to account for 40-50% of GABA_{A} receptors in the rat. Subtype assemblies containing α2 and α3 subunits respectively are thought to account for about 25% and 17% GABA_{A} of the receptors in the rat. Subtype assemblies containing an α5 subunit (α5β3γ2) are expressed predominately in the hippocampus and cortex and are thought to represent about 5% of GABA_{A} receptors in the rat. Two other major populations are the α2β2/3γ2 and α3β2/3γ2/3 subtypes. Together these constitute approximately a further 35% of the total GABA_{A} receptor population. Pharmacologically this combination appears to be equivalent to the BENZODIAZEPINE2 subtype as defined previously by radioligand binding, although the BENZODIAZEPINE2 subtype may also include certain α5-containing subtype assemblies.

The present pharmacology of agonists acting at the BZ site of GABA_{A} receptors suggests that α1 containing receptors mediate sedation, ataxia, anticonvulsant activity and anterograde amnesia, while α2 and/or α3 GABA_{A} receptors mediate anxiolytic activity and some anticonvulsant activity. The α5 containing GABA_{A} receptors are involved in memory functions (U. Rudolph et al., Nature 1999, 401, 796; K. Low et al., Science 2000, 290, 131; McKernan Nature Neurosci. 2000, 3, 587; F. Crestani et al., Proc. Nat. Acad. Sci. USA 2002, 99, 8980; M. S. Chambers et al., J. Med. Chem. 2003, 46, 2227).

It is believed that agents acting selectively as benzodiazepine agonists at GABA_{A}/α2, GABA_{A}/α3, and/or GABA_{A}/α5 receptors possess desirable properties. Compounds which are modulators of the benzodiazepine binding site of the GABA_{A} receptor by acting as benzodiazepine agonists are referred to hereinafter as “GABA_{A} receptor agonists.” The GABA_{A}/α1-selective (α1β2γ2) agonists alpidem and zolpidem are clinically prescribed as hypnotic agents, suggesting that at least some of the sedation associated with known anxiolytic drugs which act at the BENZODIAZEPINE1 binding site is mediated through GABA_{A} receptors containing the α1 subunit.

It is also known that some benzodiazepine derivatives, such as QH-ii-066, bind with high affinity to GABA_{A}/α5 receptors (Ki<10 nM), intermediate affinity to GABA_{A}/α2 and GABA_{A}/α3 (Ki<50 nM), and lower affinity to GABA_{A}/α1 receptors (Ki>70 nM), unlike diazepam which binds with high affinity to all four diazepam-sensitive GABA_{A} receptors (Ki<25 nM), as disclosed in Huang, et al., J. Med. Chem. 2000, 43, 71-95.

### SUMMARY OF THE INVENTION

In preferred embodiments, the present invention provides methods of treatment and pharmaceutical compositions for the suppression, alleviation and prevention of seizures. The preferred embodiments of the present invention further relate to methods of treatment and pharmaceutical compositions using benzodiazepine derivatives that provide suppression, alleviation and prevention of seizures with reduced sedative and ataxic side effects.

In certain aspects, the present invention provides methods for the treatment and prevention of seizures comprising administering to a subject in need of such treatment an effective amount of a compound of the formula

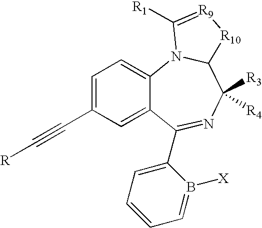

or a salt thereof, where R is H or Si(CH₃)₃,
R₁ is —H, —CH₃, ═O, or —CF₃, R₃ and R₄ are both H, or R₃ is H and R₄ is S(CH₃), or R₃ is R(CH₃) and R₄ is H,

### R₉ is N, or

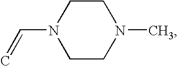

R₁₀ is N, C—CO₂CH₂CH₃, C—CO₂CH₂CF₃, C—CO₂C(CH₃)₃, C—R₁₅, C—R₁₆,

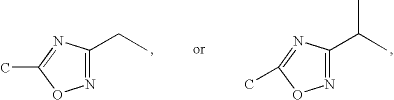

and B is C or N, and X is absent, H, F, Cl, or Br, and when R₁₀ is C—R₁₅,

### R₁₅ is

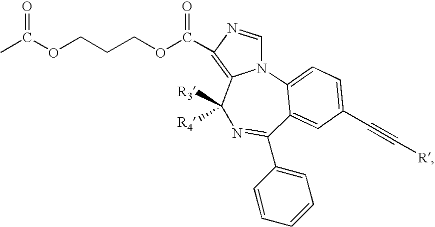

R₁ is H, R′ is H or Si(CH₃)₃, R′₃ and R′₄ are both H, or R′₃ is H and R′₄ is S(CH₃), or R′₃ is R(CH₃), and R′₄ is H, and when R₁₀ is C—R₁₅, R₁₅ is R₁₆ is

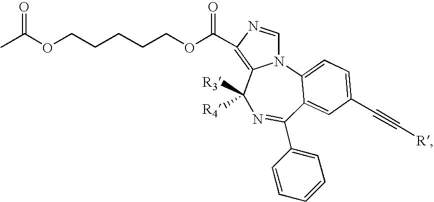

R₁ is H, R′ is H or Si(CH₃)₃, R′₃ and R′₄ are both H, or R′₃ is H and R′₄ is S(CH₃), or R′₃ is R(CH₃), and R′₄ is H.

In preferred embodiments, preferred compounds for the practice of the present invention include

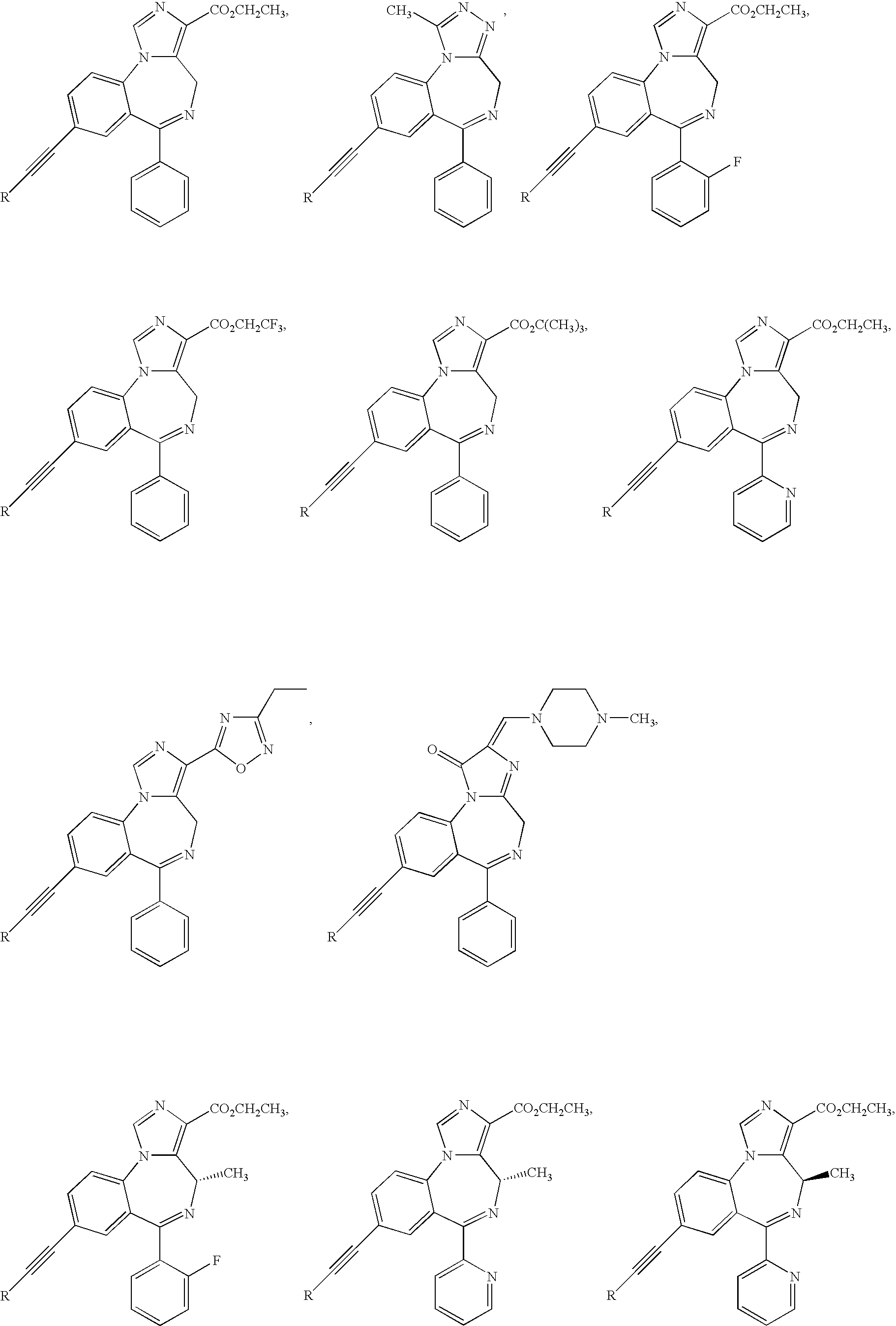

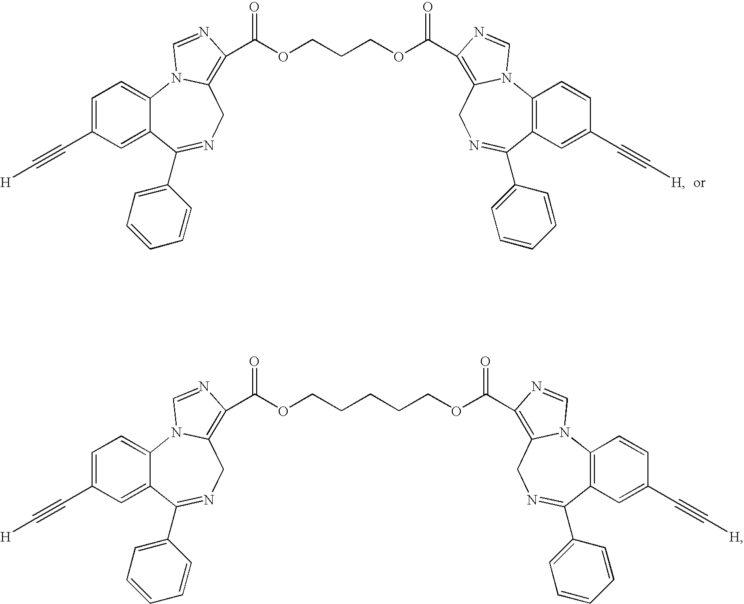

and salts thereof, where R is H or Si(CH₃)₃.

Particularly preferred compounds are characterized in Table 1, below.

TABLE 1

| Compound | R | R₁ | R₃ | R₄ | R₉ | R₁₀ | B | X |
|---|---|---|---|---|---|---|---|---|
| XHE-II-053 | H | —H | H | H | N | C—CO₂CH₂CH₃ | C | H |
| XHE-II-048 | Si(CH₃)₃ | —H | H | H | N | C—CO₂CH₂CH₃ | C | H |
| XLI-270 | H | —CH₃ | H | H | N | N | C | H |
| JY-XHE-053 | H | —H | H | H | N | C—CO₂CH₂CH₃ | C | F |
| JY-038 | Si(CH₃)₃ | —H | H | H | N | C—CO₂CH₂CH₃ | C | F |
| dm-II-20 | H | —H | H | H | N | C—CO₂CH₂CF₃ | C | H |
| XLI-225 | H | —H | H | H | N | C—CO₂C(CH₃)₃ | C | H |
| HZ-166 | H | —H | H | H | N | C—CO₂CH₂CH₃ | N | — |
| PS-I-26 | H | —H | H | H | N | 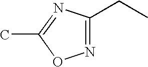 | C | H |
| PS-I-37 | H | ═0 | H | H | 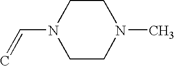 | N | C | H |
| PS-I-36 | Si(CH₃)₃ | ═0 | H | H | 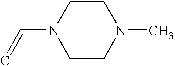 | N | C | H |
| DMH-D-053 | H | —H | H | H | N | C—R₁₅ | C | H |
| dm-III-97 | H | —H | H | H | N | C—R₁₆ | C | H |
| SH-053-2′F-S-CH₃ | H | —H | H | SCH₃ | N | C—CO₂CH₂CH₃ | C | F |
| SH-I-055 | Si(CH₃)₃ | —H | H | SCH₃ | N | C—CO₂CH₂CH₃ | C | F |
| SH-053-2′N-S-CH₃ | H | —H | H | SCH₃ | N | C—CO₂CH₂CH₃ | N | — |
| SH-053-2′N-R-CH₃ | H | —H | RCH₃ | H | N | C—CO₂CH₂CH₃ | N | — |
| SH-I-061 | H | —H | H | SCH₃ | N | C—CO₂CH₂CH₃ | N | — |
| SH-053-2′F-R-CH₃ | H | —H | RCH₃ | H | N | C—CO₂CH₂CH₃ | C | F |
| SH-I-060 | Si(CH₃)₃ | —H | RCH₃ | H | N | C—CO₂CH₂CH₃ | C | F |

In other aspects, the present invention provides methods for the treatment and prevention of seizures comprising administering to a subject in need of such treatment an effective amount of a compound of the formula

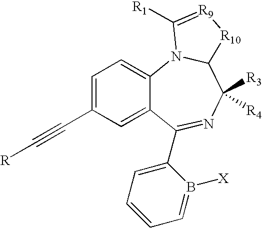

or a salt thereof, where R is H or Si(CH₃)₃,
R₁ is —H, —CH₃, ═O, or —CF₃, R₃ and R₄ are both H, or R₃ is H and R₄ is S(CH₃), or R₃ is R(CH₃) and R₄ is H,
R₉ is N, R₁₀ is N, C—CO₂CH₂CH₃, C—CO₂CH₂CF₃, or C—CO₂C(CH₃)₃,
and B is C or N, and X is absent, H, F, Cl, or Br.

In preferred embodiments, a preferred compound for the practice of the present invention is

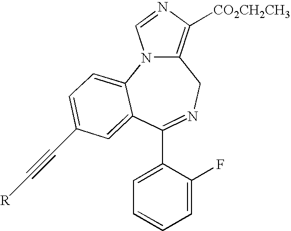

where R is H or Si(CH₃)₃.

In other preferred embodiments, a preferred compound for the practice of the present invention is

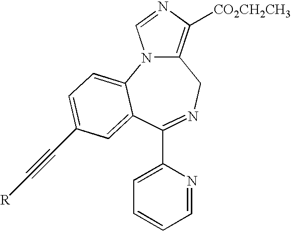

where R is H or Si(CH₃)₃.

In further preferred embodiments, a preferred compound for the practice of the present invention is

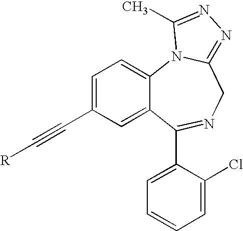

where R is H or Si(CH₃)₃.

In yet other preferred embodiments, a preferred compound for the practice of the present invention is

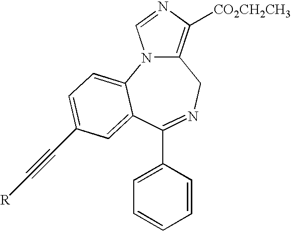

where R is H or Si(CH₃)₃.

In other aspects, the present invention provides methods for the treatment and prevention of seizures comprising administering to a subject in need of such treatment an effective amount of a compound selected from the group consisting of compounds according to Formulas A, B, C, D, I, III, or IV or a salt thereof,

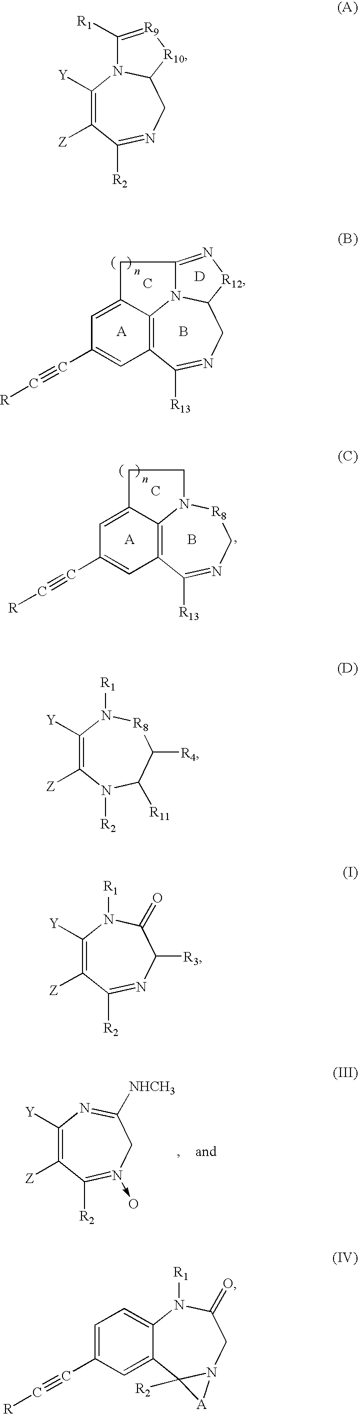

wherein Y and Z are taken together with the two intervening carbon atoms to form a ring selected from phenyl and thienyl, which ring is substituted at the C(8) position with at least the substituent —C≡C—R, where R is H, Si(CH₃)₃, t-butyl, isopropyl, methyl, or cyclopropyl;
R₂ is a substituted or unsubstituted at least partially unsaturated 5 or 6 membered cyclic or heterocyclic ring, wherein if substituted the substituent is one or more of F, Cl, Br, or NO₂ at the 2′-position; and R₁₀ is N, CH, C—CO₂—R₅,

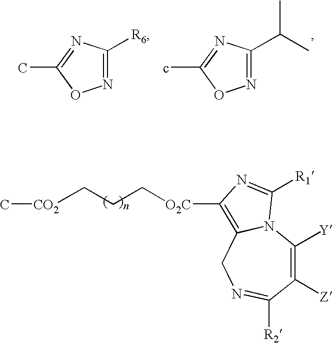

where n is 0 to 4 inclusive,

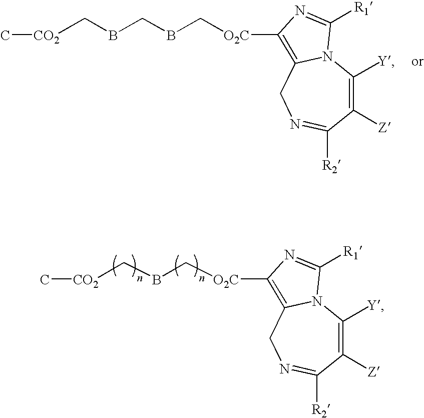

where n is 1 or 2 inclusive,

wherein Y′ and Z′ are taken together with the two intervening carbon atoms to form a ring selected from phenyl and thienyl, which ring is substituted at the C(8)′ position with at least the substituent —C≡C—R′, where R′ is H, Si(CH₃)₃, t-butyl, isopropyl, methyl, or cyclopropyl;

R₅ is a branched or straight chain C₁ to C₄ halogenated or unhalogenated alkyl or a methyl cyclopropyl;

R₆ is a branched or straight chain C₁ to C₄ alkyl or a methyl cyclopropyl;

R₁′ is one of H, CH₃, CF₃, CH₂CF₃, CH₂CH₃, or cyclopropyl;

R₂′ is a substituted or unsubstituted at least partially unsaturated 5 or 6 membered cyclic or heterocyclic ring, wherein if substituted the substituent is one or more of F, Cl, Br, or NO₂ at the 2′-position; and wherein when R₁₀ is C—CO₂—R₅, R₁ is one of H, CH₃, CF₃, CH₂CF₃, CH₂CH₃, CH₂C≡CH, or cyclopropyl;

otherwise, R₁ is one of H, CH₃, CF₃, CH₂CH₃, CH₂CF₃, or cyclopropyl; and wherein when R₁₀ is

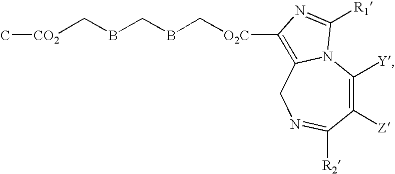

B is O or NH and wherein —BCH₂B— is optionally replaced with —N(R₇)—N(R₇)—, where R₇ is one of H, CH₃, alkyl, or cycloalkyl; and when R₁₀ is

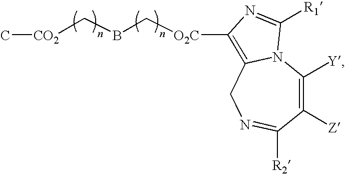

B is O or NH or —N(R₇)—N(R₇)—, where R₇ is one of H, CH₃, alkyl, or cycloalkyl;

### and R₉ is N or

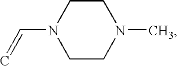

wherein when R₉ is

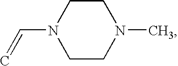

### R₁ is ═O and R₁₀ is N; R₃ is one of —H, —OH, —OCON(CH₃)₂, —COOH, —COOCH₃, —COOC₂H₅,

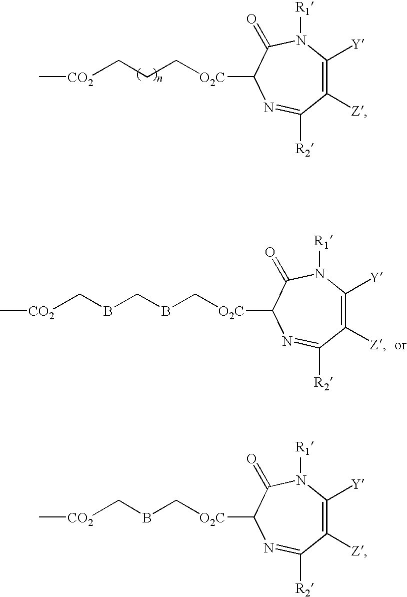

wherein if R₃ is

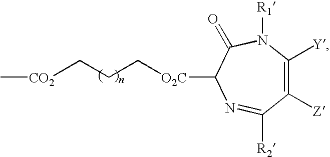

n is 0 to 4; Y′ and Z′ are taken together with the two intervening carbon atoms to form a ring selected from phenyl and thienyl, which ring is substituted at the C(7)′ position with at least the substituent —C≡C—R′, where R′ is H, Si(CH₃)₃, t-butyl, isopropyl, methyl, or cyclopropyl;
R₁ and R₁′ are independently one of H, CH₃, CF₃, CH₂CF₃, CH₂CH₃, or cyclopropyl;
R₂ and R₂′ are independently a substituted or unsubstituted at least partially unsaturated 5 or 6 membered cyclic or heterocyclic ring, wherein if substituted the substituent is at least one of F, Cl, Br, or NO₂ at the 2′-position, or
wherein if R₃ is

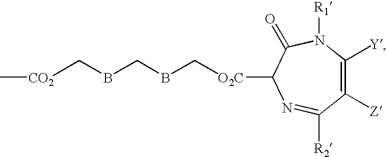

Y′ and Z′ are taken together with the two intervening carbon atoms to form a ring selected from phenyl and thienyl, which ring is substituted at the C(7)′ position with at least the substituent —C≡C—R′, where R′ is H, Si(CH₃)₃, t-butyl, isopropyl, methyl, or cyclopropyl; R₁ and R₁′ are independently one of H, CH₃, CF₃, CH₂CH₃, CH₂CF₃ or cyclopropyl; R₂ and R₂′ are independently a substituted or unsubstituted at least partially unsaturated 5 or 6 membered cyclic or heterocyclic ring, wherein if substituted the substituent is at least one of F, Cl, Br, or NO₂ at the 2′-position; B is O or NH and wherein —BCH₂B— is optionally replaced with —N(R₇)—N(R₇)—, where R₇ is one of H, branched or straight chain C₁ to C₄ alkyl, cycloalkyl or methyl cyclopropyl,
wherein if R₃ is

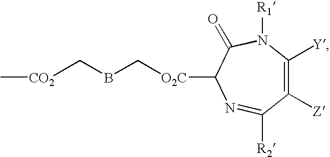

Y′ and Z′ are taken together with the two intervening carbon atoms to form a ring selected from phenyl and thienyl, which ring is substituted at the C(7)′ position with at least the substituent —C≡C—R′, where R′ is H, Si(CH₃)₃, t-butyl, isopropyl, methyl, or cyclopropyl;
R₁ and R₁′ are independently one of H, CH₃, CF₃, CH₂CH₃, CH₂CF₃ or cyclopropyl;
R₂ and R₂′ are independently a substituted or unsubstituted at least partially unsaturated 5 or 6 membered cyclic or heterocyclic ring, wherein if substituted the substituent is one or more of F, Cl, Br, or NO₂ at the 2′-position;
B is O, NH, or —N(R₇)—N(R₇)—, where R₇ is one of H, branched or straight chain C₁ to C₄ alkyl, cycloalkyl or methyl cyclopropyl, wherein R₂ is not phenyl when R₃ is H, R₁ is H or CH₃ and Y and Z taken together form a phenyl ring; wherein R₈ is C═O, C═S, or C—NHCH₃;
R₁₁ is O or N and R₄ is H, O or C,
wherein when R₁₁ is O, R₄ is H, and R₈ is C═O;
when R₁₁ is N, R₄ is C and R₁₁ and R₄ are taken together with an intervening nitrogen atom to form a pyrazole ring;
when R₁₁ is N, R₄ is O and R₁₁ and R₄ are taken together with an intervening carbon atom to form an oxazole ring; or
when R₁₁ is N, R₄ is C and R₁₁ and R₄ are taken together with two intervening carbon atoms to form a pyridine ring;
   and R₁₂ is N or CH, R₁₃ is phenyl, 2-thienyl, 3-thienyl, 2-pyridyl, or 2-pyridyl N—O, and if the compound is according to Formula B or C, n is an integer from 1 to 2 inclusive and ring C is saturated or unsaturated.

The compounds described herein have been synthesized based on a modified version of the computer modeling disclosed in Cook, et al J. Med. Chem., 1996, 39, 1928-1934. These compounds obtained by modifying elements, described herein, of the known benzodiazepine agents, have increased binding selectivity for the GABA_{A}/α2, GABA_{A}/α3, and/or GABA_{A}/α5 receptors described above, and/or altered efficacy at one or more GABA_{A} receptors described above, and/or altered selectivity at one or more ion channels. Further details of the synthesis of these compounds are found in U.S. Pat. No. 7,119,196, and published patent application WO 2003/082832, WO 2006/004945, and US 2006/0003995, all of which are incorporated by reference herein in their entirety.

Suitable compounds for the practice of embodiments of the present invention have binding selectivity for the GABA_{A}/α2, GABA_{A}/α3 receptors over GABA_{A}/α1 receptors. In another aspect, suitable compounds for the practice of embodiments of the present invention have higher physiological efficacy at GABA_{A}/α2, GABA_{A}/α3 receptors than at GABA_{A}/α1 receptors.

Suitable compounds for the practice of embodiments of the present invention include compounds of formula I, or a salt or prodrug thereof,

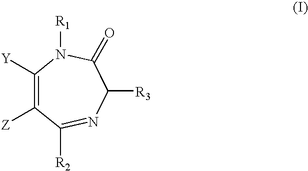

wherein Y and Z are taken together with the two intervening carbon atoms to form a ring selected from phenyl and thienyl, which ring is substituted at the C(7) position with at least the substituent —C≡C—R, where R is H, Si(CH₃)₃, isopropyl, methyl, or cyclopropyl; R₁ is one of H, CH₃, C₂H₄N(C₂H₅)₂, CH₂CF₃, CH₂C≡CH, or an alkyl cyclopropyl; R₂ is a substituted or unsubstituted at least partially unsaturated 5 or 6 membered cyclic or heterocyclic ring, wherein if substituted the substituent is one or more of F, Cl, Br, or NO₂ at the 2′-position; and R₃ is one of H, OH, CH₃, CF₃, OCON(CH₃)₂, COOCH₃, or COOC₂H₅. Preferred compounds according to formula I include:

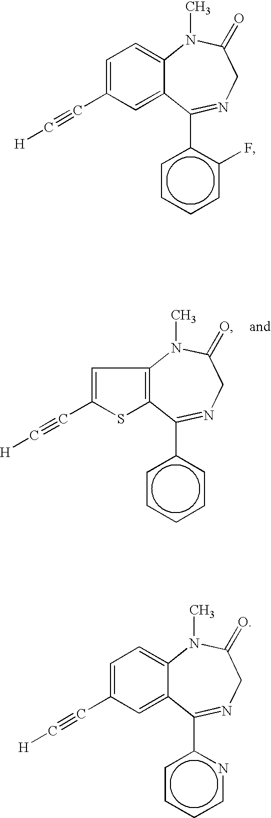

Suitable compounds for the practice of embodiments of the present invention include compounds of formula II, or a salt or prodrug thereof,

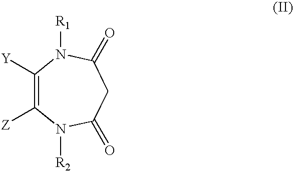

wherein Y and Z are taken together with the two intervening carbon atoms to form a ring selected from phenyl and thienyl, which ring is substituted at the C(7) position with at least the substituent —C≡C—R, where R is H, Si(CH₃)₃, isopropyl, methyl, or cyclopropyl; R₁ is one of H, CH₃, C₂H₄N(C₂H₅)₂, CH₂CF₃, CH₂C≡CH, or an alkyl cyclopropyl; and R₂ is a substituted or unsubstituted at least partially unsaturated 5 or 6 membered cyclic or heterocyclic ring, wherein if substituted the substituent is one or more of F, Cl, Br, or NO₂ at the 2′-position. Preferred compounds according to formula II include:

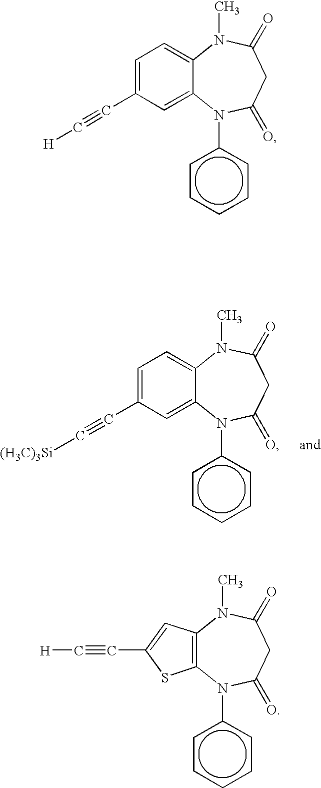

Suitable compounds for the practice of embodiments of the present invention include compounds of formula III, or a salt or prodrug thereof,

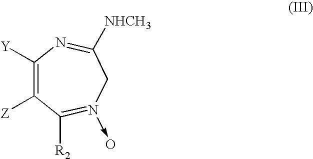

wherein Y and Z are taken together with the two intervening carbon atoms to form a ring selected from phenyl and thienyl, which ring is substituted at the C(7) position with at least the substituent —C≡C—R, where R is H, Si(CH₃)₃, isopropyl, methyl, or cyclopropyl; and R₂ is a substituted or unsubstituted at least partially unsaturated 5 or 6 membered cyclic or heterocyclic ring, wherein if substituted the substituent is one or more of F, Cl, Br, or NO₂ at the 2′-position. Preferred compounds according to the formula III include:

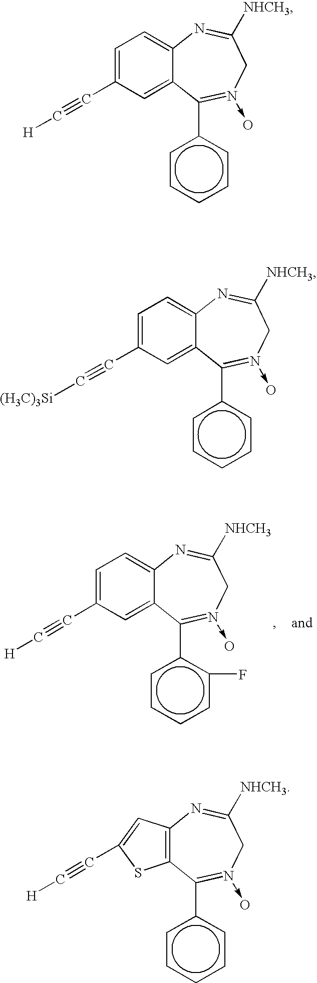

Suitable compounds for the practice of embodiments of the present invention include compounds of formula IV, or a salt or prodrug thereof,

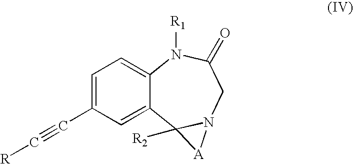

wherein R is H, Si(CH₃)₃, isopropyl, methyl, or cyclopropyl; R₁ is one of H, CH₃, C₂H₄N(C₂H₅)₂, CH₂CF₃, CH₂C≡CH, or an alkyl cyclopropyl; R₂ is a substituted or unsubstituted at least partially unsaturated 5 or 6 membered cyclic or heterocyclic ring, wherein if substituted the substituent is one or more of F, Cl, Br, or NO₂ at the 2′-position; and A is an ethoxide or a propoxide. Preferred compounds according to the formula IV include:

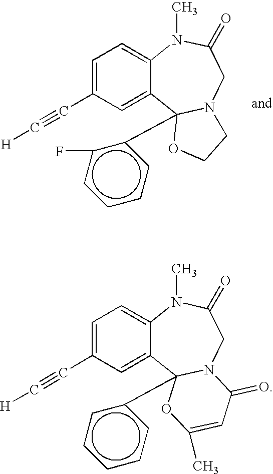

Suitable compounds for the practice of embodiments of the present invention include compounds of formula V, or a salt or prodrug thereof,

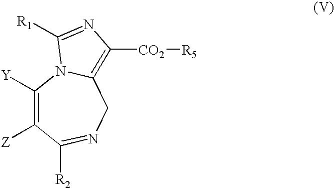

wherein Y and Z are taken together with the two intervening carbon atoms to form a ring selected from phenyl and thienyl, which ring is substituted at the C(8) position with at least the substituent —C≡C—R, where R is H, Si(CH₃)₃, isopropyl, methyl, or cyclopropyl; R₁ is one of H, CH₃, CF₃, CH₂CH₃, CH₂CF₃, CH₂C≡CH, an alkyl, or cyclopropyl; R₂ is a substituted or unsubstituted at least partially unsaturated 5 or 6 membered cyclic or heterocyclic ring, wherein if substituted the substituent is one or more of F, Cl, Br, or NO₂ at the 2′-position; and R₅ is a branched or straight chain C₁ to C₄ halogenated or unhalogenated alkyl or a methyl cyclopropyl. Preferred compounds according to formula V include:

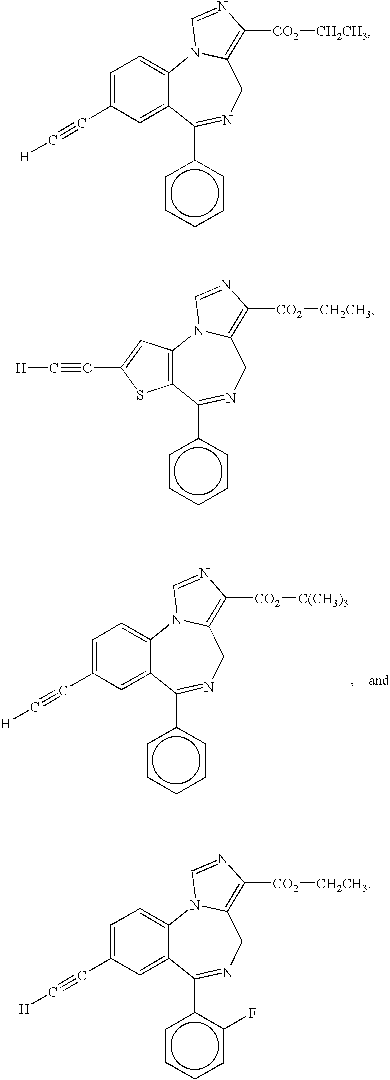

Suitable compounds for the practice of embodiments of the present invention include compounds of formula VI, or a salt or prodrug thereof,

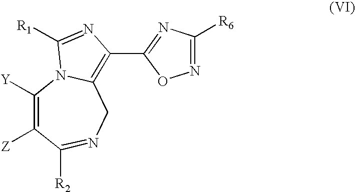

wherein Y and Z are taken together with the two intervening carbon atoms to form a ring selected from phenyl and thienyl, which ring is substituted at the C(8) position with at least the substituent —C≡C—R, where R is H, Si(CH₃)₃, isopropyl, methyl, or cyclopropyl; R₁ is one of H, CH₃, CF₃, CH₂CH₃, CH₂CF₃, or cyclopropyl; R₂ is a substituted or unsubstituted at least partially unsaturated 5 or 6 membered cyclic or heterocyclic ring, wherein if substituted the substituent is one or more of F, Cl, Br, or NO₂ at the 2′-position; and R₆ is a branched or straight chain C₁ to C₄ alkyl or a methyl cyclopropyl. Preferred compounds according to formula VI include:

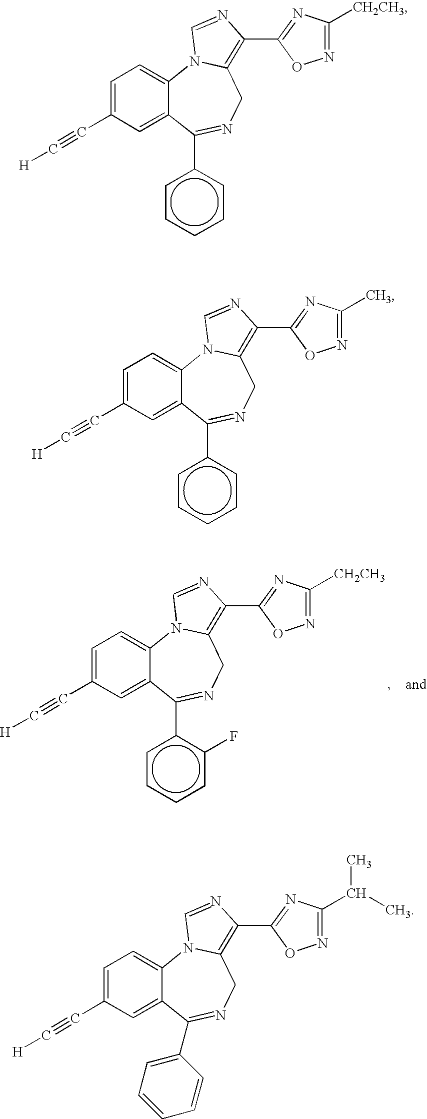

Suitable compounds for the practice of embodiments of the present invention include compounds of formula VII, or a salt or prodrug thereof,

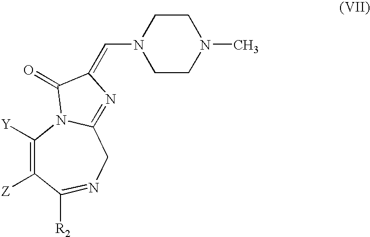

wherein Y and Z are taken together with the two intervening carbon atoms to form a ring selected from phenyl and thienyl, which ring is substituted at the C(8) position with at least the substituent —C≡C—R, where R is H, Si(CH₃)₃, isopropyl, methyl, or cyclopropyl; and R₂ is a substituted or unsubstituted at least partially unsaturated 5 or 6 membered cyclic or heterocyclic ring, wherein if substituted the substituent is one or more of F, Cl, Br, or NO₂ at the 2′-position. Preferred compounds according to formula VII include:

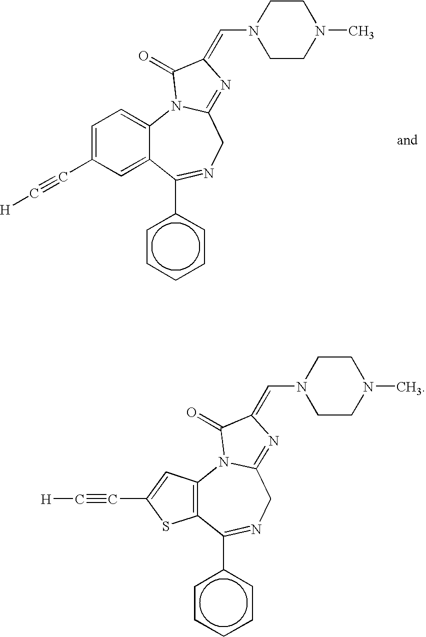

Suitable compounds for the practice of embodiments of the present invention include compounds of formula VIII, or a salt or prodrug thereof,

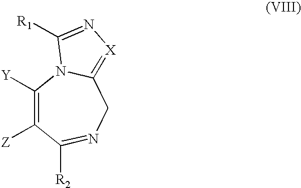

wherein Y and Z are taken together with the two intervening carbon atoms to form a ring selected from phenyl and thienyl, which ring is substituted at the C(8) position with at least the substituent —C≡C—R, where X is N or CH, where R is H, Si(CH₃)₃, isopropyl, methyl, or cyclopropyl; R₁ is H, CH₃, CF₃, CH₂CH₃, CH₂CF₃, or cyclopropyl; R₂ is a substituted or unsubstituted at least partially unsaturated 5 or 6 membered cyclic or heterocyclic ring, wherein if substituted the substituent is one or more of F, Cl, Br, or NO₂ at the 2′-position. Preferred compounds according to formula VIII include:

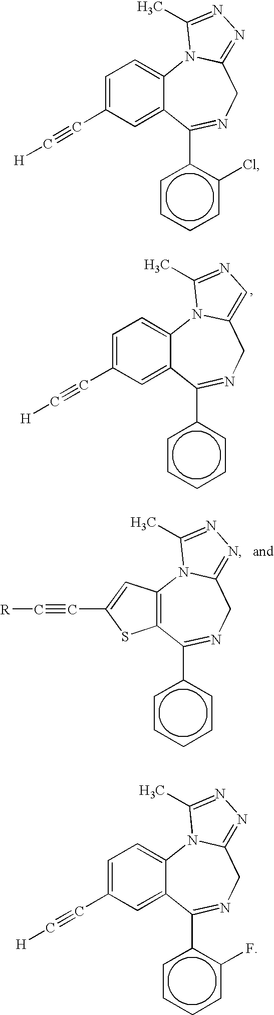

Suitable compounds for the practice of embodiments of the present invention include compounds of formula IX, or a salt or prodrug thereof,

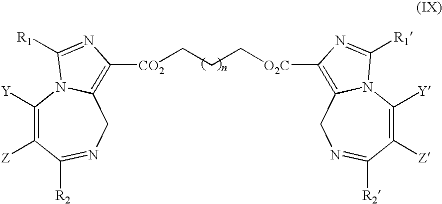

wherein n is 0 to 4; Y and Z are taken together with the two intervening carbon atoms to form a ring selected from phenyl and thienyl, which ring is substituted at the C(8) position with at least the substituent —C≡C—R, where R is H, Si(CH₃)₃, isopropyl, methyl, or cyclopropyl; Y′ and Z′ are taken together with the two intervening carbon atoms to form a ring selected from phenyl and thienyl, which ring is substituted at the C(8)′ position with at least the substituent
—C≡C—R′, where R′ is H, Si(CH₃)₃, isopropyl, methyl, or cyclopropyl; R₁ and R₁′ are independently one of H, CH₃, CF₃, CH₂CF₃, CH₂CH₃, or cyclopropyl; and R₂ and R₂′ are independently a substituted or unsubstituted at least partially unsaturated 5 or 6 membered cyclic or heterocyclic ring, wherein if substituted the substituent is one or more of F, Cl, Br, or NO at the 2′-position. Preferred compounds according to

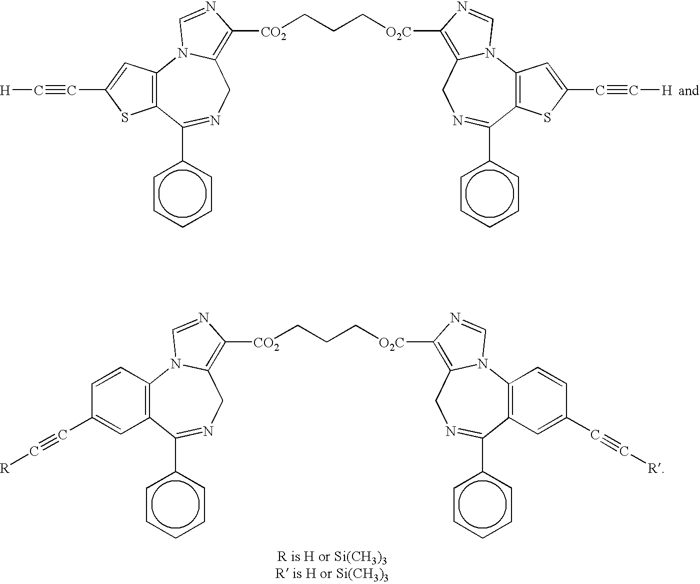

formula IX include:

Suitable compounds for the practice of embodiments of the present invention include compounds of formula X, or a salt or prodrug thereof,

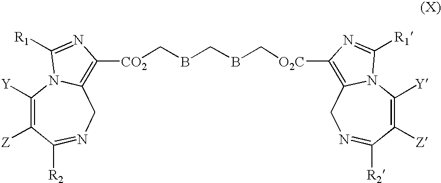

wherein Y and Z are taken together with the two intervening carbon atoms to form a ring selected from phenyl and thienyl, which ring is substituted at the C(8) position with at least the substituent —C≡C—R, where R is H, Si(CH₃)₃, t-butyl, isopropyl, methyl, or cyclopropyl; Y′ and Z′ are taken together with the two intervening carbon atoms to form a ring selected from phenyl and thienyl, which ring is substituted at the C(8)′ position with at least the substituent —C≡C—R′ where R′ is H, Si(CH₃)₃, t-butyl, isopropyl, methyl, or cyclopropyl; R₁ and R₁′ are independently one of H, CH₃, CF₃, CH₂CH₃, CH₂CF₃, or cyclopropyl; R₂ and R₂′ are independently a substituted or unsubstituted at least partially unsaturated 5 or 6 membered cyclic or heterocyclic ring, wherein if substituted the substituent is one or more of F, Cl, Br, or NO₂ at the 2′-position; and B is O or NH and wherein —BCH₂B— is optionally replaced with —N(R₇)—N(R₇)—, where R₇ is one of H, CH₃, alkyl, or cycloalkyl. Preferred compounds according to formula X include:

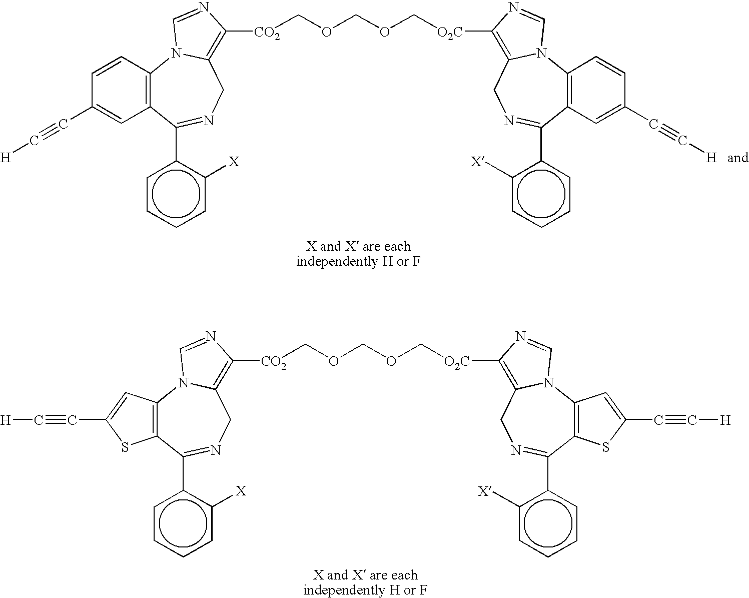

Suitable compounds for the practice of embodiments of the present invention also include compounds of formula XI, or a salt or prodrug thereof,

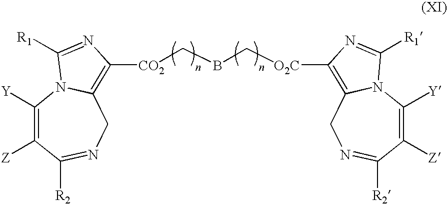

wherein n is 1 or 2; wherein Y and Z are taken together with the two intervening carbon atoms to form a ring selected from phenyl and thienyl, which ring is substituted at the C(8) position with at least the substituent —C≡C—R, where R is H, Si (CH₃)₃, isopropyl, methyl, or cyclopropyl; Y′ and Z′ are taken together with the two intervening carbon atoms to form a ring selected from phenyl and thienyl, which ring is substituted at the C(8)′ position with at least the substituent —C≡C—R′, where R′ is H, Si(CH₃)₃, isopropyl, methyl, or cyclopropyl; R₁ and R₁′ are independently one of H, CH₃, CF₃, CH₂CH₃, CH₂CF₃, or cyclopropyl; R₂ and R₂′ are independently a substituted or unsubstituted at least partially unsaturated 5 or 6 membered cyclic or heterocyclic ring, wherein if substituted the substituent is one or more of F, Cl, Br, or NO₂ at the 2′-position; and B is O, NH, or —N(R₇)—N(R₇)—, where R₇ is one of H, CH₃, alkyl, or cycloalkyl. Preferred compounds according to formula XI include:

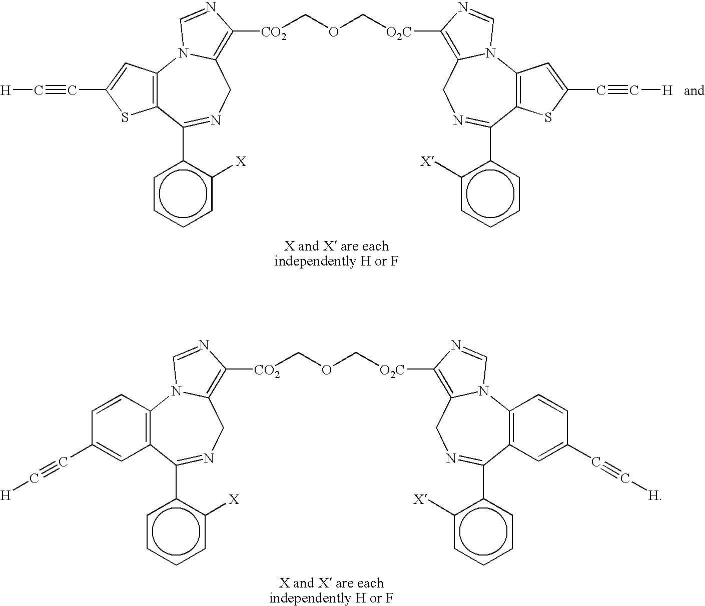

Suitable compounds for the practice of embodiments of the present invention include compounds of formula XII, or a salt or prodrug thereof,

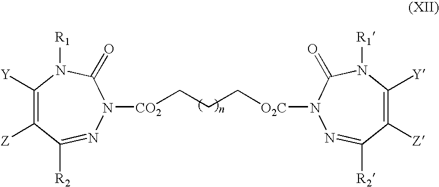

wherein n is 0 to 4; Y and Z are taken together with the two intervening carbon atoms to form a ring selected from phenyl and thienyl, which ring is substituted at the C(7) position with at least the substituent —C≡C—R, where R is H, Si(CH₃)₃, isopropyl, methyl, or cyclopropyl; Y′ and Z′ are taken together with the two intervening carbon atoms to form a ring selected from phenyl and thienyl, which ring is substituted at the C(7)′ position with at least the substituent —C≡C—R′, where R′ is H, Si(CH₃)₃, isopropyl, methyl, or cyclopropyl; R₁ and R₁′ are independently one of H, CH₃, CF₃, CH₂CF₃, CH₂CH₃, or cyclopropyl; and R₂ and R₂′ are independently a substituted or unsubstituted at least partially unsaturated 5 or 6 membered cyclic or heterocyclic ring, wherein if substituted the substituent is one or more of F, Cl, Br, or NO₂ at the 2′-position. Preferred compounds according to formula XII include:

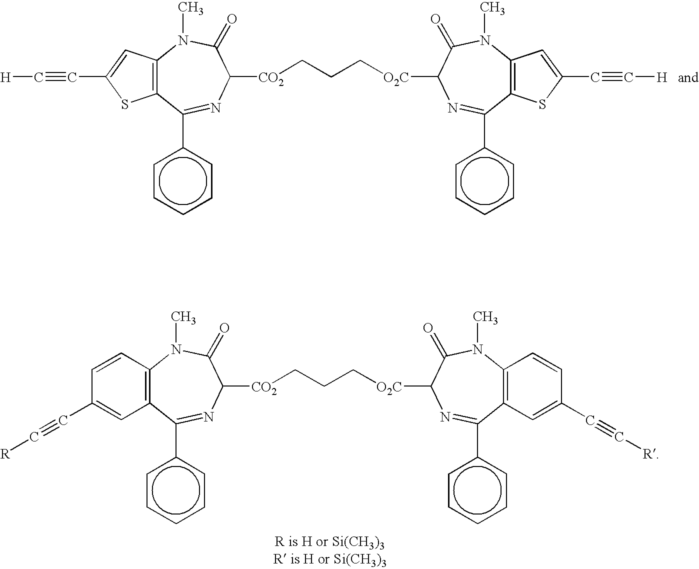

Suitable compounds for the practice of embodiments of the present invention include compounds of the formula XIII, or a salt or prodrug thereof,

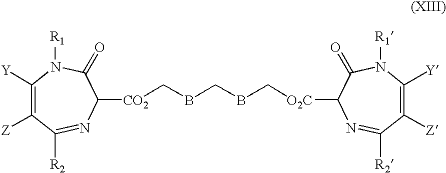

wherein Y and Z are taken together with the two intervening carbon atoms to form a ring selected from phenyl and thienyl, which ring is substituted at the C(7) position with at least the substituent —C≡C—R, where R is H, Si(CH₃)₃, isopropyl, methyl, or cyclopropyl; Y′ and E are taken together with the two intervening carbon atoms to form a ring selected from phenyl and thienyl, which ring is substituted at the C(7)' position with at least the substituent —C≡C—R′, where R′ is H, Si(CH₃)₃, isopropyl, methyl, or cyclopropyl; R₁ and R₁′ are independently one of H, CH₃, CF₃, CH₂CH₃, CH₂CF₃, or cyclopropyl; R₂ and R₂′ are independently a substituted or unsubstituted at least partially unsaturated 5 or 6 membered cyclic or heterocyclic ring, wherein if substituted the substituent is one or more of F, Cl, Br, or NO₂ at the 2′-position; and B is O or NH and wherein —BCH₂B— is optionally replaced with —N(R₇)—N(R₇)—, where R₇ is one of H, CH₃, alkyl, or cycloalkyl. Preferred compounds according to formula XIII include:

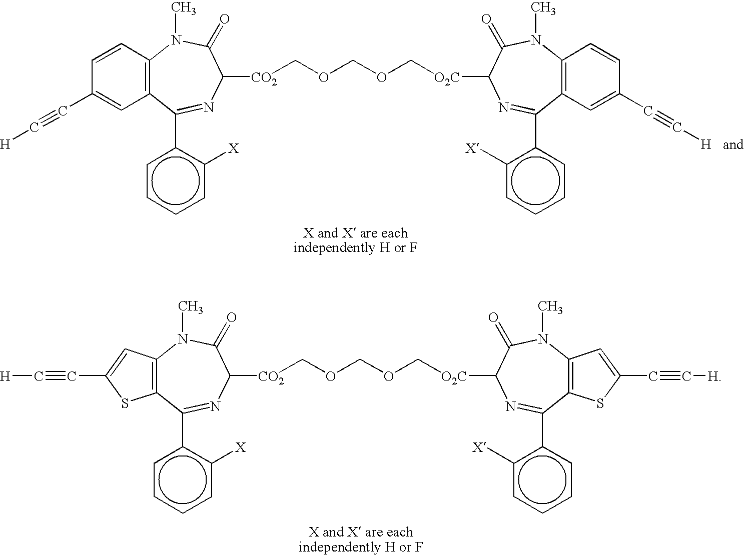

Yet other suitable compounds for the practice of embodiments of the present invention include compounds of the formula XIV, or a salt or prodrug thereof,

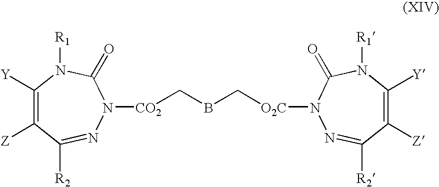

wherein Y and Z are taken together with the two intervening carbon atoms to form a ring selected from phenyl and thienyl, which ring is substituted at the C(7) position with at least the substituent —C≡C—R, where R is H, Si(CH₃)₃, isopropyl, methyl, or cyclopropyl; Y′ and Z′ are taken together with the two intervening carbon atoms to form a ring selected from phenyl and thienyl, which ring is substituted at the C(7)' position with at least the substituent —C≡C—R′, where R′ is H, Si(CH₃)₃, isopropyl, methyl, or cyclopropyl; R₁ and R₁′ are independently one of H, CH₃, CF₃, CH₂CH₃, CH₂CF₃, or cyclopropyl; R₂ and R₂′ are independently a substituted or unsubstituted at least partially unsaturated 5 or 6 membered cyclic or heterocyclic ring, wherein if substituted the substituent is one or more of F, Cl, Br, or NO₂ at the 2′-position; and B is O, NH, or —N(R₇)—N(R₇)—, where R₇ is one of H, CH₃, alkyl, or cycloalkyl. Preferred compounds according to formula XIV include:

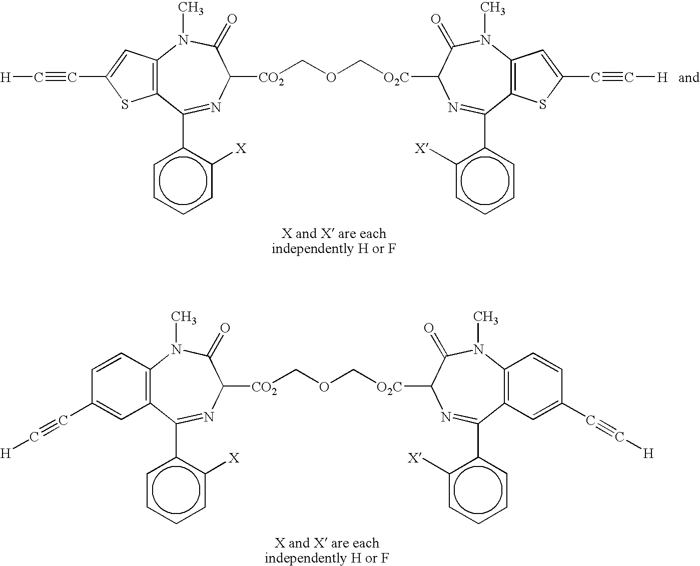

Yet other suitable compounds for the practice of embodiments of the present invention include compounds of the formula XV, or a salt or prodrug thereof,

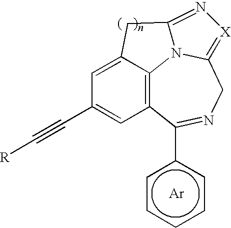

where n is 1-2 inclusive, R is H, Si(CH₃)₃, or cyclopropyl, Ar is phenyl, 2′-fluorophenyl, 2-thienyl, 3-thienyl, 2-pyridyl, 2-pyridyl N—O, and X is N or CH.

Yet other suitable compounds for the practice of embodiments of the present invention include compounds of the formula XVI, or a salt or prodrug thereof,

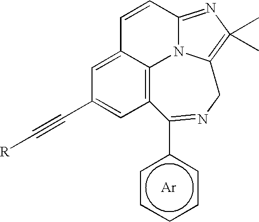

where R is H, Si(CH₃)₃, or cyclopropyl, Ar is phenyl, 2′-fluorophenyl, 2-thienyl, 3-thienyl, 2-pyridyl, 2-pyridyl N—O, and X is N or CH.

Further suitable compounds for the practice of embodiments of the present invention include compounds of the formula XVII, or a salt or prodrug thereof,

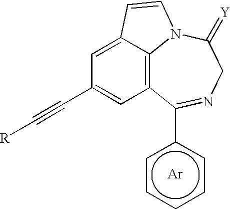

where R is H, Si(CH₃)₃, t-butyl, or cyclopropyl, Ar is phenyl, 2′-fluorophenyl, 2-thienyl, 3-thienyl, 2-pyridyl, 2-pyridyl N—O, and Y is O, S or NCH₃.

Further suitable compounds for the practice of embodiments of the present invention include compounds of the formula XVIII, or a salt or prodrug thereof,

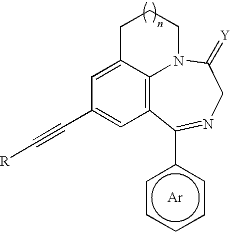

where n is 1-2 inclusive, R is H, Si(CH₃)₃, or cyclopropyl, Ar is phenyl, 2′-fluorophenyl, 2-thienyl, 3-thienyl, 2-pyridyl, 2-pyridyl N—O, and Y is O, S or NCH₃.

Other suitable compounds for the practice of embodiments of the present invention include compounds of the formula XIX, or a salt or prodrug thereof,

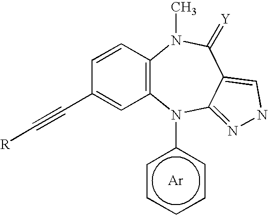

where R is H, Si(CH₃)₃, or cyclopropyl, Ar is phenyl, 2′-fluorophenyl, 2-thienyl, 3-thienyl, 2-pyridyl, 2-pyridyl N—O, and Y is O, S or NCH₃.

Other suitable compounds for the practice of embodiments of the present invention include compounds of the formula XX, or a salt or prodrug thereof,

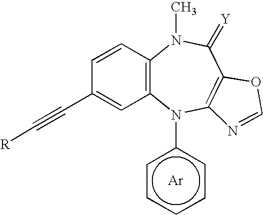

where R is H, Si(CH₃)₃, or cyclopropyl, Ar is phenyl, 2′-fluorophenyl, 2-thienyl, 3-thienyl, 2-pyridyl, 2-pyridyl N—O, and Y is O, S or NCH₃.

Further suitable compounds for the practice of embodiments of the present invention include compounds of the formula XXI, or a salt or prodrug thereof,

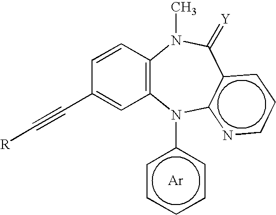

where R is H, Si(CH₃)₃, or cyclopropyl, Ar is phenyl, 2′-fluorophenyl, 2-thienyl, 3-thienyl, 2-pyridyl, 2-pyridyl N—O, and Y is O, S or NHCH₃.

Compounds (XV) to (XXI) above can also have R as CF₃, CCl₃, or CBr₃.

A still further aspect of the present invention provides compositions comprising compounds of the above kind in a pharmaceutically acceptable carrier. Such pharmaceutically acceptable carriers are well known in the art.

In the above embodiments by “alkyl” we mean a straight or branched halogenated or unhalogenated alkyl group having 1-6 carbon atoms. By “cycloalkyl” we mean one containing 3-7 carbon atoms. Also, in the above embodiments by “cyclic” we prefer a phenyl group and by “heterocyclic” we prefer a 2-pyridine or a 2- or 3-thiophene.

The compounds of the present invention are GABA_{A} receptor ligands which exhibit anxiolytic activity due to increased agonist efficacy at GABA_{A}/α2, GABA_{A}/α3 and/or GABA_{A}/α5 receptors. The compounds in accordance with this invention may possess at least 2-fold, suitably at least 5-fold, and advantageously at least a 10-fold, selective efficacy for the GABA_{A}/α2, GABA_{A}/α3, and/or GABA_{A}/α5 receptors relative to the GABA_{A}/α1 receptors. However, compounds which are not selective in terms of their agonist efficacy for the GABA_{A}/α2, GABA_{A}/α3, and/or GABA_{A}/α5 receptors are also encompassed within the scope of the present invention. Such compounds will desirably exhibit functional selectivity by demonstrating anxiolytic activity with decreased sedative-hypnotic/muscle relaxant/ataxic activity due to decreased efficacy at GABA_{A}/α1 receptors.

For use in medicine, the salts of the compounds of formulas (I)-(XXI) will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts, alkaline earth metal salts, e.g. calcium or magnesium salts; and salts formed with suitable organic ligands, e.g. quaternary ammonium salts.

The present invention includes within its scope prodrugs of the compounds of formulas (I)-(XXI) above. In general, such prodrugs will be functional derivatives of the compounds of formulas (I)-(XXI) which are readily convertible in vivo into the required compound of formulas (I)-(XXI). Conventional procedures for the selection and preparation of suitable prodrug derivatives are described, for example, in Design of Prodrugs, ed. H. Bundgaard, Elsevier, 1985.

Where the compounds according to the invention have at least one asymmetric center, they may accordingly exist as enantiomers. Where the compounds according to the invention possess two or more asymmetric centers, they may additionally exist as diastereoisomers. It is to be understood that all such isomers and mixtures thereof in any proportion are encompassed within the scope of the present invention. The examples of the use of stereroisomeric compounds in the practice of the present invention disclosed herein are illustrative examples, and are not limiting.

Practice of the embodiments of the invention also involves pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that the compounds of the present invention may be incorporated into transdermal patches designed to deliver the appropriate amount of the drug in a continuous fashion. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture for a compound of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example, 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium caboxymethylcellulose, methylcellulose, polyvinylpyrrolidone or gelatin.

Suitable dosage level is about 0.01 to 250 mg/kg per day, preferably about 0.05 to 100 mg/kg per day, and especially about 0.05 to 5 mg/kg per day. The compounds may be administered on a regimen of 1 to 4 times per day, or on a continuous basis via, for example, the use of a transdermal patch.

### BRIEF DESCRIPTION OF THE DRAWINGS

FIG. 1 is an ORTEP (Oak Ridge Thermal Ellipsoid Plot) drawing of the crystal structure of by-product SH—I-085 (ByP)

FIG. 2 is an ORTEP drawing of the crystal structure of JY-XHe-053 (15).

FIG. 3 is an ORTEP drawing of the crystal structure of SH-TS-CH₃ (143).

FIG. 4 is an ORTEP drawing of the crystal structure of SH-TR-CH₃ (146).

FIG. 5 is a graph of dose-response curves of the patch clamp current (expressed as percent of the control current) produced by various concentrations of diazepam when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 6 is a graph of dose-response curves of the patch clamp current (expressed as percent of the control current) produced by various concentrations of JC-221 when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 7 is a graph of dose-response curves of the patch clamp current (expressed as percent of the control current) produced by various concentrations of Hz-166 when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 8 is a graph of dose-response curves of the patch clamp current (expressed as percent of the control current) produced by various concentrations of JY-XHe-053 (15) when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 9 is a graph of dose-response curves of the patch clamp current (expressed as percent of the control current) produced by various concentrations of XLi-JY-DMH (23) when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 10 is a graph of dose-response curves of the patch clamp current (expressed as percent of the control current) produced by various concentrations of compound 147, an 8-iodo-imidazobenzodiazepine (the iodo analog of compound 5), when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 11 is a graph of dose-response curves of the patch clamp current (expressed as percent of the control current) produced by various concentrations of SH-053-S—CH₃ when applied to Xenopus oocytes expressing recombinant α1β3γ2 (open diamond), α2β3γ2 (▪), α3β3γ2 (*) or α5β3γ2 (▴) GABA_{A} receptors.

FIG. 12 is a graph of dose-response curves of the patch clamp current (expressed as percent of the control current) produced by various concentrations of SH-053-R—CH₃ when applied to Xenopus oocytes expressing recombinant α1β3γ2 (▴), α2β3γ2 (▪), α3β3γ2 (▾) or α5β3γ2 (♦) GABA_{A} receptors.

FIG. 13 is a graph of dose-response curves of the patch clamp current (expressed as percent of the control current) produced by various concentrations of SH-053-2′F—S—CH₃ when applied to Xenopus oocytes expressing recombinant α1β3γ2 (▪), α2β3γ2 (▴), α3β3γ2 (♦) or α5β3γ2 (▾) GABA_{A} receptors.

FIG. 14 is a graph of dose-response curves of the patch clamp current (expressed as percent of the control current) produced by various concentrations of SH-053-2′F—R—CH₃ when applied to Xenopus oocytes expressing recombinant α1β3γ2 (▪), α2β3γ2 (▴), α3β3γ2 () or α5β3γ2 (▾) GABA_{A} receptors.

FIG. 15 is a graph of dose-response curves of the patch clamp current (expressed as percent of the control current) produced by various concentrations of SH-TS-CH₃ when applied to Xenopus oocytes expressing recombinant α1β3γ2 (▪), α2β3γ2 (▴), α3β3γ2 (♦) or α5β3γ2 (▾) GABA_{A} receptors.

FIG. 16 is a graph of dose-response curves of the patch clamp current (expressed as percent of the control current) produced by various concentrations of SH-TR-CH₃ when applied to Xenopus oocytes expressing recombinant α1β3γ2 (▪), α2β3γ2 (▴), α3β3γ2 (♦) or α5β3γ2 (▾) GABA_{A} receptors.

FIG. 17 shows the in vivo anticonvulsant activity of compound XHe-II-053 in the maximum electroconvulsive shock (ECS, ▪) and subcutaneous metrazole seizure (PTZ, ) models of epilepsy.

FIG. 18 shows the in vivo anticonvulsant activity of alprazolam in the maximum electroconvulsive shock (ECS, ▪) and subcutaneous metrazole seizure (PTZ, ) models of epilepsy.

FIG. 19 shows the in vivo anticonvulsant activity of compound XLi—XHe-II-048 in the maximum electroconvulsive shock (ECS, ▪) and subcutaneous metrazole seizure (PTZ, ) models of epilepsy.

### DETAILED DESCRIPTION OF THE INVENTION

In embodiments of the present invention, ligands of GABA_{A} receptor subtypes are shown to modulate and/or regulate seizures. Compounds which are ligands of the GABA_{A} receptors acting as agonists or partial agonists are referred to hereinafter as “GABA_{A} receptor agonists” or “GABA_{A} receptor partial agonists” or “agonists” or “partial agonists”. In particular these are compounds which are ligands of the benzodiazepine (BZ) binding site of the GABA_{A} receptors, and hence acting as BZ site agonists or partial agonists. Such ligands also include compounds acting at the GABA site or at modulatory sites other than the benzodiazepine site of GABA_{A} receptors.

The novel anticonvulsant agents act preferably by selectively or preferentially acting as agonists or partial agonists at the GABA_{A}/α₂ receptors and/or GABA_{A}/α₃ receptors as compared to the GABA_{A}/α₁ receptors. A selective or preferential therapeutic agent has less binding affinity to the GABA_{A}/α₁ receptors compared to the GABA_{A}/α₂ or GABA_{A}/α₃ receptors. Alternatively, the agent binds to GABA_{A}/α₁, GABA_{A}/α₂ and GABA_{A}/α₃ receptors with a comparable affinity but exerts preferential efficacy of receptor activation at GABA_{A}/α₂ and GABA_{A}/α₃ receptors compared to the GABA_{A}/α₁ receptors. A selective agent of the present invention can also have a greater or lesser ability to bind or to activate GABA_{A}/α₅ receptors relative to GABA_{A}/α₂ and GABA_{A}/α₃ receptors. The anticonvulsant agent acts at the benzodiazepine site of the respective GABA_{A} receptors but is not restricted to this drug binding domain in its receptor interactions.

The treatment may be for prophylactic or therapeutic purposes. For administration to a subject, preferably to a mammalian subject, more preferably to a human subject in need of treatment, the subunit selective GABA_{A} receptor agonist is preferably in the form of a pharmaceutical preparation comprising the subunit selective GABA_{A} receptor agonist in chemically pure form and optionally a pharmaceutically acceptable carrier and optionally adjuvants. The subunit selective GABA_{A} receptor agonist is used in an amount effective against seizures. The dosage of the active ingredient depends upon the species, its age, weight, and individual condition, the individual pharmacokinetic data, the mode of administration, and whether the administration is for prophylactic or therapeutic purposes. In the case of a subject having a body weight of about 70 kg, the daily dose administered is from approximately 1 mg to approximately 500 mg, preferably from approximately 1 mg to approximately 100 mg, of a subunit selective GABA_{A} receptor agonist.

Pharmaceutical compositions for enteral administration, such as nasal, buccal, rectal or, especially, oral administration, and for parenteral administration, such as intravenous, intramuscular, subcutaneous, peridural, epidural or intrathecal administration, are especially preferred. The pharmaceutical compositions comprise from approximately 1% to approximately 95% active ingredient, preferably from approximately 20% to approximately 90% active ingredient.

For parenteral administration including intracoronary, intracerebrovascular, or peripheral vascular injection/infusion preference is given to the use of solutions of the subunit selective GABA_{A} receptor agonist, and also suspensions or dispersions, especially isotonic aqueous solutions, dispersions or suspensions which, for example, can be made up shortly before use. The pharmaceutical compositions may be sterilized and/or may comprise excipients, for example preservatives, stabilizers, wetting agents and/or emulsifiers, solubilizers, viscosity-increasing agents, salts for regulating osmotic pressure and/or buffers and are prepared in a manner known per se, for example by means of conventional dissolving and lyophilizing processes.

For oral pharmaceutical preparations suitable carriers are especially fillers, such as sugars, for example lactose, saccharose, mannitol or sorbitol, cellulose preparations and/or calcium phosphates, and also binders, such as starches, cellulose derivatives and/or polyvinylpyrrolidone, and/or, if desired, disintegrators, flow conditioners and lubricants, for example stearic acid or salts thereof and/or polyethylene glycol. Tablet cores can be provided with suitable, optionally enteric, coatings. Dyes or pigments may be added to the tablets or tablet coatings, for example for identification purposes or to indicate different doses of active ingredient. Pharmaceutical compositions for oral administration also include hard capsules consisting of gelatin, and also soft, sealed capsules consisting of gelatin and a plasticizer, such as glycerol or sorbitol. The capsules may contain the active ingredient in the form of granules, or dissolved or suspended in suitable liquid excipients, such as in oils.

Transdermal application is also considered, for example using a transdermal patch, which allows administration over an extended period of time, e.g. from one to twenty days.

Another aspect of the invention relates to the use of a subunit selective GABA_{A} receptor agonist in the method of treatment or prevention of seizures in a subject, preferably a mammalian subject, more preferably a human subject in need of treatment, and in the manufacture of medicaments for treating seizures in such a subject. Such medicaments are manufactured by methods known in the art, especially conventional mixing, coating, granulating, dissolving or lyophilizing. A subunit selective GABA_{A} receptor agonist can be administered alone or in combination with one or more other therapeutic agents, possible combination therapy taking the form of fixed combinations of a subunit selective GABA_{A} receptor agonist and one or more other therapeutic agents known in the treatment of seizures, the administration being staggered or given independently of one another, or being in the form of a fixed combination.

The following examples serve to illustrate the invention without limiting the invention in its scope.

### Synthesis of GABA_{A} Receptor Subunit Selective Benzodiazepine Derivatives

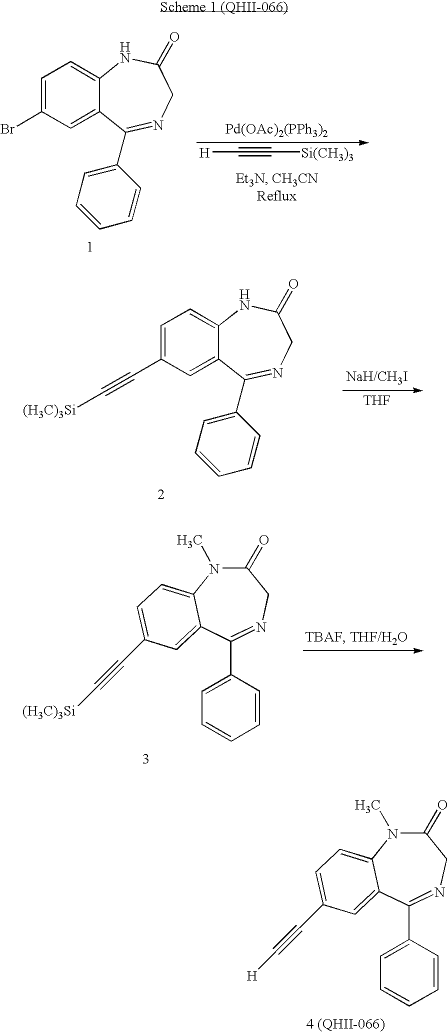

The bromide 1 available was reacted with trimethylsilylacetylene in the presence of a palladium catalyst to provide trimethylsilyl analog 2. This product was methylated with methyl iodide/sodium hydride to give the N-methyl benzodiazepine 3. This was subjected to fluoride-mediated desilylation to furnish 4 (QHII-066).

### Procedure for QHII-066

7-Trimethylsilylacetyleno-5-phenyl-1,3-dihydrobenzo[e]-1,4-diazepin-2-one (2). A mixture of 1 (1 g, 3.17 mmol) in triethyl amine (30 mL) and CH₃CN (20 mL) with trimethylsilylacetylene (622.7 mg, 6.34 mmol) and bis(tri-phenylphosphine)-palladium (II) acetate (118 mg, 0.16 mmol) was heated to reflux under nitrogen. After 12 hours, the reaction mixture was cooled to room temperature and filtered. The filtrate was concentrated in vacuum and the residue was treated with a saturated aqueous solution of NaHCO₃ (30 mL), and extracted with CH₂Cl₂ (3×50 mL). The organic layers were combined and washed with brine and dried (Na₂SO₄). After removal of solvent under reduced pressure, the residue was purified via flash chromatography (silica gel, EtOAc/hexanes: 1/1) to furnish 2 as a yellow powder (791 mg, 75%): mp: 190-191.5° C.; IR (KBr) 3011, 2281, 1686, 1610, 1486, 1325, 1249, 839, 700 cm⁻¹; ¹H NMR (CDCl₃) δ 0.21 (s, 9H), 4.31 (s, 2H), 7.09 (d, 1H, J=8.25 Hz), 7.21-7.61 (br, 7H), 10.17 (s, 1H); MS (CI) m/e (relative intensity) 333 (M⁺+1, 100). This material was used in the next step.

1-Methyl-7-trimethylsilylacetyleno-5-phenyl-1,3-dihydrobenzo[e]-1,4-diazepin-2-one (3). A mixture of 2 (485 mg, 1.46 mmol) was dissolved in dry THF (20 mL) at 0° C. and NaH (60% in mineral oil, 70 mg, 1.75 mmol) was added to the solution in one portion. The slurry was then stirred for 20 min at 0° C. and CH₃I (311 mg, 2.19 mmol) was added to the mixture and it was warmed up to room temperature. After the mixture stirred for 3 hours at room temperature, the THF was then removed under reduced pressure. The residue was purified by flash chromatography [hexanes/EtOAc (1:4)] to provide the title compound 3 (303 mg, 60%) as a white solid: mp: 177-178° C.; IR (KBr) 2954, 2147, 1687, 1612, 1491, 1382, 1115, 1075, 839, 700 cm⁻¹; 1HNMR (CDCl₃), δ (ppm), 0.21 (s, 9H), 3.18 (s, 3H), 3.54 (d, 1H, J=10.8 Hz), 4.60 (d, 1H. J=10.8 Hz), 7.05 (s, 1H), 7.07 (d, 1H, J=8.58 Hz), 7.20-7.27 (m, 3H), 7.37-7.42 (m, 3H); MS (EI) m/e 346 (M⁺, 90), 318 (100), 303 (19), 165 (22), 151 (20). Anal. Calcd. for C₂₁H₂₂N₂OSi: C, 72.79; H, 6.40; N, 8.08; Found: C, 72.50; H, 6.68; N, 8.04.

1-Methyl-7-acetyleno-5-phenyl-1,3-dihydro-benzo[e]-1,4-diazepin-2-one (4, QHII-066). A solution of 3 (100 mg,) in THF (30 mL) was treated with tetrabutylammonium fluoride (1M in THF). The mixture was stirred for 20 minutes at room temperature before water (30 mL) was added. The mixture was then extracted with EtOAc (3×30 mL). The combined organic extracts were washed with brine and dried (Na₂SO₄). The solvent was removed under vacuum and the residue which resulted was passed through a wash column (silica gel, EtOAc/hexanes: 4/1) to give 4 (QHII-066) as light yellow crystals (71 mg, 90%): mp: 163-165° C.; IR (KBr) 2965, 1680, 1605, 1387, 1121, 833, 747 cm⁻¹; 1HNMR (CDCl₃) δ (ppm) 3.38 (s, 3H), 3.75 (d, 1H, J=10.8 Hz), 4.80 (d, 1H, J=10.9 Hz), 5.28 (s, 1H), 7.29 (d, 1H, J=8.5 Hz), 7.35-7.45 (m, 4H), 7.55-7.59 (m, 2H), 7.62 (dd, 1H, J=8.5 Hz, 2.0 Hz); MS (EI) m/e (relative intensity) 274 (M⁺, 100), 259 (12), 246 (100), 189 (12), 122(19), 105 (42). Anal. Calcd. for C₁₈H₁₄N₂O.⅔H₂O, Calculated: C, 75.51; H, 4.89; N, 9.78. Found: C, 75.59; H, 5.17; N, 9.62.

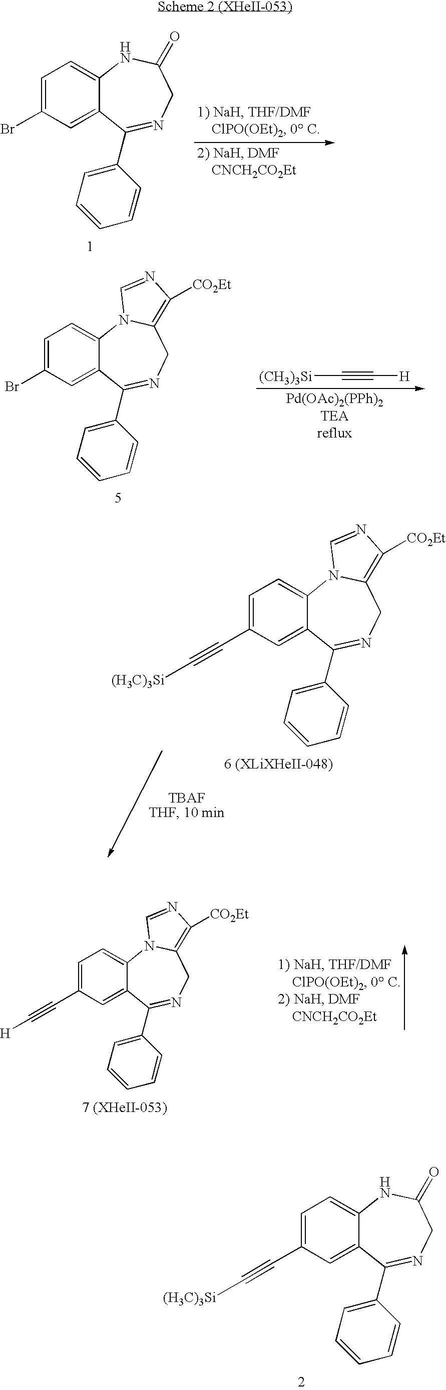

The bromide 1 was reacted with diethylphosphorochloridate in the presence of sodium hydride, followed by addition of ethyl isocyanoacetate to provide the ester 5. This was converted to the trimethylsilylacetyleno compound 6 (XLiXHeII-048) under standard conditions (Pd-mediated, Heck-type coupling). Treatment of 6 with fluoride gave the title compound 7 (XHeII-053).

### Procedure for XHe-II-053

Ethyl 8-bromo-6-phenyl-4H-benzo[f]imidazo[1,5-a][1,4]diazepine-3-carboxylate 5. This benzodiazepine 5 was obtained in 45% yield from 1 analogous to the literature procedure as a white solid. 5: mp: 174-175° C.; IR (KBr) 2978, 1712, 1609, 1491 cm⁻¹; ¹H NMR (CDCl₃) δ 1.44 (t, 3H, J=7.1 Hz), 4.09 (d, 1H, J=12.1 Hz), 4.38-4.49 (m, 2H), 6.08 (d, 1H, J=12.3 Hz), 7.40-7.53 (m, 6H), 7.60 (d, 1H, J=2.2 Hz), 7.82 (dd, 1H, J=8.6 Hz and 2.2 Hz), 7.95 (s, 1H); MS (EI) m/e (relative intensity) 411 (34), 410 (M⁺, 8), 409 (34), 365 (61), 363 (61), 337 (100), 335 (100), 285 (21), 232, (17). Anal. Calcd. for C₂₀H₁₆BrN₃O₂: C, 58.55; H, 3.93; N, 10.24. Found: C, 58.30, H, 3.91; N, 9.90.

Ethyl 8-trimethylsilylacetylenyl-6-phenyl-4H-benzo[f]imidazo[1,5-a][1,4]diazepine-3-carboxylate 6 (XLiXHeII-048). A mixture of bromide 5 (0.3 g, 0.73 mmol), trimethylsilylacetylene (0.143 g, 1.46 mmol) and bis(triphenylphosphine)-palladium-(II) acetate (55 mg, 0.073 mmol) in a mixed solvent system of toluene (20 mL) and anhydrous TEA (50 mL) was heated to reflux under argon. After stirring for 12 hours at reflux, the mixture was cooled to room temperature and the precipitate which formed was removed by filtration. The filtrate was concentrated under reduced pressure and the residue was treated with a saturated aqueous solution of NaHCO₃ (20 mL), and extracted with CHCl₃ (3×25 mL). The combined extracts were washed with brine and dried (Na₂SO₄). After removal of solvent under reduced pressure, the residue was purified by flash chromatography (silica gel, EtOAc) to afford 6 (XLiXHeII-048) as a white solid (0.29 g, 93%). This benzodiazepine can also be obtained from 2 in 45% yield by following the same procedure 6 (XLiXHeII-048): mp: 170-172° C.; IR (KBr) 2958, 2152, 1718 cm⁻¹; ¹H NMR (CDCl₃) δ 0.23 (s, 9H), 1.42 (t, 3H, J=7.2 Hz), 4.04 (d, 1H, J=12.6 Hz), 4.41 (m, 2H, J=7.2 Hz), 6.23 (d, 1H, J=12.6 Hz), 7.35-7.55 (m, 7H), 7.73 (dd, 1H, J=8.3 Hz, J=1.9 Hz), 7.93 (s, 1H); MS (EI) m/e (relative intensity) 427 (M⁺, 76), 412 (5), 381 (55), 353 (100) 303 (10), 287 (7). Anal. Calcd. for C₂₅H₂₅N₃O₂Si.⅓ EtOAc: C, 69.22; H, 6.01; N, 9.20. Found: C, 68.87; H, 5.81; N, 9.37.

Ethyl 8-acetylenyl-6-phenyl-4H-benzo[f]imidazo[1,5-a][1,4]diazepine-3-carboxylate 7 (XHeII-053). A solution of 6 (XLiXHeII-048) (0.17 g, 0.41 mmol), in THF (15 mL) was treated with Bu₄NF.H₂O (0.16 g, 0.62 mmol). The mixture which resulted was allowed to stir for 30 min at room temperature after which the mixture was added to H₂O (10 mL) and extracted with EtOAc (3×25 mL). The combined organic extracts were washed with brine (25 mL) and dried (Na₂SO₄). After removal of solvent under reduced pressure, the residue was purified by a wash column (silica gel, EtOAc) to furnish 7 (XHeII-053) (0.12 g, 85%) as a white solid: mp 237-239° C.; IR (KBr) 3159, 3107, 2092, 1721, 1606 cm⁻¹; ¹H NMR (CDCl₃) δ 1.44 (t, 3H, J=7.1 Hz), 3.20 (s, 1H), 4.13 (d, 1H, J=10.22 Hz), 4.41-4.48 (m, 2H), 6.11 (d, 1H, J=12 Hz), 7.42-7.63 (m, 7H), 7.81 (dd, 1H, J=8.3 Hz and 1.8 Hz), 8.03 (s, 1H); MS (EI) m/e (relative intensity) 355 (M⁺, 83), 309 (70), 281 (100), 253 (12), 231 (18), 178 (20). Anal. Calcd. for C₂₂H₁₇N₃O₂.3/4H₂O: C, 71.63; H, 5.05; N, 11.39. Found: C, 71.27; H, 4.71; N, 11.03.

The bromide 1, available from reference 1, was stirred with the di-4-morpholino-phosphinic chloride, followed by addition of acetylhydrazide to furnish triazolo-benzodiazepine 8. This material 8 was subjected to a Heck-type coupling reaction (TMS—C≡CH, Pd-mediated) to furnish ligand 9. This analog was converted into 10 (XLi270) on stirring with fluoride anion as shown in Scheme 3.

### Procedure for XLi 270:

8-Bromo-1-methyl-6-phenyl-41′-s-triazolo[4,3-a][1,4]benzodiazepine 8. A solution of 1 (1 g, 3.07 mmol of 7-bromo-5-phenyl-1,4-benzodiazepine-2-one) in dry THF (20 mL) was cooled in an ice-water bath and a 60% dispersion of sodium hydride (152.2 mg) was added in one portion. After 20 minutes, di-4-morpholinylphosphinic chloride³ (943.9 mg, 4.76 mmol) was added at 0° C. and this was stirred for 30 minutes and allowed to warm to room temperature. The mixture was stirred for 1.5 hours. To this mixture was then added a solution of acetylhydrazide (521.9 mg, 7.14 mmol) in dry butanol (5 mL) and stirring was continued at room temperature for 10 min. The solvents were evaporated and the residue was dissolved in butanol (10 mL) and heated to reflux for 5 hours. Butanol was removed under reduced pressure and the residue was partitioned between CH₂Cl₂ (50 mL) and water (50 mL). The water layer was extracted by CH₂Cl₂ (3×30 mL). The combined organic layer was washed by brine (30 mL). The organic layer was dried (Na₂SO₄) and the solvent was removed under vacuum. The residue was purified by flash chromatography (silica gel) to provide pure 8 [539.5 mg (40% yield)] as a white solid: mp 268.5-270° C.; IR (KBr) 2358, 1607, 1538, 1484, 1311, 1000, 801, 697 cm⁻¹; ¹H NMR (CDCl₃) δ 2.82 (s, 3H), 4.11 (d, 1H, J=12.8 Hz), 5.49 (d, 1H, J=12.8 Hz), 7.21-7.68 (m, 7H), 7.75 (dd, 1H, J=0.58 Hz, J=1.5 Hz); MS (EI) m/e (relative intensity) 354 (34), (M⁺, 16), 352 (34), 325(33), 323 (34), 273 (63), 245 (31), 232 (19), 204 (100), 183(23), 177 (36), 151 (24). Anal. Calcd. for C₁₇H₁₃BrN₄: C, 57.81; H, 3.71; N, 15.86. Found C, 57.57; H, 3.64: N, 15.70.

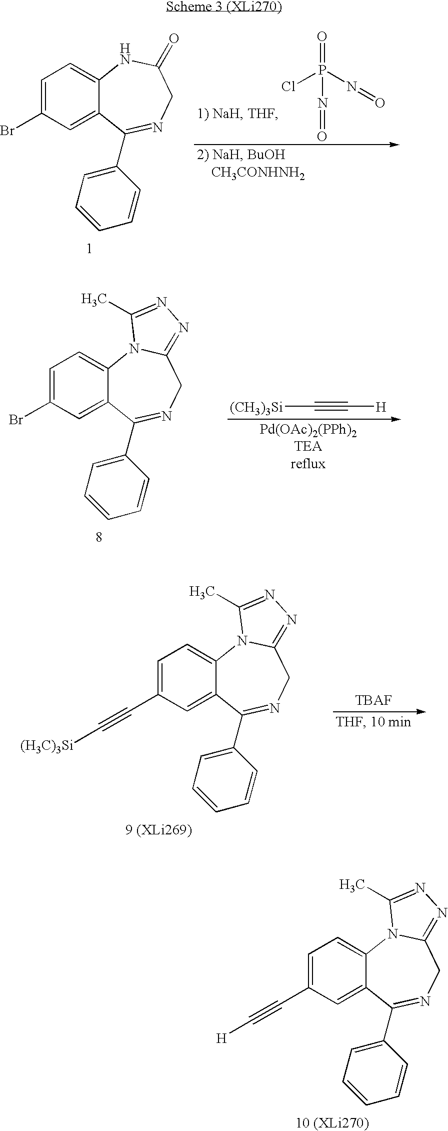

8-Trimethylsilylacetylenyl-1-methyl-6-phenyl-4H-s-triazolo[4,3-a][1,4]-benzodiazepine 9. (XLi269). A mixture of 8 (8-bromo-1-methyl-6-phenyl-4-H-s-triazolo-[4,3-a][1,4]benzodiazepine, 300 mg, 0.85 mmol), trimethylsilylacetylene (208.5 mg, 2.12 mmol) and bis(triphenylphosphine)-palladium(II) acetate in a mixed solvent system of Et₃N (5 mL) and CH₃CN (8 mL) was heated to reflux under nitrogen. After stirring for 6 hours at reflux. The mixture was cooled to room temperature. The mixture was concentrated under reduced pressure and H₂O (30 mL) was added. The mixture was extracted with CH₂Cl₂ (3×50 mL). The combined extracts were washed with brine and dried (Na₂SO₄). After removal of solvent under reduced pressure, the residue was purified by flash chromatography (silica gel, EtOH/EtOAc) to afford benzodiazepine 9 (185 mg, 60% yield) as a white solid: mp 229-233° C.; IR (KBr) 2957, 2156, 1609, 1537, 1491, 1424, 1315, 1249, 881, 844, 750 cm⁻¹; ¹H NMR (CDCl₃) δ 0.23 (s, 9H), 2.68 (s, 3H), 4.11 (d, 1H, J=12.5 Hz), 5.49 (d, 1H, J=13.0 Hz), 7.21-7.68 (m, 7H), 7.75 (dd, 1H, J=8.5 Hz, J=1.5 Hz); MS (EI) m/e (relative intensity) 370 (M⁺, 80), 355 (44), 341 (60), 286 (34), 177 (51), 163 (52) 143 (100), 129 (19), 115 (28). Anal. Calcd. for C₂₂H₂₂N₄Si: C, 71.31; H, 5.98; N, 15.12. Found: C, 70.90; H, 5.93; N, 15.08.

8-Acetylenyl-1-methyl-6-phenyl-4H-s-triazolo[4,3-a][1,4]benzodiazepine 10 (XLi-270). A solution of 9 [trimethylsilylacetylenyl-1-methyl-6-phenyl-4H-s-triazolo-[4,3-a]-[1,4]-benzodiazepine (106.4 mg, 0.288 mmol)] in dry THF (20 mL) was treated with Bu₄NF (1.0 M in THF, 112.8 mg, 0.431 mmol). The mixture which resulted was allowed to stir for 5 min at room temperature after which the mixture was added to H₂O (10 mL) and extracted with CH₂Cl₂ (3×25 mL). The combined organic extracts were washed with brine (25 mL) and dried (Na₂SO₄). After removal of solvent under reduced pressure, the residue was crystallized from EtOAc to provide benzodiazepine 10 (XLi270) (66.8 mg, 80% yield) as a white solid: mp>250° C. (dec); IR (KBr) 3198, 2158, 1609, 1538, 1491, 1425, 1317, 1002, 838, 748, 695 cm⁻¹; ¹H NMR (CDCl₃) δ 2.78 (s, 3H), 3.15 (s, 1H), 4.11 (d, 2H, J=12.8 Hz), 5.91 (d, 1H, J=12.8 Hz), 7.35-7.85 (m, 8H); MS (EI) (relative intensity) 298 (M⁺, 100), 269 (78), 230 (48), 228 (65), 201 (20), 127 (65), 115 (42), 101 (54). Anal. Calcd. for C₁₉H₁₄N₄.1/2 CH₃OH: C, 74.50; N, 17.82. Found: C, 74.33; H, 4.83; N, 17.77.

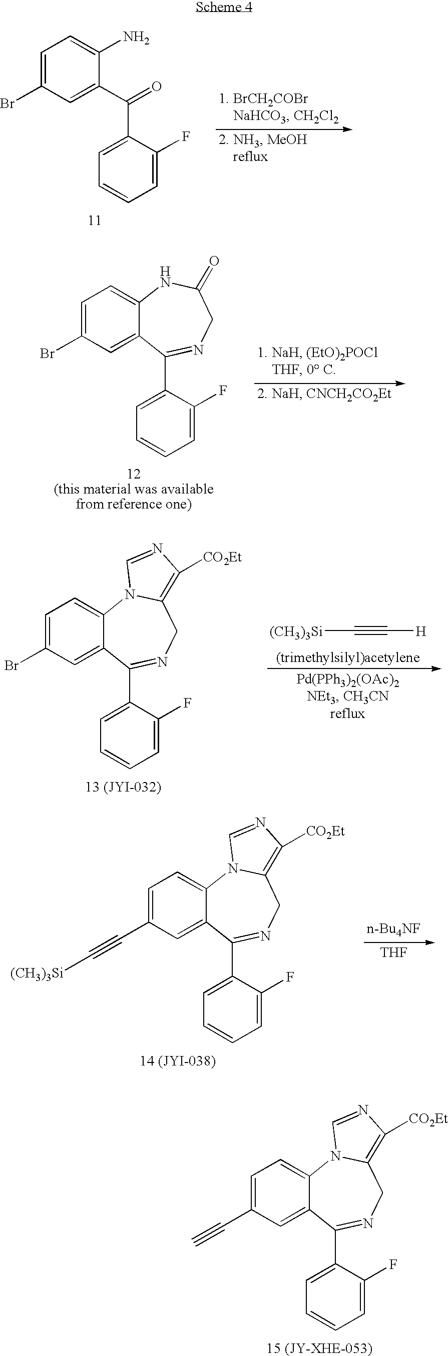

The 7-bromo-2′-fluorobenzodiazepine 12 was reacted with sodium hydride and diethylphosphorochloridate and this was followed by addition of ethyl isocyanoacetate to provide benzimidazo intermediate 13 (JYI-032), as illustrated in Scheme 4. This material was heated with trimethysilylacetylene in a Heck-type coupling reaction to provide the trimethylsilyl analog 14 (JYI-038). The silyl group was removed from 14 on treatment with fluoride anion to furnish 15, a 2′-fluoro analog of XHeII-053, in excellent yield.

### Procedure:

Ethyl 8-bromo-6-(2′-fluorophenyl)-4H-benzo[f]imidazo[1,5-a][1,4]diazepine-3-carboxylate 13 (JYI-032). A solution of 12 (7.0 g, 21.0 mmol) in THF (50 mL) was cooled in ice-water, and sodium hydride (1.0 g, 25.2 mmol) was added in one portion. After 30 min, diethyl phosphorochloridate (5.62 g, 31.5 mmol) was added dropwise, and the solution which resulted was stirred continuously for 30 min with cooling from an ice bath. A solution of ethyl isocyanoacetate (4.22 g, 25.2 mmol) and sodium hydride (1.17 g, 29.4 mmol) in THF (10 mL), which had stirred for 30 min with ice-bath cooling, was added slowly via a cannula. After stirring for another 30 min with cooling, the reaction mixture was allowed to stir at room temperature overnight. The mixture was then added to H₂O (10 mL) and extracted with EtOAc (3×50 mL). The combined organic extracts were washed with brine (2×50 mL) and dried (Na₂SO₄). The solvent was evaporated under reduced pressure and the residue was purified by flash chromatography (silica gel, hexanes/EtOAc: 2/1) to afford 13 (JYI-032, 5.2 g, 58%) as a white solid: mp 200-201.5° C.; IR (KBr) 2977, 1718, 1610, 1491, 1450 cm⁻¹; ¹H NMR (DMSO-d₆) δ 1.30 (t, 3H, J=4.2 Hz), 4.28 (bs, 1H), 4.30 (q, 2H, J=4.2 Hz), 5.75 (bs, 1H), 7.20 (t, 1H, J=5.6 Hz), 7.30 (t, 1H, J=4.5 Hz), 7.40 (s, 1H), 7.54 (m, 2H), 7.85 (d, 1H, J=5.2 Hz), 7.96 (dd, 1H, J=5.2 Hz and 1.3 Hz), 8.44 (s, 1H); MS (EI) m/e (relative intensity) 428 (7), 381 (58), 355 (100), 303 (37), 274 (36), 247 (35), 234 (52), 154 (71), 127 (62). Anal Calcd. for C₂₀H₁₅N₃O₂FBr: C, 56.09; H, 3.53; N, 9.81. Found: C, 56.02; H, 3.51; N, 9.58.

Ethyl 8-trimethylsilylacetylenyl-6-(2′-fluorophenyl)-4H-benzo[f]-imidazo[1,5-a][1,4]diazepine-3-carboxylate 14 (JYI-038). A mixture of bromide 13 (JYI-032, 1.40 g, 3.3 mmol), trimethylsilylacetylene (0.65 g, 6.6 mmol) and bis(triphenylphosphine)-palladium (II) acetate (0.25 g, 0.33 mmol) in a mixed solvent system of CH₃CN (80 mL) and anhydrous triethylamine (50 mL) was heated to reflux under argon. After stirring for 2 h at reflux, the mixture was cooled to room temperature and the precipitate which formed was removed by filtration. The filtrate was concentrated under reduced pressure and the residue was treated with a saturated aqueous solution of NaHCO₃ (40 mL), and extracted with CHCl₃ (3×50 mL). The combined organic extracts were washed with brine (2×20 mL) and dried (Na₂SO₄). After removal of solvent under reduced pressure, the residue was purified by flash chromatography (silica gel, hexanes/EtOAc: 3/1) to afford 14 (JYI-038, 1.2 g, 82%) as a white solid: mp 196-197.5° C.; IR (KBr) 2959, 2157, 1709, 1613, 1494, 1451, 1252 cm⁻¹; ¹H NMR (DMSO-d₆) δ 0.20 (s, 9H), 1.32 (t, 3H, J=7.1 Hz), 4.18 (bs, 1H), 4.32 (q, 2H, J=7.1 Hz), 5.78 (bs, 1H), 7.25 (t, 1H, J=11.5 Hz), 7.30-7.35 (m, 4H), 7.81 (d, 1H, J=6.6 Hz), 7.93 (d, 1H, J=8.4 Hz), 8.49 (s, 1H); MS (EI) m/e (relative intensity) 445 (37), 399 (51), 371 (100), 235 (71), 192 (66), 178 (75). Anal. Calcd. for C₂₅H₂₄N₃O₂FSi: C, 67.39; H, 5.42; N, 9.43. Found: C, 66.98; H, 5.46; N, 9.19.

8-Acetyleno-6-(2′-fluorophenyl)-4H-benzo[f]imidazo[1,5-a][1,4]diazepine-3-carboxylate 15 (JY-XHE-053). A solution of 14 (JYI-038, 80 mg, 0.18 mmol) in THF (5 mL) was treated with Bu₄NF (0.5 mL, 1.0M solution in THF). The mixture which resulted was allowed to stir for 5 min at room temperature after which the mixture was added to H₂O (5 mL) and extracted with EtOAc (3×10 mL). The combined organic extracts were washed with brine (2×10 mL) and dried (Na₂SO₄). The solvent was removed under reduced pressure and the residue was purified by flash chromatography (silica gel, EtOAc) to afford 15 (JY-XHE-053, 67 mg, 80%) as a white solid: mp 223.5-224.5° C.; IR (KBr) 3288, 2979, 1712, 1621, 1491, 1255, 1190 cm⁻¹; ¹H NMR (DMSO-d₆) δ 1.34 (t, 3H, J=7.1 Hz), 4.27 (bs, 1H), 4.36 (q, 2H, J=7.1 Hz), 4.47 (s, 1H), 5.80 (bs, 1H), 7.22 (t, 1H, J=8.4 Hz), 7.30-7.60 (m, 4H), 7.85 (d, 1H, J=6.6 Hz), 7.92 (d, 1H, J=8.4 Hz), 8.83 (s, 1H); MS (EI) m/e (relative intensity) 373 (28), 327 (47), 299 (100), 249(22), 178 (50). Anal. Calcd. for C₂₂H₁₆N₃O₂F.½H₂O: C, 69.10; H, 4.48; N, 10.99. Found: C, 69.19; H, 4.39; N, 10.68.

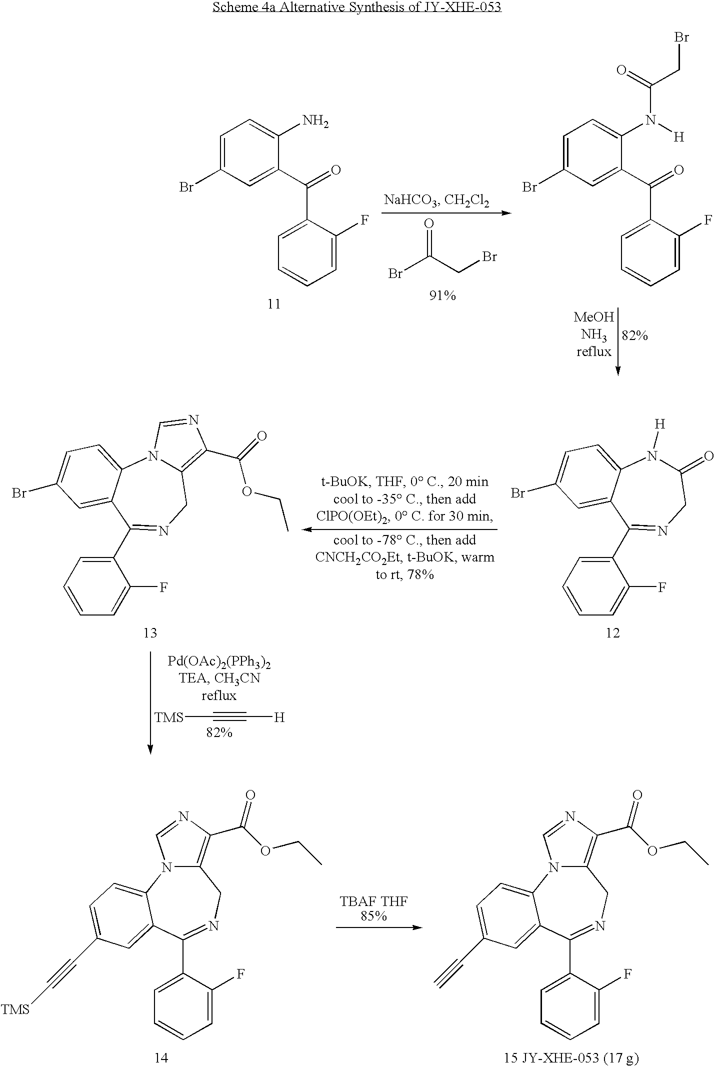

### Old Procedure:

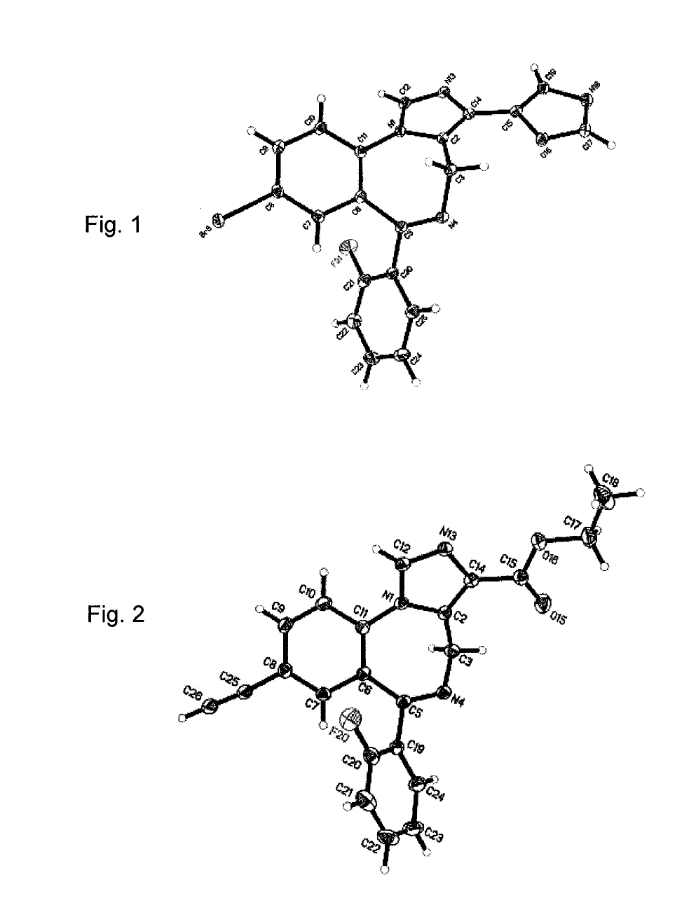

The 7-bromo-2′-fluorobenzodiazepine 12 was stirred with sodium hydride and diethylphosphorochloridate and this was followed by addition of ethyl isocyanoacetate to provide benzimidazo intermediate 13, as illustrated in Scheme 4a. In the latter step, the solution of ethyl isocyanoacetate and sodium hydride in THF, should be stirred for 60 minutes with ice bath cooling before it is added slowly via a cannula to the another cold reaction flask. If it was stirred for more than 180 minutes, the reaction will provide a lower yield and produce a byproduct ByP. This material was crystallized from EtOAc and hexane solvent mixture to provide crystals. The structure was solved by X-ray crystallography by Dechamps et al. FIG. 1. is an ORTEP drawing of the crystal structure of SH—I-085 (ByP)]. The desired intermediate 13 was heated with trimethysilylacetylene in a Heck-type coupling reaction to provide the trimethylsilyl analog 14. The silyl group was removed from 14 on treatment with fluoride anion to furnish 15 (JY-XHE-053), which was crystallized from EtOAc and hexane. FIG. 2 is an ORTEP drawing of the crystal structure of JY-XHe-053 (15).

Ethyl-bromo-6-(2′-fluorophenyl)-4H-benzo[f]imidazo[1,5-a][1,4]diazepine-3-carboxylate (13). (New Process) A solution of 12 (7.0 g, 21.0 mmol) in THF (525 mL) was cooled to 0° C., and potassium t-butoxide (2.59 g, 23.1 mmol) was added to it in one portion. After stirring for 20 minutes at 0° C., the reaction was cooled to −35° C. and diethyl chlorophosphate (4.71 g, 27.3 mmol) was added slowly. After stirring at 0° C. for 30 min, the reaction mixture was cooled to −78° C. and ethyl isocyanoacetate (2.61 g, 23.1 mmol) was added, followed by potassium t-butoxide (2.59 g, 23.1 mmol). After stirring at ambient temperature for 4 hours, the reaction was quenched with a saturated aqueous solution of NaHCO₃ (4 mL) and extracted with EtOAc. The combined organic layers were dried (Na₂SO₄), concentrated and crystallized from ether to give the ester 13. The mother liquor was purified by flash chromatography on (silica gel, hexane/EtOAc: 2/1) to afford 13 (combined yield, 6.95 g, 78%) as a white solid: mp 200-201.5° C.; IR (KBr) 2977, 1718, 1610, 1491, 1450 cm⁻¹; ¹H NMR (DMSO-d₆) δ 1.30 (t, 3H, J=4.2 Hz), 4.28 (bs, 1H), 4.30 (q, 2H, J=4.2 Hz), 5.75 (bs, 1H), 7.20 (t, 1H, J=5.6 Hz), 7.30 (t, 1 H, J=4.5 Hz), 7.40 (s, 1H), 7.54 (m, 2H), 7.85 (d, 1H, J=5.2 Hz), 7.96 (dd, 1H, J=5.2 Hz and 1.3 Hz), 8.44 (s, 1H); MS (EI) m/e (relative intensity) 428 (7), 381 (58), 355 (100), 303 (37), 274 (36), 247 (35), 234 (52), 154 (71), 127 (62). Anal Calcd. for C₂₀H₁₅N₃O₂FBr: C, 56.09; H, 3.53; N, 9.81. Found: C, 56.02; H, 3.51; N, 9.58.

Ethyl 8-trimethylsilylacetylenyl-6-(2′-fluorophenyl)-4H-benzo[f]-imidazo[1,5-a][1,4]diazepine-3-carboxylate (14). A mixture of bromide 13 (1.40 g, 3.3 mmol) and bis(triphenylphosphine)-palladium (II) acetate (0.25 g, 0.33 mmol) was dissolved in a mixed solvent system of CH₃CN (80 mL) and triethylamine (50 mL). The mixture was degassed under vacuum and argon gas. The process was repeated three more times, after which trimethylsilylacetylene (0.65 g, 6.6 mmol) was added into the mixture and then heated to reflux under argon. After stirring for 2 h at reflux, the mixture was cooled to rt and the precipitate which formed was removed by filtration through celite. The filtrate was concentrated under reduced pressure and the residue was treated with a saturated aqueous solution of NaHCO₃ (40 mL), and extracted with CHCl₃ (3×50 mL). The combined organic extracts were washed with brine (2×20 mL) and dried (Na₂SO₄). After removal of the solvent under reduced pressure, the residue was purified by flash chromatography (silica gel, hexanes/EtOAc: 3/1) to afford 14 (1.2 g, 82%) as a white solid: mp 196-197.5° C.; IR (KBr) 2959, 2157, 1709, 1613, 1494, 1451, 1252 cm⁻¹; ¹H NMR (DMSO-d₆) δ 0.20 (s, 9H), 1.32 (t, 3H, J=7.1 Hz), 4.18 (bs, 1H), 4.32 (q, 2H, J=7.1 Hz), 5.78 (bs, 1H), 7.25 (t, 1H, J=11.5 Hz), 7.30-7.35 (m, 4H), 7.81 (d, 1H, J=6.6 Hz), 7.93 (d, 1H, J=8.4 Hz), 8.49 (s, 1H); MS (EI) m/e (relative intensity) 445 (37), 399 (51), 371 (100), 235 (71), 192 (66), 178 (75). Anal. Calcd. for C₂₅H₂₄N₃O₂FSi: C, 67.39; H, 5.42; N, 9.43. Found: C, 67.46; H, 5.44; N, 9.45.

Ethyl-8-acetyleno-6-(2′-fluorophenyl)-4H-benzo[f]imidazo[1,5-a][1,4]diazepine-3-carboxylate (15). A solution of 14 (80 mg, 0.18 mmol) in THF (5 mL) was treated with Bu₄NF.H₂O (0.5 mL, 1.0 M solution in THF). The mixture which resulted was allowed to stir for 5 minutes at rt after which the mixture was added to H₂O (5 mL) and extracted with EtOAc (3×10 mL). The combined organic extracts were washed with brine (2×10 mL) and dried (Na₂SO₄). The solvent was removed under reduced pressure and the residue was purified by flash chromatography (silica gel, EtOAc) to afford 5 (67 mg, 85%) as a white solid: mp 235-236° C.; IR (KBr) 3288, 2979, 1712, 1621, 1491, 1255, 1190 cm⁻¹; ¹H NMR (DMSO-d₆) δ 1.34 (t, 3H, J=7.1 Hz), 4.27 (bs, 1H), 4.36 (q, 2H, J=7.1 Hz), 4.47 (s, 1H), 5.80 (bs, 1H), 7.22 (t, 1H, J=8.4 Hz), 7.30-7.60 (m, 4H), 7.85 (d, 1H, J=6.6 Hz), 7.92 (d, 1H, J=8.4 Hz), 8.83 (s, 1H); MS (EI) m/e (relative intensity) 373 (28), 327 (47), 299 (100), 249(22), 178 (50). Anal. Calcd. for C₂₂H₁₆N₃O₂F: C, 70.77; H, 4.32; N, 11.25. Found: C, 70.81; H, 4.35; N, 11.28.

The yields obtained with the synthetic procedure of Scheme 4a were an improvement over the prior art.

Many procedures have been reported to synthesize imidazo[1,5-a][1,4]benzodiazepines. (F. Hoffmann-La Roche, & Co. A.-G., Switz. Diazepine derivatives. Neth. Appl. 7803585, 1978; Chem. Abstr. 1978, 90:152254g. Rogers-Evans, M.; Spurr, P.; Hennig, M. The isolation and use of a benzodiazepine iminochloride for the efficient construction of flumazenil. Tetrahedron Lett. 2003, 44(11), 2424-2428). Among them, a general method was used to form imidazo[1,5-a][1,4]benzodiazepines: first form the phosphate, followed by reacting with ethyl isocyanoacetate and different bases. Potassium tert-butoxide has been reported in some cases as the base in this conversion. However, none of reported procedures gave satisfactory yield. Herein, we report an improved procedure for the synthesis of imidazo[1,5-a][1,4]benzodiazepines in good yield.

As shown in Scheme 4b, potassium tert-butoxide was used as a base in the second step of the reaction (Watjen, F.; Baker, R.; Engelstoff, M.; Herbert, R.; MacLeod, A.; Knight, A.; Merchant, K.; Moseley, J.; Saunders, J. Novel benzodiazepine receptor partial agonists: oxadiazolylimidazobenzodiazepines. J. Med. Chem. 1989, 32(10), 2282-2291). This procedure gave only 47% yield. Fryer et al reported their method, which used potassium tert-butoxide as the base in two steps. DMF (Scheme 4c, Fryer, R. I.; Walser, A. Imidazobenzodiazepine derivatives and medicaments containing them. Eur. Pat. Appl. EP 135770, 1985; Chem. Abstr. 1985, 103:160545) and THF (Scheme 4d, Fryer, R. I.; Gu, Z-Q.; Wang, C-G. Synthesis of novel, substituted 4H-imidazo[1,5-a][1,4]benzodiazepines. J. Heterocyclic Chem. 1991, 28(7), 1661-1669.) were chosen as the solvent in these cases; however, the reported yield of this procedure was only 44%.

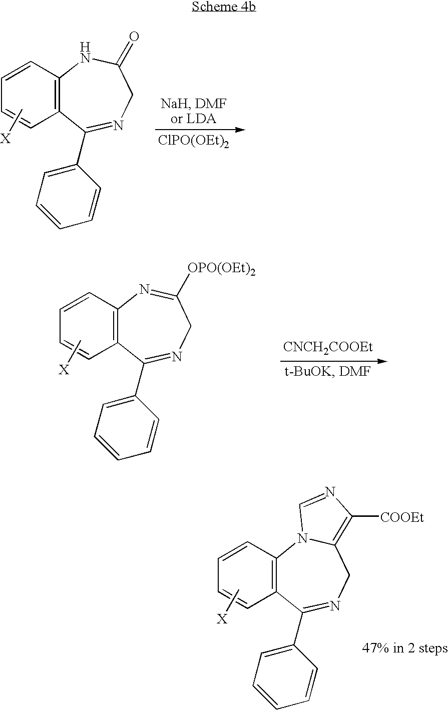

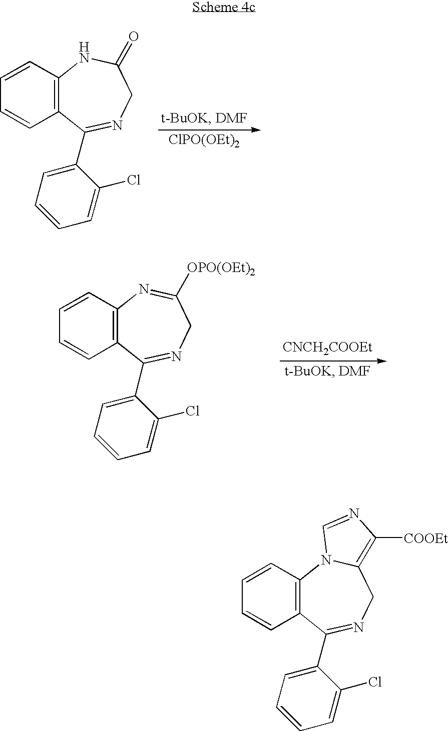

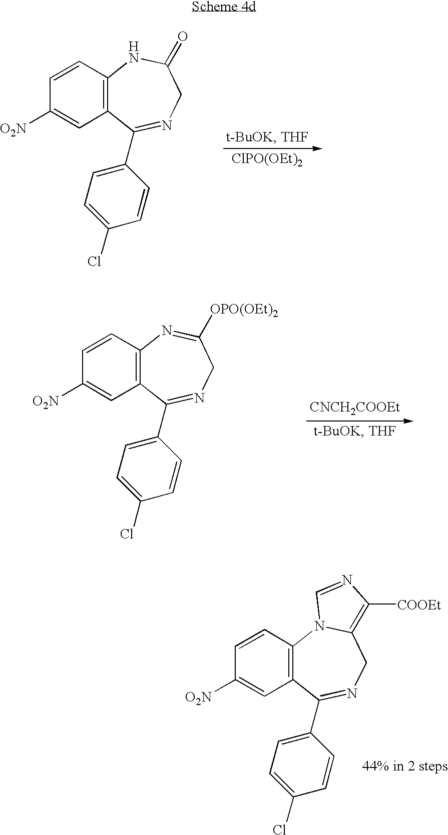

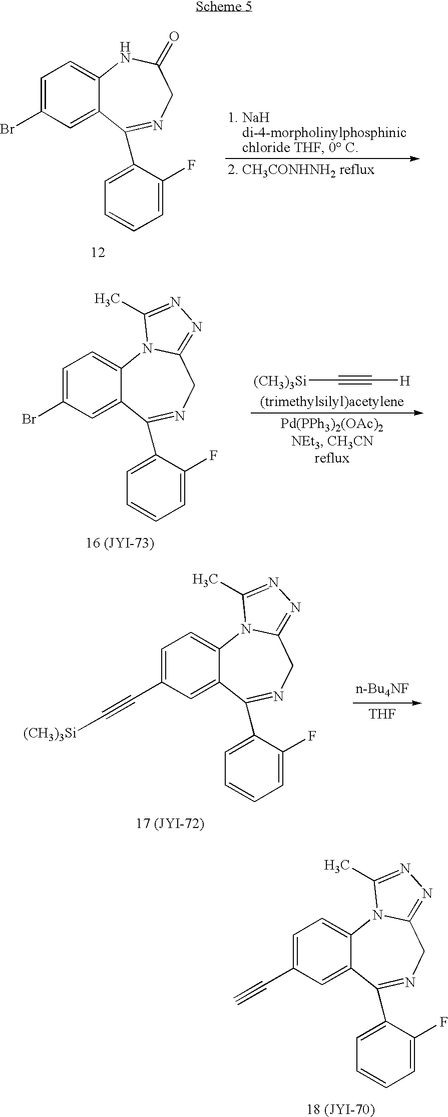

The 7-bromo-2′-fluorobenzodiazepine 12 was stirred with sodium hydride and di-4-morpholinylphosphinic chloride, followed by addition of acetic hydrazide, according to the published procedure to provide triazolobenzodiazepine 16 (JYI-73), as illustrated in Scheme 5. This compound 16 underwent the palladium-mediated Heck-type coupling reaction with trimethylsilylacetylene to furnish the 8-trimethylsilyl substituted analog 17 (JYI-72). Removal of the silyl group from 17 furnished the 8-acetyleno triazolobenzodiazepine 18 (JYI-70).

### Procedure:

8-Bromo-1-methyl-6-(2′-fluorophenyl)-4H-s-triazolo[4,3-a][1,4]benzodiazepine 16 (JYI-73). A solution of 12 (JYI-032, 7.0 g, 21.0 mmol) in THF (50 mL) was cooled in ice-water, and sodium hydride (0.72 g, 18 mmol) was added in one portion. After 1 hour, di-4-morpholinylphosphinic chloride (4.84 g, 22.5 mmol) was added, and the solution which resulted was stirred continuously for 2 hours at room temperature. To this mixture was then added a solution of acetic hydrazide (2.47 g, 30 mmol) in n-BuOH (20 mL) and stirring was continued at room temperature for 15 min. The solvents were evaporated and the residue was dissolved in n-BuOH (25 mL) and heated to reflux for 2 hours. n-Butanol was evaporated and the residue was partitioned between CH₂Cl₂ and brine. The CH₂Cl₂ layer was dried and removed under reduced pressure after which the residue was purified by flash chromatography (silica gel, EtOAc) to afford 16 (JYI-73, 2.2 g, 40%) as a white solid: mp 213-214° C.; IR (KBr) 1610, 1484, 1426, 1314 cm⁻¹; ¹H NMR (DMSO-d₆) δ 2.56 (s, 3H), 4.28 (d, 1H, J=12.9 Hz), 5.26 (d, 1H, J=12.9 Hz), 7.24 (t, 1H, J=8.3 Hz), 7.29 (t, 1H, J=7.2 Hz), 7.35 (s, 1H), 7.43-7.60 (m, 2H), 7.83 (d, 1H, J=8.7 Hz), 7.98 (dd, 1H, J=8.7 Hz and 2.3 Hz); MS (EI) m/e (relative intensity) 371 (5), 341 (34), 222 (100), 195 (19), 181 (28), 111 (72). Anal. Calcd. for C₁₇H₁₂N₄FBr: C, 55.01; H, 3.26; N, 15.09. Found: C, 54.76; H, 3.29; N, 14.74.

8-Trimethylsilylacetylenyl-1-methyl-6-(2′-fluorophenyl)-4H-s-triazolo[4,3-a][1,4]-benzodiazepine 17 (JYI-72). A mixture of bromide 16 (JYI-73, 1.40 g, 3.8 mmol), trimethylsilylacetylene (0.65 g, 6.6 mmol) and bis(triphenylphosphine)palladium (II) acetate (0.25 g, 0.33 mmol) in a mixed solvent system of CH₃CN (80 mL) and anhydrous triethylamine (50 mL) was heated to reflux under argon. After stirring for 2 hours at reflux, the mixture was cooled to room temperature and the precipitate which formed was removed by filtration. The filtrate was concentrated under reduced pressure and the residue was treated with a saturated aqueous solution of NaHCO₃ (40 mL), and extracted with CHCl₃ (3×50 mL). The combined organic extracts were washed with brine (2×10 mL) and dried (Na₂SO₄). After removal of solvent under reduced pressure, the residue was purified by flash chromatography (silica gel, EtOAc) to afford 17 (JYI-72, 1.15 g, 77%) as a gray solid: mp 218-219° C.; IR (KBr) 2958, 2157, 1612, 1537, 1493, 1452, 1317, 1249 cm⁻¹; ¹H NMR (DMSO-d₆) δ 0.21 (s, 9H), 2.56 (s, 3H), 4.23 (s, 1H, J=12.9 Hz), 7.26 (t, 1H, J=8.4 Hz), 7.29-7.83 (m, 6H); MS (EI) m/e (relative intensity) 388 (65), 373 (14), 359 (77), 304 (44), 152 (100). Anal. Calcd. for C₂₂H₂₁N₄SiF.0.7H₂O: C, 65.87; H, 5.62; N, 13.94. Found: C, 65.88; H, 5.34; N, 13.94.

8-Acetyleno-1-methyl-6-(2′-fluorophenyl)-4H-s-triazolo[4,3-a][1,4]benzodiazepine 18 (JYI-70). A solution of 17 (JYI-72, 2.0 g, 5 mmol) in THF (20 mL) was treated with Bu₄NF (4 mL, 1.0M solution in THF). The mixture which resulted was allowed to stir for 5 min at room temperature after which the mixture was added to H₂O (20 mL) and extracted with CH₂Cl₂ (3×50 mL). The combined organic extracts were washed with brine (2×15 mL) and dried (Na₂SO₄). After removal of solvent under reduced pressure, the residue was purified by flash chromatography (silica gel, EtOAc/MeOH: 100/1) to afford 18 (JYI-70, 1.1 g, 70%) as a pale yellow solid: mp>250° C. (dec); IR (KBr) 3205, 1612, 1493, 1426, 1317 cm⁻¹; ¹H NMR (DMSO-d₆) δ 2.54 (s, 3H), 4.22 (d, 1H, J=12.9 Hz), 4.39 (s, 1H), 5.26 (d, 1H, J=12.9 Hz), 7.22 (t, 1H, J=8.3 Hz), 7.32-7.55 (m, 4H), 7.97 (m, 2H); MS (EI) m/e (relative intensity) 316 (72), 287 (100), 246 (69), 153 (16), 127 (62). Anal. Calcd. for C₁₉H₁₃N₄F.0.6 CH₃OH: C, 70.16; H, 4.37; N, 16.55. Found: C, 69.98; H, 4.31; N, 16.70.

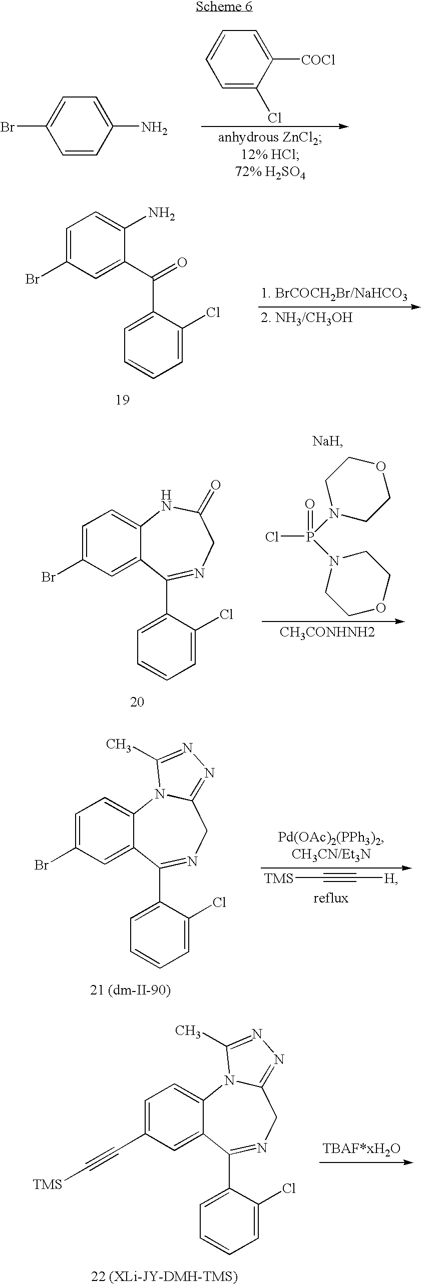

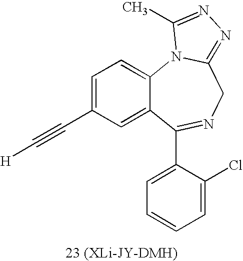

2-Amino-5-bromo-2′-chlorobenzophenone 19 was obtained from simple starting materials, 4-bromoaniline and 2-chlorobenzoyl chloride, according to the improved conditions in the literature. The benzodiazepine 20, available from reference 1, was stirred with sodium hydride and di-4-morpholinophosphinic chloride, followed by addition of acetylhydrazide to furnish triazolobenzodiazepine 21 (dm-II-90). The ligand 22 (XLi-JY-DMH-TMS) was obtained by a Heck coupling reaction of 21 (dm-II-90) with trimethylsilylacetylene. This compound was converted into acetylene 23 (XLi-JY-DMH) on stirring with fluoride anion as shown in Scheme 6.

2-Amino-5-bromo-2′-chlorobenzophenone 19. 2-Chlorobenzoyl chloride (177 mL, 1.4 mol) was cooled in a 2-L flask equipped with a condenser and a thermometer to 0° C. with an ice-water bath and 4-bromoaniline (100 g, 0.58 mol) was added to the cooled solution. The mixture was heated to 120° C. and kept at this temperature for 1 h until analysis by TLC indicated 4-bromoaniline had been consumed (EtOAc:hexane, 1:4). The solution was heated to 160° C. and anhydrous ZnCl₂ (95 g, 0.70 mol, flamed dried) was added in one portion. The temperature was increased to 195° C. and stirring was maintained at this temperature for 3 hr until no more bubbles were evolved. The mixture was cooled to 120° C. and aq HCl (12%, 350 mL) was added dropwise slowly. The mixture was kept at reflux for 20 min, after which the aq layer was poured off. This procedure with aq HCl was repeated 4 times. Water (350 mL) was then added, and the mixture held at reflux for 20 min and then the water was poured off. This was repeated several times until the solid was not a block any more. Then H₂SO₄ (72%, 700 mL) was added to the residue and the mixture was heated to reflux for about 1 hr until the reaction mixture became a homogeneous dark colored solution. The hot acidic solution was poured into a mixture of ice and water with stirring. The precipitate which resulted was filtered and washed with a large amount of cold water until the pH value of the solid was about 6. The solid was then suspended in ice water and aq NaOH (40%, 290 mL) was added carefully. The mixture which resulted was stirred for 2 hrs. The solid was filtered and washed with ice water. The suspension of the solid in ice water was adjusted carefully to approximately pH=3 with aq H₂SO₄ (40%) dropwise. The solid which remained was filtered and washed with water to neutrality. The yellow solid 19 (66.1 g, 37.0%) was dried and used directly in the next step without further purification. ¹H NMR (300 MHz, CDCl₃) δ 6.49 (s, br, 2H), 6.65 (d, 1H, J=8.82 Hz), 7.26-7.8 (m, 6H).

8-Bromo-5-(2′-chlorophenyl)-1-methyl-4H-s-triazolo[4,3-a]-1,4-benzodiazepine 21 (dm-II-90). A solution of benzodiazepine 20 (20 g, 57 mmol, available from reference 1) in dry THF (250 mL) was cooled to −5° C. and a 60% dispersion of sodium hydride (3.66 g, 92 mmol) was added in one portion. The mixture was allowed to warm to rt with stirring and the stirring was continued at rt until no more bubbles were evolved. The suspension was cooled to −5° C. after which di-4-morpholinylphosphinic chloride (21.8 g, 86 mmol) was added and this mixture was stirred for 30 min and allowed to warm to rt. The mixture was stirred for an additional 1.5 hr. To the mixture was then added a solution of acetylhydrazide (9.42 g, 114 mmol) in butanol (60 mL) and stirring was continued at rt for 10 min. The solvent was removed under reduced pressure and the residue was taken up in butanol (100 mL) and held at reflux for 2 hr. Butanol was removed under reduced pressure and the residue was partitioned between CH₂Cl₂ (200 mL) and H₂O (100 mL). The aq layer was extracted 4 times and the organic layers combined. The organic layer was washed with brine and dried (Na₂SO₄). After the solvent was removed under reduced pressure, the residue was crystallized from EtOAc-Et₂O to provide the pure triazolobenzodiazepine 21 (dm-II-90, 14 g, 63.2%) as a yellow solid: mp 265-267° C. [lit 274-275° C.]⁽¹⁰⁾; IR (KBr) 3120 (br.), 1686, 1479, 1386, 1014, 827, 747 cm⁻¹; ¹H NMR (300 MHz, CDCl₃) δ 2.42 (s, 1H), 2.60 (s, 3H), 4.18 (d, 1H, J=12.9 Hz), 5.56 (d, 1H, J=12.9 Hz), 7.36 (m, 3H), 7.43 (m, 2H), 7.61 (m, 1H), 7.80 (dd, 1H, J=2.1 Hz, 8.7 Hz); MS (EI) m/e (rel intensity) 386 (M⁺, 45), 357 (100); Anal. Calcd. For C₁₇H₁₂N₄BrCl.0.5H₂O: C, 51.65; H, 3.32; N, 14.18; Found C, 51.95; H, 2.97; N, 13.91.

8-Trimethylsilylacetylenyl-5-(2′-chlorophenyl)-1-methyl-4H-s-triazolo-[4,3-a]-1,4-benzodiazepine 22 (XLi-JY-DMH-TMS). A mixture of 21 (7.75 g, 20 mmol), acetonitrile (600 mL), triethylamine (500 mL) and bis(triphenylphosphine)-palladium (II) acetate (1.2 g, 1.6 mmol) was degassed. Tri-methylsilylacetylene (5.65 mL, 40 mmol) was then added and the solution was degassed again. The solution was then heated to reflux for 4 hr until analysis by TLC indicated the starting material had disappeared. The mixture was cooled to rt and concentrated under reduced pressure. The residue was partitioned between H₂O (50 mL) and EtOAc (2×200 mL). The combined organic layer was washed with brine and dried (Na₂SO₄). The residue was purified by flash chromatography on silica gel (CHCl₃) to furnish the trimethylsilyl analogue 22 (XLi-JY-DMH-TMS, 3 g, 37.0%) as white solid: mp 265-267° C.; IR (KBr) 2930, 1618, 1554, 1497, 1429, 1316, 885, 847 cm⁻¹; ¹H NMR (300 MHz, CDCl₃) δ 0.24 (s, 9H), 2.65 (s, 3H), 4.15 (d, 1H, J=12.9 Hz), 5.52 (d, 1H, J=12.9 Hz), 7.35-7.45 (m, 5H), 7.61 (m, 1H), 7.72 (dd, 1H, J=1.8 Hz, 8.4 Hz); MS (EI) m/e (rel intensity) 404 (M⁺, 90), 375 (100); Anal. Calcd. For C₂₂H₂₁N₄SiCl: C, 65.33; H, 5.24; N, 13.86. Found: C, 64.99; H, 4.98; N, 13.79.

8-Acetyleno-5-(2′-chlorophenyl)-1-methyl-4H-s-triazolo-[4,3-a]-1,4-benzodiazepine 23 (XLi-JY-DMH). A solution of benzodiazepine 22 (1.25 g, 31 mmol) in THF (250 mL) was cooled to −30° C. and treated with Bu₄NF.xH₂O (0.97 g, 37 mmol). After the mixture was stirred for 5 min, analysis by TLC (silica gel; EtOAc:EtOH 4:1) indicated starting material had disappeared. Water (70 mL) was then added and the mixture was allowed to warm to rt. The mixture was then extracted with EtOAc (2×200 mL). The organic layer was washed with brine and dried (Na₂SO₄). After removal of the solvent under reduced pressure, the residue was washed successively with ethyl ether, ethyl acetate and chloroform. After drying, the title compound 23 (XLi-JY-DMH) was obtained (1.0 g, 97.3%) as a white solid: mp>250° C. (dec); IR (KBr) 3185, 1623, 1543, 1497, 1429, 756 cm⁻¹; ¹H NMR (300 MHz, CDCl₃) δ 2.65 (s, 3H), 3.17 (s, 1H), 4.18 (d, 1H, J=12.9 Hz), 5.54 (d, 1H, 12.9 Hz), 7.34 (m, 2H), 7.41-7.45 (m, 3H), 7.6 (m, 1H), 7.75 (dd, 1H, J=1.8 Hz, 8.4 Hz); MS (EI) m/e (rel intensity) 332 (M⁺, 78) 303 (100).

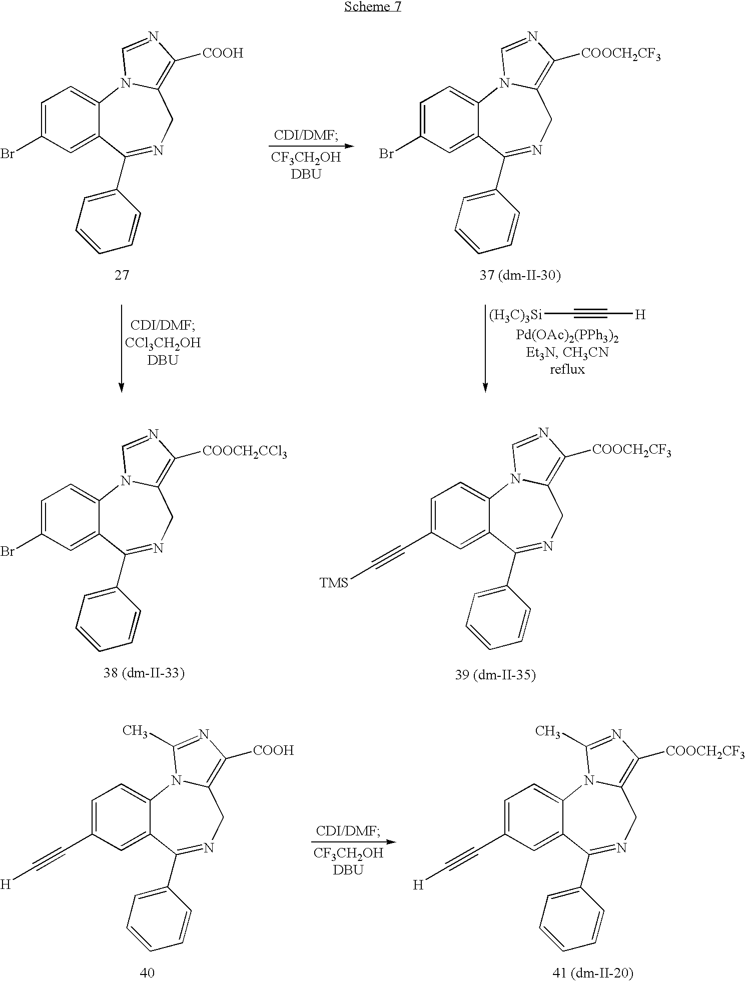

Esters 37 (dm-II-30), 38 (dm-II-33) and 41 (dm-II-20) were prepared according to the general procedure described from the starting acids and different alcohols, respectively. The bromide 37 was converted into the trimethlylacetylenyl compound 39 (dm-II-35) under standard conditions (Pd-mediated, Heck-type coupling) (Scheme 7).

General Procedure for Preparing the Esters.

The acid was dissolved in DMF (10 mL/mmol S.M.) and CDI (1.2 eq) was added. The reaction mixture was stirred at room temperature for 3 h followed by addition of the alcohol (10 eq) and DBU (1 eq). The stirring was maintained until the disappearance of all the starting material as determined by TLC (EtOAc:EtOH 4:1). The reaction mixture was then quenched by adding water. The solid which precipitated was filtered and washed with ethyl ether. It was purified by flash chromatography (EtOAc) on silica gel or neutral aluminum oxide for ester 38.

Trifluoroethyl 8-bromo-6-phenyl-4H-benzo[f]imidazo[1,5-a][1,4]diazepine-3-carboxylate 37 (dm-II-30). A white solid (69.1%) from acid 27 and 2,2,2-trifluoroethanol: mp 202-204° C.; IR (KBr) 3114, 1711, 1608, 1495, 1368, 1288, 1158 cm⁻¹; ¹H NMR (300 MHz, CDCl₃) δ 4.10 (d, 1H, J=12.6 Hz), 4.68 (m, 1H), 4.85 (m, 1H), 6.02 (d, 1H, J=12.6 Hz), 7.41-7.54 (m, 6H), 7.62 (d, 1H, J=2.1 Hz), 7.83 (dd, 1H, J=2.1 Hz, 8.4 Hz), 7.97 (s, 1H); MS (EI) m/e (rel intensity) 463 (M⁺, 14), 465 (14).

Trichloroethyl 8-bromo-6-phenyl-4H-benzo[f]imidazo[1,5-a][1,4]diazepine-3-carboxylate 38 (dm-II-33). A white solid (90.9%) from acid 27 and 2,2,2-trichloroethanol: mp 113-116° C.; IR (KBr) 3434, 1728, 1610, 1493, 1270, 1146, 1128 cm⁻¹; ¹H NMR (300 MHz, CDCl₃) δ 4.11 (d, 1H, J=12.6 Hz), 4.91 (d, 1H, J=12.0 Hz), 5.19 (d, 1H, J=12.0 Hz), 6.12 (d, 1H, J=12.6 Hz), 7.41-7.54 (m, 6H), 7.61 (d, 1H, J=2.1 Hz), 7.83 (dd, 1H, J=2.1 Hz, 8.4 Hz); MS (EI) m/e (rel intensity) 511 (M⁺, 45).

Trifluoroethyl 8-trimethylsilylacetylenyl-6-phenyl-4H-benzo[f]imidazo[1,5-a][1,4]diazepine-3-carboxylate 39 (dm-II-35). A white solid (49.8%): mp 107-110° C.; IR (KBr) 2961, 1734, 1611, 1560, 1497, 1251, 1159, 1120, 846 cm⁻¹; ¹H NMR (300 MHz, CDCl₃) δ 0.25 (s, 9H), 4.08 (d, 1H, J=12.3 Hz), 4.69 (m, 1H), 4.84 (m, 1H), 5.98 (d, 1H, J=12.3 Hz), 7.39-7.57 (m, 7H), 7.76 (dd, 1H, J=1.8 Hz, 8.4 Hz); MS (EI) m/e (rel intensity) 481 (M⁺, 100).

Trifluoroethyl 8-acetylenyl-6-phenyl-4H-benzo[f]imidazo[1,5-a][1,4]diaze-pine-3-carboxylate 41 (dm-II-20). A white solid (36.9%) from acid 40 and 2,2,2-trifluoroethanol: mp 188-190° C.; IR (KBr) 3443, 3277, 1710, 1600, 1492, 1366, 1280, 1156 cm⁻¹; ¹H NMR (500 MHz, CDCl₃) δ 3.18 (s, 1H), 4.08 (d, 1H, J=12.5 Hz), 4.67 (m, 1H), 4.82 (m, 1H), 5.98 (d, 1H, J=12.5 Hz), 7.37-7.40 (m, 2H), 7.44-7.51 (m, 3H), 7.56-7.59 (m, 2H), 7.78 (dd, 1H, J=1.5 Hz, 8.5 Hz); MS (EI) m/e (rel intensity) 409 (M⁺, 28). Anal. Calcd. For C₂₂H₁₄N₃O₂F₃.0.25H₂O: C, 63.82; H, 3.72; N, 10.16. Found: C, 63.89; H, 3.37; N, 9.94.

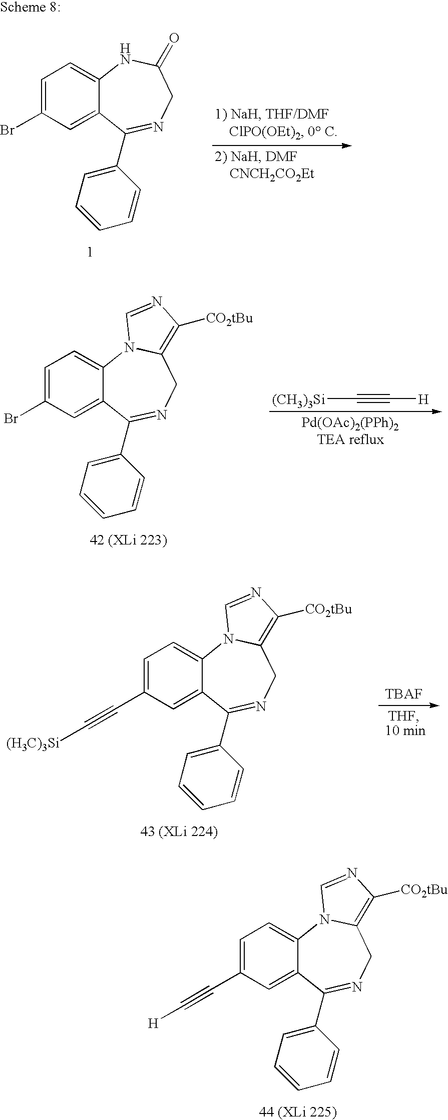

The bromide 1 was reacted with diethylphosphorochloridate in the presence of sodium hydride, followed by addition of t-butyl isocyanoacetate to provide the ester 42. This was converted into the trimethylsilylacetyleno compound 43 under standard conditions (Pd-mediated, Heck-type coupling). Treatment of 43 with fluoride gave the title compound 44.

### Procedure for XLi225:

t-Butyl 8-bromo-6-phenyl-4H-benzo[f]imidazo[1,5-a][1,4]diazepine-3-carboxylate 42. This benzodiazepine 42 was obtained in 40% yield from 1 analogous to the literature procedure as a white solid. 42 (XLi223): mp: 222°-223° C.; IR (KBr) 2975, 2358, 1717, 1608, 1557, 1277, 1073, 908, 696, 652 cm⁻¹; ¹H NMR (CDCl₃) δ 1.60 (s, 9H), 4.03 (d, 1H, J=12.5 Hz), 6.08 (d, 1H, J=12.4 Hz), 7.35-7.52 (m, 7H), 7.58 (d, 1H, J=2.2 Hz), 7.80 (dd, 1H, J=2.22 Hz and 8.55 Hz), 7.93 (s, 1H);

t-Butyl-8-trimethylsilylacetylenyl-6-phenyl-4H-benzo[f]imidazo[1,5-a][1,4]-diazepine-3-carboxylate 43 (XLi 224). A mixture of bromide 42 (1 g, 2.28 mmol), trimethylsilylacetylene (559 mg, 5.69 mmol) and bis(triphenylphosphine)-palladium-(II) acetate (55 mg, 0.073 mmol) in a mixed solvent system of CH₃CN (15 mL) and anhydrous TEA (25 mL) was heated to reflux under argon. After stirring for 6 hours at reflux, the mixture was cooled to room temperature and the precipitate which formed was removed by filtration. The filtrate was concentrated under reduced pressure and the residue was treated with a saturated aqueous solution of NaHCO₃ (20 mL), and extracted with CHCl₃ (3×25 mL). The combined extracts were washed with brine and dried (Na₂SO₄). After removal of solvent under reduced pressure, the residue was purified by flash chromatography (silica gel, EtOAc) to afford 43 (XLi224) as a white solid (710 mg, 68.9%). mp: 234°-236° C.; IR (KBr) 2973, 2357, 2154, 1719, 1611, 1493, 1366, 1250, 1152, 1075, 946, 880 cm⁻¹; ¹H NMR (CDCl₃) δ 0.23 (s, 9H), 1.64 (s, 9H), 4.05 (d, 1H, J=12.7 Hz), 6.06 (d, 1H, J=12.4), 7.37-7.53 (m, 7H), 7.73 (dd, 1H, J=1.95 and 8.25 Hz), 7.92 (s, 1H); MS (EI) m/e (relative intensity) 427 (M⁺, 76), 412 (5), 381 (55), 353 (100) 303 (10), 287 (7).

t-Butyl 8-acetylenyl-6-phenyl-4H-benzo[f]imidazo[1,5-a][1,4]diazepine-3-carboxylate 44 (XLi 225). A solution of 43 (128 mg, 0.281 mmol), in THF (15 mL) was treated with Bu₄NF.H₂O (100.04 mg, 0.38 mmol). The mixture which resulted was allowed to stir for 5 min at room temperature after which the mixture was added to H₂O (10 mL) and extracted with EtOAc (3×15 mL). The combined organic extracts were washed with brine (15 mL) and dried (Na₂SO₄). After removal of solvent under reduced pressure, the residue was purified by a wash column (silica gel, EtOAc) to furnish 44 (XLi225) (92 mg, 85.4%) as a white solid: mp: 221°-223° C.; IR (KBr) 3159, 3107, 2092, 1721, 1606 cm⁻¹; ¹H NMR (CDCl₃) δ 1.62 (s, 9H), 3.21 (s, 1H), 4.12 (d, 1H, J=10.2 Hz), 6.07 (d, 1H, J=12.5 Hz), 7.35-7.53 (m, 7H), 7.73 (dd, 1H, J=1.8 Hz and 8.3 Hz), 7.92 (s, 1H).

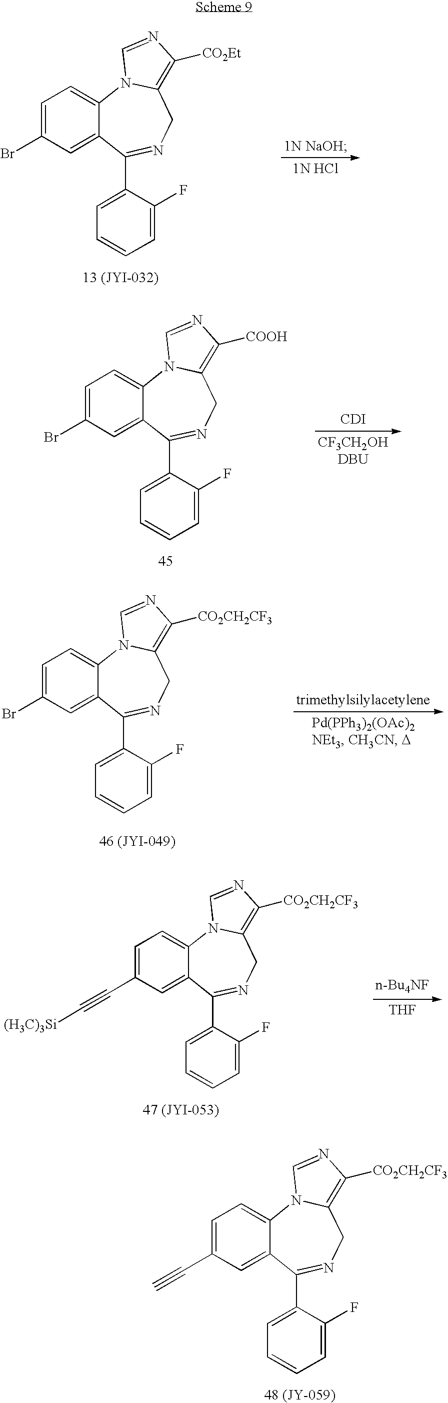

7-Bromo-2′-fluorobenzodiazepine 13 was hydrolyzed with aq 2 N sodium hydroxide in EtOH and acidified to pH 4 by adding 1 N HCl to afford the acid 45. The acid, obtained from the ester 13, was stirred with CDI in DMF, followed by stirring with trifluoroethanol and DBU to provide the ester 46 (JYI-049). This material 46 was heated with trimethylsilylacetylene in a Heck-type coupling reaction⁸ to provide the trimethylsilyl analog 47 (JYI-053). The silyl group was removed from 47 on treatment with tetrabutylammonium fluoride to furnish 48 (JYI-059) in 70% yield.

### Procedure:

8-Bromo-6-(2′-fluorophenyl)-4H-benzo[f]imidazo[1,5-a][1,4]diaze-pine-3-carboxylic acid 45. The ester 13 (1.0 g, 2.36 mmol) was dissolved in EtOH (80 mL) and 2 N aq NaOH (8 mL) was added to the solution. The mixture was stirred at rt for 4 hours. After the EtOH was removed under reduced pressure, the solution was allowed to cool. The pH value was adjusted to 4 by adding 1 N HCl dropwise. The mixture was filtered and the solid was washed with cold water and ethyl ether. The solid was dried to afford 45 (0.96 g, 97%) as a white solid: mp 280° C. (dec); IR (KBr) 3419, 1740, 1611, 1491 cm⁻¹; ¹HNMR (DMSO-d₆) δ 4.11 (bs, 1H), 5.99 (bs, 1H), 7.20 (t, 1H, J=8.5 Hz), 7.32 (t, 1H, J=7.5 Hz), 7.38 (d, 1H, J=1.8 Hz), 7.55 (m, 2H), 7.84 (d, 1H, J=8.7 Hz), 7.95 (dd, 1H, J=8.6, 1.9 Hz), 8.35 (s, 1H). MS (EI) m/e (relative intensity) 400 (72), 399 (85), 381 (100), 355 (82).

Trifluoroethyl-8-bromo-6-(2′-fluorophenyl)-4H-benzo[f]imidazo[1,5-a][1,4]diazepine-3-carboxylate 46 (JYI-049). The carboxylic acid 45 (0.89 g, 2.23 mmol) was dissolved in dry DMF (20 mL), after which CDI (0.72 g, 4.45 mmol) was added at rt and the mixture was stirred for 12 hours. The trifluoroethanol (0.49 mL, 6.68 mmol) in DMF (1 mL) and DBU (0.37 mL, 2.45 mmol) in DMF (1 mL) were then added to the mixture and stirring continued overnight. The solvent was evaporated under reduced pressure and the residue was purified by flash chromatography (silica gel, hexanes/EtOAc: 3/1) to afford 46 (JYI-049, 0.81 g, 76%) as a white solid: mp 223-224° C.; IR(CHCl₃) 3063, 1732, 1611, 1492 cm⁻¹; ¹HNMR (CDCl₃) δ 4.16 (bs, 1H), 4.80 (bs, 2H), 6.07 (bs, 1H), 7.06 (dt, 1H, J=8.3, 0.9 Hz), 7.30 (m, 2H), 7.48 (m, 2H), 7.68 (dt, 1H, J=7.6, 1.8 Hz), 7.80 (dd, 1H, J=8.6, 2.1 Hz), 8.11 (s, 1H). MS (EI) m/e (relative intensity) 483 (38), 383 (64), 355 (100). Anal. Calcd. for C₂₀H₁₂N₃O₂F₄Br: C, 49.81; H, 2.51; N, 8.71. Found: C, 49.97; H, 2.44; N, 8.68.

Trifluoroethyl-8-trimethylsilylacetylenyl-6-(2′-fluorophenyl)-4H-benzo[f]imidazo[1,5-a][1,4]diazepine-3-carboxylate 47 (JYI-053). A mixture of bromide 46 (JYI-049, 482 mg, 1.0 mmol), trimethylsilylacetylene (0.28 mL, 2.0 mmol) and bis(triphenylphosphine)palladium (II) acetate (75 mg, 0.1 mmol) in a mixed solvent system of CH₃CN (25 mL) and anhydrous triethylamine (25 mL) was heated to reflux under argon. After stirring for 12 h at reflux, the mixture was cooled to room temperature and the precipitate which formed was removed by filtration. The filtrate was concentrated under reduced pressure and the residue was treated with a saturated aq solution of NaHCO₃ (40 mL), and extracted with CHCl₃ (3×100 mL). The combined organic extracts were washed with brine (2×50 mL) and dried (Na₂SO₄). After removal of solvent under reduced pressure, the residue was purified by flash chromatography (silica gel, hexanes/EtOAc: 3/1) to afford 47 (JYI-053, 360 mg, 76%) as a gray solid: mp 220-221° C.; IR(CHCl₃) 2960, 1741, 1612, 1496 cm⁻¹; ¹H NMR (CDCl₃) δ 0.25 (s, 9H), 4.12 (bs, 1H), 4.82 (bs, 2H), 6.10 (bs, 1H), 7.06 (t, 1H, J=8.3 Hz), 7.30 (m, 1H), 7.48 (m, 2H), 7.56 (d, 1H, J=8.3 Hz), 7.67 (m, 1H), 7.73 (dd, 1H, J=8.3, 1.8 Hz), 8.02 (s, 1H); MS (EI) m/e (relative intensity) 499 (52), 399 (45), 371 (100), 235 (21), 178 (36). Anal. Calcd. for C₂₅H₂₁N₃O₂F₄Si: C, 60.11; H, 4.24; N, 8.41. Found: C, 60.27; H, 4.22; N, 8.33.

Trifluoroethyl-8-acetyleno-6-(2′-fluorophenyl)-4H-benzo[f]imidazo-[1,5-a][1,4]diazepine-3-carboxylate 48 (JYI-059). A solution of 47 (JYI-053, 475 mg, 1.0 mmol) in THF (15 mL) was treated with Bu₄NF (2 mL, 1.0M solution in THF). The mixture, which resulted, was allowed to stir for 5 min at room temperature after which the mixture was added to H₂O (5 mL) and extracted with EtOAc (3×10 mL). The combined organic extracts were washed with brine (2×10 mL) and dried (Na₂SO₄). The solvent was removed under reduced pressure and the residue was recrystallized from ethyl acetate/hexanes to afford 48 (JYI-059, 299 mg, 70%) as a pale yellow solid: mp 192-193° C.; IR(CHCl₃) 3295, 3052, 1741, 1612, 1494, 1277, 1159 cm⁻¹; ¹H NMR (CDCl₃) δ 3.14 (s, 1H), 4.17 (bs, 1H), 4.78 (bs 2H), 4.47 (s, 1H), 6.05 (bs, 1H), 7.05 (dt, 1H, J=8.3, 0.8 Hz), 7.30 (m, 1H), 7.48 (m, 2H), 7.60 (d, 1H, J=8.3 Hz), 7.68 (dt, 1H, J=7.6, 1.8 Hz), 7.76 (dd, 1H, J=10.1, 1.8 Hz), 8.02 (s, 1H); MS (EI) m/e (relative intensity) 427 (37), 327 (26), 299 (100), 178 (50). Anal. Calcd. for C₂₂H₁₃N₃O₂F₄: C, 61.83; H, 3.07; N, 9.83. Found: C, 61.94; H, 3.03; N, 9.68.

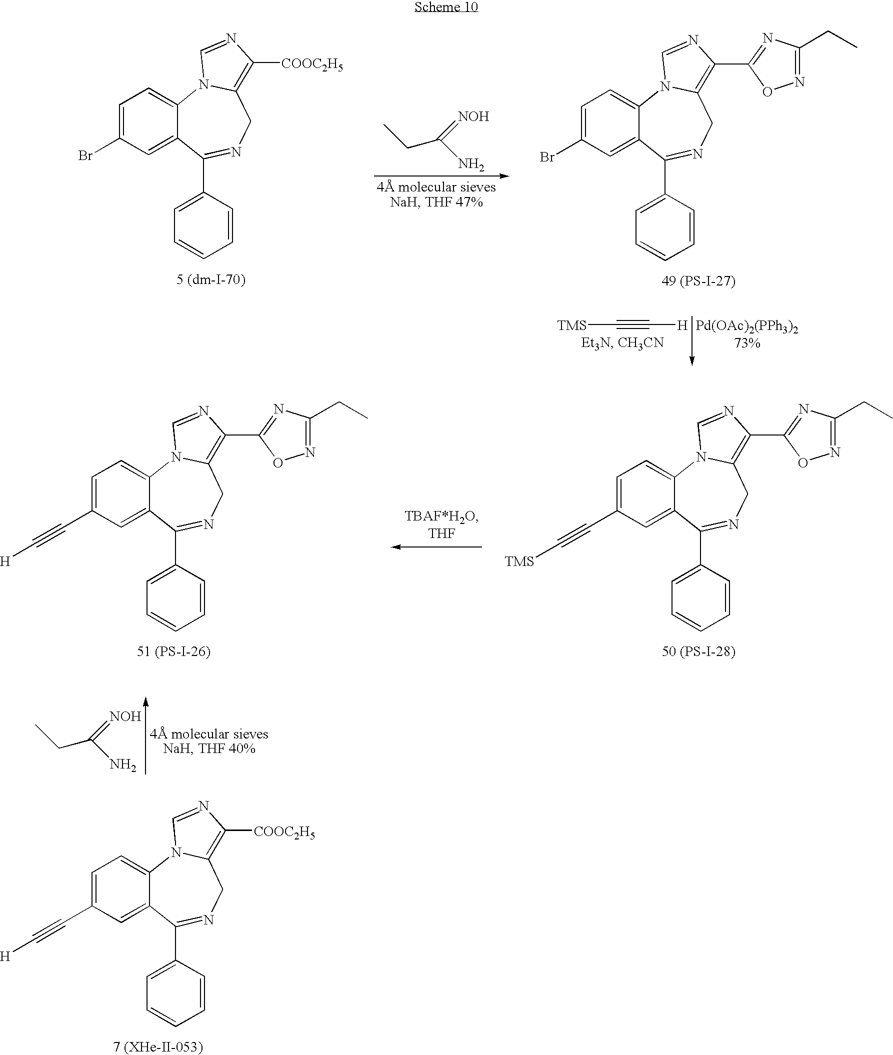

Ethyl amido oxime (59.5 mg, 0.676 mmol) was added to a stirred suspension of powdered 4 Å molecular sieves (75 mg) in anhydrous THF (15 mL) under nitrogen. After the mixture was stirred at rt for 10 min, NaH (27 mg of 60% in mineral oil, 0.676 mmol) was added to the mixture. After the mixture was stirred for a further 30 min, a solution of the forgoing ester 7 (XHeII-053, 120 mg, 0.338 mmol) in THF (20 mL) was added. The mixture which resulted was heated to reflux for 8 hr. It was cooled to rt, after which acetic acid (40.6 mg, 0.676 mmol) was added. After the solution was stirred for 10 min, the mixture was filtered through celite. The filtrate was diluted with CH₂Cl₂ (50 mL) and washed with water, brine and dried (K₂CO₃). Evaporation of the solvent under reduced pressure afforded a pale yellow solid, which was purified by flash column chromatography (silica gel, EtOAc/hexane, 2:3) to furnish 51 as a white solid (PS—I-26, 52 mg, 40%). mp: 221-222° C.; IR (KBr) 3297, 3105, 1631, 1570, 1495, 1310, 938 cm⁻¹; ¹H NMR (CDCl₃) δ 8.07 (s, 1H), 7.80 (dd, 1H, J=8.4 Hz, J=1.8 Hz), 7.64-7.60 (m, 2H), 7.53-7.37 (m, 5H), 6.12 (d, 1H, J=12.9 Hz), 4.21 (d, 1H, J=12.9 Hz), 3.20 (s, 1H), 2.88 and 2.83 (ABq, 2H, J=7.6 Hz), 1.41 (t, 3H, J=7.6 Hz); ¹³C NMR (CDCl₃) δ 171.8, 170.6, 168.8, 139.1, 136.6, 135.8, 135.4 (2C), 135.1, 130.7, 129.3 (2C), 128.3 (2C), 128.1, 124.7, 122.7, 121.6, 81.2, 80.0, 44.7, 19.7, 11.5; MS (m/z) 379 (100).

This compound 49 (PS—I-27) was obtained in 47% yield from 5 (dm-1-70) (using a method analogous to the production of 51 from 7, described above) as a white solid. mp: 210° C.; IR (KBr) 3106, 1631, 1563, 1493, 1147, 931, 698 cm⁻¹; ¹H NMR (CDCl₃) δ 8.06 (s, 1H), 7.84 (dd, 1H, J=8.6 Hz, j=2.25 Hz), 7.63-7.38 (m, 7H), 6.13 (d, 1H, J=12.9 Hz), 4.21 (d, 1H, J=12.9 Hz), 3.20 (s, 1H), 2.88 and 2.83 (ABq, 2H, J=7.6 Hz), 1.41 (t, 3H, J=7.6 Hz); MS (m/z) 435 (100).

To the suspension of compound 49 (PS—I-27, 0.5 g, 1.15 mmol) in acetonitrile (30 mL) and triethylamine (80 mL) was added bis(triphenylphosphine)palladium (II) acetate (0.086 g, 0.115 mmol). The solution was degassed and trimethylsilylacetylene (0.33 mL, 2.3 mmol) added. The mixture was heated to reflux and stirred overnight. After removal of the solvent, the residue was dissolved in CH₂Cl₂ and washed with a saturated aqueous solution NaHCO₃ and brine. The organic layer was dried (Na₂SO₄) filtered and concentrated under vacuum. The residue was purified by flash column chromatography (EtOAc:hexane 2:3) to furnish the trimethylsilyl analog 50 (PS—I-28, 380 mg, 73%) as a pale yellow solid: mp: 193-194° C.; IR (KBr) 3106, 2960, 2149, 1630, 1567, 1493, 938, 851, 701 cm⁻¹; ¹H NMR (300 Hz, CDCl₃) δ 8.07 (s, 1H), 7.78 (dd, 1H, J=1.86, 8.34 Hz), 7.61-7.38 (m, 7H), 6.11 (d, J=12.78 Hz), 4.19 (d, J=12.78 Hz), 2.88 and 2.83 (ABq, 2H, J=7.56 Hz), 1.41 (t, 3H, J=7.56 Hz), 0.25 (s, 9H).

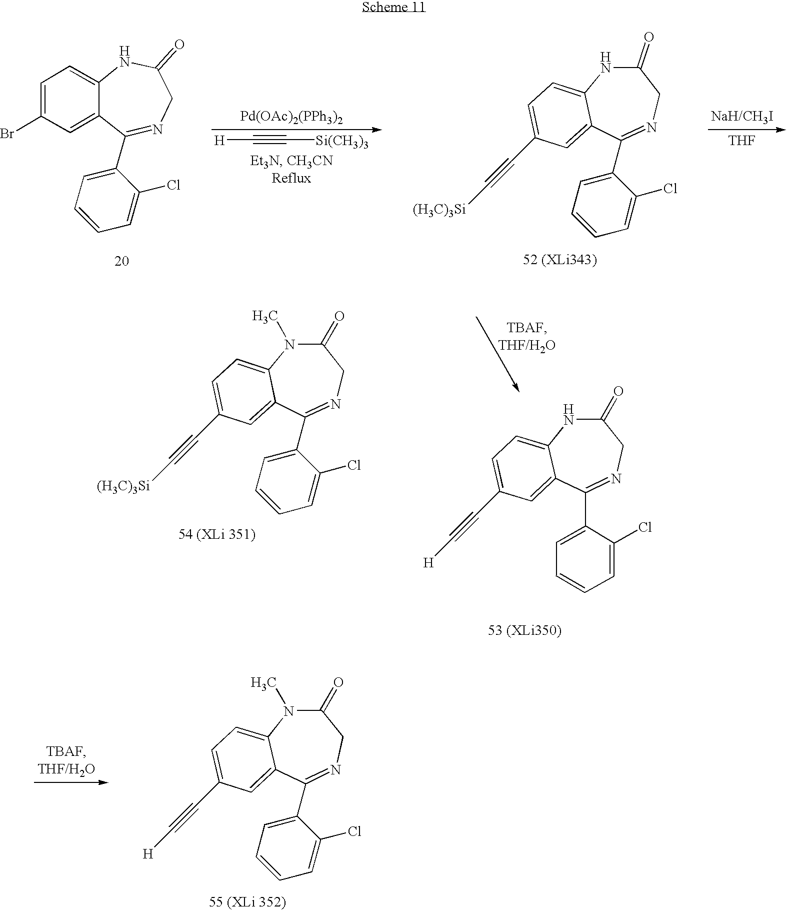

The bromide 20 was reacted with trimethylsilylacetylene in the presence of a palladium catalyst to provide trimethylsilyl analog 52. This product was methylated with methyl iodide/sodium hydride to give the N-methyl benzodiazepine 54 (XLi 351). This was subjected to fluoride-mediated desilylation to furnish 53 (XLi 350) from 52 and 55 (XLi 352) from 54.

### Procedure for XLi 350 and XLi 352:

7-Trimethylsilylacetyleno-5-phenyl-(2′-chlorophenyl)1,3-dihydrobenzo[e]-1,4-diazepin-2-one 52 (XLi 343). A mixture of 20 (500 mg, 1.43 mmole) available in triethyl amine (10 mL) and CH₃CN (16 mL) with trimethyl-silylacetylene (126 mg, 1.28 mmole) and bis(triphenylphosphine)palladium (II) acetate (64.3 mg, 0.086 mmol) was heated to reflux under nitrogen. After 6 hours, the reaction mixture was cooled to room temperature and filtered. The filtrate was concentrated under vacuum and the residue was treated with a saturated aqueous NaHCO₃ solution (15 mL), and extracted with CH₂Cl₂ (3×20 mL). The organic layers were combined and washed with brine and dried (Na₂SO₄). After removal of solvent under reduced pressure, the residue was purified via flash chromatography (silica gel, EtOAc/hexanes: 1/1) to furnish 52 as a yellow powder (310 mg, 59%): mp: 225.8-228.2° C.; IR (KBr) 2953, 2358, 1685, 1616, 1490, 1328, 1248, 1058, 1011, 841, 746 cm⁻¹, ¹H NMR (CDCl₃) δ 0.21 (s, 9H), 4.38 (s, 2H), 7.41 (d. 1H, J=8.37 Hz), 7.19-7.52 (br, 7H), 8.11 (s, 1H); MS (EI) m/e (relative intensity) 366 (M⁺, 100), 331 (59), 229 (18), 161 (26).

7-Acetyleno-5-phenyl-(2′-chlorophenyl)-1,3-dihydro-benzo[e]-1,4-diazepin-2-one 53 (XLi 350): A solution of 52 (150 mg, 0.408 mol) in THF (30 mL) was treated with tetrabutylammonium fluoride (1M in THF). The mixture was stirred for 20 minutes at room temperature before water (30 mL) was added. The mixture was then extracted with EtOAc (3×30 mL). The combined organic extracts were washed with brine and dried over (Na₂SO₄). The solvent was removed under vacuum and the residue which resulted was passed through a wash column (silica gel, EtOAc/hexanes: 4/1) to give 53 as light yellow crystals (110 mg, 95.2%); mp: 215° C.; IR (KBr) 3290, 1685, 1615, 1491, 1328, 731 cm⁻¹, ¹H NMR (CDCl₃) δ 3.06 (s, 1H), 4.40 (s, 3H), 7.03-7.61 (m, 7H), 7.58-7.86 (m, 2H), 7.99 (s, 1H); MS (EI) m/e (relative intensity) 294 (M⁺, 100), 266 (75), 265 (87), 259 (83), 231 (40), 201 (24), 176 (23).

1-Methyl-7-trimethylsilylacetyleno-5-phenyl-(2′-chlorophenyl)-1,3-dihydro-benzo[e]-1,4-diazepin-2-one 54 (XLi 351). A mixture of 52 (300 mg, 0.82 mmol) was dissolved in dry THF (40 mL) at 0° C. and NaH (60% in mineral oil, 50 mg, 1.25 mmol) was added to the solution in one portion. The slurry was then stirred for 20 min at O′C and CH₃I (139 mg, 0.98 mmol) was added to the mixture and it was warmed up to room temperature. After the mixture stirred for 3 hours at room temperature, the THF was then removed under reduced pressure. The residue was purified by flash chromatography [hexanes/EtOAc (1:4)] to provide the title compound 54 (260 mg, 83%) as a white solid: mp: 196.9-198° C.; IR (KBr) 2953, 1676, 1611, 1489, 1346, 1125, 1078, 913, 742 cm⁻¹; ¹HNMR (CDCl₃) □δ(ppm) 0.21 (s, 9H) 3.46 (s, 3H), 3.54 (d, 1H, J=10.9 Hz), 4.60 (d, 1H. J=10.8 Hz), 7.20-7.43 (m, 5H), 7.58-7.65 (m, 3H). MS (EI) m/e (relative intensity) 380 (M⁺, 8), 366 (10), 308 (100), 280 (88), 273 (97), 245 (61).

1-Methyl′-7-acetyleno-5-phenyl-(2′-chlorophenyl)-1,3-dihydro-benzo[e]-1,4-diazepin-2-one 55 (XLi 352): A solution of 54 (100 mg, 0.262) in THF (30 mL) was treated with tetrabutylammonium fluoride (1M in THF). The mixture was stirred for 20 minutes at room temperature before water (30 mL) was added. The mixture was then extracted with EtOAc (3×30 mL). The combined organic extracts were washed with brine and dried (Na₂SO₄). The solvent was removed under vacuum and the residue which resulted was passed through a wash column (silica gel, EtOAc/hexanes: 4/1) to give 55 as light yellow crystals (71 mg, 90%): mp: 95.6-98.1° C.; IR (KBr) 2953, 1677, 1489, 1346, 1091, 791, 749 cm⁻¹, ¹H NMR (CDCl₃) (ppm) 3.05 (s, 1H), 3.46 (s, 3H), 3.83 (d, 1H, J=10.5 Hz), 4.87 (d, 1H, J=9.33 Hz), 5.28 (s, 1H), 7.20-7.43 (m, 5H), 7.58-7.86 (m, 2H); MS (EI) m/e (relative intensity) 308 (M⁺, 100), 294 (19), 280 (82), 273 (99), 249 (28), 245 (61), 229 (29), 201 (32), 189 (43).

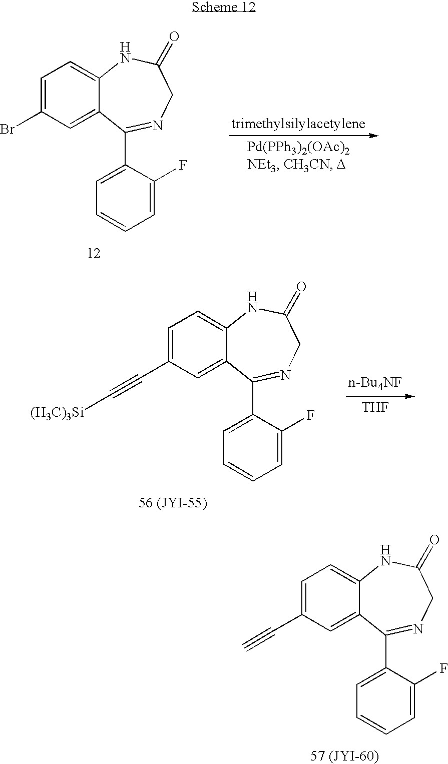

7-Trimethylsilylacetyleno-5-(2′-fluorophenyl)-1,3-dihydrobenzo[e]-1,4-diazepine-2-one 56 (JYI-55). A mixture of bromide 12 (1.6 g, 5.0 mmol), trimethylsilyl-acetylene (3.0 mL, 21.0 mmol) and bis(triphenylphosphine)palladium (II) acetate (375 mg, 0.5 mmol) in a mixed solvent system of CH₃CN (60 mL) and anhydrous triethylamine (40 mL) was heated to reflux under argon. After stirring for 3 h at reflux, the mixture was cooled to room temperature and the precipitate which formed was removed by filtration. The filtrate was concentrated under reduced pressure and the residue was treated with a saturated aq solution of NaHCO₃ (100 mL), and extracted with CHCl₃ (3×200 mL). The combined organic extracts were washed with brine (2×100 mL) and dried (Na₂SO₄). After removal of solvent under reduced pressure, the residue was purified by flash chromatography (silica gel, hexanes/EtOAc: 2/1) to afford 56 (JYI-55, 794 mg, 47%) as a gray solid: mp 168.5-169.5° C.; IR(CHCl₃) 3202, 3113, 2955, 1686, 1612, 1490 cm⁻¹; ¹H NMR (CDCl₃) δ 0.22 (s, 9H), 4.38 (s, 2H), 7.04-7.33 (m, 3H), 7.34 (s, 1H), 7.45-7.53 (m, 1H), 7.56-7.62 (m, 2H), 8.73 (bs, 1H). MS (EI) m/e (relative intensity) 350 (94), 322 (100), 167 (41), 153 (37). Anal. Calcd. for C₂₀H₁₉N₂OFSi: C, 68.54; H, 5.46; N, 7.99. Found: C, 68.23; H, 5.40; N, 8.34.

7-Acetyleno-5-(2′-fluorophenyl)-1,3-dihydrobenzo[e]1,4-diazepine-2-one 57 (JYI-60). A solution of 56 (JYI-55, 700 mg, 2.0 mmol) in THF (200 mL) was treated with Bu₄NF (2 mL, 1.0M solution in THF). The mixture, which resulted, was allowed to stir for 5 min at room temperature after which the mixture was added to H₂O (5 mL) and extracted with EtOAc (3×10 mL). The combined organic extracts were washed with brine (2×10 mL) and dried (Na₂SO₄). After the solvent was removed under reduced pressure, the residue was purified by flash chromatography (silica gel, hexanes/EtOAc: 2/1) to afford 57 (JYI-60, 400 mg, 72%) as a pale yellow solid: mp 208-209.5° C.; IR(CHCl₃) 3290, 3110, 2930, 1685, 1612, 1489 cm⁻¹; ¹H NMR (CDCl₃) δ 3.04 (s, 1H), 4.40 (s, 2H), 7.06-7.28 (m, 3H), 7.38 (s, 1H), 7.44-7.51 (m, 1H), 7.59-7.62 (m, 2H), 9.43 (bs, 1H). MS (EI) m/e (relative intensity) 278 (80), 250 (100). Anal. Calcd. for C₁₇H₁₁N₂OF.: C, 73.37; H, 3.98; N, 10.07. Found: C, 73.64; H, 3.92; N, 9.78.

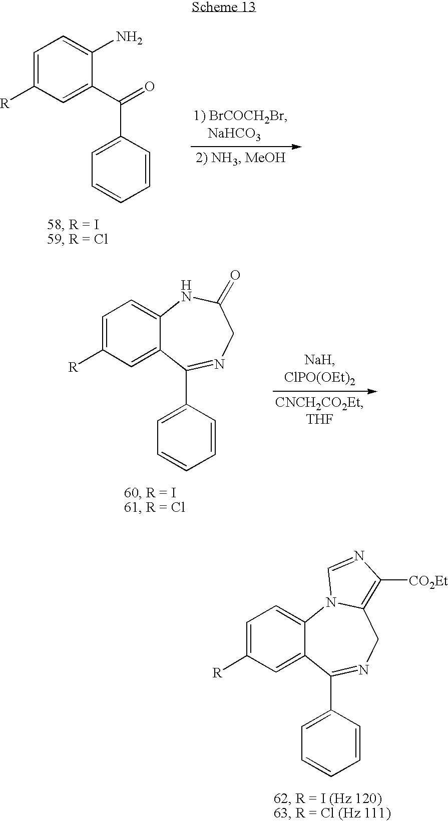

2-Amino-5-iodo-benzophenone 58 was prepared from p-iodonitrobenzene and phenylacetonitrile according to the literature. 2-Amino-5-chloro-benzophenone 59 was commercially available from Acros. The benzodiazepine 60 was reacted with diethylphosphorochloridate in the presence of sodium hydride, followed by the addition of ethyl isocyanoacetate to provide the ester 62 (Hz120), as shown in Scheme 13.

Ethyl 8-iodo-6-phenyl-4H-benzo[f]imidazo[1,5-a][1,4]diazepine-3-carboxylate 62. A solution of benzodiazepine 60 (3 g, 8.3 mmol) in dry THF (36 mL) was cooled to 0° C. and a 60% dispersion of sodium hydride (0.70 g, 17.4 mmol) was added in one portion. The mixture was allowed to warm to rt with stirring and the stirring was continued at rt until no more bubbles were evolved. The suspension was cooled to 0° C. after which diethylphosphorochloridate (2.29 g, 13.3 mmol) was added and this mixture was stirred for 30 min and allowed to warm to rt. The mixture was stirred for an additional 1.5 hr. In another flask, a 60% dispersion of sodium hydride (0.70 g, 17.4 mmol) in mineral oil was added in dry THF (36 mL) and cooled to 0° C. Ethyl isocyanoacetate (1.13 g, 9.94 mmol) was added and the stirring was continued until no more bubbles were evolved. This mixture was transferred to the above mixture at 0° C. The mixture was then stirred at rt for 6 h and quenched with HOAc (3.2 mL). The mixture was partitioned between EtOAc (200 mL) and H₂O (50 mL). The organic layer was washed with brine and dried (Na₂SO₄). After the solvent was removed under reduced pressure, the residue was purified by flash chromatography (silica gel, gradient elution, EtOAc:hexane 1:4, 1:1, 4:1) to provide the ester 62 (Hz120) in 43% yield as a light brown solid. mp: 221-222° C.; IR (KBr) 2977, 1717, 1608, 1489 cm⁻¹; ¹H NMR (DMSO-d₆) δ 1.31 (t, 3H, J=7.1 Hz), 4.10 (d, 1H, J=12.5 Hz), 4.29 (q, 2H, J=6.7 Hz), 5.75 (d, 1H, J=12.4 Hz), 7.40-7.50 (m, 5H), 7.63 (d, 1H, J=1.8 Hz), 7.69 (d, 1H, J=8.5 Hz), 8.13 (dd, 1H, J=1.9, 8.5 Hz), 8.36 (s, 1H); MS (EI) m/e (relative intensity) 458 (23), 457 (M⁺, 100), 411 (62), 384 (29), 383 (100), 257 (29). Anal. Calcd. for C₂₀H₁₆IN₃O₂: C, 52.53; H, 3.53; N, 9.19. Found: C, 52.57, H, 3.73; N, 8.64.

Ethyl 8-chloro-6-phenyl-4H-benzo[f]imidazo[1,5-a][1,4]diazepine-3-carboxylate 63. This ester 63 was obtained in 52% yield from 61 analogous to the procedure employed in the preceding paragraph as a white solid. mp: 174-175° C. (lit.¹² 174-175° C.); ¹H NMR (DMSO-d₆) δ 1.32 (t, 3H, J=7.1 Hz), 4.13 (d, 1H, J=12.3 Hz), 4.32 (q, 2H, J=6.7 Hz), 5.76 (d, 1H, J=12.3 Hz), 7.37-7.50 (m, 6H), 7.86-8.38 (m, 2H), 8.74 (s, 1H).

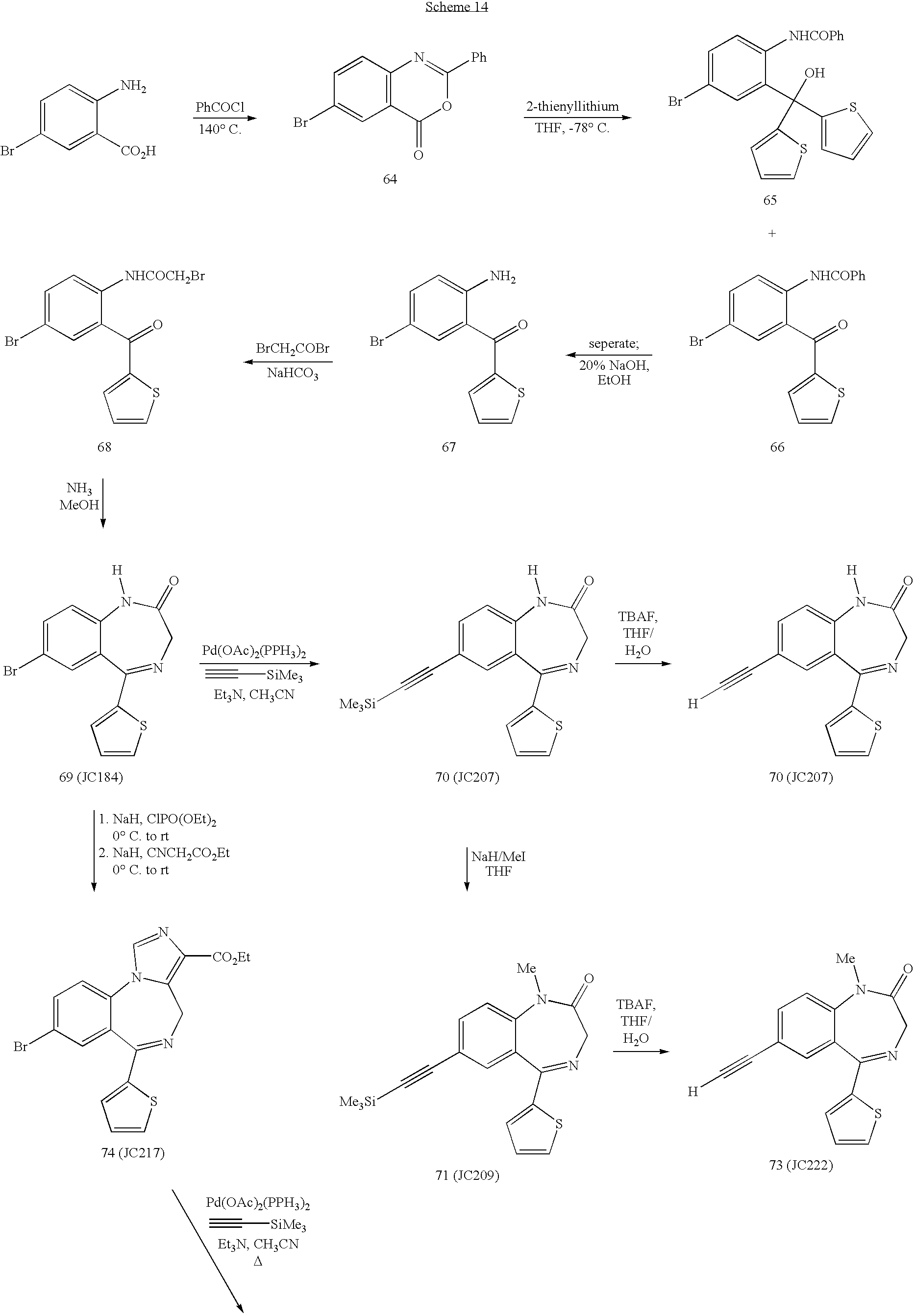

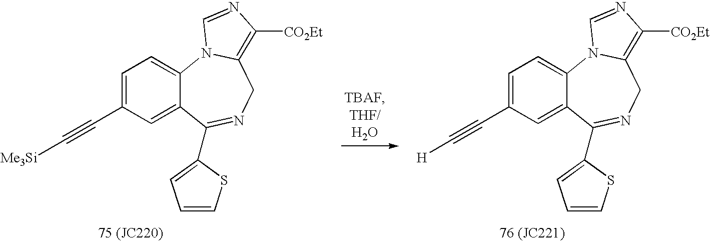

6-Bromo-2-phenyl-4H-benzo[2,3-d]-1,3-oxazin-4-one 64. The 2-amino-5-bromobenzoic acid (5 g, 23.1 mmol) was treated with benzoyl chloride (237 mL, 2.04 mol) at 140° C. for 3 h. After the reaction mixture was cooled to rt, the crystals that formed were collected by filtration and were washed with hexanes to provide 64 as light brown needles (6.8 g, 97%): ¹H NMR (CDCl₃) δ 7.51-7.2 (m, 4H), 7.9 (dd, 1H, J=2.3, 8.6 Hz), 8.30-8.33 (m, 2H), 8.8 (d, 1H, J=2.2 Hz); ¹³C NMR (CDCl₃) δ 158.19, 157.35, 145.75, 139.58, 132.82, 130.97, 129.77, 128.82, 128.73, 128.29, 121.37, 118.27; MS (EI) m/e (relative intensity) 303 (M⁺, 36), 301 (M⁺, 36), 259 (14), 257 (14), 226 (6), 224 (6), 178 (9), 170 (9), 168 (9), 151 (4), 105 (100).

4-Bromo-2-(2′-thienylcarbonyl)-N-benzoylaniline 66 and bis-(2′-thienyl)-[5-bromo-2-(N-benzoyl)-amino]phenylmethanol 65. The benzoxazinone 64 (5.0 g, 16.6 mmol) was dissolved in dry THF (250 mL) and cooled to −78° C. for 45 min. The 2-thienyllithium (18.21 mL of 1M solution in THF) was added dropwise over 35 min and the reaction was stirred at −78° C. for 1.2 h. Saturated aq NH₄Cl solution (25 mL) and Et₂O (30 mL) were then added. The organic layer was separated, washed with brine and dried (MgSO₄). The solvent was removed under reduced pressure, and the residue was purified via flash chromatography (silica gel, hexanes/EtOAc: 1:0, 49:1, 20:1, 11:1, 5:1) to provide 66 as yellow crystals and the alcohol 65. 66: ¹H NMR (CDCl₃) δ 7.23 (dd, 1H), 7.52-7.56 (m, 3H), 7.66 (dd, 1H, J=0.99, 3.8 Hz), 7.82 (d, 1H, J=5.0 Hz), 7.99-8.02 (m, 3H), 7.75 (d, 1H, J=9.0 Hz), 11.2 (s, 1H); ¹³C NMR (CDCl₃) δ 188.82, 165.45, 143.24, 138.79, 136.57, 135.90, 135.51, 134.25, 134.03, 132.17, 128.81, 128.31, 127.26, 125.65, 123.45, 114.95; MS (EI) m/e (relative intensity) 387 (M⁺, 12), 385 (M⁺, 12), 276 (18), 274 (18), 201 (7), 172 (7), 105 (100). 65: ¹H NMR (CDCl₃) δ 4.20 (s, 1H), 6.82 (s, 2H), 6.96-7.01 (m, 3H), 7.33-7.38 (m, 7H), 7.65 (d, 2H, J=7.23 Hz), 8.43 (d, 1H, J=8.8 Hz), 9.92 (s, 1H); ¹³C NMR (CDCl₃) δ 165.04, 148.94, 136.44, 135.49, 134.49, 132.34, 131.59, 131.40, 128.40, 127.20, 126.89, 126.58, 124.18, 116.00, 79.35, 76.92, 76.50; MS (EI) m/e (relative intensity) 471 (M⁺, 54), 469 (M⁺, 51), 453 (100), 451 (93), 348 (98), 346 (92), 316 (54), 314 (58), 282 (20), 280 (19), 267 (88), 235 (12), 234 (12), 223 (15), 222 (17), 201 (56), 173 (20), 172 (12), 158 (10), 129 (10).

5-Bromo-2-(2′-thienylcarbonyl)aniline 67. The amide 66 (2 g, 635 mmol) was dissolved in EtOH (150 mL) and 20% NaOH solution (30 mL) was added. The mixture was heated to reflux for 5 h and the EtOH was removed under reduced pressure. The mixture was extracted with EtOAc and the organic phases were combined, washed with brine and dried (Na₂SO₄). The solvent was removed under reduced pressure, and the residue was purified via a wash column (silica gel, hexanes/EtOAc: 11:1 to 4:1) to provide 67 as a bright yellow solid: ¹H NMR (DMSO-d₆) δ 6.28 (br s, 2H), 6.82 (s, 1H), 6.90 (s, 1H), 7.26 (dd, 1H, J=3.8, 5.0 Hz), 7.42 (dd, 1H, J=2.4, 8.9 Hz), 7.61 (dd, 1H, J=1.1, 3.8 Hz), 7.69 (dd, 1H, J=2.4 Hz), 8.04 (dd, 1H, J=1.1, 5.0 Hz); ¹³C NMR (DMSO) δ 187.42, 150.09, 143.87, 136.46, 134.75, 134.41, 133.93, 128.78, 119.36, 119.17, 104.95; MS (EI) m/e (relative intensity) 283 (M⁺, 59), 282 (M⁺, 87), 281 (M⁺, 59), 280 (M⁺, 79), 250 (23), 248 (23), 201 (13), 199 (49), 197 (48), 172 (25), 170 (23), 145 (13), 140 (1), 111 (100), 101 (33).

4-Bromo-2-(2′-thienylcarbonyl)-N-bromoacetylaniline 68. The thienylaniline 67 (3.3 g, 11.7 mmol) and NaHCO₃ (2.9 g, 34.5 mmol) were suspended in dry CHCl₃ (180 mL) and cooled to 0° C. A solution of bromoacetyl bromide (1.12 mL, 12.9 mmol) in dry CHCl₃ (30 mL) was added dropwise over 20 min at 0° C. and the mixture was stirred at rt for 3 h. The CHCl₃ solution was then washed with aq NaHCO₃ (5%) and dried (Na₂SO₄). The CHCl₃ was removed under reduced pressure, and Et₂O was added to the flask. The solution was sonicated and filtered to provide 68 as a light solid: mp: 144.0-146.5° C.; ¹H NMR (CDCl₃) δ 4.01 (s, 2H), 7.23-7.26 (m, 1H), 7.24 (d, 1H), 7.65 (d, 1H), 7.74 (d, 1H), 7.84 (d, 1H), 8.46 (d, 1H), 10.85 (br s, 1H); MS (EI) m/e (relative intensity) 405 (M⁺, 69), 404 (40), 403 (M⁺, 100), 401 (M+, 66), 324 (39), 322 (38), 310 (33), 308 (33), 292 (32), 283 (65), 282 (72), 281 (65), 280 (67), 266 (10), 264 (10), 250 (34), 248 (35), 226 (55), 224 (55), 201 (43), 199 (27), 197 (27), 173 (32), 111 (73).

7-Bromo-5-(2′-thienyl)-1,3-dihydrobenzo[e][1,4]diazepine 69 (JC184). The bromoacetyl amide 68 (0.236 g, 0.586 mmol) was dissolved in a saturated solution of anhydrous ammonia in MeOH (50 mL) and the mixture was heated to reflux for 6 h. After the MeOH was removed under reduced pressure, EtOAc was added to the residue. The solution was sonicated and then filtered to provide 69 (JC184) as a light solid: MS (EI) m/e (relative intensity) 322 (M⁺, 54), 320 (M⁺, 53), 294 (100), 292 (98), 211 (24), 185 (31), 140 (21). The material was used directly in the next step.

7-Trimethylsilylacetylenyl-5-(2′-thienyl)-1,3-dihydrobenzo[e][1,4]diazepine 70 (JC207). A mixture of 69 (1 g, 3.12 mmol) in CH₃CN (20 mL) and Et₃N (30 mL) was degassed and heated to reflux under nitrogen. Bis(triphenylphosphine)-palladium (II) acetate (0.26 g, 0.347 mmol) was then quickly added, followed by the addition of TMS acetylene (0.76 g, 7.78 mmol). The mixture was stirred at reflux for 4 h and the solvent was removed under reduced pressure. Water (25 mL) and EtOAc (25 mL) were added to the residue and the mixture was filtered through celite to remove the organometallic species. The filtrate was then extracted with EtOAc and the organic phases were combined, washed with brine and dried (Na₂SO₄). The solvent was removed under reduced pressure and the residue was purified via flash chromatography (silica gel, hexanes/EtOAc: 11:1, 5:1) to provide 70 (JC207) as a light yellow solid: mp: 198.5-201° C.; MS (EI) m/e (relative intensity) 338 (M⁺, 68), 337 (M⁺, 28), 310 (100), 295 (13), 161 (13), 147 (33), 105 (17). The material was used directly in the next step.

7-Acetylenyl-5-(2′-thienyl)-1,3-dihydrobenzo[e][1,4]diazepine 72(JC208). A solution of 70 (150 mg, 0.457 mmol) in THF (30 mL) was treated with tetrabutylammonium fluoride (1M in THF) at 0° C. for 5 minutes. Water (20 mL) was subsequently added to quench the reaction and the THF was removed under reduced pressure. The remaining aq solution was then extracted with EtOAc and the organic phases were combined, washed with brine and dried (Na₂SO₄). Upon removal of the solvent, Et₂O was added to the residue which was sonicated and then filtered to provide the title compound 72 (JC208, 111 mg, 91%) as an ivory colored solid: mp: 214-216° C.; MS (EI) m/e (relative intensity) 266 (M⁺, 61), 265 (M⁺, 30), 238 (100), 237 (49), 210 (13), 209 (10), 164 (6), 153 (7), 139 (7). This material was used in the next step.

1-N-methyl-7-trimethylsilylacetylenyl-5-(2′-thienyl)-1,3-dihydrobenzo[e][1,4]diazepine 71 (JC 209). Thiophene 70 (500 g, 1.52 mmol) was dissolved in dry THF (25 mL) at 0° C. and NaH (60% in mineral oil, 76 mg, 1.50 mmol) was added to the solution in one portion. After the mixture was stirred at 0° C. for 30 min, Me (0.14 mL, 2.25 mmol) was added and the ice bath was allowed to warm to rt. The mixture was allowed to stir for 3 h and the THF was then removed under reduced pressure. The residue was purified via flash chromatography (silica gel, hexanes/EtOAc 8:1, 4:1) to provide the title compound 71 (JC209) as a white solid: mp: 171.3-173.6° C.; ¹HNMR (CDCl₃) δ 0.26 (br s, 9H), 3.38 (s, 3H), 4.71 (d, 1H), 7.09 (dd, 1H, J=3.7, 5.0 Hz), 7.17 (dd, 1H, J=1.1, 3.7 Hz), 7.30 (s, 1H), 7.49 (dd, 1H, J=1.1, 5.0 Hz), 7.65 (dd, 1H, J=2.0, 8.5 Hz), 7.75 (d, 1H); ¹³C NMR (CDCl₃) δ(CDCl₃) δ 170.12, 163.22, 143.65, 143.14, 134.69, 133.12, 131.38, 130.14, 127.77, 127.47, 121.01, 119.10, 103.01, 95.66, 56.38, 34.67; MS (EI) m/e (relative intensity) 352 (M⁺, 71), 351 (M⁺, 60), 337 (10), 324 (100), 309 (24), 168 (28), 154 (38).

1-N-methyl-7-acetyleno-5-(2′-thienyl)-1,3-dihydrobenzo[e][1,4]diazepine 73 (JC 222). The same procedure for preparing 72 (JC208) was applied to 73 (JC222) and a very light brown solid resulted: mp: 218.3-220.4° C.; ¹H NMR (CDCl₃) δ 3.16 (s, 1H), 3.39 (s, 3H), 3.78 (d, 1H, J=11.07 Hz), 4.72 (d, 1H, J=5.9 Hz), 7.08 (dd, 1H, J=3.8, 5.0 Hz), 7.31 (d, 1H, J=8.6 Hz), 7.49 (dd, 1H, J=1.0, 5.0 Hz), 7.67 (dd, 1H, J=2.0, 8.5 Hz), 7.79 (d, 1H, J=1.9 Hz); ¹³C NMR (CDCl₃)□171.04, 170.07, 163.12, 143.49, 134.79, 133.50, 131.34, 130.25, 127.85, 127.46, 121.16, 117.99, 81.83, 78.30, 56.34, 34.69. MS (EI) m/e (relative intensity) 281 (13), 280 (M⁺, 60), 279 (51), 253 (19), 252 (100), 251 (2), 235 (11), 209 (10).

Ethyl 8-bromo-6-(2′-thienyl)-4H-benzo[f]imidazo[1,5-a][1,4]diazepine-3-carboxylate 74 (JC 217). Dry THF (30 mL) was added to a flask containing the benzodiazepine 69 (1.27 g, 3.96 mmol) and the solution was allowed to cool to 0° C. and NaH (60% in mineral oil, 0.191 g, 4.76 mmol) was quickly added. The mixture was stirred for 30 min at 0° C. and then removed from an ice bath to stir another 1 h at rt. Prior to adding ClPO(OEt)₂ (1.06 g, 6.35 mmol), the mixture was again pre-cooled to 0° C. The solution was stirred another 3 h as the ice bath warmed to rt. Meanwhile, dry THF (10 mL) was added to a second flask containing NaH (60% in mineral oil, 0.229 g, 5.72 mmol). After the second mixture was cooled to 0° C., CNCH₂CO₂Et was added dropwise and the solution continued to stir for 30 min at 0° C. After both reaction mixtures were again pre-cooled to 0° C., the two solutions were combined under Ar via cannula and the solution stirred at rt overnight. The reaction was quenched with ice water and worked up with EtOAc, and the combined organic phases were washed with brine and dried (Na₂SO₄). The solvent was removed under reduced pressure and the residue was purified via flash chromatography (silica gel, hexanes:EtOAc 4:1, 1:1, 1:3) to provide the title compound 74 (JC217) as an ivory solid (500 mg, 30% yield): mp: 204.0-205.3° C.; ¹H NMR (CDCl₃) δ 1.45 (t, 3H, J=7.1, 14.3 Hz), 4.07 (d, 1H, J=8.8 Hz), 4.44 (dd, 2H, J=3.8, 4.7 Hz), 5.98 (d, 1H, J=12.8 Hz), 7.05 (d, 1H, J=1.0 Hz), 7.07 (s, 1H), 7.46-7.49 (m, 2H), 7.83 (dd, 1H, J=2.2, 8.5 Hz), 7.91 (s, 1H), 7.96 (d, 1H, J=2.2 Hz): MS (EI) m/e (relative intensity) 418 (M⁺, 15), 417 (M⁺, 68), 416 (M⁺, 15), 415 (M⁺, 64), 407 (22), 344 (26), 343 (100), 342 (30), 341 (93), 293 (15), 291 (21), 262 (18), 235 (15), 211 (12), 154 (10), 127 (11).

Ethyl 8-trimethylsilylacetylenyl-6-(2-thienyl)-4H-benzo[f]imidazo[1,5-a][1,4]diazepine-3-carboxylate 75 (JC 220). The same procedure for preparing 70 (JC 207) was applied to 75 (JC 220) and an ivory colored solid resulted: ¹H NMR (CDCl₃) δ 0.29 (s, 9H), 1.45 (t, 3H, J=7.1, 14.3 Hz), 4.0 (d, 1H, J=18.1 Hz), 4.45 (dd, 2H, J=7.2, 8.5 Hz), 5.97 (d, 1H, J=12.8 Hz), 7.06-7.11 (m, 2H), 7.49 (dd, 1H, J=1.2, 5.0 Hz), 7.52 (d, 1H, J=8.3 Hz), 7.77 (dd, 1H, J=1.9, 8.3 Hz), 7.90 (d, 1H, J=1.8 Hz), 7.93 (s, 1H). MS (EI) m/e (relative intensity) 433 (M⁺, 74), 387 (49), 359 (100), 277 (28), 262 (19), 235 (24), 172 (19), 129 (17).

Ethyl 8-acetyleno-6-(2′-thienyl)-4H-benzo[f]imidazo[1,5-a][1,4]diazepine-3-carboxylate 76 (JC 221). The same procedure for preparing 72 (JC 208) was applied to 76 (JC 221) and an ivory colored solid resulted: mp: >198° C.; ¹H NMR (CDCl₃) δ 1.43 (t, 3H, J=4.3, 11.4 Hz), 3.25 (s, 1H), 4.10 (d, 1H, J=12.8 Hz), 4.40-4.49 (m, 2H), 5.99 (d, 1H, J=12.9 Hz), 7.50 (d, 1H, J=5.0 Hz), 7.56 (d, 1H, J=8.3 Hz), 7.81 (dd, 1H, J=1.8, 8.3 Hz), 7.95 (s, 1H); MS (EI) m/e (relative intensity) 361 (M⁺, 24), 315 (35), 287 (100), 237 (26), 178 (30), 153 (21), 126 (18).

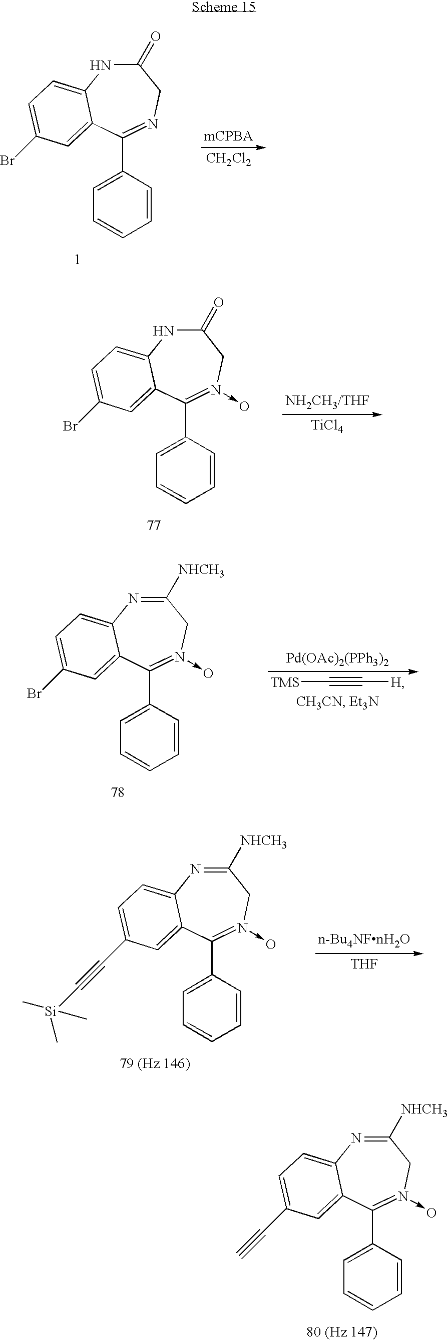

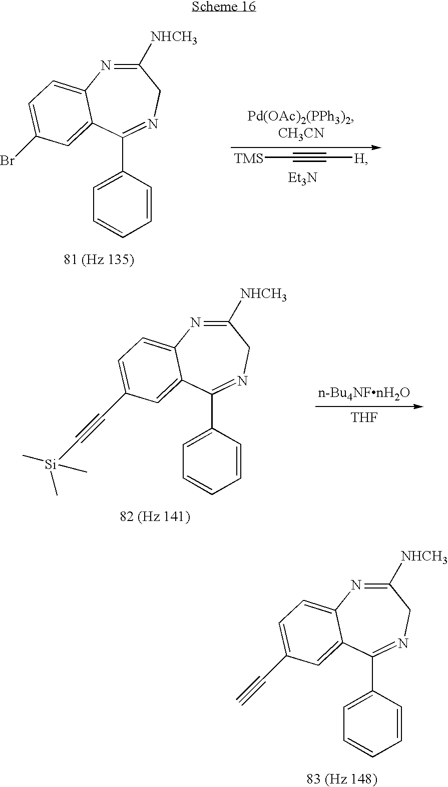

The benzodiazepine 1 was oxidized with 3-chloroperoxybenzoic acid (mCPBA) to form 77, followed by the addition of methylamine to afford amidine 78. N-Oxide 78 was reacted with trimethylsilylacetylene in the presence of a palladium catalyst to provide the trimethylsilyl analog 79 (Hz146) which was subjected to fluoride-mediated desilylation to afford 80 (Hz147), as shown in Scheme 15. In a related route, bromide 81 was converted into the trimethylsilylacetylene 82 (Hz141). This analog was then transformed into target 79 (Hz146) with mCPBA or the key target (Hz148) on treatment with fluoride (Scheme 16).

7-Bromo-4-oxy-5-phenyl-1,3-dihydro-benzo[e][1,4]diazepin-2-one 77. Bromide 1 (1.88 g, 5.95 mmol) was dissolved in CH₂Cl₂ (50 mL) and mCPBA (77% max) (1.76 g) was added at rt. The reaction mixture was stirred overnight. The mixture was diluted with CH₂Cl₂ (80 mL) and washed with a sat. solution of NaHCO₃ (50 mL), water (50 mL) and brine (40 mL). The organic layer was dried (Na₂SO₄) and concentrated. The residue was purified by flash chromatography (silica gel, EtOAc) to afford compound 77 in 90% yield as a white solid. mp: 230-231° C. (lit.¹³ 230-231° C.); ¹H NMR (CDCl₃) δ 4.69 (s, 2H), 7.16 (d, 1H, J=8.7 Hz), 7.24 (d, 1H, J=2.1 Hz), 7.45 (m, 3H), 7.54 (dd, 1H, J=8.6, 2.2 Hz), 7.64 (dd, 2H, J=7.3, 3.6 Hz), 10.02 (s, 1H).

(7-Bromo-4-oxy-5-phenyl-3H-benzo[e][1,4]diazepin-2-yl)-methyl-amine 78. Methylamine (50 mL, 2 M in THF) was added to 77 (1.9 g, 5.7 mmol) in a 100 mL round-bottom flask. The mixture was cooled to 0° C. after which TiCl₄ (0.54 g, 2.86 mmol) was added dropwise. The reaction mixture was allowed to warm to rt and stirred for 4 h. The mixture was quenched with water (5 mL), diluted with EtOAc (100 mL) and washed with dilute NH₄OH. The organic layer was washed with water, brine and dried (Na₂SO₄). After the solvent was removed under reduced pressure, the residue was purified by flash chromatography (silica gel, gradient elution, EtOAc, EtOAc:MeOH 10:1) to provide 78 in 86% yield as a white solid. mp: 236-237° C. (lit.¹⁴ 242-243° C.); ¹H NMR (300 MHz, CDCl₃) δ 0.21 (s, 9H), 2.91 (s, 3H), 4.17 (s, 1H), 4.85 (s, 1H), 7.13-7.66 (m, 9H).

(7-Trimethylsilylacetylenyl-4-oxy-5-phenyl-3H-benzo[e][1,4]diazepin-2-yl)-methyl-amine 79 (Hz146). Trimethylsilylacetylenyl analog 79 (Hz146) was obtained in 58% yield from 78 analogous to the procedure employed in Scheme 1 to produce 2 as a light gray solid. mp: 239-240° C.; IR (KBr) 3229, 3060, 2952, 2149, 1616, 1593, 1462, 1238, 868 cm⁻¹; ¹H NMR (300 MHz, CDCl₃) δ 2.89 (d, 3H, J=4.4 Hz), 4.14 (d, 1H, J=10.6 Hz), 4.78 (d, 1H, J=10.4 Hz), 7.15 (d, 1H, J=1.7 Hz), 7.24-7.28 (m, 2H), 7.45 (m, 4H), 7.66 (m, 2H); MS (EI) m/e (relative intensity) 361 (M⁺, 48), 344 (100), 303 (31), 165 (33).

(7-Acetylenyl-4-oxy-5-phenyl-3H-benzo[e][1,4]diazepin-2-yl)-methyl-amine 80 (Hz147). The 7-acetyleno target 80 was obtained in 90% yield from 79 analogous to the procedure employed in Scheme 1 to produce 4 as a light yellow solid. mp: 213-214° C.; IR (KBr) 3242, 3068, 2977, 1619, 1589, 1460, 1414 cm⁻¹; ¹H NMR (300 MHz, CDCl₃) δ 2.89 (d, 2H, J=3.7 Hz), 2.98 (s, 1H), 4.13 (bs, 1H), 4.78 (bs, 1H), 7.18-7.71 (m, 9H); MS (EI) m/e (relative intensity) 289 (M⁺, 47), 272 (100), 231 (42).

(7-Bromo-5-phenyl-3H-benzo[e][1,4]diazepin-2-yl)-methyl-amine 81 (Hz135). Bromide 81 was obtained in 70% yield from 1 analogous to the procedure employed in Scheme 15 as a white solid. mp: 234-235° C.; IR (KBr) 3253, 3076, 1609, 1571, 1415, 1326, 1230 cm⁻¹; ¹H NMR (300 MHz, CDCl₃) δ 2.62 (s, 3H), 3.56 (bs, 1H), 4.68 (bs, 1H), 6.34 (s, 1H), 7.17 (d, 1H, J=8.7 Hz), 7.36-7.81 (m, 7H); MS (EI) m/e (relative intensity) 329 (80), 328 (M⁺, 100), 327 (82), 326 (92), 220 (38), 219(48), 218(46), 205 (38).

(7-Trimethylsilylacetylenyl-5-phenyl-3H-benzo[e][1,4]diazepin-2-yl)-methyl-amine 82 (Hz141). Trimethylsilylacetylenyl analog 82 (Hz141) was obtained in 73% yield from 81 analogous to the procedure employed in Scheme 15 as a light yellow solid. mp: 210-211° C.; IR (KBr) 3257, 3079, 2956, 2150, 1619, 1610, 1580, 1416, 1237, 880, 843 cm⁻¹; ¹H NMR (300 MHz, CDCl₃) δ 0.22 (s, 9H), 2.59 (d, 3H, J=3.5 Hz), 3.56 (bs, 1H), 4.66 (bs, 1H), 6.39 (s, 1H), 7.21 (d, 1H, J=8.4 Hz), 7.39-7.65 (m, 7H); MS (EI) m/e (relative intensity) 345 (M⁺, 100), 344 (98), 164 (50).

(7-Acetylenyl-4-oxy-5-phenyl-3H-benzo[e][1,4]diazepin-2-yl)-methylamine 83 (Hz148). The 7-acetyleno analog 83 (Hz148) was obtained in 92% yield from 82 analogous to the procedure employed in Scheme 15 as a white solid. mp: 226-227° C.; IR (KBr) 3275, 3245, 3075, 2102, 1618, 1599, 1580, 1467, 1416, 1333, 1235 cm⁻¹; ¹H NMR (300 MHz, CDCl₃) δ 2.65 (d, 3H, J=4.4 Hz), 2.97 (s, 1H), 3.57 (bs, 1H), 4.65 (bs, 1H), 6.20 (d, 1H, J=3.7 Hz), 7.22 (d, 1H, J=8.4 Hz), 7.42-7.58 (m, 7H). MS (EI) m/e (relative intensity) 273 (M⁺, 100), 272 (98).

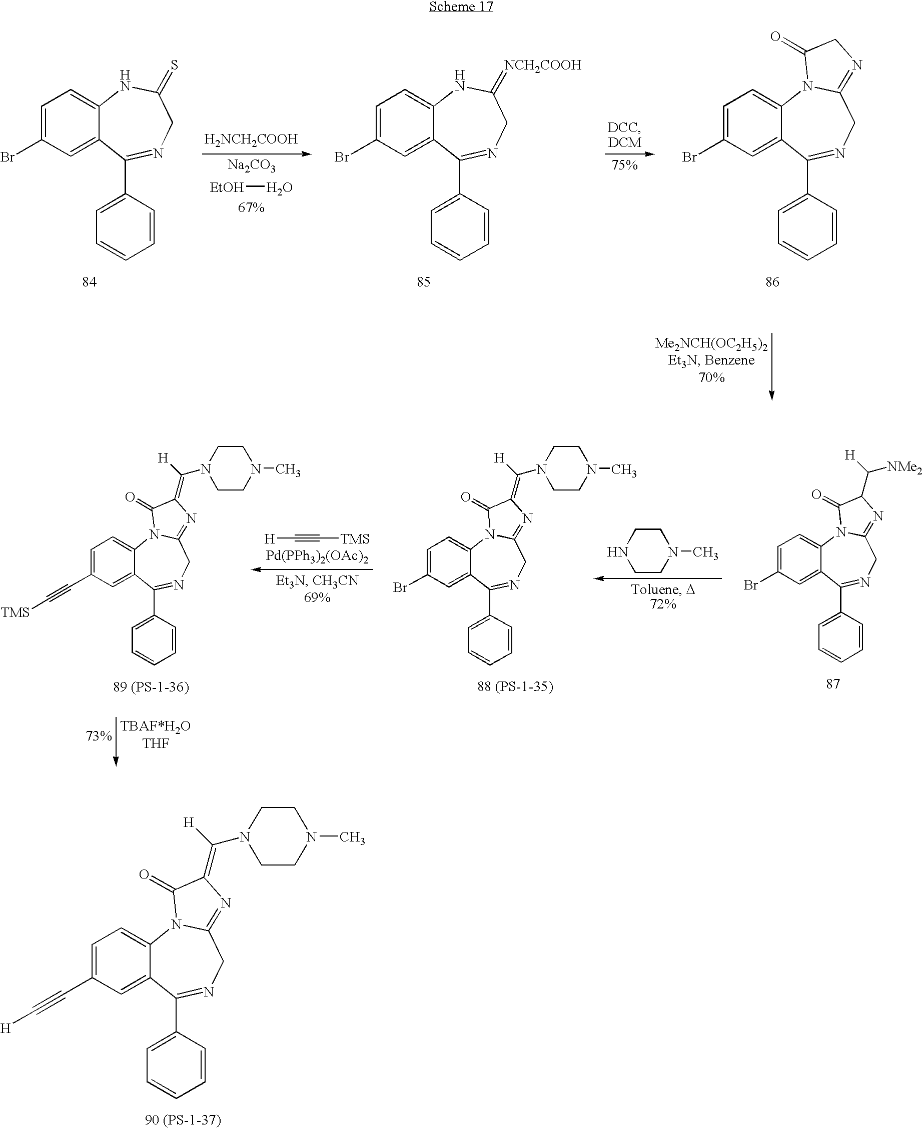

A suspension of 7-bromo-1,3-dihydro-5-phenyl-2H-1,4-benzodiazepin-2-thione 84 (1.6 g, 4.83 mmol), glycine (1.81 g, 24.2 mmol) and Na₂CO₃ (1.84 g, 17.4 mmol) in EtOH (38 mL)-H₂O (16 mL) was stirred at reflux for h, poured into water (100 mL), and then filtered to remove a small amount of 7-bromo-1,3-dihydro-5-phenyl-2H-1,4-benzodiazepin-2-one which remained. The filtrate was extracted with CHCl₃. The CHCl₃ extract was discarded; the aqueous layer was adjusted to pH 4 with 2N HCl and then extracted with CHCl₃ (3×25 mL). Evaporation of the CHCl₃ solution gave pure acid 85 (1.2 g, 67%) as a yellow solid. Acid 85 (350 mg, 0.941 mmol) was suspended in dry CH₂Cl₂ (10 mL) and DCC (223 mg, 1.08 mmol) was added. The suspension which resulted was stirred at 40° C. for 2 h and then cooled to 0° C. It was filtered, and the solvent was removed in vacuum to give 8-bromo-2,4-dihydro-6-phenyl-1H-imidazo[1,2-a][1,4]benzodiazepin-1-one 86 as a brown oil. The cyclized product 86 (ca. 250 mg) was dissolved in dry benzene (6 mL), dimethylformamide diethylacetal (130 mg, 0.883 mmol) and triethylamine (89 mg, 0.883 mmol) were added. The solution which resulted was stirred at room temperature for 1 h and the solvent was removed in vacuum, The residue was then crystallized from EtOAc-MeOH to give 87 (200 mg, 70%). A solution of 87 (180 mg, 0.440 mmol) in dry toluene (5 mL) was treated with 1-methyl piperazine (1 mL) and heated to reflux for 5 h. The solvent was removed in vacuum to give a gum which crystallized from CH₂Cl₂-Et₂O to furnish 88 (PS—I-35, 146 mg, 72%). mp>250° C.; IR (KBr) 3324, 2932, 2787, 1692, 1624, 1475, 1402, 1297, 1137, 933 cm⁻¹; ¹HNMR (CDCl₃) δ 7.95 (d, 1H, J=8.8 Hz), 7.72 (dd, 1H, J=2.3 Hz, J=8.8 Hz), 7.58-7.55 (m, 2H), 7.49-7.37 (m, 4H), 7.17 (s, 1H), 5.01 (d, 1H, J=12 Hz), 4.50-4.60 (m, 1H), 4.20-4.30 (m, 1H), 4.16 (d, 1H, J=12 Hz), 3.50-3.58 (m, 2H), 2.40-2.60 (m, 4H), 2.34 (s, 3H); MS (m/z) 465 (100).

To the suspension of compound 88 (PS—I-35, 140 mg, 0.302 mmol) in acetonitrile (4 mL) and triethylamine (3 mL) was added bis(triphenylphosphine)-palladium (II) acetate (22.6 mg, 0.03 mmol). The solution was degassed and trimethylsilylacetylene (0.1 mL, 0.7 mmol) was added. The mixture was heated to reflux and stirred overnight. After removal of the solvent in vacuum, the residue was dissolved in CH₂Cl₂ and washed with a saturated aqueous solution of NaHCO₃ and brine. The organic layer was dried (Na₂CO₃), filtered and concentrated under vacuum. The residue was purified by flash column chromatography (EtOAc:MeOH 9:1) to furnish the trimethylsilyl analogue 89 (PS—I-36, 100 mg, 69%) as a pale yellow solid. mp>250° C.; IR (KBr) 3436, 2936, 2794, 2154, 1682, 1625, 1489, 1136, 847 cm⁻¹; ¹H NMR (CDCl₃) δ 8.0 (d, 1H, J=8.5 Hz), 7.68 (dd, 1H, J=1.9 Hz, J=8.5 Hz), 7.55-7.59 (m, 2H), 7.37-7.49 (m, 4H), 7.16 (s, 1H), 4.99 (d, 1H, J=12 Hz), 4.50-4.60 (m, 1H), 4.20-4.30 (m, 1H), 4.13 (d, 1H, J=12.4 Hz), 3.48-3.58 (m, 2H), 2.4-2.6 (m, 4H), 2.35 (s, 3H), 0.23 (s, 9H); MS (m/z) 482 (100).

A solution of the trimethylsilyl analog 89 (PS—I-36, 65 mg, 0.135 mmol) in THF (15 mL) was stirred with tetrabutylammonium fluoride hydrate (45 mg, 0.175 mmol) at −5° C. for 5 min. After this, H₂O (5 mL) was added to the solution to quench the reaction and stirring continued at low temperature for one half hour. The solution was extracted with EtOAc (3×40 mL), and the organic layer was washed with water. After removal of the solvent under reduced pressure, ethyl ether was added to the residue to precipitate a solid. The mixture was filtered and the solid was washed with CHCl₃-Et₂O (ca 1:15) to provide the acetyl target 90 (PS—I-37, 40 mg, 73%). mp 223-224° C.; IR (KBr) 3298, 2935, 2786, 1695, 1628, 1364, 1136, 1002, 778 cm⁻¹; ¹H NMR (CDCl₃) δ 8.04 (d, 1H, J=8.5 Hz), 7.71 (dd, 1H, J=1.9 Hz, J=8.5 Hz), 7.55-7.58 (m, 2H), 7.36-7.48 (m, 4H), 7.17 (s, 1H), 5.0 (d, 1H, J=12.1 Hz), 4.5-4.6 (m, 1H), 4.2-4.3 (m, 1H), 4.16 (d, 1H, J=12.1 Hz), 3.5-3.6 (m, 2H), 3.08 (s, 1H), 2.4-2.6 (m, 4H), 2.35 (s, 3H); MS (m/z) (100).

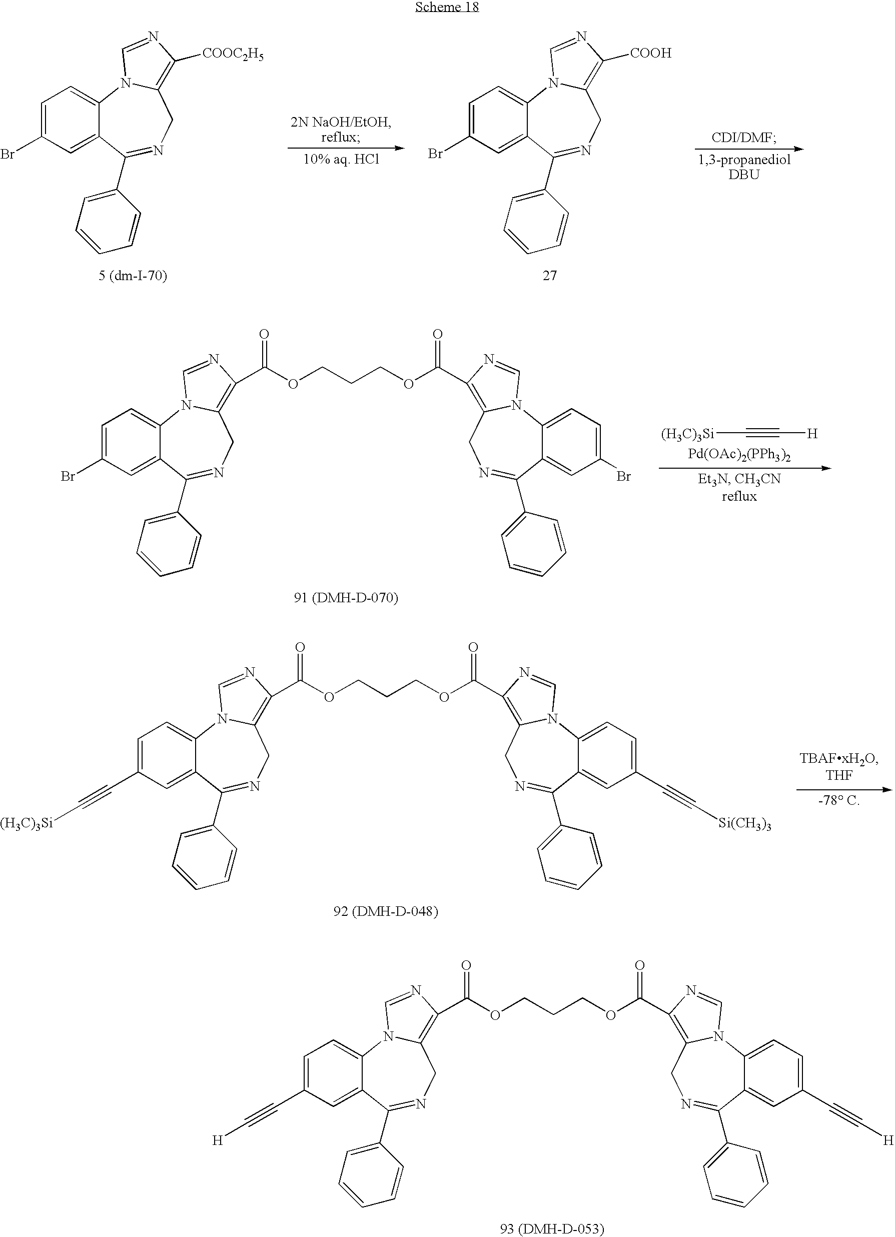

The acid 27, obtained from the ester 5 (dm-I-70), was stirred with CDI in DMF, followed by stirring with 1,3-propanediol and DBU to provide 91 (DMH-D-070, the dimer of dm-I-70). This was converted into the trimethylsilylacetylenyl compound 92 (DMH-D-048, the dimer of XLiXHe048) under standard conditions (Pd-mediated, Heck-type coupling). The bisacetylene 93(DMH-D-053, the dimer of XHeII-053) was easily obtained by treatment of trimethylsilyl compound 92 with fluoride anion as shown in Scheme 18.

8-Bromo-6-phenyl-4H-benzo[f]imidazo[1,5-a][1,4]diazepine-3-carboxylic acid 27. The ester 5 (2 g) was dissolved in EtOH (50 mL) and aq sodium hydroxide (10 mL, 2N) was added to the solution. The mixture was heated to reflux for half an hour. After the EtOH was removed under reduced pressure, the solution was allowed to cool. The pH value was adjusted to 4 by adding 10% aq HCl dropwise. The mixture was filtered and the solid was washed with water and ethyl ether. The solid was dried to provide 27 (1.8 g, 96.6%): mp>250° C.; IR (KBr) 3450 (b), 2844, 1707, 1615, 1493, 1166, 700 cm⁻¹, ¹H NMR (300 MHz, DMSO-d₆) δ 4.14 (d, 1H, J=12.6 Hz), 5.79 (d, 1H, 12.6 Hz), 7.41-7.54 (m, 6H), 7.88 (d, 1H, J=8.7 Hz), 8.03 (dd, 1H, J=8.7 Hz, J=2.1 Hz), 8.47 (s, 1H); MS (EI) m/e (rel intensity) 381 (M⁺, 20), 383 (19).

1,3-Bis(8-bromo-6-phenyl-4H-benzo[f]imidazo[1,5-a][1,4]diazepine-3-carb-oxy) propyl diester 91 (DMH-D-070). The carboxylic acid 27 (2 g, 5.2 mmol) was dissolved in DMF (20 mL), after which CDI (1.02 g, 6.3 mmol) was added at rt and the mixture was stirred for 2 h. Then 1,3-propanediol (0.19 mL, 2.6 mmol) and DBU (0.78 mL, 5.2 mmol) were added to the mixture and stirring continued overnight. The reaction solution was then cooled with an ice-water bath, after which water was added to precipitate a solid. This material was purified further by flash chromatography on silica gel (gradient elution, EtOAc:EtOH 20:1, 15:1, 10:1) to provide the bisbromide 91 (DMH-D-070) as a white solid (1.3 g, 61.9%): mp 187.5-189° C.; IR (KBr) 3112, 2968, 1708, 1610, 1559, 1491, 1269, 1160, 1123, 1073 cm⁻¹; ¹H NMR (300 MHz, CDCl₃) δ 2.35 (m, 2H), 4.08 (d, 2H, J=12.6 Hz), 4.55 (m, 4H), 6.05 (d, 2H, J=12.6 Hz), 7.37-7.53 (m, 12H), 7.6 (d, 2H, J=2.1 Hz), 7.81 (dd, 2H, J=2.1 Hz, 8.6 Hz), 7.93 (s, 2H); ¹³C NMR (75.5 MHz, CDCl₃) δ 28.2, 44.9, 61.4, 120.7, 124.2, 128.3, 129.0, 129.3, 129.6, 130.6, 134.1, 134.4, 134.7, 135.0, 138.9, 138.9, 162.6, 167.9; MS (FAB, NBA) m/e (rel intensity) 803 (M⁺+1, 15); Anal. Calcd. For C₃₉H₂₈N₆O₄Br₂: C, 58.23; H, 3.51; N, 10.45. Found: C, 57.92; H, 3.43; N, 10.29.

1,3-Bis(8-trimethylsilylacetylenyl-6-phenyl-4H-benzo[f]imidazo[1,5-a][1,4]-diazepine-3-carboxy) propyl diester 92 (DMH-D-048). To a suspension of bisbromide 91 (1.005 g, 1.25 mmol) in acetonitrile (50 mL) and triethylamine (65 mL), was added bis(triphenylphosphine)-palladium (II) acetate (0.15 g, 0.2 mmol). The solution was degassed and trimethylsilylacetylene (0.7 mL, 5 mmol) was added after which it was degassed again. The mixture was heated to reflux and stirring maintained overnight. After removal of the solvent under reduced pressure, the residue was dissolved in CH₂Cl₂ and washed with water. 3-Mercaptopropyl functionalized silica gel (0.6 g) was added into the organic layer and stirring continued for 1 hour. The silica gel/Pd complex was removed by filtration and the filtrate was concentrated under reduced pressure. The residue was purified by flash column chromatography on silica gel (gradient elution, EtOAc:EtOH 20:1, 15:1, 10:1) to furnish the bistrimethylsilyl dimer 92 (DMH-D-048, 680 mg, 60.8%) as a white solid: mp 169-172° C.; IR (KBr) 3449, 2950, 1725, 1720, 1715, 1496, 1250, 1160, 1080, 847 cm⁻¹; ¹H NMR (300 MHz, CDCl₃) δ 0.25 (s, 18H), 2.35 (m, 2H), 4.05 (d, 2H, J=12.6 Hz), 4.55 (m, 4H), 6.02 (d, 2H, J=12.6 Hz), 7.37-7.55 (m, 14H), 7.75 (dd, 2H, J=1.8 Hz, 8.4 Hz), 7.94 (s, 2H); ¹³C NMR (75.5 MHz, CDCl₃) 8-0.3, 28.3, 44.9, 61.4, 97.4, 102.3, 122.4, 122.6, 128.0, 128.3, 129.0, 129.4, 130.5, 134.1, 134.9, 135.1, 139.0, 139.2, 139.2, 162.6, 168.5; MS (FAB, NBA) ink (rel intensity) 839 (M⁺+1, 100); Anal. Calcd. For C₄₉H₄₆N₆O₄Si₂: C, 70.14; H, 5.53; N, 10.02. Found: C, 69.97; H, 5.35; N, 9.77.

1,3-Bis(8-acetylenyl-6-phenyl-4H-benzo[f]imidazo[1,5-a][1,4]diazepine-3-carboxy) propyl diester 93 (DMH-D-053). A solution of bistrimethylsilyl dimer 92 (330 mg, 0.4 mmol) in THF (70 mL) was stirred with tetrabutylammonium fluoride hydrate (250 mg, 0.96 mmol) at −78° C. for 5 min. After this, H₂O (35 mL) was added to the solution to quench the reaction and stirring continued at low temperature for one half hour. The solution was extracted with EtOAc (3×100 mL), and the organic layer was washed with water. After removal of the solvent under reduced pressure, ethyl ether was added to the residue to precipitate a solid. The mixture was filtered and the solid was washed with CHCl₃-Et₂O (ca 1:15), the bisacetylenyl dimer 93 (DMH-D-053, 220 mg, 80%) was obtained as a yellow solid: mp 172-175° C.; IR (KBr) 3450, 3280, 2950, 1720, 1715, 1495, 1250, 1120, 1050 cm⁻¹; ¹H NMR (300 MHz, CDCl₃) δ 2.35 (m, 2H), 3.18 (s, 2H), 4.08 (d, 211, J=12.3 Hz), 4.56 (m, 4H), 6.04 (d, 2H, J=12.6 Hz), 7.36-7.59 (m, 14H), 7.78 (dd, 2H, J=8.4 Hz, 1.7 Hz), 7.95 (s, 21-1); ¹³C NMR (75.5 MHz, CDCl₃) δ 28.8, 45.4, 61.9, 80.2, 81.3, 121.4, 122.7, 128.1, 128.3, 129.0, 129.3, 130.5, 134.2, 135.2, 135.3, 135.6, 138.9, 139.2, 162.6, 168.5; MS (FAB, NBA) m/e (rel intensity) 695 (M⁺+1, 100).

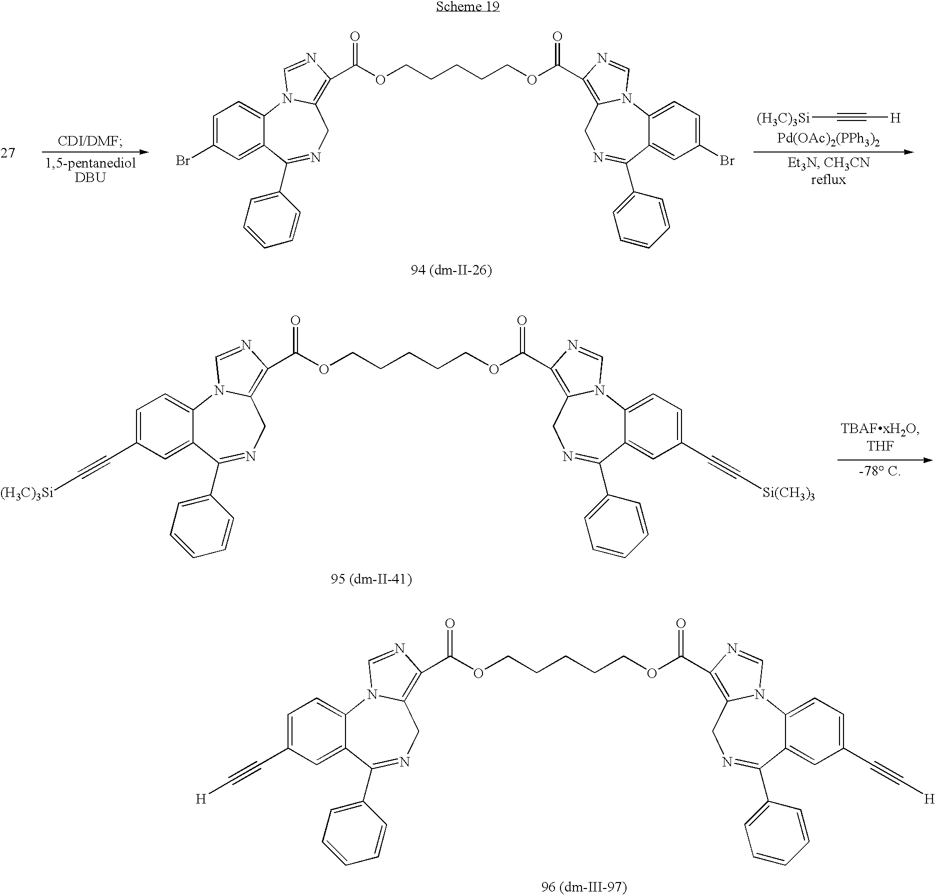

The 5-carbon linker bisbromide 94 (dm-II-26), bis-trimethylsilylacetylenyl dimer 95 (dm-II-41) and bisacetylene dimer 96 (dm-III-97), which are analogues of dimers DMH-D-070, DMH-D-048 and DMH-D-053, respectively, were prepared from acid 27 under the same conditions employed for preparing dimers 91 (DMH-D-070), 92 (DMH-D-048) and 93 (DMH-D-053), respectively, by using 1,5-pentanediol in place of 1,3-propanediol (Scheme 19).

1,5-Bis(8-bromo-6-phenyl-4H-benzo[f]imidazo[1,5-a][1,4]diazepine-3-carb-oxy) pentyl diester 94 (dm-H-26). A yellow powder (63.2%): mp 172-175° C.; IR (KBr) 3112, 2970, 1721, 1609, 1490, 1267, 1158, 1075, 754, 697 cm⁻¹; ¹H NMR (300 MHz, CDCl₃) δ 1.62 (m, 2H), 1.90 (m, 4H), 4.07 (d, 2H, J=12.6 Hz), 4.39 (m, 4H), 6.05 (d, 2H, J=12.6 Hz), 7.37-7.53 (m, 12H), 7.58 (d, 2H, J=2.1 Hz), 7.78 (dd, 2H, J=2.1 Hz, 8.6 Hz), 7.92 (s, 2H); ¹³C NMR (75.5 MHz, CDCl₃) δ 22.5, 28.4, 44.9, 64.5, 120.7, 124.2, 128.3, 129.2, 129.3, 129.6, 130.6, 134.0, 134.5, 134.6, 135.0, 138.8, 138.9, 162.8, 167.9; MS (FAB, NBA) m/e (rel intensity) 831 (M⁺+1, 5). Anal. Calcd. For C₄₁H₃₂N₆O₄Br₂.0.25H₂O: C, 58.95; H, 3.89; N, 10.07; Found: C, 58.69; H, 3.74; N, 9.70.

1,5-Bis(8-trimethylsilylacetylenyl-6-phenyl-4H-benzo[f]imidazo[1,5-a][1,4]-diazepine-3-carboxy) pentyl diester 95 (dm-II-41). A yellow solid (58.1%): mp 154-156° C.; IR (KBr) 3426, 2955, 1727, 1720, 1612, 1495, 1251, 1174, 1076, 846 cm⁻¹; ¹H NMR (300 MHz, CDCl₃) δ 0.25 (s, 18H), 1.63 (m, 2H), 1.90 (m, 4H), 4.05 (d, 2H, J=12.6 Hz), 4.39 (m, 4H), 6.03 (d, 2H, J=12.6 Hz), 7.40-7.54 (m, 14H), 7.75 (dd, 2H, J=1.8 Hz, 8.4 Hz), 7.93 (s, 2H); ¹³C NMR (75.5 MHz, CDCl₃) δ −0.3, 22.5, 28.4, 44.9, 64.5, 97.4, 102.3, 122.4, 122.6, 128.0, 128.3, 129.2, 129.4, 130.5, 134.1, 135.0, 135.1, 135.1, 138.9, 139.3, 162.8, 168.5; MS (FAB, NBA) m/e (rel intensity) 867 (M⁺+1, 100).

1,5-Bis(8-acetylenyl-6-phenyl-4H-benzo[f]imidazo[1,5-a][1,4]diazepine-3-carboxy) pentyl diester 96 (dm-III-97). A yellow solid: mp 150-153° C.; IR (KBr) 3290, 2953, 1718, 1611, 1493, 1253, 1172, 1120, 1076 cm⁻¹; ¹H NMR (300 MHz, CDCl₃) δ 1.62 (m, 2H), 1.90 (m, 4H), 3.18 (s, 2H), 4.07 (d, 2H, J=12.3 Hz), 4.38 (m, 4H), 6.04 (d, 2H, J=12.3 Hz), 7.36-7.58 (m, 14H), 7.77 (dd, 2H, J=8.4 Hz, 1.6 Hz), 7.94 (s, 2H); ¹³C NMR (75.5 MHz, CDCl₃) δ 22.5, 28.4, 44.9, 64.5, 79.8, 81.3, 121.3, 122.7, 128.1, 128.3, 129.2, 129.3, 130.5, 134.1, 135.2, 135.3, 135.6, 138.8, 139.2, 162.8, 168.5; MS (FAB, NBA) m/e (rel intensity) 723 (M⁺+1, 13).

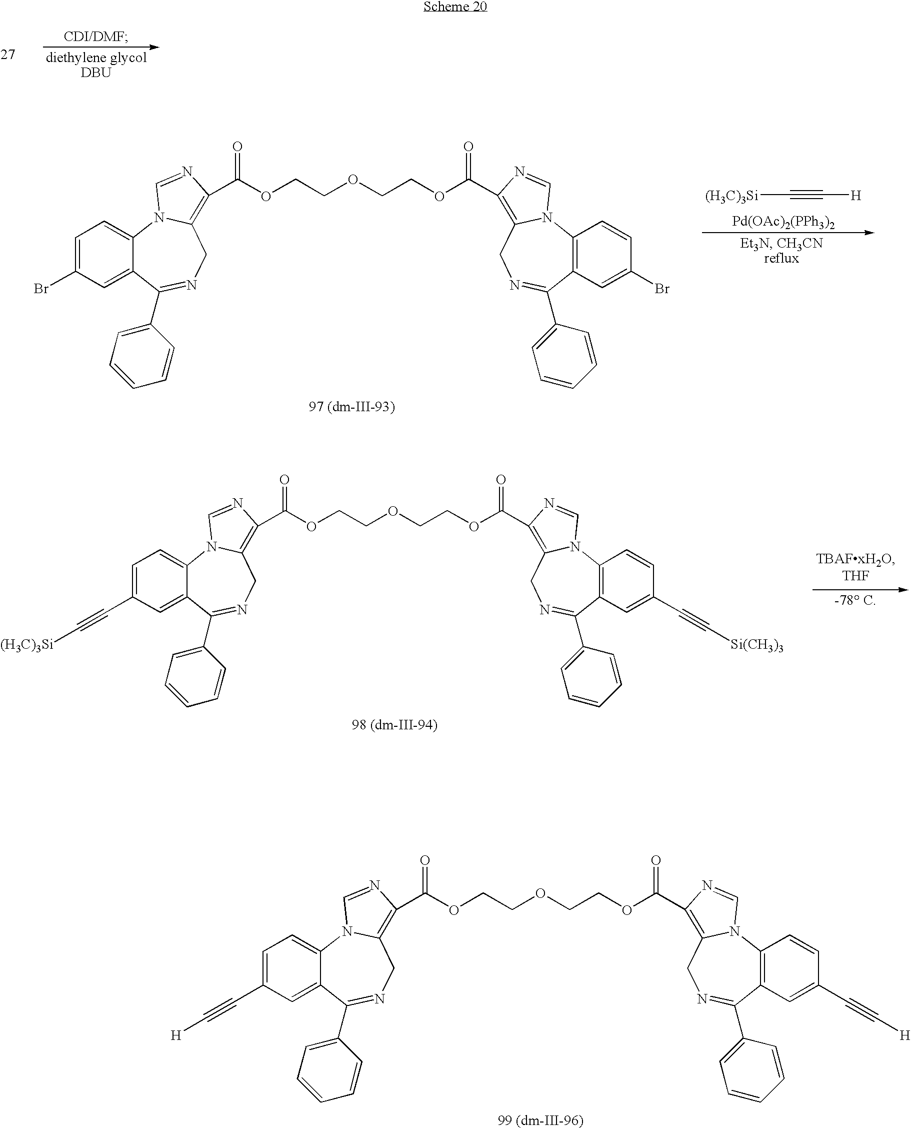

In order to improve the water solubility of the dimers, the oxygen-containing 5-atom linked dimers 97 (dm-III-93), 98 (dm-III-94) and 99 (dm-III-96), were designed and prepared from acid 27 under the same conditions employed for preparation of dimers DMH-D-070, DMH-D-048 and DMH-D-053, respectively, by replacing 1,3-propanediol with diethylene glycol (Scheme 20).

Bis(8-bromo-6-phenyl-4H-benzo[f]imidazo[1,5-a][1,4]diazepine-3-carboxy) diethylene glycol diester 97 (dm-III-93). A yellow solid (93.7%): mp 165-168° C.; IR (KBr) 3060, 2956, 1725, 1610, 1558, 1491, 1267, 1161, 1123, 1074 cm⁻¹; ¹H NMR (300 MHz, CDCl₃) δ 3.93 (t, 4H, J=4.8 Hz), 4.06 (d, 2H, J=12.6 Hz), 4.54 (m, 4H), 6.05 (d, 2H, J=12.6 Hz), 7.39-7.50 (m, 12H), 7.57 (d, 2H, J=2.7 Hz), 7.80 (dd, 2H, J=2.1 Hz, 8.4 Hz), 7.90 (s, 2H); ¹³C NMR (75.5 MHz, CDCl₃) δ 44.9, 63.6, 69.0, 120.7, 124.2, 128.3, 129.0, 129.3, 129.6, 130.6, 134.1, 134.4, 134.6, 135.0, 138.9, 139.0, 162.5, 167.9; MS (FAB, NBA) m/e (rel intensity) 833 (M⁺+1, 5).

Bis(8-trimethylsilylacetylenyl-6-phenyl-4H-benzo[f]imidazo[1,5-a][1,4]diazepine-3-carboxy) diethylene glycol diester 98 (dm-III-94). A yellow solid (49.5%): mp 205-208° C.; IR (KBr) 3433, 2960, 1730, 1700, 1612, 1493, 1255, 1169, 1120, 1071, 847 cm⁻¹; ¹H NMR (300 MHz, CDCl₃) δ 0.25 (s, 18H), 3.93 (t, 4H, J=5.4 Hz), 4.04 (d, 2H, J=12.6 Hz), 4.55 (m, 4H), 6.04 (d, 2H, J=12.6 Hz), 7.37-7.53 (m, 14H), 7.74 (dd, 2H, J=1.2 Hz, 8.4 Hz), 7.91 (s, 2H); ¹³C NMR (75.5 MHz, CDCl₃) δ −0.3, 45.0, 63.6, 69.0, 97.5, 102.4, 122.5, 122.7, 128.1, 128.3, 129.0, 129.4, 130.5, 134.2, 135.0, 135.1, 135.2, 139.1, 139.3, 162.7, 168.6; MS (FAB, NBA) m/e (rel intensity) 869 (M⁺+1, 100).

Bis(8-acetylenyl-6-phenyl-4H-benzo[f]imidazo[1,5-a][1,4]diazepine-3-carb-oxy) diethylene glycol diester 99 (dm-III-96). A yellow solid (81.6%): mp 173-177° C.; IR (KBr) 3432, 3280, 1720, 1715, 1496, 1254, 1175, 1120, 1074 cm⁻¹; ¹H NMR (300 MHz, CDCl₃) δ 3.12 (s, 2H), 3.93 (t, 4H, J=4.5 Hz), 4.06 (d, 2H, J=12.6 Hz), 4.55 (m, 4H), 6.05 (d, 2H, J=12.6 Hz), 7.38-7.56 (m, 1411), 7.75 (dd, 2H, J=8.4 Hz, 1.8 Hz), 7.91 (s, 2H); ¹³C NMR (75.5 MHz, CDCl₃) δ 45.0, 63.6, 69.0, 79.8, 81.3, 121.3, 122.7, 128.1, 128.3, 129.0, 129.3, 130.5, 134.2, 135.2, 135.3, 135.6, 139.0, 139.1, 162.6, 168.4; MS (FAB, NBA) m/e (rel intensity) 725 (M⁺+1, 63).

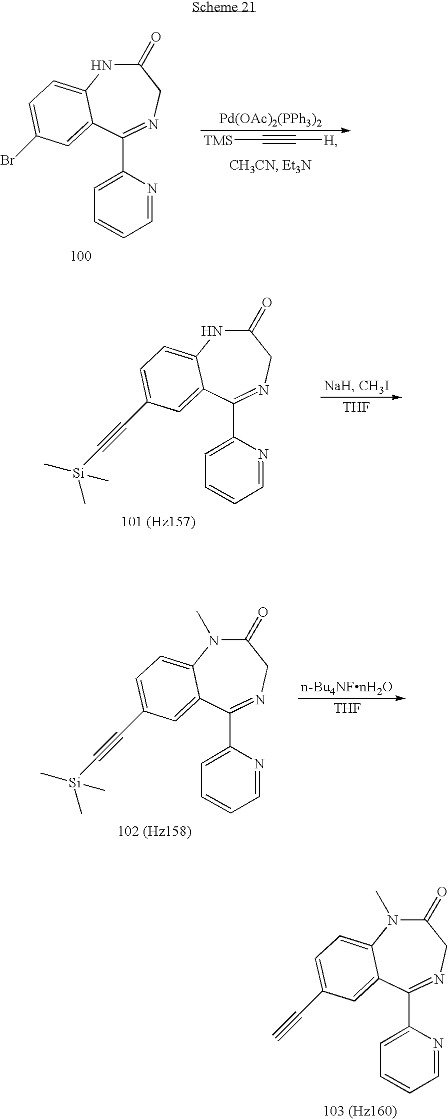

The benzodiazepine 100 (bromazepam) was reacted with trimethylsilyacetylene in the presence of a palladium catalyst to provide trimethylsilyl analog 101 (Hz157) that was methylated with methyl iodide/sodium hydride to afford analog 102 (Hz158). This was subjected to fluoride-mediated desilylation to achieve analog 103 (Hz160).

7-Trimethylsilylacetylenyl-5-pyridin-2-yl-1,3-dihydro-benzo[e][1,4]diazepin-2-one 101 (Hz157). Trimethylsilylacetylenyl analog 101 (Hz157) was obtained in 76% yield from 100 analogous to the procedure employed above as a light gray solid. mp: 242-243° C.; IR (KBr) 2956, 2155, 1690, 1616, 1492, 1332, 1248, 1018, 842, 754 cm⁻¹; ¹H NMR (300 MHz, CDCl₃) δ 0.23 (s, 9H), 4.39 (s, 2H), 7.06 (d, 1H, J=8.4 Hz), 7.41 (ddd, 1H, J=7.5, 4.8, 1.2 Hz), 7.46 (d, 1H, J=1.8 Hz), 7.57 (dd, 1H, J=8.4, 1.9 Hz), 7.83 (td, 1H, J=7.7, 1.7 Hz), 7.97 (d, 1H, J=7.9 Hz), 8.41 (bs, 1H), 8.68 (d, 1H, J=4.2 Hz); MS (EI) m/e (relative intensity) 334 (35), 333 (M⁺, 100), 332 (57), 318 (21), 304 (31).

7-Trimethylsilylacetylenyl-1-methyl-5-pyridin-2-yl-1,3-dihydro-benzo[e][1,4]diazepin-2-one 102 (Hz158). Trimethylsilylacetylenyl analog 102 (Hz158) was obtained in 74% yield from 101 analogous to the procedure employed above as a light grey solid. mp: 194-195° C.; IR (KBr) 2956, 2154, 1682, 1614, 1491, 1335, 1249, 881, 844, 747 cm⁻¹; ¹H NMR (300 MHz, CDCl₃) δ 0.24 (s, 9H), 3.42 (s, 3H), 3.84 (d, 1H, J=10.6 Hz), 4.89 (d, 1H, J=10.6 Hz), 7.29 (d, 1H, J=7.6 Hz), 7.40 (m, 1H), 7.46 (d, 1H, J=1.9 Hz), 7.63 (dd, 1H, J=8.5, 1.9 Hz), 7.84 (td, 1H, J=7.7, 1.7 Hz), 8.09 (d, 1H, J=7.9 Hz), 8.68 (d, 1H, J=4.3 Hz); MS (EI) m/e (relative intensity) 348 (28), 347 (M⁺, 100), 346 (44), 318 (34), 291 (23).

7-Acetylenyl-1-methyl-5-pyridin-2-yl-1,3-dihydro-benzo[e][1,4]diazepin-2-one 103 (Hz160). The 7-acetyleno analog 103 (Hz160) was obtained in 63% yield from 102 analogous to the procedure employed above as a white solid. mp: 190-191° C.; IR (KBr) 3286, 3233, 1678, 1614, 1491, 1344, 1126, 750 cm⁻¹; ¹H NMR (300 MHz, CDCl₃) δ 3.07 (s, 1H), 3.86 (d, 1H, J=10.6 Hz), 4.93 (d, 1H, J=10.2 Hz), 7.32 (d, 1H, J=8.6 Hz), 7.39 (m, 1H), 7.51 (d, 1H, J=1.8 Hz), 7.65 (dd, 1H, J=8.5, 1.9 Hz), 7.83 (td, 1H, J=7.7, 1.7 Hz), 8.11 (d, 1H, J=7.9 Hz), 8.65 (d, 1H, J=4.7 Hz); MS (EI) m/e (relative intensity) 275 (M⁺, 100), 274 (35), 246 (43), 219 (30).

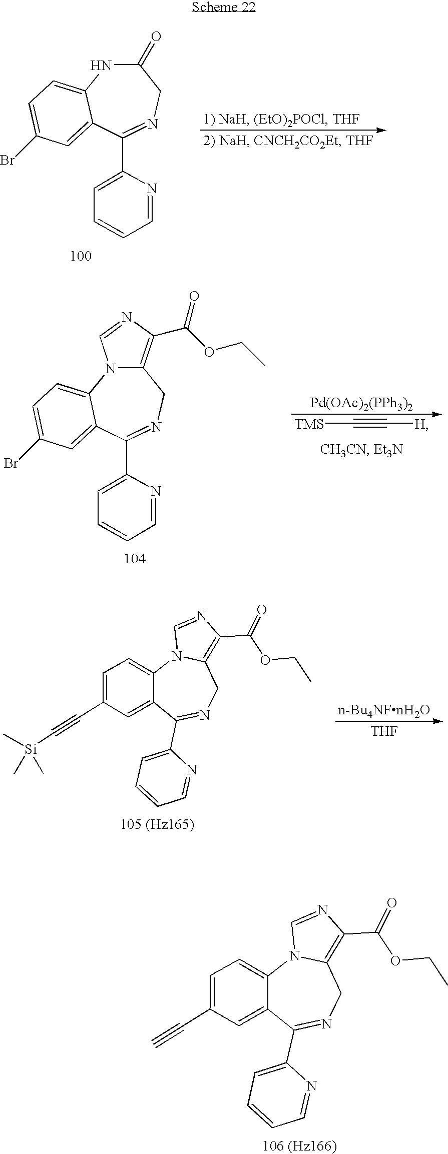

The benzodiazepine 100 (bromazepam) was reacted with diethylphosphorochloridate, followed by the addition of ethyl isocyanoacetate to provide the ester 104. This was then reacted with trimethylsilyacetylene in the presence of a palladium catalyst to provide trimethylsilyl analog 105 (Hz165) which was subjected to fluoride-mediated desilylation to furnish analog 106 (Hz166).

8-Trimethylsilylacetylenyl-6-pyridin-2-yl-4H-benzo[f]imidazo[1,5-a][1,4]diazepine-3-carboxylic acid ethyl ester 105 (Hz165). Trimethylsilyacetylenyl analog 105 (Hz165) was obtained in 73% yield from 104 analogous to the procedure employed in Scheme 1 as a white solid. mp: 205-206° C.; ¹H NMR (300 MHz, CDCl₃) δ 0.25 (s, 9H), 1.44 (t, 3H, J=7.1 Hz), 4.14 (d, 1H, J=11.0 Hz), 4.44 (m, 2H), 6.11 (d, 1H, J=10.9 Hz), 7.38 (ddd, 1H, J=7.5, 4.8, 1.1 Hz), 7.51 (s, 1H), 7.54 (d, 1H, J=8.4 Hz), 7.74 (dd, J=8.3, 1.8 Hz), 7.83 (td, 1H, J=7.7, 1.7 Hz), 7.93 (s, 1H), 8.05 (m, 1H), 8.61 (m, 1H).

8-Acetylenyl-6-pyridin-2-yl-4H-benzo[f]imidazo[1,5-a][1,4]diazepine-3-carboxylic acid ethyl ester 106 (Hz166). The 7-acetyleno analog 106 (Hz166) was obtained in 98% yield from 105 analogous to the procedure employed in Scheme 1 as a white solid. mp: 243-244° C.; ¹H NMR (300 MHz, CDCl₃) δ 1.45 (t, 3H, J=7.1 Hz), 3.17 (s, 1H), 4.17 (d, 1H, J=10.0 Hz), 4.45 (m, 2H), 6.13 (d, 1H, J=10.4 Hz), 7.38 (ddd, 1H, J=7.5, 4.8, 1.1 Hz), 7.56 (d, 1H, J=8.2 Hz), 7.58 (s, 1H), 7.77 (dd, 1H, J=8.6, 1.8 Hz), 7.83 (td, 1H, J=7.7, 1.8 Hz), 7.93 (s, 1H), 8.08 (m, 1H), 8.59 (m, 1H).

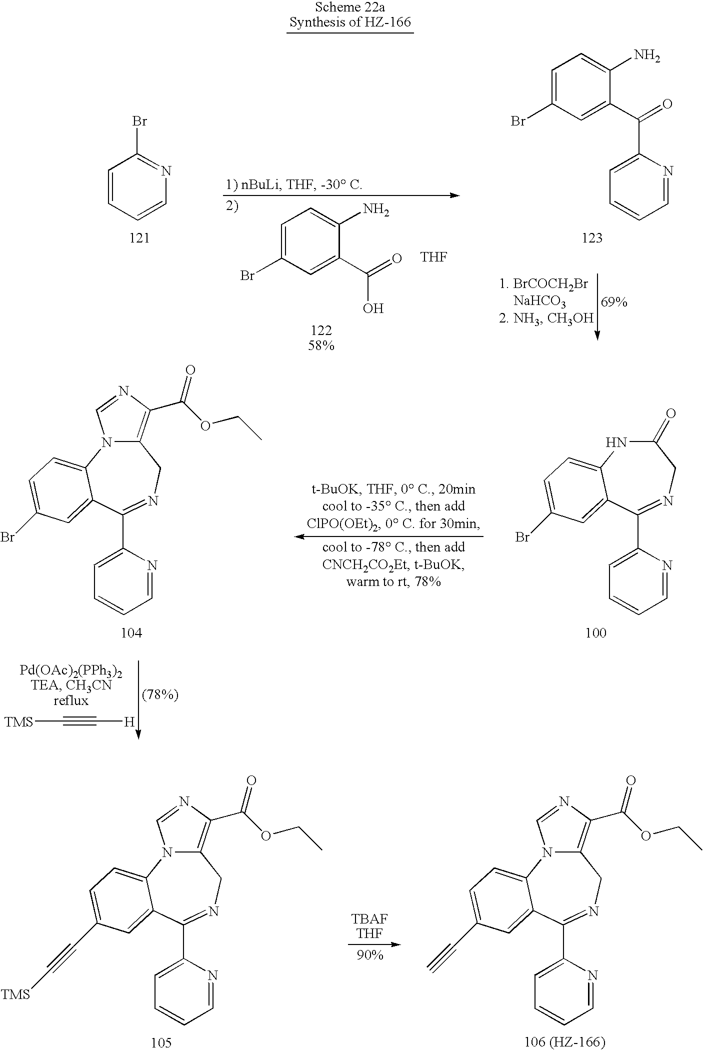

Alternative Process For The Synthesis Of HZ-166 (106): The 7-bromo-5-(pyridin-2-yl)-1H-benzo[e][1,4]diazepin-2(3H)-one 100 was stirred with potassium t-butoxide and diethylphosphorochloridate and this was followed by addition of ethyl isocyanoacetate to provide benzimidazo intermediate 104, as illustrated in Scheme 22a. This is a new process which is much easier to carry out in much higher yield (78% vs 38% for the old process). This material was heated with trimethysilylacetylene in a Heck-type coupling reaction to provide the trimethylsilyl analog 105, as illustrated in Scheme 22a. The silyl group was removed from 105 on treatment with fluoride anion to furnish 106 (HZ-166) in good yield.

Ethyl-8-bromo-6-(pyridin-2-yl)-4H-benzo[f]imidazo[1,5-a][1,4]diazepine-3-carboxylate (104). A solution of 100 (18.0 g, 56.96 mmol) in THF (1.424 L) was cooled to 0° C., and potassium t-butoxide (7.40 g, 62.66 mmol) was added to it in one portion. After stirring for 20 minutes at 0° C., the reaction was cooled to −35° C. and diethyl chlorophosphate (12.77 g, 74.05 mmol) was added slowly. After stirring at 0° C. for 30 min, the reaction mixture was cooled to −78° C. and ethyl isocyanoacetate (7.46 g, 62.66 mmol) was added followed by potassium t-butoxide (7.40 g, 62.66 mmol). After stirring at ambient temperature for 4 hours, the reaction was quenched with a saturated aqueous solution of NaHCO₃ (1.9 L) and extracted with EtOAc. The combined organic layers were dried (Na₂SO₄), concentrated and crystallized from ether to give 104 as a solid. The mother liquor was further purified by flash chromatography (silica gel, hexanes/EtOAc: 2/1) to afford 104 (combined yield, 17.79 g, 78%) as a white solid: mp 209-211° C.; ¹H NMR (300 MHz, CDCl₃) δ 1.44 (t, 3H, J=7.1 Hz), 4.13 (d, 1H, J=10.0 Hz), 4.45 (m, 2H), 6.11 (d, 1H, J=10.4 Hz), 7.28 (t, 1H, J=7.7, 2.1 Hz), 7.55 (d, 1H, J=31 Hz), 7.60 (d, 1H, J=2.2 Hz) 7.83 (td, 2H, J=7.7, 1.8 Hz), 7.90 (s, 1H), 8.09 (d, 1H, J=7.9 Hz), 8.57 (d, 1H, J=4.11 Hz). MS (ET) m/e (relative intensity) 411 (25), 410 (78); Anal. Calcd. for C₁₉H₁₅BrN₄O₂; C, 55.49; H, 3.68; N, 13.62. Found: C, 55.54; H, 3.71; N, 13.64.

Ethyl-6-(pyridin-2-yl)-8-((trimethylsilyl)ethynyl)-4H-benzo[f]imidazo[1,5-a][1,4]diazepine-3-carboxylate (105). A mixture of bromide 104 (10 g, 24.3 mmol), and bis(triphenylphosphine)-palladium (II) acetate (1 g, 1.33 mmol) was dissolved in a mixed solvent system of CH₃CN (625 mL) and triethylamine (525 mL). The mixture was degassed under vacuum and argon gas. The process was repeated three more times, after which trimethylsilylacetylene (4.5 mL, 31.8 mmol) was added into the mixture and this solution was then heated to reflux under argon. After stirring overnight at reflux, the mixture was cooled to room temperature and the precipitate which formed was removed by filtration through celite. The celite was washed with ethyl acetate. The combined filtrate was concentrated under reduced pressure and the residue was purified by flash chromatography (silica gel, hexanes/EtOAc: 3/1) to afford 105 (8.12 g, 78%) as a white solid: mp 205-206° C.; ¹H NMR (300 MHz, CDCl₃) δ 0.25 (s, 9H), δ 1.45 (t, 3H, J=7.1 Hz), 4.17 (d, 1H, J=10.0 Hz), 4.45 (m, 2H), 6.13 (d, 1H, J=10.4 Hz), 7.38 (ddd, 1H, J=7.5, 4.8, 1.1 Hz), 7.55 (dd, 2H, J=8.6, 1.8 Hz), 7.83 (td, 2H, J=7.7, 1.8 Hz), 7.92 (s, 1H), 8.06 (d, 1H, J=7.9 Hz), 8.56 (d, 1H, J=4.11 Hz). MS (EI) m/e (relative intensity) 428 (100), 382 (34), 354 (75); Anal. Calcd. for C₂₄H₂₄N₄O₂Si: C, 67.26; H, 5.64; N, 13.07; Found: C, 67.31; H, 5.68; N, 13.11.

Ethyl-8-ethynyl-6-(pyridin-2-yl)-4H-benzo[f]imidazo[1,5-a][1,4]diazepine-3-carboxylate (106, HZ-166). A solution of trimethylsilyl intermediate 105 (6 g, 1.4 mmol) in THF (1000 mL) was cooled to −78° C. and treated with solid Bu₄NF.H₂O (4.42 g, 1.68 mmol). The mixture which resulted was allowed to warm and continuously monitored by TLC for disappearance of starting material. The reaction temperature was not allowed to rise above −10° C. The reaction was quenched by slow addition of H₂O (500 mL) and extracted with EtOAc (3×800 mL). The combined organic extracts were washed with brine (2×500 mL) and dried (Na₂SO₄). The solvent was removed under reduced pressure and the residue was purified by flash chromatography (silica gel, EtOAc) to afford 106 (4.49 g, 90%) as a white solid: mp 243-244° C.; ¹H NMR (300 MHz, CDCl₃) δ 1.45 (t, 3H, J=7.1 Hz), 3.17 (s, 1H), 4.17 (d, 1H, J=10.0 Hz), 4.45 (m, 2H), 6.13 (d, 1H, J=10.4 Hz), 7.38 (ddd, 1H, J=7.5, 4.8, 1.1 Hz), 7.55 (dd, 2H, J=8.6, 1.8 Hz), 7.83 (td, 2H, J=7.7, 1.8 Hz), 7.92 (s, 1H), 8.06 (d, 1H, J=7.9 Hz), 8.56 (d, 1H, J=4.11 Hz). MS (EI) m/e (relative intensity) 356 (100), 310 (16), 282 (13), 254 (14), 200 (14); Anal. Calcd. for C₂₁H₁₆N₄O₂, C, 70.77; H, 4.53; N, 15.72. Found: C, 70.81; H, 4.55; N, 15.70.

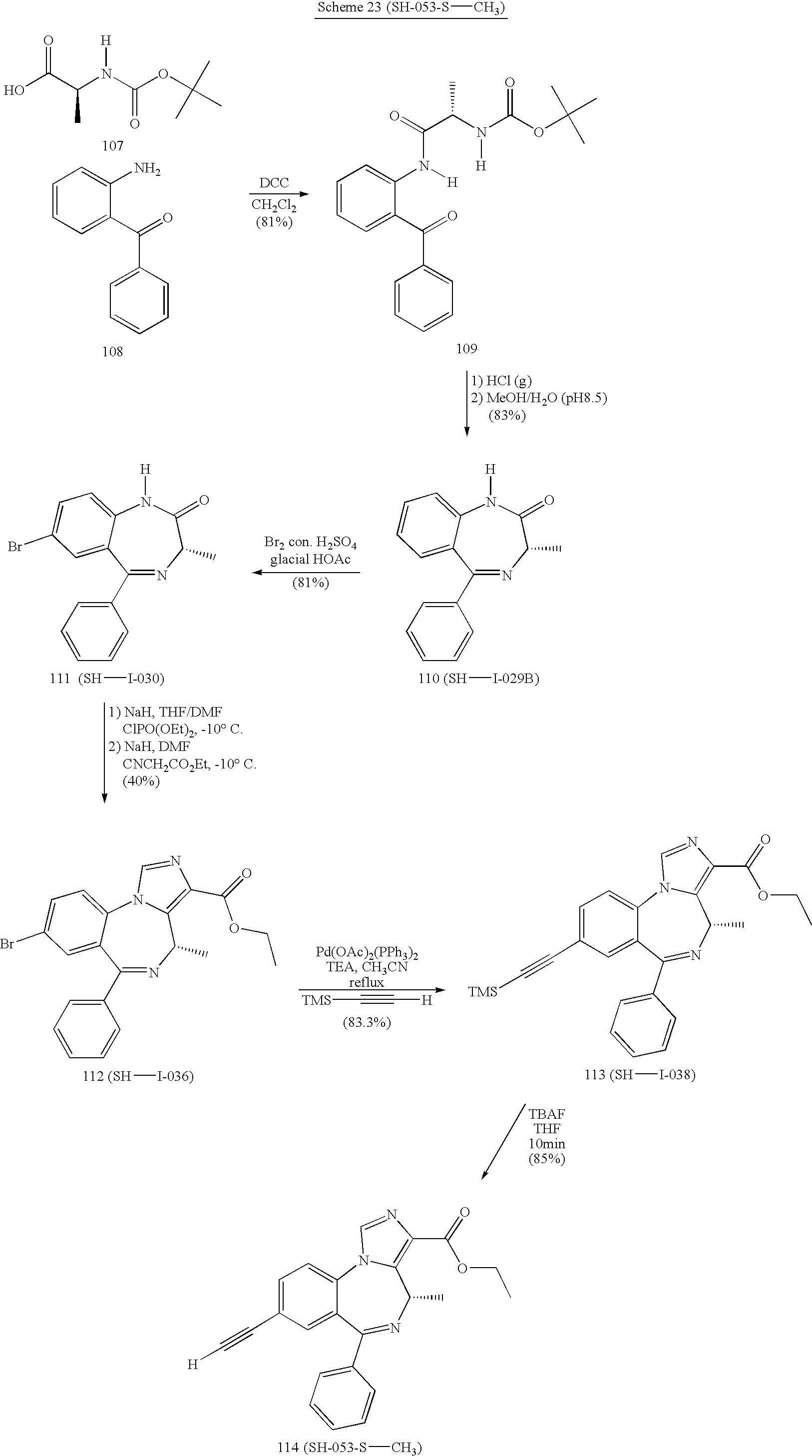

The benzophenone 108 was reacted with N—BOC-L-alanine 107 to give [1-(2-benzoyl-phenylcarbamyol)-ethyl]-carbamic acid tert-butyl ester 109. See Bradley, R., et al., (2000) J. Am. Chem. Soc. 122: 460-465. This ester was treated with HCl(g) in CHCl₃ and then cyclized under basic conditions to give benzodiazepine 110. This amide 110 was regioselectively brominated at position-7 to give bromide 111. The bromide 111 was stirred with diethylphosphorochloridate in the presence of sodium hydride, followed by addition of ethyl isocyanoacetate to provide the ethyl ester 112 (SH—I-036). This was converted into the trimethylsilylacetylene analog 113 (SH—I-038) under standard conditions (Pd-mediated, Heck-type coupling). Treatment of 113 with fluoride anion gave the title compound 114 (SH-053-S—CH₃). The other analogs were prepared via the same process.

### Procedure for SH-053-S—CH₃ (114):

[1-(2-Benzoyl-phenylcarbamoyl)-ethyl]-carbamic acid tert-butyl ester 109. To a stirred solution of 2-amino-5-bromobenzophenone 108 (5.73 g, 29.07 mmol) and the N-Boc-L-alanine 107 (5 g, 26.43 mmol) in CH₂Cl₂ (200 mL) was added dicyclohexylcarbodiimide (DCC) (5.99 g, 29.07 mmol) in CH₂Cl₂ (100 mL) dropwise, over 30 min at 0° C. The reaction mixture was stirred an additional 8 h at rt. The dicyclohexyl urea which formed was filtered off and the filtrate concentrated under reduced pressure. The crude solid 109 was purified by recrystallization from hexane to afford 109 (7.88 g, 81%), mp 127-129° C.; IR (KBr, cm⁻¹) 3288, 2475, 2352, 1684, 1636, 1576, 1507, 1447, 1264, 1165, 700; ¹H NMR (CDCl₃) δ 11.48 (s, 1H), 8.67 (d, J=8.22 Hz, 1H), 7.71-7.43 (m, 7H), 7.13-7.08 (m, 1H) 5.06 (br s, 1H), 4.36 (br s, 1H), 1.50 (d, J=7.1 Hz, 3H), 1.44 (s, 9H); MS (EI) m/e (relative intensity) 368 (M⁺, 6), 295 (10), 225 (27), 224 (79), 197 (83), 196 (77), 167 (15), 145 (46), 144 (88), 126 (17), 105 (38), 88 (94), 77(37), 57 (100); [α]²⁶_{D}=−67.7 (c 0.88, EtOAc).

3-Methyl-5-phenyl-1,3-dihydro-benzo[e][1,4]diazepin-2-one 110. To a stirred solution of the benzophenone 109 (10.65 g 29.38 mmol) in CHCl₃ (400 mL) at rt, hydrogen chloride gas was added in slowly. After 20 min, the addition was stopped and the solution was stirred overnight at rt. The reaction mixture was washed with a saturated solution of sodium bicarbonate (2×50 mL) and water (2×50 mL). The organic layer was concentrated under reduced pressure. The residual oil was dissolved in methanol-water (2:1, 500 mL) and the pH was adjusted to 8.5 by the addition of aqueous sodium hydroxide (1 N). The reaction mixture was stirred for 10 h at rt. The solution was concentrated under reduced pressure and water (100 mL) was added. The solution was extracted with CH₂Cl₂ (3×100 mL) and concentrated under reduced pressure. The crude solid 110 was purified by recrystallization from methanol/water to provide 110 (6.10 g, 83%). mp 160-162° C.; IR (KBr, cm⁻¹) 3215, 3059, 2974, 2932, 1681, 1574, 1478, 1445, 1372, 1321, 1242, 1160, 1131; ¹H NMR (CDCl₃) δ 9.65 (s, 1H), 7.54-7.13 (m, 9H), 3.78 (q, J=6.5 Hz, 1H), 1.78 (d, J=7.1 Hz 3H); MS (EI) m/e (relative intensity) 250 (M⁺, 40), 249 (83), 234 (15), 209 (75), 208 (76), 207 (100), 180 (17), 152 (19) 103 (23), 77 (40); [α]²⁶_{D}=290.2 (c 0.78, EtOAc).

7-Bromo-3-methyl-5-phenyl-1,3-dihydro-benzo[e][1,4]diazepin-2-one 111. To a stirred solution of 110 (66.5 g, 265 mmol) in glacial acetic acid (400 mL), sulfuric acid (80 mL) was added to the solution. Bromine (28 mL, 530 mmol) dissolved in acetic acid (150 mL) was added dropwise into the mixture. The reaction mixture was allowed to stir until NMR spectroscopy indicated that all of the starting material 110 had been consumed. The solution was concentrated under reduced pressure and then neutralized by addition of 1N NaOH solution and then extracted with EtOAc. The crude product 111 was purified by recrystallization from CH₂Cl₂ to afford 111 (70.4 g, 81%). mp 210-212° C.; IR (KBr, cm⁻¹) 2931, 1692, 1603, 1563, 1475, 1445, 1375, 1259, 1235, 1130, 1090; ¹H NMR (CDCl₃) δ 9.36 (s, 1H), 7.63 (dd, J=2.22, 8.58 Hz, 1H), 7.54-7.39 (m, 6H), 7.12 (d, J=8.61 1H), 3.76 (q, J=6.3 Hz, 1H), 1.76 (d, J=6.45 Hz, 3H). MS (EI) m/e (relative intensity) 330 (M⁺+1, 21), 329 (M⁺, 50), 328 (22), 327 (48), 289 (44), 288 (46), 287 (100), 286 (49), 285 (60), 205 (25), 77 (49); [α]²⁶_{D}=313.1 (c 0.34, EtOAc).

8-Bromo-4-methyl-6-phenyl-4H-2,5,10b-triaza-benzo[e]azulene-3-carboxylic acid ethyl ester 112. 7-Bromo-3-methyl-5-phenyl-1,3-dihydrobenzo[e][1,4]diazepin-2-one 111 (16.6 g, 0.052 mol) was suspended in dry THF (250 mL) and cooled to −10° C. Sodium hydride (60% dispersion in mineral oil, 4.36 g, 0.109 mol) was added into the suspension in one portion. The reaction mixture was allowed to stir and warm to rt over a 3 h period. The reaction mixture was again cooled to −10° C. and diethyl chlorophosphate (12.7 ml, 0.09 mol) was added. The cooling bath was then removed and stirring continued for 3 h. At this time, sodium hydride (60% dispersion in mineral oil, 4.2 g, 0.1 mol) was suspended in dry THF (250 mL) at −10° C. in another flask. Ethyl isocyanoacetate (6.78 mL, 0.06 mol) was added to the NaH/THF suspension, the solution which resulted was allowed to stir for 3 h. After 3 h the first flask was cooled to −30° C. and the solution in the 2nd reaction flask was added to the first flask via a cannula. The reaction mixture was allowed to stir for 24 h, and then cooled with an ice-water bath and slowly quenched with acetic acid (10 mL). Water was added to the reaction mixture after which it was extracted with EtOAc. The EtOAc layers were combined, washed with aq NaHCO₃, brine and dried (Na₂SO₄). After removal of the solvent under reduced pressure, the solid was purified by flash chromatography (silica gel, EtOAc:hexane, gradient elution 1:2, 1:1, 2:1). The ester 112 was a white solid (8.76 g, 40%). mp 164-165° C.; IR (KBr, cm⁻¹) 2925, 1706, 1622, 1557, 1495, 1266, 1185; ¹H NMR (CDCl₃) δ 7.89 (s, 1H), 7.73 (dd, J=1.73 Hz, 1H), 7.51-7.36 (m, 7H), 6.66 (q, J=7.30, 1H), 4.45-4.30 (m, 2H), 1.40 (t, J=7.11, 3H), 1.25 (d, J=7.38 Hz, 3H). MS (EI) m/e (relative intensity) 426 (M⁺+2, 15), 425 (M⁺+1, 58), 424 (M⁺, 15), 423 (58), 380 (24), 379 (71), 378 (35), 377 (69), 352 (50), 351(100), 350 (67), 349 (92), 270 (38), 229 (16); [α]²⁶_{D}=−38.0 (c 0.45, EtOAc).

4-Methyl-6-phenyl-8-trimethylsilanylethynyl-4H-2,5,10b-triazabenzo[e]azulene-3-carboxylic acid ethyl ester 113. A mixture of ester 112 (3.0 g, 7.07 mmol) and bis(triphenylphosphine)palladium(II) acetate (0.42 g, 0.57 mmol) was dissolved in a mixed solvent system of acetonitrile (80 mL) and TEA (120 mL). The mixture was degassed under vacuum and argon gas was added, after which trimethylsilylacetylene (2 mL, 14.14 mmol) was added into the mixture. The mixture was degassed again under vacuum (argon) and heated to reflux. The mixture was heated at reflux until analysis by TLC (EtOAc) indicated that all of the starting material 112 had been consumed. The mixture was cooled to rt and the precipitate which formed was removed by filtration through celite. The filtrate was concentrated under reduced pressure and partitioned between H₂O and EtOAc. The combined layers of EtOAc were washed with brine and dried (Na₂SO₄), after which the solvent was removed under reduced pressure and the residue was purified by flash chromatography (silica gel, EtOAc:hexane 1:1). Conditions for TLC were EtOAc. A white solid 113 (2.60 g, 83.3%) was obtained. mp 160-162° C.; IR (KBr, cm⁻¹) 3365, 2925, 1706, 1616, 1553, 1498; ¹H NMR (CDCl₃) δ 7.89 (s, 1H), 7.73 (dd, J=1.73 Hz, 1H), 7.51-7.36 (m, 7H), 6.66 (q, J=7.30, 1H), 4.45-4.30 (m, 2H), 1.40 (t, J=7.11, 3H), 1.25 (d, J=7.38 Hz, 3H), 0.15 (s, 9H). MS (EI) m/e (relative intensity) 441 (M⁺, 15), 369 (25), 323 (55), 295 (100), 267 (15).

8-Ethynyl-4-methyl-6-phenyl-4H-2,5,10b-triaza-benzo[e]azulene-3-carboxylic acid ethyl ester 114 (SH-053-S—CH₃). The trimethylsilylacetylene intermediate 113 (SH—I-038) (2.8 g, 6.3 mmol) was dissolved in THF (60 mL) and was then treated with Bu₄NF.H₂O (1.9 g, 7.56 mmol). The mixture was allowed to stir for 30 min at rt, after which H₂O (10 mL) was added and the mixture was extracted with EtOAc (3×50 mL). The combined organic extracts were washed with brine (25 mL) and dried (Na₂SO₄). After removal of the solvent under reduced pressure, the residue was purified by a wash column (silica gel, EtOAc) to furnish 114 (SH-053-S—CH₃) (1.9 g, 85%) as a white solid: mp 197-199° C.; IR (KBr, cm⁻¹) 3285, 2928, 1708, 1616, 1553, 1498, 1445, 1374; ¹H NMR (CDCl₃) δ 7.92 (s, 1H), 7.73 (dd, J=1.72, 8.32 Hz, 1H), 7.58-7.36 (m, 7H), 6.67 (q, J=7.35, 1H), 4.46-4.34 (m, 2H), 3.16 (s, 1H), 1.41 (t, J=7.11, 3H), 1.25 (d, J=7.38, 3H); MS (EI) m/e (relative intensity) 369 (M⁺, 30), 323 (55), 295 (100), 267 (15). Anal. Calcd. for C₂₃H₁₉N₃O₂: C, 74.78; H, 5.18; N, 11.37. Found: C, 74.22; H, 5.11; N, 11.41. [α]²⁶_{D}=−74.8 (c 0.8, EtOAc).

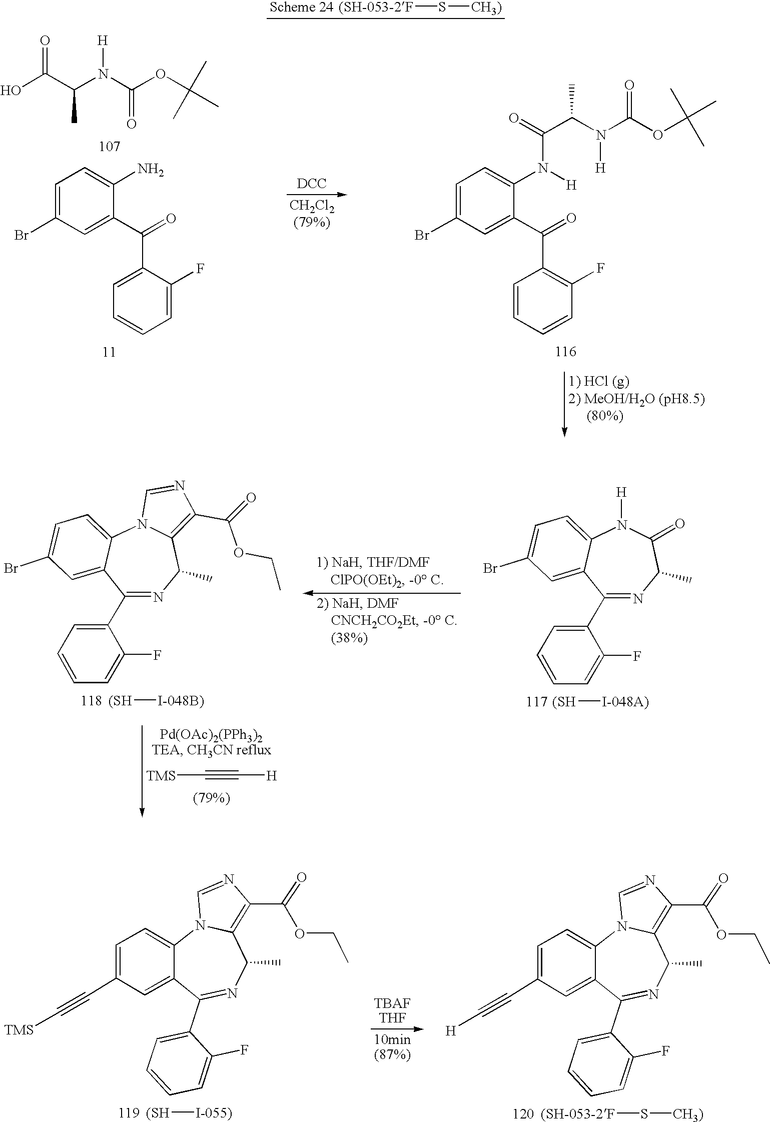

### Procedure for SH-053-2′F—S—CH₃:

{1-[4-Bromo-2-(2-fluoro-benzoyl)-phenylcarbamoyl-ethyl}-carbamic acid tert-butyl ester 116. To a stirred solution of (2-amino-5-bromophenyl)-(2-fluoro-phenyl)-methanone (60 g, 204 mmol) 11 and the N-Boc-L-alanine 107 (38.59 g, 204 mmol) in CH₂Cl₂ (500 mL) was added dicyclohexylcarbodiimide (DCC) (42.09 g, 204 mmol) in CH₂Cl₂ (200 mL) dropwise, over a 30 min period at 0° C. The reaction mixture was allowed to stir an additional 8 h at rt. The dicyclohexyl urea which formed was filtered off and the filtrate concentrated under reduced pressure. The crude solid residue 116 was purified by recrystallization from hexane and EtOAc to afford 116 (74.9 g, 79%) mp 158-159° C.; IR (KBr, cm⁻¹) 3332, 2931, 255, 1694, 1643, 1613, 1582, 1537, 1450; ¹H NMR (CDCl₃) δ 11.68 (s, 1H), 8.71 (d, j=9.0 Hz, 1H), 7.69 (dd, J=9.0, 2.3 Hz, 1H), 7.55-7.62 (m, 2H), 7.46 (td, J=7.6, 1.4 Hz, 1H), 7.30 (t, J=7.5 Hz, 1H), 7.21 (t, j=9.1 Hz, 1H), 5.13 (b, 1H), 4.37 (b, 1H), 1.51 (d, J=7.2 Hz, 3H), 1.45 (s, 9H). MS (EI) m/e (relative intensity) 467 (M⁺+2, 14), 466 (M⁺+1, 44), 465 (M⁺, 14), 464 (42), 329 (15), 321 (60), 295 (100), 224 (26); [α]²⁶_{D}=−59.1 (c 0.51, EtOAc).

7-Bromo-5-(2-fluoro-phenyl)-3-methyl-1,3-dihydrobenzo[e][1,4]diazepin-2-one 117. To a stirred solution of the benzophenone 116 (30 g, 64.4 mmol) in CHCl₃ (300 mL) at rt, hydrogen chloride gas was added slowly. After 20 min, the addition was stopped and the solution was allowed to stir overnight at rt. The reaction mixture was washed with a saturated solution of sodium bicarbonate solution (2×70 mL) and water (2×70 mL). The organic layer was concentrated under reduced pressure. The residual oil was dissolved in methanol/water (1:1, 300 mL) and the pH was adjusted to 8.5 by the addition of aq sodium hydroxide (1 N). The reaction mixture was allowed to stir for 10 hr at rt. The solution was then concentrated under reduced pressure and water (100 mL) was added. The solution was extracted with CH₂Cl₂ (3×50 mL), the organic layer was dried (Na₂SO₄) and concentrated under reduced pressure. The crude solid 117 was purified by recrystallization from methanol/water to provide 117 (17.8 g, 80%). mp 183-185° C.; IR (KBr, cm⁻¹) 2928, 1694, 1611, 1479, 1450, 1377, 1315, ¹H NMR (CDCl₃) δ 9.50 (bs, 1H), 7.62-7.65 (m, 2H), 7.50 (q, J=6.5 Hz, 1H), 7.40 (d, J=2.0 Hz, 1H), 7.29 (t, J=7.5 Hz 1H) 7.15 (d, J=8.6 Hz, 1H), 7.11 (t, J=8.9 Hz, 1H), 3.84 (q, J=6.5 Hz, 1H), 1.82 (d, J=6.5 Hz, 3H); MS (EI) m/e (relative intensity) 348 (M⁺+1, 23), 347 (M⁺, 38), 346 (24), 345 (36), 329 (19), 327 (20), 307 (40), 306 (41), 305 (100), 304 (37), 303 (63); [α]²⁶_{D}=168.8 (c 0.73, EtOAc).

8-Bromo-6-(2-fluorophenyl)-4-methyl-4H-2,5,10b-triaza-benzo[e]azulene-3-carboxylic acid ethyl ester 118. 7-Bromo-5-(2-fluorophenyl)-3-methyl-1,3-dihydro-benzo[e][1,4]diazepin-2-one 117 (3.78 g, 10.88 mmol) was suspended in dry THF (150 mL) and cooled to −10° C. Sodium hydride (60% dispersion in mineral oil, 0.52 g, 13.07 mol) was added into the suspension in one portion. The reaction mixture was allowed to stir and then warm to rt over a 3 h period. The reaction mixture was again cooled to −10° C. and diethyl chlorophosphate (2.65 mL, 17.42 mmol) was added. The cooling bath was then removed and stirring continued for 3 h. During this time, sodium hydride (60% dispersion in mineral oil, 0.61 g, 15.24 mmol) was suspended in dry THF (60 mL) at −10° C. in another flask. Ethyl isocyanoacetate (1.43 ml, 13.07 nmol) was added to the NaH/THF suspension and this mixture was stirred for 3 h. After 3 h the first flask was cooled to −30° C. with a cooling bath and the mixture in the second reaction flask was added to the first flask via a cannula. The reaction mixture was allowed to stir for 24 h, after which it was cooled to 0° C. with an ice-water bath and slowly quenched with acetic acid (5 mL). Water was then added to the reaction mixture and this was extracted with EtOAc. The EtOAc layers were combined, washed with aq NaHCO₃, brine and dried (Na₂SO₄). After removal of the solvent under reduced pressure, the solid was purified by flash chromatography (silica gel, EtOAc:hexane: gradient elution 1:2, 1:1, 2:1). The ethyl ester 118 was a white solid (1.82 g, 38%). mp 190-192° C.; IR (KBr, cm⁻¹) 3316, 2925, 1693, 1621, 1485, 1448, 1371; ¹H NMR (CDCl₃) δ 7.92 (s, 1H), 7.72 (dd, J=8.5, 1.5 Hz, 1H), 7.6 (t, J=6.9 Hz, 1H), 7.48 (d, J=8.5 Hz, 1H), 7.42-7.49 (m, 2H), 7.23-7.29 (m, 1H), 7.05 (t, J=9.3 Hz, 1H), 6.71 (q, J=7.3 Hz, 1H), 4.41 (m, 2H), 1.42 (t, J=7.1 Hz, 3H), 1.29 (d, J=7.2, 3H). MS (EI) m/e (relative intensity) 442 (M⁺, 5), 428 (7), 381 (58), 355 (100), 303 (37); [α]²⁶_{D}=10.6 (c 0.53, EtOAc).

6-(2-Fluorophenyl)-4-methyl-8-trimethylsilanylethynyl-4H-2,5,10b-triaza-benzo[e]azulene-3-carboxylic acid ethyl ester 119. A mixture of 118 (69.3 mg, 0.16 mmol) and bis(triphenylphosphine) palladium(II) acetate (11.68 mg, 0.015 mmol) was dissolved in a mixed solvent system of acetonitrile (120 mL) and TEA (80 mL). The mixture was degassed under vacuum and argon gas was added, after which trimethylsilylacetylene (0.044 mL, 0.31 mmol) was added into the mixture. The mixture was degassed again under vacuum (argon) and heated to reflux. The mixture was heated at reflux until analysis by TLC (EtOAc) indicated that all of the starting material 118 had been consumed. The mixture was cooled to rt and the precipitate which formed was removed by filtration through celite. The filtrate was concentrated under reduced pressure and partitioned between H₂O and EtOAc. The combined layers of EtOAc were washed with brine and dried (Na₂SO₄), after which the solvent was removed under reduced pressure and the residue was purified by flash chromatography (silica gel, EtOAc:hexane 1:1). The conditions for TLC were EtOAc on silica gel. A white solid 119 (58.1 mg, 7 9%) was obtained. mp 186-187° C.; IR (KBr, cm⁻¹) 2410, 2358, 1716, 1497, 1253; ¹H NMR (CDCl₃) δ 7.92 (s, 1H), 7.72 (dd, J=8.5, 1.5 Hz, 1H), 7.6 (t, J=6.9 Hz, 1H), 7.48 (d, J=8.5 Hz, 1H), 7.42-7.49 (m, 2H), 7.23-7.29 (m, 1H), 7.05 (t, J=9.3 Hz, 1H), 6.71 (q, J=7.3 Hz, 1H), 4.41 (m, 2H), 1.42 (t, J=7.1 Hz, 3H), 1.29 (d, J=7.2, 3H), 0.24 (s, 9H). MS (EI) m/e (relative intensity) 459 (M⁺, 28), 445 (32), 399 (51), 371 (100), 235 (71), 178 (75); [α]²⁶_{D}=−27.8 (c 0.46, EtOAc).

8-Ethynyl-6-(2-fluorophenyl)-4-methyl-4H-2,5,10b-triazabenzo[e]azulene-3-carboxylic acid ethyl ester 120 (SH-053-2′F—S—CH₃). The trimethylsilylacetylene intermediate 119 (SH—I-055) (0.17 g, 0.37 mmol) was dissolved in THF (30 mL) and was then treated with Bu₄NF.H₂O (0.114 g, 0.44 mmol). The mixture was allowed to stir for 30 min at rt, after which H₂O (10 mL) was added and the mixture was extracted with EtOAc (3×25 mL). The combined organic extracts were washed with brine (25 mL) and dried (Na₂SO₄). After removal of the solvent under reduced pressure, the residue was purified by a wash column (silica gel, EtOAc) to furnish 120 (SH-053-2′F—S—CH₃) (0.12 g, 87%) as a white solid: mp 212-214° C.; IR (KBr, cm⁻¹) 3288, 2979, 1716, 1497, 1257, 1255; ¹H NMR (CDCl₃) δ 7.98 15 (s, 1H), 7.72 (d, J=8.3 Hz, 1H) 77.63 (d, J=8.5 Hz, 1H), 7.59 (d, J=8.3 Hz, 1H), 7.42-7.49 (m, 1H), 7.42 (s, 1H), 7.23-7.28 (m, 1H), 7.05 (t, J=9.2 Hz, 1H), 6.71 (q, J=7.2, 1H), 4.41 (m, 2H), 3.16 (s, 1H), 1.42 (t, J=7.1 Hz, 3H) 1.29 (d, J=7.3 Hz, 3H). MS (EI) m/e (relative intensity) 387 (M⁺, 20), 373 (21), 327 (47), 299 (100); [α]²⁶_{D}=−0.95 (c 0.84, EtOAc).

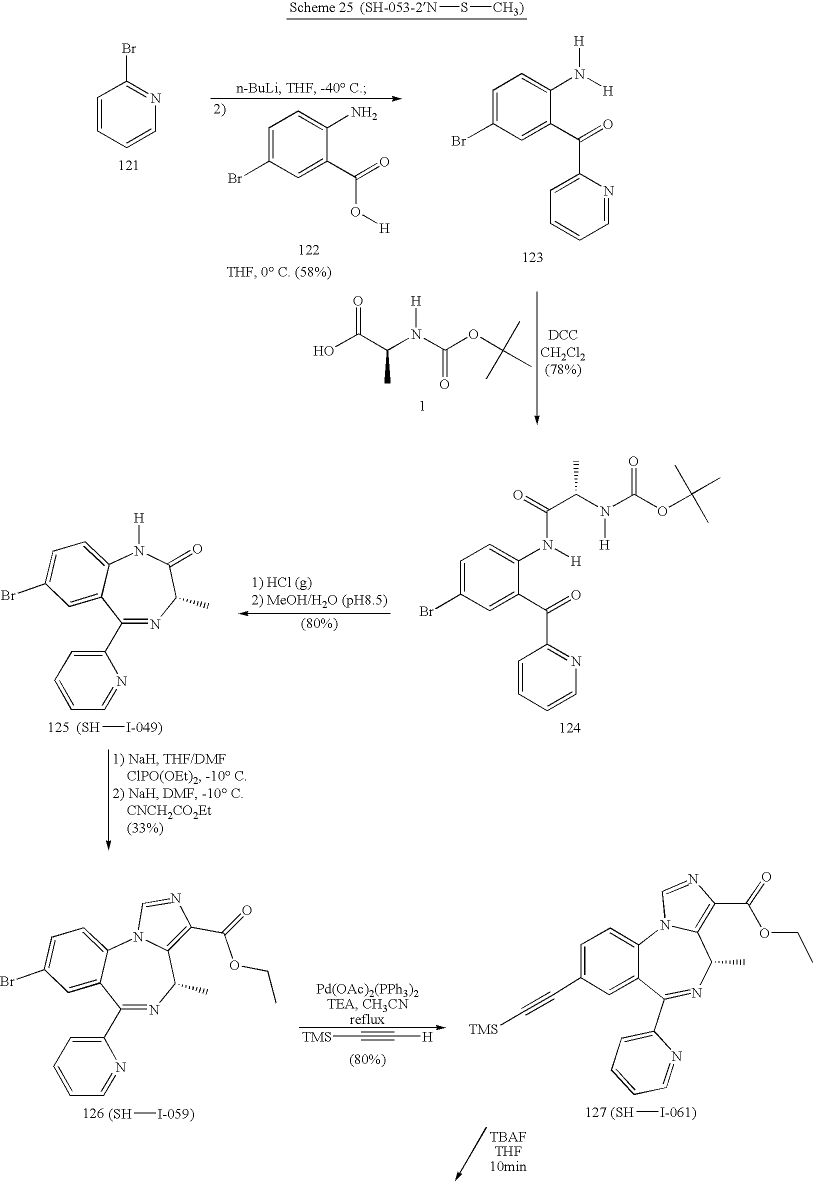

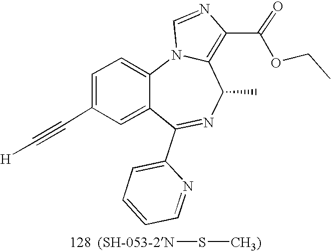

### Procedure for SH-053-2′N—S—CH₃ (128):

(2-Amino-5-bromo-phenyl)-pyridin-2-yl-methanone 123. The anion of 2-bromo-pyridine 121 and 2-amino-5-bromobenzoic acid 122 were condensed to provide the 2′-pyridylketone 123.

{1-[4-Bromo-2-(pyridine-2-carbonyl)-phenylcarbamoyl]-ethyl)-carbamic acid tert-butyl ester 124. To a stirred solution of (2-amino-5-bromophenyl)-pyridin-2-yl-methanone 123 (16 g, 57.33 mmol) and the N-Boc-L-alanine 107 (10.92 g, 57.73 mmol) in CH₂Cl₂ (100 mL) was added dicyclohexylcarbodiimide (DCC) (11.91 g, 57.73 mmol) in CH₂Cl₂ (60 mL) dropwise, over a 30 min period at 0° C. The reaction mixture was allowed to stir an additional 8 h at rt. The dicyclohexyl urea which formed was filtered off and the filtrate concentrated under reduced pressure. The crude solid 124 was purified by recrystallization from hexane and EtOAc to afford 124 (7.88 g, 81%). mp 208-210° C.; IR (KBr, cm⁻¹) 3332, 2931, 1694, 1507, 1287, 1163; ¹H NMR (CDCl₃) δ 11.68 (s, 1H), 8.71 (d, J=9.0 Hz, 1H), 7.69 (dd, J=9.0, 2.3 Hz, 1H), 7.55-7.62 (m, 2H), 7.46 (td, J=7.6, 1.4 Hz, 1H), 7.30 (t, J=7.5 Hz, 1H), 7.21 (t, J=9.1 Hz, 1H), 5.13 (b, 1H), 4.37 (b, 1H), 1.51 (d, J=7.2 Hz, 3H), 1.45 (s, 9H). MS (EI) m/e (relative intensity) 449 (M⁺+1, 5), 448 (M⁺, 5), 376 (10), 329 (20), 304 (100), 228 (25); [α]²⁶_{D}=−36.1 (c 0.61, EtOAc).

7-Bromo-3-methyl-5-pyridin-2-yl-1,3-dihydro-benzo[e][1,4]diazepin-2-one 125. To a stirred solution of ester 124 (16 g, 35.69 mmol) in CHCl₃ (300 mL) at rt, hydrogen chloride gas was added in slowly. After 20 min, the addition was stopped and the solution was allowed to stir overnight at rt. The reaction mixture was washed with a saturated solution of sodium bicarbonate (2×50 mL) and water (2×50 mL). The organic layer was concentrated under reduced pressure. The residual oil was dissolved in methanol-water (3:1, 300 mL) and the pH was adjusted to 8.5 by the addition of aq sodium hydroxide (1 N). The reaction mixture was stirred for 10 h at rt. The solution was concentrated under reduced pressure and water (80 mL) was added. The solution was extracted with CH₂Cl₂ (3×70 mL) and concentrated under reduced pressure. The crude solid 125 was purified by recrystallization from methanol/water to provide pure 125 (9.42 g, 80%). mp 227-229° C.; IR (KBr, cm⁻¹) 2928, 1684, 1611, 1476; ¹HNMR (CDCl₃) δ 9.47 (bs, 1H), 8.62 (d, J=4.7 Hz, 1H), 8.0 (d, J=7.90 Hz, 1H), 7.81 (td, J=7.7, 1.6 Hz, 1H), 7.56 (dd, J=8.6, 2.2 Hz 1H), 7.50 (dd, J=2.1 Hz, 1H), 7.36 (dd, J=7.4 Hz, 1H), 7.06 (d, J=8.6 Hz, 1H), 3.85 (q, J=6.5 Hz, 1H), 1.76 (d, J=6.5 Hz, 3H); MS (EI) m/e (relative intensity) 330 (M⁺, 50), 329 (50), 314 (52), 288 (100), 250 (30), 208 (32), 179 (50), 88 (72); [α]²⁶_{D}=403.2 (c 0.50, EtOAc).

8-Bromo-4-methyl-6-pyridin-2-yl-4H-2,5,10b-triaza-benzo[e]azulene-3-carboxylic acid ethyl ester 126. The 7-bromo-3-methyl-5-pyridin-2-yl-1,3-dihydro-benzo[e][1,4]diazepin-2-one 125 (3.3 g, 10 mmol) was suspended in dry THF (200 mL) and cooled to −10° C. Sodium hydride (60% dispersion in mineral oil, 0.48 g, 12 mmol) was added into the suspension in one portion. The reaction mixture was allowed to stir and warm to rt over a 3 h period. The reaction mixture was again cooled to −10° C. and diethyl chlorophosphate (2.31 mL, 16 mmol) was added. The cooling bath was then removed and stirring continued for 3 h. At this time, sodium hydride (60% dispersion in mineral oil, 0.56 g, 14 mmol) was suspended in dry THF (150 mL) at −10° C. in another flask. Ethyl isocyanoacetate (1.31 mL, 12 mmol) was added to the NaH/THF suspension, the solution which resulted was allowed to stir for 3 h. After 3 h, the first flask was cooled to −30° C. and the solution in the 2nd reaction flask was added to the first flask via a cannula. The reaction mixture was allowed to stir for 24 h, and then cooled with an ice-water bath and slowly quenched with acetic acid (10 mL). Water was added to the reaction mixture after which it was extracted with EtOAc. The EtOAc layers were combined, washed with aq NaHCO₃, brine and dried (Na₂SO₄). After removal of the solvent under reduced pressure, the solid was purified by flash chromatography (silica gel, EtOAc:hexane, gradient elution 1:1, 2:1, 3:1). The ester 126 was a white solid (1.40 g, 33%). mp 193-195° C.; IR (KBr, cm⁻¹) 2962, 1719, 1260, 1021; ¹H NMR (CDCl₃) δ 7.92 (s, 1H), 7.72 (dd, J=8.5, 1.5 Hz, 1H), 7.6 (t, J=6.9 Hz, 1H), 7.48 (d, J=8.5 Hz, 1H), 7.42-7.49 (m, 2H), 7.23-7.29 (m, 1H), 7.05 (t, J=9.3 Hz, 1H), 6.71 (q, J=7.3 Hz, 1H), 4.41 (m, 2H), 1.42 (t, J=7.1 Hz, 3H), 1.29 (d, J=7.2, 3H).

4-Methyl-6-pyridin-2-yl-8-trimethylsilanylethynyl-4H-2,5,10b-triazabenzo[e]azulene-3-carboxylic acid ethyl ester 127 (SH—I-061). A mixture of 126 (2.73 g, 6.42 mmol) and bis(triphenylphosphine)palladium(II) acetate (0.48 g, 0.64 mmol) was dissolved in a mixed solvent system of acetonitrile (80 mL) and TEA (120 mL). The mixture was degassed under vacuum and argon gas was added, after which trimethylsilylacetylene (1.8 mL, 12.85 mmol) was added into the mixture. The mixture was degassed again under vacuum (argon) and heated to reflux. The mixture was heated at reflux until analysis by TLC (EtOAc) indicated that all of the starting material 126 had been consumed. The mixture was cooled to rt and the precipitate which formed was removed by filtration through celite. The filtrate was concentrated under reduced pressure and partitioned between H₂O and EtOAc. The combined layers of EtOAc were washed with brine and dried (Na₂SO₄), after which the solvent was removed under reduced pressure and the residue was purified by flash chromatography (silica gel, EtOAc:hexane 2:1). A white solid 127 (SH—I-061) (2.27 g, 80%) was obtained. mp 197-198° C.; IR (KBr, cm⁻¹) 3477, 2982, 2158, 1713, 1643; ¹H NMR (CDCl₃) δ 7.92 (s, 1H), 7.72 (dd, J=8.5, 1.5 Hz, 1H), 7.6 (t, J=6.9 Hz, 1H), 7.48 (d, J=8.5 Hz, 1H), 7.42-7.49 (m, 2H), 7.23-7.29 (m, 1H), 7.05 (t, J=9.3 Hz, 1H), 6.71 (q, J=7.3 Hz, 1H), 4.41 (m, 2H), 1.42 (t, J=7.1 Hz, 3H), 1.29 (d, J=7.2, 3H), 0.24 (s, 9H); [α]²⁶_{D}=304.4 (c 0.41, EtOAc).

8-Ethynyl-4-methyl-6-pyridin-2-yl-4H-2,5,10b-triaza-benzo[e]azulene-3-carboxylic acid ethyl ester 128 (SH-053-2′N—S—CH₃). The trimethylsilylacetylene intermediate 127 (1.5 g, 3.39 mmol) was dissolved in THF (150 mL) and was then stirred with Bu₄NF.H₂O (1.06 g, 4.06 mmol). The mixture was allowed to stir for 30 min at rt, after which H₂O (10 mL) was added and the mixture was extracted with EtOAc (3×50 mL). The combined organic extracts were washed with brine (25 mL) and dried (Na₂SO₄). After removal of the solvent under reduced pressure, the residue was purified by a wash column (silica gel, EtOAc) to furnish 128 (SH-053-2′N—S—CH₃) (1.01 g, 81%) as a white solid: mp 235-237° C.; IR (KBr, cm⁻¹) 3321, 2979, 2933, 1647, 1597; ¹H NMR (CDCl₃) δ 8.61 (d, J=4.2 Hz, 1H), 8.01 (d, J=7.8 Hz, 1H), 7.91 (s, 1H), 7.81 (t, J=7.8 Hz, 1H), 7.72 (d, J=9.5 Hz, 1H), 7.55 (d, J=12 Hz, 1H), 7.59 (s, 1H), 7.37 (t, J=9.8 Hz, 1H), 6.71 (q, J=7.3, 1H), 4.41 (m, 2H), 3.16 (s, 1H), 1.42 (t, J=7.1 Hz, 3H) 1.28 (d, J=7.3 Hz, 3H); [α]²⁶_{D}=−85.2 (c 0.69, EtOAc).

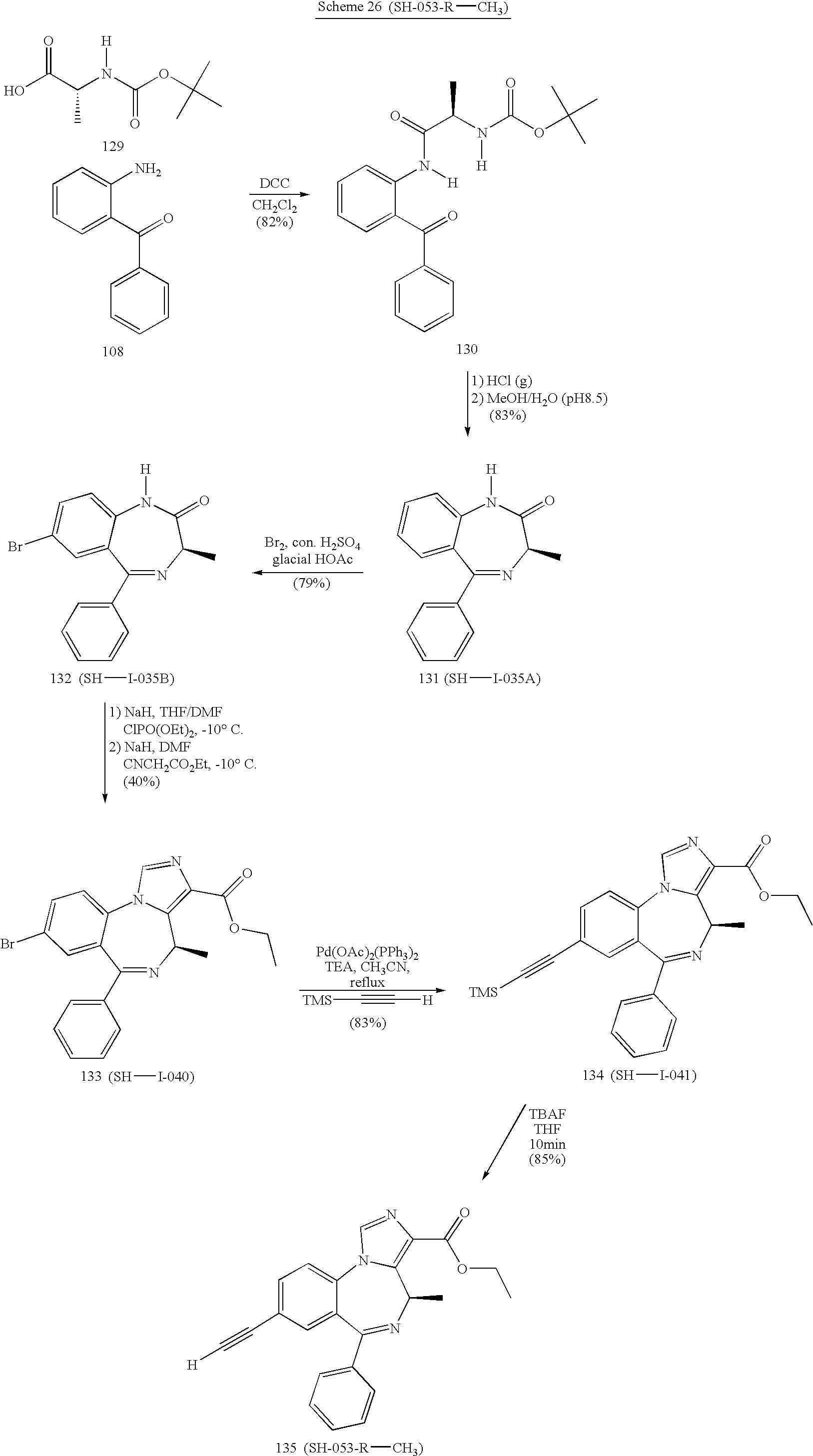

### Procedure for SH-053-R—CH₃ (135):

1-(2-Benzoyl-phenylcarbamoyl)-ethyl]-carbamic acid tert-butyl ester 130. To a stirred solution of 2-amino-5-bromobenzophenone (5.73 g, 29.07 mol) and the N-Boc-D-alanine 129 (5 g, 26.43 mmol) in CH₂Cl₂ (200 mL) was added dicyclohexylcarbodiimide (DCC) (5.99 g, 29.07 mmol) in CH₂Cl₂ (100 mL) dropwise, over a 30 min period at 0° C. The reaction mixture was allowed to stir an additional 8 h at rt. The dicyclohexyl urea which formed was filtered off and the filtrate concentrated under reduced pressure. The crude solid 130 was purified by recrystallization from hexane to afford 130 (7.97 g, 82%). mp 127-129° C.; IR (KBr, cm⁻¹) 3288, 2475, 2352, 1684, 1636, 1576, 1507, 1447, 1264, 1165, 700; ¹H NMR (CDCl₃) δ 11.48 (s, 1H), 8.67 (d, J=8.22 Hz, 1H), 7.71-7.43 (m, 7H), 7.13-7.08 (m, J=1H) 5.06 (br s, 1H), 4.36 (br s, 1H), 1.50 (d, J=7.1 Hz, 3H), 1.44 (s, 9H); MS (EI) m/e (relative intensity) 368 (M⁺, 6), 295 (10), 225 (27), 224 (79), 197 (83), 196 (77), 167 (15), 145 (46), 144 (88), 126 (17), 105 (38), 88 (94), 77 (37), 57 (100); [α]²⁶_{D}=67.3 (c 0.44, EtOAc).

3-Methyl-5-phenyl-1,3-dihydro-benzo[e][1,4]diazepin-2-one 131. To a stirred solution of the benzophenone 130 (10.65 g, 29.38 mmol) in CHCl₃ (400 mL) at rt, hydrogen chloride gas was added in slowly. After 20 min, the addition was stopped and the solution was allowed to stir overnight at rt. The reaction mixture was then washed with a saturated solution of sodium bicarbonate (2×50 mL) and water (2×50 mL). The organic layer was concentrated under reduced pressure. The residual oil was dissolved in methanol-water (2:1, 500 mL) and the pH was adjusted to 8.5 by the addition of aq sodium hydroxide (1 N). The reaction mixture was stirred for 10 h at rt. The solution was concentrated under reduced pressure and water (100 mL) was added. The solution was extracted with CH₂Cl₂ (3×100 mL) and concentrated under reduced pressure. The crude solid 131 was purified by recrystallization from methanol/water to provide 131 (6.10 g, 83%). mp 160-162° C.; IR (KBr, cm⁻¹) 3215, 3059, 2974, 2932, 1681, 1574, 1478, 1445, 1372, 1321, 1242, 1160, 1131; ¹H NMR (CDCl₃) δ 9.65 (s, 1H), 7.54-7.13 (m, 9H), 3.78 (q, J=6.45 Hz, 1H), 1.78 (d, J=7.1 Hz, 3H); MS (EI) m/e (relative intensity) 250 (M⁺, 40), 249 (83), 234 (15), 209 (75), 208 (76), 207 (100), 180 (17), 152 (19) 103 (23), 77 (40).

7-Bromo-3-methyl-5-phenyl-1,3-dihydro-benzo[e][1,4]diazepin-2-one 132. To a stirred solution of 131 (66.5 g, 265 m mol) in glacial acetic acid (400 mL), sulfuric acid (80 mL) was added to the solution. Bromine (28 mL, 530 mmol) dissolved in acetic acid (150 mL) was added dropwise into the mixture. The reaction mixture was allowed to stir until analysis by NMR spectroscopy indicated that all of the starting material 131 had been consumed. The solution was concentrated under reduced pressure and then brought to pH 7 by addition of 1N aq NaOH solution and then extracted with EtOAc. The crude product 132 was purified by recrystallization from CH₂Cl₂ to afford 132 (68.66 g, 79%). mp 210-212° C.; IR (KBr, cm⁻¹) 2931, 1692, 1603, 1563, 1475, 1445, 1375, 1259, 1235, 1130, 1090; ¹H NMR (CDCl₃) δ 9.36 (s, 1H), 7.63 (dd, J=2.22, 8.58 Hz, 1H), 7.54-7.39 (m, 6H), 7.12 (d, J=8.61 1H), 3.76 (q, J=6.3 Hz, 1H), 1.76 (d, J=6.45 Hz, 3H). MS (EI) m/e (relative intensity) 330 (M⁺+1, 21), 329 (M⁺, 50), 328 (22), 327 (48), 289 (44), 288 (46), 287 (100), 286 (45), 285 (60), 205 (25), 77 (49).

8-Bromo-4-methyl-6-phenyl-4H-2,5,10b-triaza-benzo[e]azulene-3-carboxylic acid ethyl ester 133. 7-Bromo-3-methyl-5-phenyl-1,3-dihydro-benzo[e][1,4]diazepin-2-one 132 (16.6 g, 0.052 mol) was suspended in dry THF (250 mL) and cooled to −10° C. Sodium hydride (60% dispersion in mineral oil, 4.36 g, 0.109 mol) was added into the suspension in one portion. The reaction mixture was allowed to stir and warm to rt over a 3 h period. The reaction mixture was again cooled to −10° C. and diethyl chlorophosphate (12.7 mL, 0.09 mol) was added. The cooling bath was then removed and stirring continued for 3 h. At this time, sodium hydride (60% dispersion in mineral oil, 4.2 g, 0.1 mol) was suspended in dry THF (250 mL) at −10° C. in another flask. Ethyl isocyanoacetate (6.78 mL, 0.06 mol) was added to the NaH/THF suspension, after which the solution which resulted was allowed to stir for 3 h. After 3 h the first flask was cooled to −30° C. and the solution in the 2nd reaction flask was added to the first flask via a cannula. The reaction mixture was allowed to stir for 24 h, and then cooled with an ice-water bath and slowly quenched with acetic acid (10 mL). Water was added to the reaction mixture after which it was extracted with EtOAc. The EtOAc layers were combined, washed with aq NaHCO₃, brine and dried (Na₂SO₄). After removal of the solvent under reduced pressure, the solid was purified by flash chromatography (silica gel, EtOAc:hexane, gradient elution 1:2, 1:1, 2:1). The ester 133 was a white solid (8.76 g, 40%). mp 164-165° C.; IR (KBr, cm⁻¹) 2925, 1706, 1622, 1557, 1495, 1266, 1185; ¹H NMR (CDCl₃) δ 7.89 (s, 1H), 7.73 (dd, J=1.73 Hz, 1H), 7.51-7.36 (m, 7H), 6.66 (q, J=7.30, 1H) 4.45-4.30 (m, 2H), 1.40 (t, J=7.11, 3H), 1.25 (d, J=7.38 Hz, 3H). MS (EI) m/e (relative intensity) 426 (M⁺+2, 15), 425 (M⁺+1, 58), 424 (M⁺, 15), 423 (58), 380 (24), 379 (71), 378 (35), 377 (69), 352 (50), 351(100), 350 (67), 349 (92), 270 (38), 229 (16); [α]²⁶_{D}=38.2 (c 0.45, EtOAc).

4-Methyl-6-phenyl-8-trimethylsilanylethynyl-4H-2,5,10b-triazabenzo[e]azulene-3-carboxylic acid ethyl ester 134 (SH—I-041). A mixture of ester 133 (3.0 g, 7.07 mmol) and bis(triphenyl phosphine) palladium(II) acetate (0.42 g, 0.57 mmol) was dissolved in a mixed solvent system of acetonitrile (80 mL) and TEA (120 mL). The mixture was degassed under vacuum and argon was added, after which trimethylsilylacetylene (2 mL, 14.14 mmol) was added into the mixture. The mixture was degassed again under vacuum (argon) and heated to reflux. The mixture was heated at reflux until analysis by TLC (EtOAc) indicated that all of the starting material 133 had been consumed. The mixture was cooled to rt and the precipitate which formed was removed by filtration through celite. The filtrate was concentrated under reduced pressure and partitioned between H₂O and EtOAc. The combined layers of EtOAc were washed with brine and dried (Na₂SO₄), after which the solvent was removed under reduced pressure and the residue was purified by flash chromatography (silica gel, EtOAc:hexane 1:1). The conditions for TLC were EtOAc on silica gel. A white solid 134 (SH—I-041) (2.59 g, 83%) was obtained. mp 160-162° C.; IR (KBr, cm⁻¹) 3365, 2925, 1706, 1616, 1553, 1498; ¹H NMR (CDCl₃) δ 7.89 (s, 1H), 7.73 (dd, J=1.73 Hz, 1H), 7.51-7.36 (m, 7H), 6.66 (q, J=7.30, 1H), 4.45-4.30 (m, 2H), 1.40 (t, J=7.11, 3H), 1.25 (d, J=7.38 Hz, 3H), 0.15 (S, 9H). MS (EI) m/e (relative intensity) 441 (M⁺, 15), 369 (25), 323 (55), 295 (100), 267 (15).

8-Ethynyl-4-methyl-6-phenyl-4H-2,5,10b-triaza-benzo[e]azulene-3-carboxylic acid ethyl ester 135 (SH-053-R—CH₃) The trimethylsilylacetylene intermediate 134 (2.8 g, 6.3 mmol) was dissolved in THF (20 mL) and was then treated with Bu₄NF.H₂O (1.9 g, 7.56 mmol). The mixture was allowed to stir for 30 min at rt, after which H₂O (10 mL) was added and the mixture was extracted with EtOAc (3×50 mL). The combined organic extracts were washed with brine (25 mL) and dried (Na₂SO₄). After removal of the solvent under reduced pressure, the residue was purified by a wash column (silica gel, EtOAc) to furnish 135 (SH-053-R—CH₃) (1.9 g, 85%) as a white solid: mp 197-199° C.; IR (KBr, cm⁻¹) 3285, 2928, 1708, 1616, 1553, 1498, 1445, 1374; ¹H NMR (CDCl₃) δ 7.92 (s, 1H), 7.73 (dd, J=1.72, 8.32 Hz, 1H), 7.58-7.36 (m, 7H), 6.67 (q, J=7.35, 1H), 4.46-4.34 (m, 2H), 3.16 (s, 1H), 1.41 (t, J=7.11, 3H), 1.25 (d, J=7.38, 3H); MS (EI) m/e (relative intensity) 369 (M⁺, 30), 323 (55), 295 (100), 267 (15). Anal. Calcd. for C₂₃H₁₉N₃O₂: C, 74.78; H, 5.18; N, 11.37. Found: C, 74.43; H, 5.67; N, 11.39. [α]²⁶_{D}=75.0 (c 0.8, EtOAc).

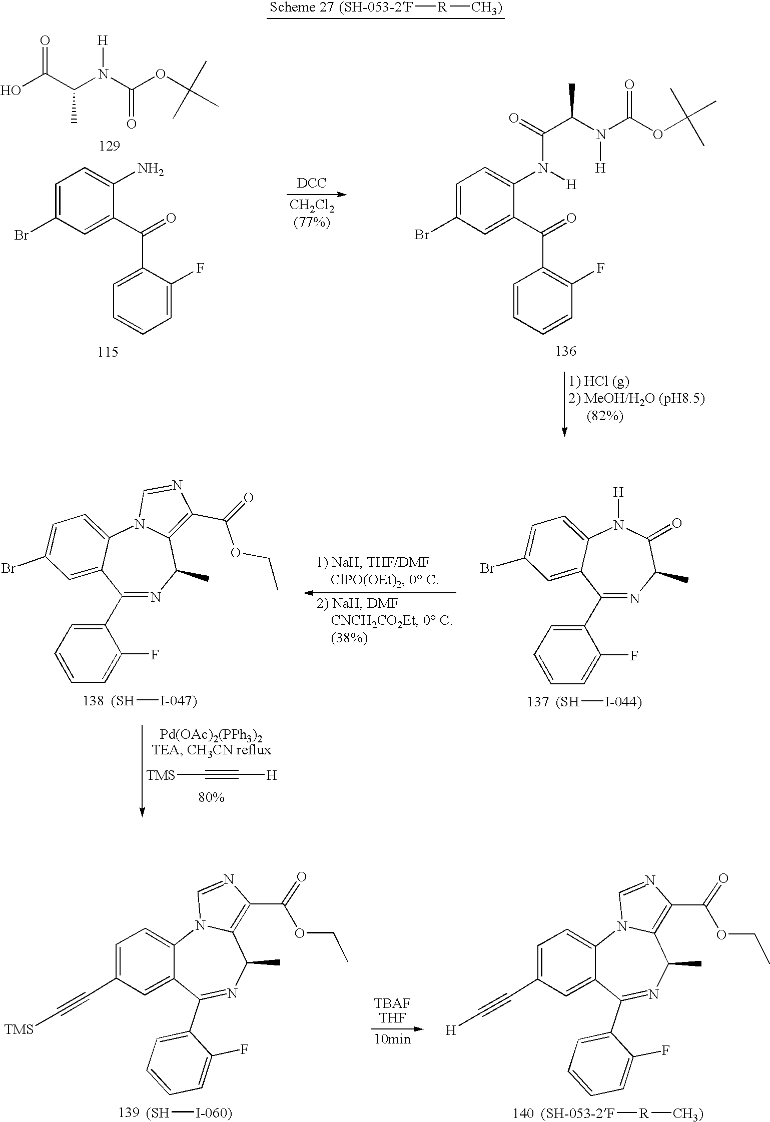

### Procedure for SH-053-2′F—R—CH₃ (140):

{1-[4-Bromo-2-(2-fluorobenzoyl)-phenylcarbamoyl]-ethyl)-carbamic acid tert-butyl ester 136. To a stirred solution of (2-amino-5-bromophenyl)-(2′-fluoro-phenyl)-methanone 11 (60 g, 204 mmol) and the N-Boc-D-alanine 129 (38.59 g, 2 04 mmol) in CH₂Cl₂ (500 mL) was added dicyclohexylcarbodiimide (DCC) (42.09 g, 204 mmol) in CH₂Cl₂ (200 mL) dropwise, over a 30 min period at 0° C. The reaction mixture was allowed to stir an additional 8 h at rt. The dicyclohexyl urea which formed was filtered off and the filtrate concentrated under reduced pressure. The crude solid product 136 was purified by recrystallization from hexane and EtOAc to afford 136 (73 g, 77%). mp 158-159° C.; IR (KBr, cm⁻¹) 3332, 2931, 255, 1694, 1643, 1613, 1582, 1537, 1450; ¹H NMR (CDCl₃) δ 11.68 (s, 1H), 8.71 (d, J=9.0 Hz, 1H), 7.69 (dd, J=9.0, 2.3 Hz, 1H), 7.55-7.62 (m, 2H), 7.46 (td, J=7.6, 1.4 Hz, 1H), 7.30 (t, J=7.5 Hz, 1H), 7.21 (t, J=9.1 Hz, 1H), 5.13 (b, 1H), 4.37 (b, 1H), 1.51 (d, J=7.2 Hz, 3H), 1.45 (s, 9H). MS (EI) m/e (relative intensity) 467 (M⁺+2, 14), 466 (M⁺+1, 44), 465 (M⁺, 14), 464 (42), 329 (15), 321 (60), 295 (100), 224 (26); [α]²⁶_{D}=59.6 (c 0.51, EtOAc).

7-Bromo-5-(2-fluoro-phenyl)-3-methyl-1,3-dihydro-benzo[e][1,4]diazepin-2-one 137. To a stirred solution of the benzophenone 136 (30 g, 64.4 mmol) in CHCl₃ (300 mL) at rt, hydrogen chloride gas was added slowly. After 20 min, the addition was stopped and the solution was allowed to stir overnight at rt. The reaction mixture was then washed with a saturated solution of sodium bicarbonate (2×70 mL) and water (2×70 mL). The organic layer was concentrated under reduced pressure. The residual oil was dissolved in methanol-water (1:1, 300 mL) and the pH was adjusted to 8.5 by the addition of aq sodium hydroxide (1 N). The reaction mixture was allowed to stir for 10 h at rt. The solution was then concentrated under reduced pressure and water (100 mL) was added. The solution was extracted with CH₂Cl₂ (3×50 mL), and the organic layer was dried (Na₂SO₄) and concentrated under reduced pressure. The crude solid 137 was purified by recrystallization from methanol/water to provided 137 (18.2 g, 82%). mp 183-185° C.; IR (KBr, cm⁻¹) 2928, 1694, 1611, 1479, 1450, 1377, 1315; ¹H NMR (CDCl₃) δ 9.50 (bs, 1H), 7.62-7.65 (m, 2H), 7.50 (q, J=6.5 Hz, 1H), 7.40 (d, J=2.0 Hz, 1H), 7.29 (t, J=7.5 Hz 1H) 7.15 (d, J=8.6 Hz, 1H), 7.11 (t, J=8.9 Hz, 1H), 3.84 (q, J=6.5 Hz, 1H), 1.82 (d, J=6.5 Hz, 3H); MS (EI) m/e (relative intensity) 348 (M⁺+1, 23), 347 (M⁺, 38), 346 (24), 345 (36), 329 (19), 327 (20), 307 (40), 306 (41), 305(100), 304 (37), 303 (63); [α]²⁶_{D}=−169.1 (c 0.71, EtOAc).

8-Bromo-6-(2-fluorophenyl)-4-methyl-4H-2,5,10b-triaza-benzo[e]azulene-3-carboxylic acid ethyl ester 138. 7-Bromo-5-(2′-fluorophenyl)-3-methyl-1,3-dihydro-benzo[e][1,4]diazepin-2-one 137 (3.78 g, 10.88 mmol) was suspended in dry THF (150 mL) and cooled to −10° C. Sodium hydride (60% dispersion in mineral oil, 0.52 g, 13.07 mol) was added into the suspension in one portion. The reaction mixture was allowed to stir and then warm to rt over 3 h period. The reaction mixture was again cooled to −10° C. and diethyl chlorophosphate (2.65 mL, 17.42 mmol) was added. The cooling bath was then removed and stirring continued for 3 h. During this time, sodium hydride (60% dispersion in mineral oil, 0.61 g, 15.24 mmol) was suspended in dry THF (60 mL) at −10° C. in another flask. Ethyl isocyanoacetate (1.43 mL, 13.07 mmol) was added to the NaH/THF suspension and this mixture was stirred for 3 h. After 3 h the first flask was cooled to −30° C. with a cooling bath and the mixture in the second reaction flask was added to the first flask via a cannula. The reaction mixture was allowed to stir for 24 h, after which it was cooled to 0° C. with an ice-water bath and slowly quenched 5 with acetic acid (5 mL). Water was then added to the reaction mixture and this was extracted with EtOAc. The EtOAc layers were combined, washed with aq NaHCO₃, brine and dried (Na₂SO₄). After removal of the solvent under reduced pressure the solid was purified by flash chromatography (silica gel, EtOAc:hexane: gradient elution 1:2, 1:1, 2:1). The ethyl ester 138 was a white solid (1.82 g, 38%). mp 190-192° C.; IR (KBr, cm⁻¹) 3316, 2925, 1693, 1621, 1485, 1448, 1371; ¹H NMR (CDCl₃) δ 7.92 (s, 1H), 7.72 (dd, J=8.5, 1.5 Hz, 1H), 7.6 (t, J=6.9 Hz, 1H), 7.48 (d, J=8.5 Hz, 1H), 7.42-7.49 (m, 2H), 7.23-7.29 (m, 1H), 7.05 (t, J=9.3 Hz, 1H), 6.71 (q, J=7.3 Hz, 1H), 4.41 (m, 2H), 1.42 (t, J=7.1 Hz, 3H), 1.29 (d, J=7.2, 3H). MS (EI) m/e (relative intensity) 442 (M⁺, 5), 428 (7), 38 1 (58), 355 (100), 303 (37); [α]²⁶_{D}=−10.9 (c 0.54, EtOAc).

6-(2-Fluorophenyl)-4-methyl-8-trimethylsilanylethynyl-4H-2,5,10b-triazabenzo[e]azulene-3-carboxylic acid ethyl ester 139. A mixture of 138 (69.3 mg, 0.16 mmol) and bis(triphenyl phosphine)palladium(II) acetate (11.68 mg, 0.015 mmol) was dissolved in a mixed solvent system of acetonitrile (120 mL) and TEA (80 mL). The mixture was degassed under vacuum and argon gas was added, after which trimethylsilylacetylene (0.044 mL, 0.31 mmol) was added into the mixture. The mixture was degassed again under vacuum (argon) and heated to reflux. The mixture was heated at reflux until analysis by TLC (EtOAc) indicated that all of the starting material 138 had been consumed. The mixture was cooled to rt and the precipitate which formed was removed by filtration through celite. The filtrate was concentrated under reduced pressure and partitioned between H₂O and EtOAc. The combined layers of EtOAc were washed with brine and dried (Na₂SO₄), after which the solvent was removed under reduced pressure and the residue was purified by flash chromatography (silica gel, EtOAc:hexane 1:1). The conditions for TLC were EtOAc on silica gel. A white solid 139 (58.8 mg, 80%) was obtained. mp 186-187° C.; IR (KBr, cm⁻¹) 2410, 2358, 1716, 1497, 1253; ¹H NMR (CDCl₃) δ 7.92 (s, 1H), 7.72 (dd, J=8.5, 1.5 Hz, 1H), 7.6 (t, J=6.9 Hz, 1H), 7.48 (d, J=8.5 Hz, 1H), 7.42-7.49 (m, 2H), 7.23-7.29 (m, 1H), 7.05 (t, J=9.3 Hz, 1H), 6.71 (q, J=7.3 Hz, 1H), 4.41 (m, 2H), 1.42 (t, J=7.1 Hz, 3H), 1.29 (d, J=7.2, 3H), 0.24 (s, 9H). MS (EI) m/e (relative intensity) 459 (M⁺, 28), 445 (32), 399 (51), 371 (100), 235 (71), 178 (75); [α]²⁶_{D}=28.2 (c 0.48, EtOAc).

8-Ethynyl-6-(2-fluorophenyl)-4-methyl-4H-2,5,10b-triaza-benzo[e]azulene-3-carboxylic acid ethyl ester 140 (SH-053-2′F—R—CH₃). The trimethylsilylacetylene intermediate 139 was converted to 140 (SH-053-2′F—R—CH₃) by using the same procedure described above for the synthesis of compound 10.

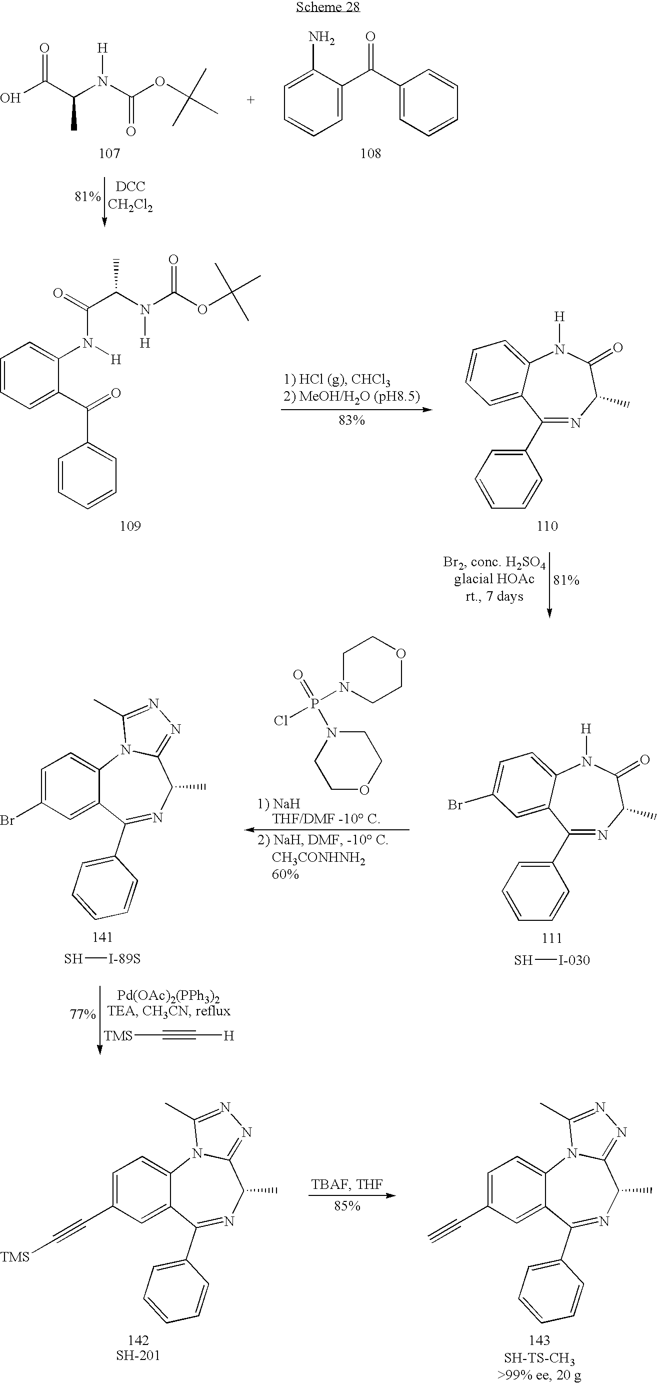

### Synthesis of S-enantiomer (SH-TS-CH₃, 143)

The analog SH-TS-CH₃ (143) is the triazolam analog of SH-053-S—CH₃ (114) which was designed to gain better water solubility, to enhance potency and to avoid the sedative, ataxic, amnesic and muscle relaxant side effects of traditional triazolam-like agents, Halcion and Xanax.

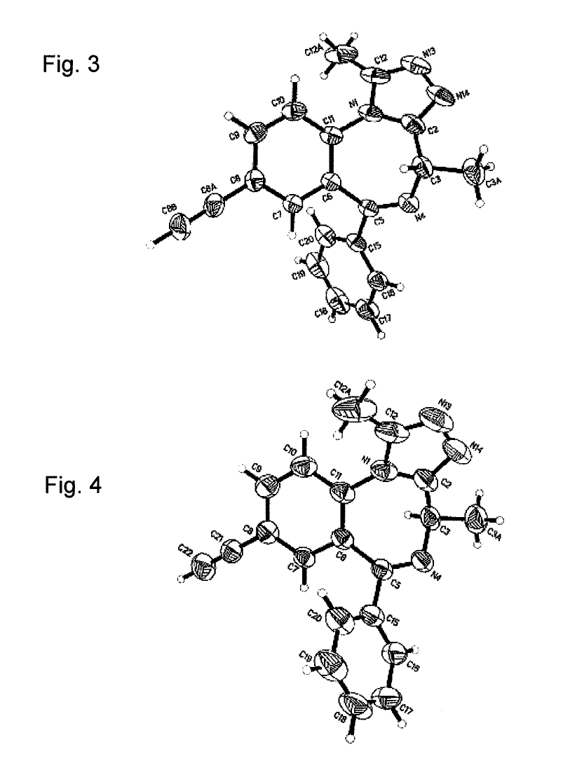

The benzophenone 108 was stirred with N-Boc-L-alanine 107 to give the S carbamic acid tert-butyl ester 109. The ester 109 was treated with HCl(g) in CHCl₃ and then cyclized under basic conditions to give S benzodiazepine 110. The amide 110 was regioselectively brominated at position-7 to give the S bromide 111. The bromide 111 was stirred with the di-4-morpholinophosphinic chloride and this was followed by the addition of acetylhydrazide to furnish triazolobenzodiazepine 141 (SH—I-89S), analogous to the procedure of Ning et al. This material 141 was then subjected to a Heck-type coupling reaction (TMS—C≡CH, Pd-mediated) to furnish prodrug 142 (SH-201). This analog 142 was converted into the 8-acetylenic analog 143 (SH-TS-CH₃) on stirring with fluoride anion. This final product 143 (SH-TS-CH₃) was crystallized from an EtOAc/hexane solvent mixture. Depicted in FIG. 3 is the structure from the ORTEP drawing of the crystal structure of SH-TS-CH₃ (143).

(S)-Tert-butyl 1-(2-benzoylphenylamino)-1-oxopropan-2-ylcarbamate (109) To a stirred solution of 2-aminobenzophenone 108 (5.73 g, 29.07 mmol) and the required N-Boc-L-alanine 107 (5 g, 26.43 mmol) in dry CH₂Cl₂ (200 mL) was added dicyclohexylcarbodiimide (DCC, 5.99 g, 29.07 mmol) in dry CH₂Cl₂ (100 mL) dropwise, over 30 min at 0° C. The reaction mixture was stirred an additional 12 h at rt. The dicyclohexyl urea which formed was filtered off and the filtrate concentrated under reduced pressure. The crude solid 109 was purified by recrystallization from hexane and ethyl acetate to afford 109 (7.88 g, 81%): mp 127-129° C.; [α]_{D}²⁶=−67.7 (c 0.88, EtOAc); IR (KBr, cm⁻¹) 3288, 2475, 2352, 1684; ¹H NMR (CDCl₃) δ 11.48 (s, 1H), 8.67 (d, J=8.22 Hz, 1H), 7.71-7.43 (m, 7H), 7.13-7.08 (m, ¹H) 5.06 (br s, 1H), 4.36 (br s, 1H), 1.50 (d, J=7.1 Hz, 3H), 1.44 (s, 9H); MS (EI) m/e (relative intensity) 368 (M⁺, 6), 295 (10), 225 (27), 224 (79). Anal. Calcd For C₂₁H₂₄N₂O₄: C, 68.46; H, 6.57; N, 7.60. Found: C, 68.59; H, 6.56; N, 7.59.

(S)-3-Methyl-5-phenyl-1H-benzo[e][1,4]diazepin-2(3H)-one (110) To a stirred solution of the benzophenone 109 (10.65 g, 29.38 mmol) in CHCl₃ (400 mL) at rt, hydrogen chloride gas was added in slowly. After 20 min, the addition was completed and the solution was stirred overnight at rt. The reaction mixture was washed with a saturated aq solution of sodium bicarbonate (2×50 mL) and water (2×50 mL). The organic layer was concentrated under reduced pressure. The residual oil was dissolved in methanol-water (2:1, 500 mL) and the pH was adjusted to 8.5 by the addition of aq sodium hydroxide (1 N). The reaction mixture was stirred for 10 h at rt. The solution was concentrated under reduced pressure and water (100 mL) was added. The solution was extracted with CH₂Cl₂ (3×100 mL) and concentrated under reduced pressure. The crude solid 110 was purified by recrystallization from methanol/water to provide 110 (6.10 g, 83%): mp 158-159° C.; [α]_{D}²⁶+290.2 (c 0.78, EtOAc); IR (KBr, cm⁻¹) 3215, 3059, 2974, 2932; ¹H NMR (CDCl₃) δ 9.65 (s, 1H), 7.54-7.13 (m, 9H), 3.78 (q, J=6.5 Hz, 1H), 1.78 (d, J=7.1 Hz, 3H); MS (EI) m/e (relative intensity) 250 (M⁺, 40), 249 (83), 234 (15), 209 (75), 208 (76), 207 (100). Anal. Calcd for C₁₆H₁₄N₂O: C, 76.78; H, 5.64; N, 11.19. Found: C, 76.71; H, 5.62; N, 11.18.

(S)-7-Bromo-3-methyl-5-phenyl-1H-benzo[e][1,4]diazepin-2(3H)-one (111) To a stirred solution of 110 (66.5 g, 265 mmol) in glacial acetic acid (400 mL), sulfuric acid (80 mL) was added to the solution. Bromine (28 mL, 530 mmol) was dissolved in acetic acid (150 mL) and was added dropwise into the mixture. The reaction mixture was allowed to stir until analysis by NMR spectroscopy indicated that all of the starting material 110 had been consumed. The solution was concentrated under reduced pressure and then neutralized by addition of cold aq 1N NaOH solution and then extracted with EtOAc. The crude product 111 was purified by recrystallization from CH₂Cl₂ to afford 111 (70.4 g, 81%): mp 210-212° C.; [α]_{D}²⁶=+313.1 (c 0.34, EtOAc); IR (KBr, cm⁻¹) 2931, 1692, 1603, 1563; ¹H NMR (CDCl₃) δ 9.36 (s, 1H), 7.63 (dd, J=2.22, 8.58 Hz, 1H), 7.54-7.39 (m, 6H), 7.12 (d, J=8.61 1H), 3.76 (q, J=6.3 Hz, 1H), 1.76 (d, J=6.45 Hz, 3H). MS (EI) m/e (relative intensity) 330 (M⁺+1, 21), 329 (M⁺, 50), 328 (22), 327 (48), 289 (44). Anal. Calcd for C₁₆H₁₃BrN₂O: C, 58.38; H, 3.98; N, 8.51. Found: C, 58.43; H, 3.97; N, 8.49.

(S)-7-Bromo-3-methyl-5-phenyl-1H-benzo[e][1,4]diazepin-2(3H)-one (141) A solution of 111 (6.9 g, 21.0 mmol) in THF (50 mL) was cooled to −10° C., and sodium hydride (60% dispersion in mineral oil, 0.72 g, 18 mmol) was added in one portion at −10° C. After 1 hour, di-4-morpholinylphosphinic chloride (4.84 g, 22.5 mmol) was added at −10° C., and the solution which resulted was stirred continuously for 2 h at rt. To this mixture was then added a solution of acetic hydrazide (2.47 g, 30 mmol) in n-BuOH (20 mL) and stirring was continued at rt for 15 min. The solvent was removed and the residue was dissolved in dry n-BuOH (25 mL) and heated to reflux for 2 h. The n-butanol was removed under reduced pressure and the residue was partitioned between CH₂Cl₂ and brine. The CH₂Cl₂ layer was dried and removed under reduced pressure after which the residue was purified by flash chromatography (silica gel, EtOAc) to afford 141 (3 g, 60%) as a white solid: mp 240-242° C.; [α]_{D}²⁶=+52.00 (c 0.50, CH₂Cl₂); IR (KBr) 3401, 3048, 2978, 2930 cm⁻¹; ¹H NMR (300 Hz. CDCl₃) δ 7.86 (d, J=6.4 Hz, 1H), 7.64 (d, J=1.9 Hz, 1H), 7.60-7.47 (m, 4H), 7.44-7.39 (m, 2H), 4.25 (q, J=6.7 Hz, 13.4 Hz, 1H), 2.80 (s, 3H), 2.10 (d, J=6.7 Hz, 3H); MS (EI) m/e (relative intensity) 368 (64), 366 (64), 353 (67), 351 (66) 325 (100), 323 (98), 217 (58). Anal. Calcd for C₁₈H₁₅BrN_{4:} C, 58.87; H, 4.12; N, 15.26. Found: C, 58.91; H, 4.13; N, 15.24.

(4S)-1,4-Dimethyl-6-phenyl-8-((trimethylsilyl)ethynyl)-4H-benzo[f][1,2,4]triazolo[4,3-a][1,4]diazepine (142) A mixture of bromide 141 (1.46 g, 3.8 mmol), trimethylsilylacetylene (0.65 g, 6.6 mmol) and bis(triphenylphosphine)palladium (II) acetate (0.25 g, 0.33 mmol) in a mixed solvent system of CH₃CN (80 mL) and anhydrous triethylamine (50 mL) was degassed and then heated to reflux under argon. After stirring for 2 h at reflux, the mixture was cooled to rt and the precipitate which formed was removed by filtration. The filtrate was concentrated under reduced pressure and the residue was treated with a saturated solution of NaHCO₃ (40 mL), and extracted with CHCl₃ (3×50 mL). The combined organic extracts were washed with brine (2×10 mL) and dried (Na₂SO₄). After removal of the solvent under reduced pressure, the residue was purified by flash chromatography (silica gel, EtOAc) to afford 142 (1.2 g, 77%) as a gray solid: mp 262-264° C.; IR (KBr) 2952, 2157, 1618 cm⁻¹; ¹H NMR (300 MHz CDCl₃) δ 7.77 (dd, J=1.9 Hz, 1.9 Hz, 1H), 7.60-7.54 (m, 3H), 7.50-7.37 (m, 4H), 4.19 (q, J=13.45 Hz, 1H), 2.66 (s, 3H), 2.18 (d, J=12.9 Hz 3H), 0.24 (S, 9H); MS (EI) m/e (relative intensity) 384 (90), 312 (100). Anal. Calcd For C₂₃H₂₄N₄Si: C, 71.84; H, 6.29; N, 14.57. Found: C, 71.88; H, 6.30; N, 14.60.

(4S)-8-Ethynyl-1,4-dimethyl-6-phenyl-4H-benzo[f][1,2,4]triazolo[4,3-a][1,4]diazepine (143, SH-TS-CH₃) A solution of 142 (2 g, 5 mmol) in THF (20 mL) was treated with Bu₄NF.H₂O (4 mL, 1.0 M solution in THF). The mixture which resulted was allowed to stir for 5 min at rt, after which the mixture was added to H₂O (20 mL) and extracted with CH₂Cl₂ (3×50 mL). The combined organic extracts were washed with brine (2×15 mL) and dried (Na₂SO₄). After removal of the solvent under reduced pressure, the residue was purified by flash chromatography (silica gel, EtOAc/MeOH: 100/1) to afford 143 (SH-TS-CH₃) (1.33 g, 85%) as a pale yellow solid: mp>210° C. (dec); [α]_{D}²⁶=+256 (c 0.33, EtOAc); IR (KBr) 3213, 3054, 2978, 2933, 2098 cm⁻¹; ¹H NMR (300 MHz CDCl₃) δ 7.77 (dd, J=1.9 Hz, 1.9 Hz, 1H), 7.60-7.54 (m, 3H), 7.50-7.37 (m, 4H), 4.19 (q, J=13.45 Hz, 1H), 3.18 (s, 1H), 2.66 (s, 3H), 2.18 (d, J=12.9 Hz 3H); ¹³C NMR (300 MHz CDCl₃) δ 166.08, 157.76, 150.11, 138.54, 135.05, 134.53, 133.43, 130.60, 129.62, 129.28, 128.30, 123.28, 121.49, 81.20, 79.76, 51.90, 17.53, 12.22. MS (EI) m/e (relative intensity) 312 (92), 297 (76), 283 (18), 269 (100), 242 (26). Anal. Calcd For C₂₀H₁₆N₄: C, 76.90; H, 5.16; N, 17.94. Found: C, 76.79; H, 5.17; N, 17.92.

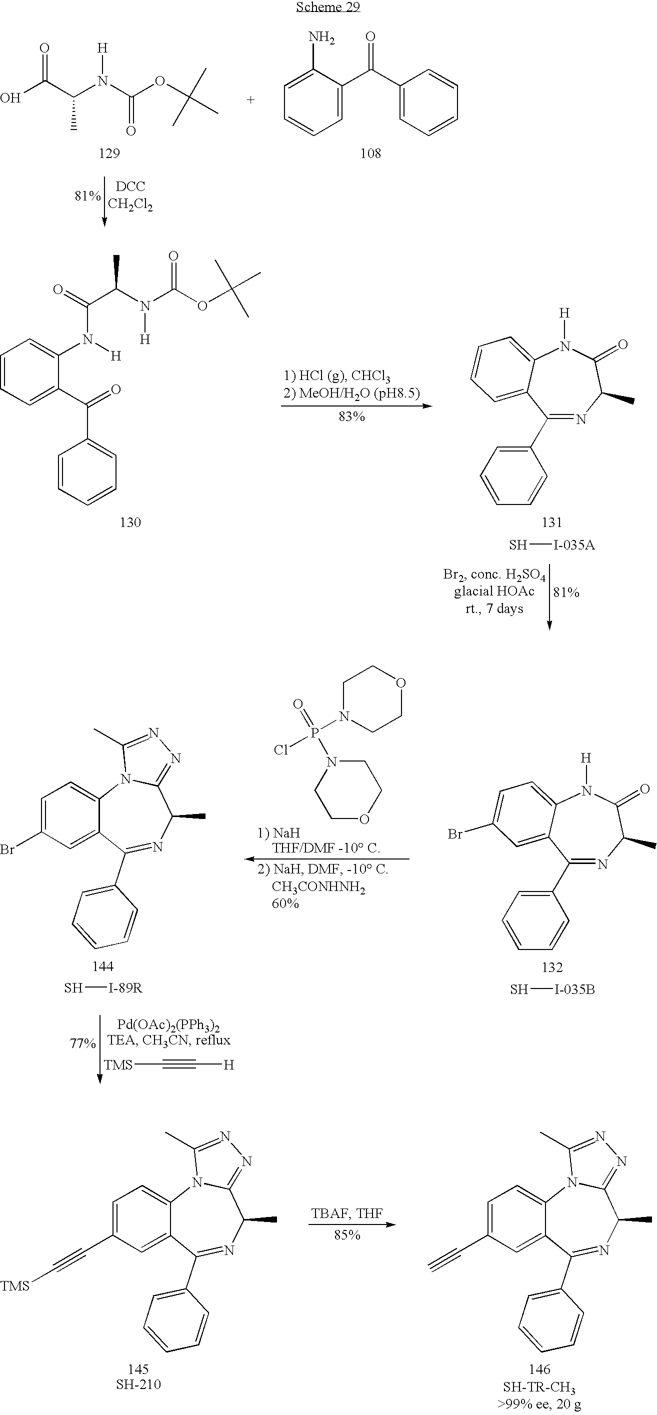

### Synthesis of the R-Enantiomer (SH-TR-CH₃ 146)

The ligand SH-TR-CH₃ 146 is the triazolam analog of SH-053-R—CH₃ 135 and was designed to gain better water solubility, and to enhance the potency as well as avoid the α1-mediated sedative, ataxic, amnesic and muscle relaxant side effects of traditional triazolam-like agents. The benzophenone 108 was stirred with N-Boc-D-alanine 129 to give the R carbamic acid tert-butyl ester 130. The ester 130 was treated with HCl(g) in CHCl₃ and then cyclized under basic conditions to give the R benzodiazepine 131 (SH—I-035A). The amide 131 was regioselectively brominated at position-7 to give the R bromide 132 (SH—I-035B). The bromide132 was then stirred with the di-4-morpholinophosphinic chloride and this was followed by the addition of acetylhydrazide to furnish triazolobenzodiazepine 144 (SH—I-89R), analogous to the procedure of Ning et al. This material 144 (SH—I-89R) was then subjected to a Heck-type coupling reaction (TMS—C≡CH, Pd-mediated) to furnish prodrug 145 (SH-210). This analog 145 was converted into the 8-acetylenic analog 146 (SH-TR-CH₃) on stirring with fluoride anion. This final product 146 (SH-TR-CH₃) was crystallized from an EtOAc/hexane mixture to provide material for X-ray crystallography. The structure of 146 is depicted in FIG. 4 from the ORTEP drawing of SH-TR-CH₃ (146).

In order to establish the enantiopurity of SH-TS-CH₃ (143) and SH-TR-CH₃ (146), the racemate of 143 and 146 were prepared and resolved by chiral HPLC employing a Regis Whelk-01 (S, S) chiral stationary phase. The enantiomeric mixture of S (143) and R (146) isomers was prepared by mixing equimolar amounts of 143 and 146 and dissolving them in CH₂Cl₂. The best mobile phase was optimized in consultation with the data in the Regis web page or handbook. The elution system that gave the best separation of S (143) and R (146) was established. The pure enantiomers were then dissolved in CH₂Cl₂ and separated with the same elution solvent as above. Observation of the retention time of the pure enantiomer versus the racemate and integration of the peaks which resulted provide the % ee value.

(R)-Tert-butyl 1-(2-benzoylphenylamino)-1-oxopropan-2-ylcarbamate (130) To a stirred solution of 2-amino-benzophenone 108 (5.73 g, 29.07 mmol) and the required N-Boc-D-alanine 129 (5 g, 26.43 mmol) in CH₂Cl₂ (200 mL), dicyclohexyl-carbodiimide (DCC, 5.99 g, 29.07 mmol) in CH₂Cl₂ (100 mL) was added dropwise, over 30 min at 0° C. The reaction mixture was stirred an additional 12 h at rt. The dicyclohexyl urea which formed was filtered off and the filtrate concentrated under reduced pressure. The crude solid 130 was purified by recrystallization from hexane and ethyl acetate to afford 130 (7.88 g, 81%): mp 127-129° C.; [α]_{D}²⁶=+67.3 (c 0.88, EtOAc); IR (KBr, cm⁻¹) 3288, 2475, 2352, 1684; ¹H NMR (CDCl₃) δ 11.48 (s, 1H), 8.67 (d, J=8.22 Hz, 1H), 7.71-7.43 (m, 7H), 7.13-7.08 (m, ¹H) 5.06 (br s, 1H), 4.36 (br s, 1H), 1.50 (d, J=7.1 Hz, 3H), 1.44 (s, 9H); MS (EI) m/e (relative intensity) 368 (M⁺, 6), 295 (10), 225 (27), 224 (79). Anal. Calcd For C₂₁H₂₄N₂O₄: C, 68.46; H, 6.57; N, 7.60. Found: C, 68.59; H, 6.56; N, 7.59.

(R)-3-Methyl-5-phenyl-1H-benzo[e][1,4]diazepin-2(3H)-one (131) To a stirred solution of the benzophenone 130 (10.65 g, 29.38 mmol) in CHCl₃ (400 mL) at rt, hydrogen chloride gas was added in slowly. After 20 min, the addition was completed and the solution was stirred overnight at rt. The reaction mixture was washed with a saturated aq solution of sodium bicarbonate (2×50 mL) and water (2×50 mL). The organic layer was concentrated under reduced pressure. The residual oil was dissolved in methanol-water (2:1, 500 mL) and the pH was adjusted to 8.5 by the addition of aq sodium hydroxide (1 N). The reaction mixture was stirred for 10 h at rt. The solution was concentrated under reduced pressure and water (100 mL) was added. The solution was extracted with CH₂Cl₂ (3×100 mL) and concentrated under reduced pressure. The crude solid 131 was purified by recrystallization from methanol/water to provide 131 (6.10 g, 83%): mp 158-159° C.; [α]_{D}²⁶=−288.2 (c 0.78, EtOAc); IR (KBr, cm⁻¹) 3215, 3059, 2974, 2932; ¹H NMR (CDCl₃) δ 9.65 (s, 1H), 7.54-7.13 (m, 9H), 3.78 (q, J=6.5 Hz, 1H), 1.78 (d, J=7.1 Hz, 3H); MS (EI) m/e (relative intensity) 250 (M⁺, 40), 249 (83), 234 (15), 209 (75), 208 (76), 207 (100). Anal. Calcd for C₁₆H₁₄N₂O: C, 76.78; H, 5.64; N, 11.19. Found: C, 76.71; H, 5.62; N, 11.16.

(R)-7-Bromo-3-methyl-5-phenyl-1H-benzo[e][1,4]diazepin-2(3H)-one (132) To a stirred solution of 131 (66.5 g, 265 mmol) in glacial acetic acid (400 mL), cone sulfuric acid (80 mL) was added to the solution. Bromine (28 mL, 530 mmol) was dissolved in acetic acid (150 mL) and was added dropwise into the mixture. The reaction mixture was allowed (approximately 7 days) to stir until analysis by NMR spectroscopy indicated that all of the starting material 131 had been consumed. The solution was concentrated under reduced pressure and then neutralized by addition of cold aq 1N NaOH solution and then extracted with EtOAc. The crude bromide 132 was purified by recrystallization from CH₂Cl₂ to afford 132 (70.4 g, 81%): mp 210-212° C.; [α]_{D}²⁶=−308.6 (c 0.35, EtOAc); IR (KBr, cm⁻¹) 2931, 1692, 1603, 1563; ¹H NMR (CDCl₃) δ 9.36 (s, 1H), 7.63 (dd, J=2.22, 8.58 Hz, 1H), 7.54-7.39 (m, 6H), 7.12 (d, J=8.61 1H), 3.76 (q, J=6.3 Hz, 1H), 1.76 (d, J=6.45 Hz, 3H). MS (EI) m/e (relative intensity) 330 (M⁺+1, 21), 329 (M⁺, 50), 328 (22), 327 (48), 289 (44). Anal. Calcd for C₁₆H₁₃BrN₂O: C, 58.38; H, 3.98; N, 8.51. Found: C, 58.43; H, 3.97; N, 8.49.

(R)-7-Bromo-3-methyl-5-phenyl-1H-benzo[e][1,4]diazepin-2(3H)-one (144) A solution of 132 (6.9 g, 21.0 mmol) in THF (50 mL) was cooled to −10° C., and sodium hydride (60% dispersion in mineral oil, 0.72 g, 18 mmol) was added in one portion. After 1 hour, di-4-morpholinylphosphinic chloride (4.84 g, 22.5 mmol) was added at −10° C., and the solution which resulted was stirred continuously for 2 h at rt. To this mixture was then added a solution of acetic hydrazide (2.47 g, 30 mmol) in n-BuOH (20 mL) and stirring was continued at rt for 15 min. The solvent was removed under reduced pressure and the residue was dissolved in n-BuOH (25 mL) and heated to reflux for 2 h. The n-butanol was removed under reduced pressure and the residue was partitioned between CH₂Cl₂ and brine. The CH₂Cl₂ layer was dried and removed under reduced pressure after which the residue was purified by flash chromatography (silica gel, EtOAc) to afford 144 (3 g, 60%) as a white solid: mp°240-242° C.; [α]_{D}²⁶=−51.20 (c 0.49, CH₂Cl₂); IR (KBr) 3401, 3048, 2978, 2930 cm⁻¹; ¹H NMR (300 Hz. CDCl₃) δ 7.86 (d, J=6.4 Hz, 1H), 7.64 (d, J=1.9 Hz, 1H), 7.60-7.47 (m, 4H), 7.44-7.39 (m, 2H), 4.25 (q, J=6.7 Hz, 13.4 Hz, 1H), 2.80 (s, 3H), 2.10 (d, J=6.7 Hz, 3H); MS (EI) m/e (relative intensity) 368 (64), 366 (64), 353 (67), 351 (66) 325 (100), 323 (98), 217 (58). Anal. Calcd for C₁₈H₁₅BrN_{4:} C, 58.87; H, 4.12; N, 15.26. Found: C, 58.91; H, 4.14; N, 15.23.

(4R)-1,4-Dimethyl-6-phenyl-8-((trimethylsilyl)ethynyl)-4H-benzo[f][1,2,4]triazolo[4,3-a][1,4]diazepine (145) A mixture of bromide144 (1.46 g, 3.8 mmol), trimethylsilylacetylene (0.65 g, 6.6 mmol) and bis(triphenylphosphine)palladium (II) acetate (0.25 g, 0.33 mmol) in a mixed solvent system of CH₃CN (80 mL) and anhydrous triethylamine (50 mL) was degassed and then heated to reflux under argon. After stirring for 2 h at reflux, the mixture was cooled to rt and the precipitate which formed was removed by filtration. The filtrate was concentrated under reduced pressure and the residue was treated with a saturated solution of NaHCO₃ (40 mL), and extracted with CHCl₃ (3×50 mL). The combined organic extracts were washed with brine (2×10 mL) and dried (Na₂SO₄). After removal of the solvent under reduced pressure, the residue was purified by flash chromatography (silica gel, EtOAc) to afford 145 (1.2 g, 77%) as a gray solid: mp 262-264° C.; IR (KBr) 2952, 2157, 1618 cm⁻¹; ¹H NMR (300 MHz CDCl₃) δ 7.77 (dd, J=1.9 Hz, 1.9 Hz, 1H), 7.60-7.54 (m, 3H), 7.50-7.37 (m, 4H), 4.19 (q, J=13.45 Hz, 1H), 2.66 (s, 3H), 2.18 (d, J=12.9 Hz 3H), 0.24 (S, 9H); MS (EI) m/e (relative intensity) 384 (90), 312 (100). Anal. Calcd For C₂₃H₂₄N₄Si: C, 71.84; H, 6.29; N, 14.57. Found: C, 71.88; H, 6.33; N, 14.62.

(4R)-8-Ethynyl-1,4-dimethyl-6-phenyl-4H-benzo[f][1,2,4]triazolo[4,3-a][1,4]diazepine (146, SH-TR-CH₃) A solution of 145 (2 g, 5 mmol) in THF (20 mL) was treated with Bu₄NF.H₂O (4 mL, 1.0 M solution in THF). The mixture which resulted was allowed to stir for 5 min at rt after which the mixture was added to H₂O (20 mL) and extracted with CH₂Cl₂ (3×50 mL). The combined organic extracts were washed with brine (2×15 mL) and dried (Na₂SO₄). After removal of the solvent under reduced pressure, the residue was purified by flash chromatography (silica gel, EtOAc/MeOH: 100/1) to afford 146 (SH-TR-CH₃) (1.33 g, 85%) as a pale yellow solid: mp>210° C. (dec); [α]_{D}²⁶=−252 (c 0.31, EtOAc); IR (KBr) 3213, 3054, 2978, 2933, 2098 cm⁻¹; ¹H NMR (300 MHz CDCl₃) δ 7.77 (dd, J=1.9 Hz, 1.9 Hz, 1H), 7.60-7.54 (m, 3H), 7.50-7.37 (m, 4H), 4.19 (q, J=13.45 Hz, 1H), 3.18 (s, 1H), 2.66 (s, 3H), 2.18 (d, J=12.9 Hz 3H); ¹³C NMR (300 MHz CDCl₃) δ 166.08, 157.76, 150.11, 138.54, 135.05, 134.53, 133.43, 130.60, 129.62, 129.28, 128.30, 123.28, 121.49, 81.20, 79.76, 51.90, 17.53, 12.22. MS (EI) m/e (relative intensity) 312 (92), 297 (76), 283 (18), 269 (100), 242 (26). Anal. Calcd For C₂₀H₁₆N₄: C, 76.90; H, 5.16; N, 17.94. Found: C, 76.89; H, 5.17; N, 17.92.

### ASSAYS OF COMPETITIVE BINDING TO αxβ3γ2 GABA_{A} RECEPTORS

The GABA_{A} subunit selectivity of several compounds prepared as described above were determined using competitive binding assays. The assays were performed in a total volume of 0.5 mL at 4° C. for 1 hour using [³H]flunitrazepam as the radiolabel. For these binding assays, 20-50 mg of membrane protein harvested with hypotonic buffer (50 mM Tris-acetate pH 7.4 at 4 degree) was incubated with the radiolabel as previously described (Choudhary, M. S., Craigo. S., Roth, B. L., Identification of receptor domains that modify ligand binding to 5-hydroxy-tryptamine-2 and 5-hydroxytryptamine 1c serotonin receptors, Mol. Pharmacol. 1992 October; 42(4):627-33). Nonspecific binding was defined as radioactivity bound in the presence of 100 μM diazepam and represented less than 20% of total binding. Membranes were harvested with a Brandel cell harvester followed by three ice-cold washes onto polyethyleneimine-pretreated (0.3%) Whatman GF/C filters. Filters were dried overnight and then soaked in Ecoscint, a liquid scintillation cocktail (National Diagnostics; Atlanta, Ga.). Bound radioactivity was quantified by liquid scintillation counting. Membrane protein concentrations were determined using an assay kit from Bio-Rad (Hercules, Calif.) with bovine serum albumin as the standard. The results are summarized in Table 2, below. Table 3 summarizes the results of similar competitive binding assays performed using stereoisomers.

TABLE 2
Results of Competitive Binding Assays
Binding Affinity (nM) at αxβ3γ2 GABA_{A} Receptors

| Compound | GABA_{A}/α1 | GABA_{A}/α2 | GABA_{A}/α3 | GABA_{A}/α4 | GABA_{A}/α5 | GABA_{A}/α6 |
|---|---|---|---|---|---|---|
| Diazepam | 14 | 20 | 15 | >1000 | 11 | >1000 |
| QH-II-066 | 76.3 | 42.1 | 47.4 | >1000 | 6.8 | >1000 |
| XHE-II-012 | 49 | 24 | 31 | 1042 | 14 | 2038 |
| XHE-II-053 | 287 | 45 | 96 | 1504 | 13.8 | 1000 |
| PS-I-37 | 193 | 35 | 434 | >5000 | 22 | >5000 |
| PS-I-72 | 123 | 31 | 386 | >5000 | 34 | >5000 |
| JC-221 | 146 | 35 | 182 | ND | 14.3 | 362 |
| DM-II-20 | 5717 | 177 | 35.68 | 5000 | 197.7 | 5000 |
| JYI-53 | 77.4 | 40.4 | 183 | 5000 | 133 | 3650 |
| JYI-49 | 1.87 | 2.38 | ND | 5000 | 6.7 | 3390 |
| JY-XHe-053 (a) | 21.99 | 12.34 | ND^{a} | ND^{a} | 0.671 | ND^{a} |
| Hz-166 (a) | 209 | 154 | 446 | 5000^{a} | 79.44 | 5000^{a} |

(a) ND, not determined yet, the binding of α4 and α6 have not been determined, but since the 6-phenyl is present, it will not bind to the α4 and α6 sites

TABLE 3
Results of Competitive Binding Assays
Binding Affinity (nM) at αxβ3γ2 GABA_{A} Receptors

| Compound | GABA_{A}/α1 | GABA_{A}/α2 | GABA_{A}/α3 | GABA_{A}/α4 | GABA_{A}/α5 | GABA_{A}/α6 |
|---|---|---|---|---|---|---|
| SH-053-S-CH₃ | 1666 | 1263 | 1249 | >5000 | 206.4 | >5000 |
| SH-053-R-CH₃ | 2026 | 2377 | 1183 | >5000 | 949.1 | >5000 |
| SH-053-2′F-S-CH₃ | 350 | 141 | 1237 | >5000 | 16 | 5000 |
| SH-053-2′F-S-CH₃ | 468.2 | 33.27 | 291.5 | >5000 | 19.2 | >5000 |
| SH-053-2′F-R-CH₃ | 759.1 | 948.2 | 768.8 | >5000 | 95.17 | >5000 |
| SH-TS-CH₃ (a) | 107.2 | 50.1 | ND | ND | 8.1 | ND |
| SH-TR-CH₃ (a) | 7367 | 1534 | ND | ND | 1948.5 | ND |

(a) ND, not determined yet, the binding of α4 and α6 have not been determined, but since the 6-phenyl is present, it will not bind to the α4 and α6 sites

Subunit Selective Efficacy Determined From Voltage Clamp Recordings From Xenopus Oocytes Expressing αxβ3γ2 GABA_{A} Receptors

The GABA_{A} subunit selectivity of several compounds prepared as described above were determined using voltage clamp recordings from Xenopus oocytes that expressed that αxβ3γ2 GABA_{A} receptor.

In Vitro Expression of GABA_{A} Receptor Subunits The methods used for isolating, culturing, injecting and defolliculating of the oocytes were identical with those described by E. Sigel. See Fuchs, K.; et al., Endogenous [3H]flunitrazepam binding in human embryonic kidney cell line 293. European journal of pharmacology 1995, 289, (1), 87-95; Sigel, E.;& Baur, R., Allosteric modulation by benzodiazepine receptor ligands of the GABA_{A} receptor channel expressed in Xenopus oocytes. J. Neurosci. 1988, 8, (1), 289-95; Sigel, E.; & Minier, F., The Xenopus oocyte: System for the study of functional expression and modulation of proteins. Molecular Nutrition & Food Research 2005, 49, (3), 228-234. Mature female Xenopus laevis (Nasco, Wis.) were anaesthetized in a bath of ice-cold 0.17% Tricain (Ethyl-m-aminobenzoate, Sigma, Mo.) before decapitation and removal of the frog ovary. Stage 5 to 6 oocytes with the follicle cell layer around them were singled out of the ovary using a platinum wire loop. Oocytes were stored and incubated at 18° C. in modified Barths medium (MB, containing 88 mM NaCl, 10 mM HEPES-NaOH (pH 7.4), 2.4 mM NaHCO₃, 1 mM KCl, 0.82 mM MgSO₄, 0.41 mM CaCl₂, 0.34 mM Ca(NO₃)₂) that was supplemented with 100 Units/mL penicillin and 100 μg/mL streptomycin. Oocytes with follicle cell layers still around them were injected with 50 mL of an aqueous solution of the cRNA. This solution contained the transcripts for the different alpha subunits and the beta 3 subunit at a concentration of 0.0065 ng/nL as well as the transcript for the gamma 2 subunit at 0.032 ng/nL. After injection of the cRNA, oocytes were incubated for at least 36 h before the enveloping follicle cell layers were removed. To this end, oocytes were incubated for 20 minutes at 37° C. in MB that contained 1 mg/mL collagenase type IA and 0.1 mg/mL trypsin inhibitor I—S (both Sigma). This was followed by osmotic shrinkage of the oocytes in doubly concentrated MB medium supplied with 4 mM Na-EGTA. Finally, the oocytes were transferred to a culture dish containing MB and were gently pushed away from the follicle cell layer that stuck to the surface of the dish. After removal of the follicle cell layer, oocytes were allowed to recover for at least 4 h before being used in electrophysiological experiments.

Voltage Clamp Recordings From Xenopus Oocytes. For electrophysiological recordings, oocytes were placed on a nylon-grid in a bath of Xenopus Ringer solution (XR, containing 90 mM NaCl, 5 mM HEPES-NaOH (pH 7.4), 1 mM MgCl₂, 1 mM KCl and 1 mM CaCl₂). The oocytes were constantly washed by a flow of 6 mL/min XR that could be switched to XR containing GABA and/or drugs. Drugs were diluted into XR from DMSO-solutions resulting in a final concentration of 0.1% DMSO perfusing the oocytes. Drugs were preapplied for 30 seconds before the addition of GABA, which was coapplied with the drugs until a peak response was observed. Between two applications, oocytes were washed in XR for up to 15 minutes to ensure full recovery from desensitization. For current measurements the oocytes were impaled with two microelectrodes (2-3 ma) that were filled with 2 mM KCl. All recordings were performed at rt at a holding potential of −60 mV using a Warner OC-725C two-electrode voltage clamp (Warner Instruments, Hamden, Conn.). Data were digitized, recorded and measured using a Digidata 1322A data acquisition system (Axon Instruments, Union City, Calif.). Results of concentration response experiments were fitted using GraphPad Prism 3.00 (GraphPad Software, San Diego, Calif.). The equation used for fitting concentration response curves was Y=Bottom+(Top-Bottom)/(1+10̂((LogEC50-X)*HillSlope)); X represents the logarithm of concentration, Y represents the response; Y starts at Bottom and goes to Top with a sigmoid shape. This is identical to the “four parameter logistic equation.”

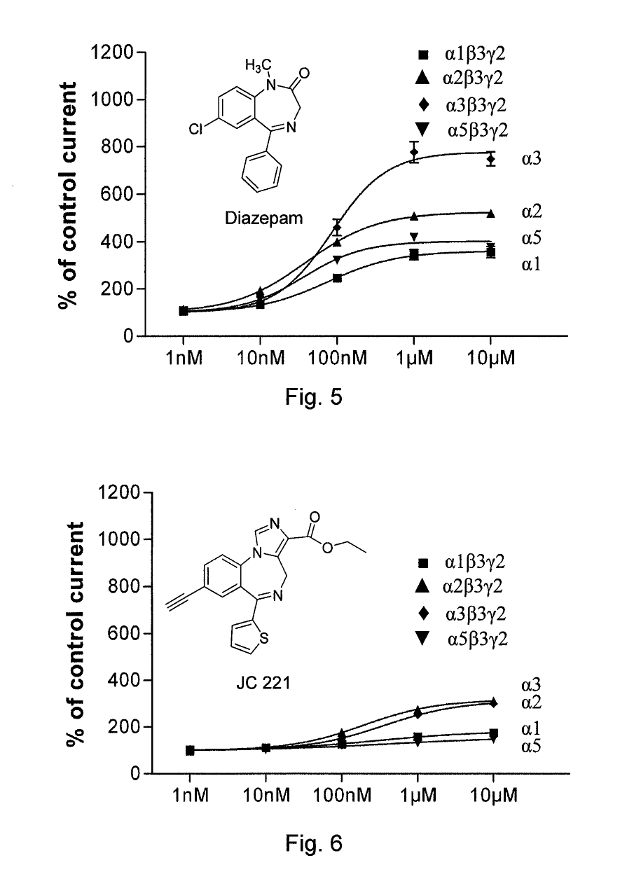

FIG. 5 is a graph of dose-response curves of the patch clamp current (expressed as percent of the control current) produced by various concentrations of diazepam when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 6 is a graph of dose-response curves of the patch clamp current (expressed as percent of the control current) produced by various concentrations of JC-221 when applied to Xenopus oocytes expressing α1μ3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

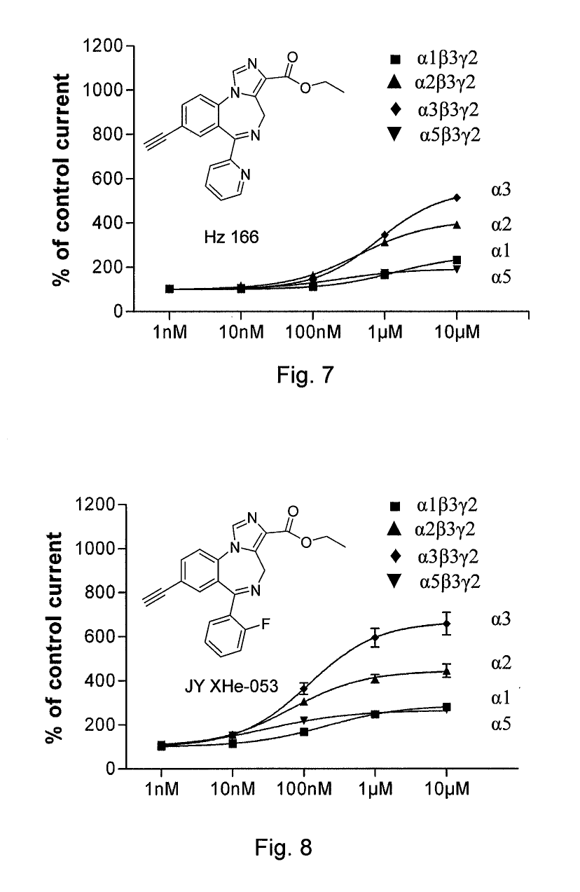

FIG. 7 is a graph of dose-response curves of the patch clamp current (expressed as percent of the control current) produced by various concentrations of Hz-166 when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 8 is a graph of dose-response curves of the patch clamp current (expressed as percent of the control current) produced by various concentrations of JY-XHe-053 (15) when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

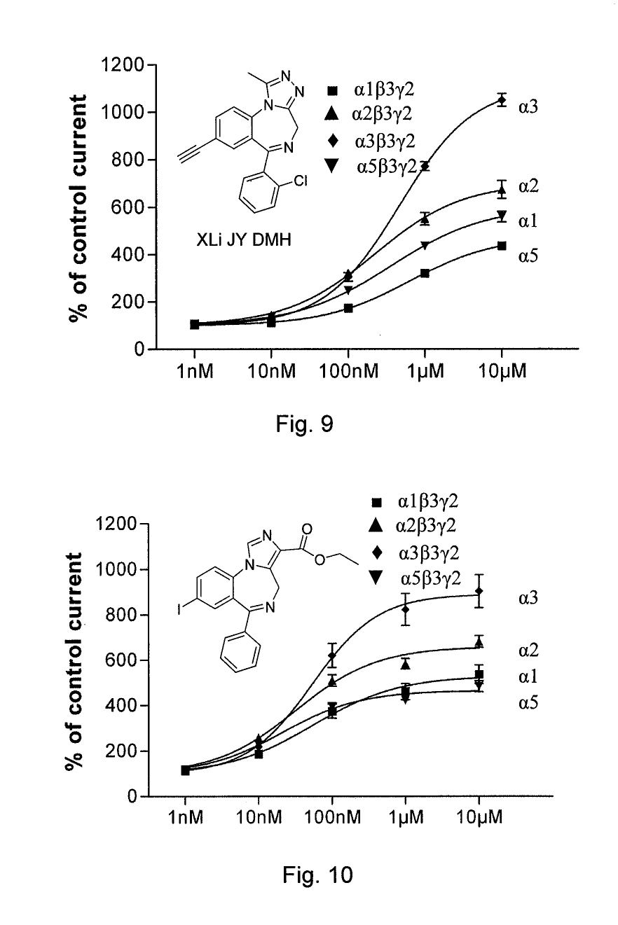

FIG. 9 is a graph of dose-response curves of the patch clamp current (expressed as percent of the control current) produced by various concentrations of XLi-JY-DMH (23) when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

FIG. 10 is a graph of dose-response curves of the patch clamp current (expressed as percent of the control current) produced by various concentrations of compound 147, an 8-iodo-imidazobenzodiazepine (the iodo analog of compound 5), when applied to Xenopus oocytes expressing α1β3γ2 (▪), α2β3γ2 (▴), α3β3γ2 (♦) or α5β3γ2 (▾) GABA_{A} receptors. Values are presented as mean±SEM of at least four oocytes from at least two batches expressing recombinant GABA_{A}/α₁ receptors, GABA_{A}/α₂ receptors, GABA_{A}/α₃ receptors or GABA_{A}/α₅ receptors.

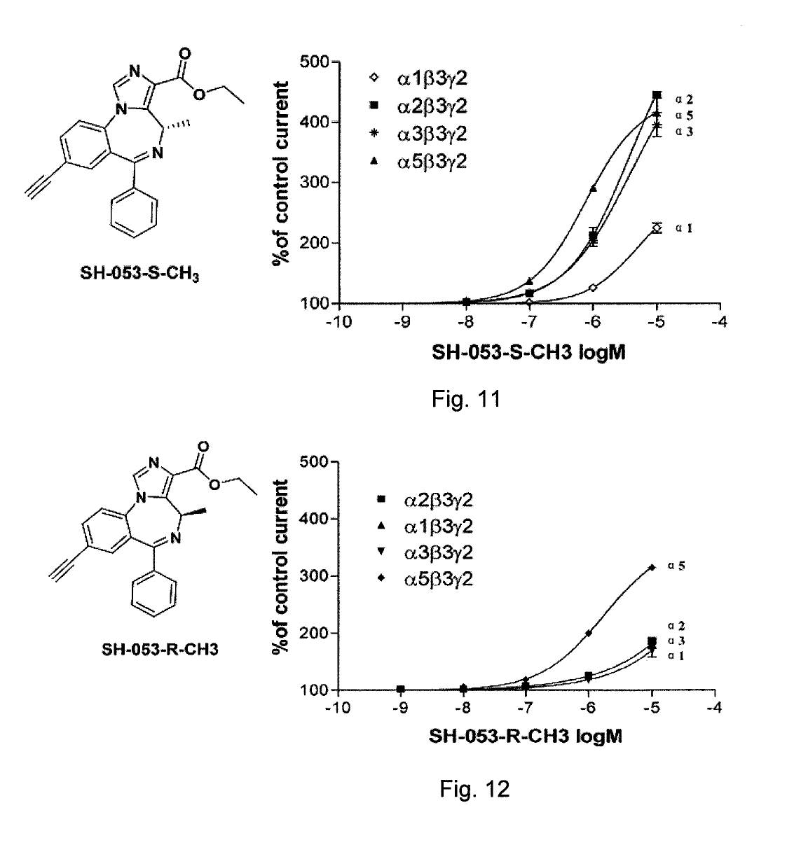

FIG. 11 is a graph of dose-response curves of the patch clamp current (expressed as percent of the control current) produced by various concentrations of SH-053-S—CH₃ when applied to Xenopus oocytes expressing recombinant α1β3γ2 (open diamond), α2β3γ2 (▪), α3β3γ2 (*) or α5β3γ2 (▴) GABA_{A} receptors.

FIG. 12 is a graph of dose-response curves of the patch clamp current (expressed as percent of the control current) produced by various concentrations of SH-053-R—CH₃ when applied to Xenopus oocytes expressing recombinant α1β3γ2 (▴), α2β3γ2 (▪), α3β3γ2 (▾) or α5β3γ2 (♦) GABA_{A} receptors.

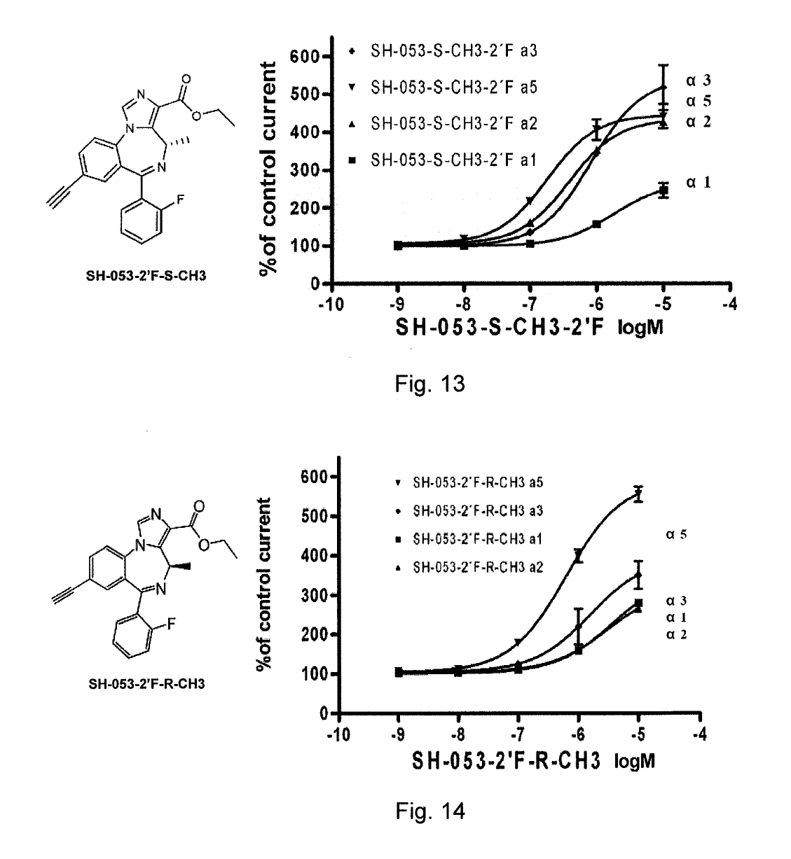

FIG. 13 is a graph of dose-response curves of the patch clamp current (expressed as percent of the control current) produced by various concentrations of SH-053-2′F—S—CH₃ when applied to Xenopus oocytes expressing recombinant α1β3γ2 (▪), α2β3γ2 (▴), α3β3γ2 (♦) or α5β3γ2 (▾) GABA_{A} receptors.

FIG. 14 is a graph of dose-response curves of the patch clamp current (expressed as percent of the control current) produced by various concentrations of SH-053-2′F—R—CH₃ when applied to Xenopus oocytes expressing recombinant α1β3γ2 (▪), α2β3γ2 (▴), α3β3γ2 () or α5β3γ2 (▾) GABA_{A} receptors.

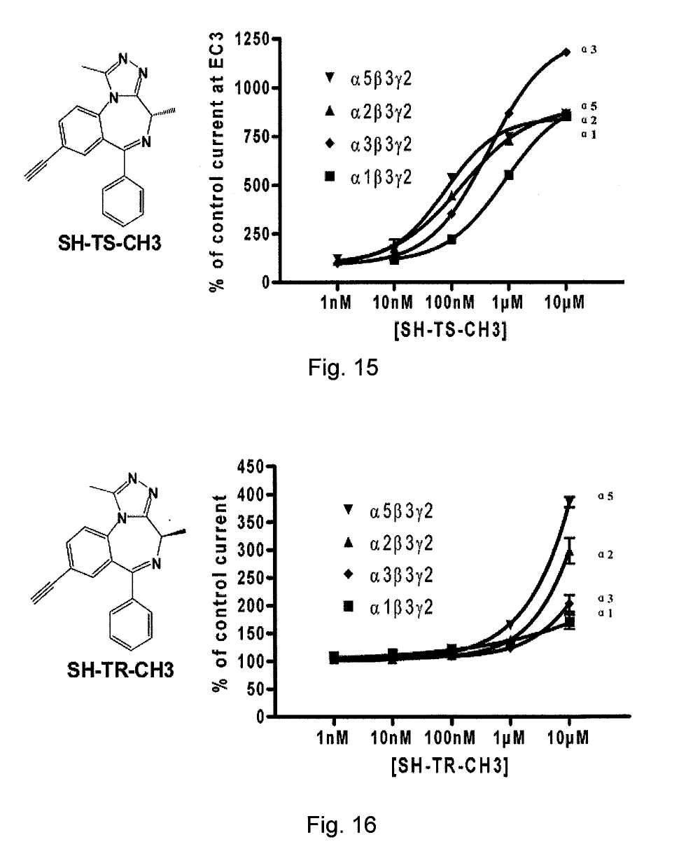

FIG. 15 is a graph of dose-response curves of the patch clamp current (expressed as percent of the control current) produced by various concentrations of SH-TS-CH₃ when applied to Xenopus oocytes expressing recombinant α1β3γ2 (▪), α2β3γ2 (▴), α3β3γ2 (♦) or α5β3γ2 (▾) GABA_{A} receptors.

FIG. 16 is a graph of dose-response curves of the patch clamp current (expressed as percent of the control current) produced by various concentrations of SH-TR-CH₃ when applied to Xenopus oocytes expressing recombinant α1β3γ2 (▪), α2β3γ2 (▴), α3β3γ2 (♦) or α5β3γ2 (▾) GABA_{A} receptors.

### Anticonvulsant Uses of Benzodiazepine Derivative Compounds Selective for GABA_{A} Receptor Subunits

The benzodiazepine derivative compounds selective for GABAₐ receptor subunits were tested for the ability to suppress seizures in several standard rat and mouse models of epilepsy. Anticonvulsant activity of several of the compounds of preferred embodiments of the present invention was compared to diazepam. The standard models incorporated into anticonvulsant screening include the maximal electroshock test (MES), the subcutaneous Metrazol test (scMet), and evaluations of toxicity (TOX). The data for each condition is presented as a ratio of either the number of animals protected or toxic (loss of locomotor activity) over the number of animals tested at a given time point and dose.

The MES is a model for generalized tonic-clonic seizures and provides an indication of a compound's ability to prevent seizure spread when all neuronal circuits in the brain are maximally active. These seizures are highly reproducible and are electrophysiologically consistent with human seizures. For all tests based on MES convulsions, 60 Hz of alternating current (50 mA in mice, 150 in rats) is delivered for by corneal electrodes which have been primed with an electrolyte solution containing an anesthetic agent (0.5% tetracaine HCL). For Test 1, mice are tested at various intervals following doses of 30, 100 and 300 mg/kg of test compound given by ip injection of a volume of 0.01 mL/g. In Test 2, rats are tested after a dose of 30 mg/kg (po) in a volume of 0.04 mL/g. Test 8 uses varying doses administered via i.p. injection, again in a volume of 0.04 ml/g. An animal is considered “protected” from MES-induced seizures upon abolition of the hindlimb tonic extensor component of the seizure (Swinyard, E. A., et al. General principles: experimental selection, quantification, and evaluation of anticonvulsants. In Antiepileptic Drugs, Levy, R. H. M., et al., Eds.; Raven Press: New York, 1989; pp 85-102; White, H. S., et al., The early identification of anticonvulsant activity: role of the maximal electroshock and subcutaneous pentylenetetrazol seizure models. Ital J Neurol Sci. 1995a, 16, 73-7; White, H. S., et al., General principles: experimental selection, quantification, and evaluation of antiepileptic drugs. In Antiepileptic Drugs, Levy, R. H. M., Meldrum, B. S., Eds.; Raven Press: New York, pp 99-110, 1995b.).

Subcutaneous injection of the convulsant Metrazol produces clonic seizures in laboratory animals. The scMet test detects the ability of a test compound to raise the seizure threshold of an animal and thus protect it from exhibiting a clonic seizure. Animals are pretreated with various doses of the test compound (in a similar manner to the MES test, although a dose of 50 mg/kg (po) is the standard for Test 2 scMet). At the previously determined TPE of the test compound, the dose of Metrazol which will induce convulsions in 97% of animals (CD₉₇: 85 mg/kg mice) is injected into a loose fold of skin in the midline of the neck. The animals are placed in isolation cages to minimize stress (Swinyard, E. A., et al., Studies on the mechanism of amphetamine toxicity in aggregated mice. J. Physiol. 1961, 132, 97-0.102) and observed for the next 30 minutes for the presence or absence of a seizure. An episode of clonic spasms, approximately 3-5 seconds, of the fore and/or hindlimbs, jaws, or vibrissae is taken as the endpoint. Animals which do not meet this criterion are considered protected.

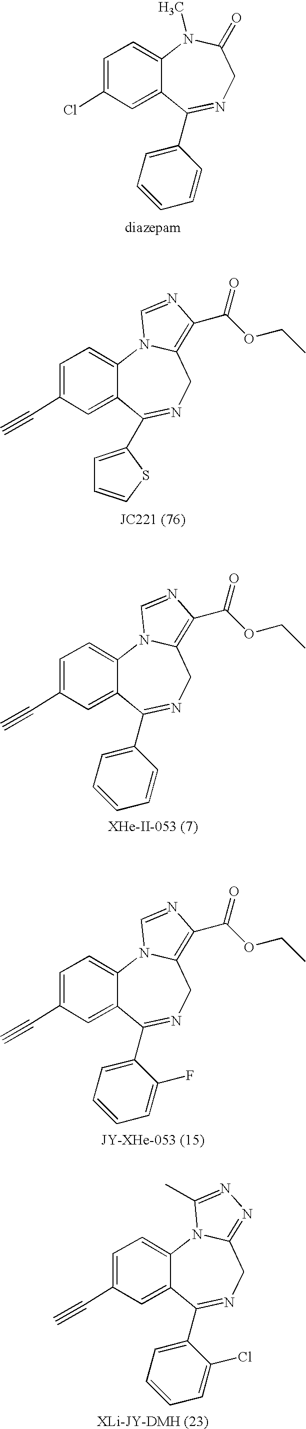

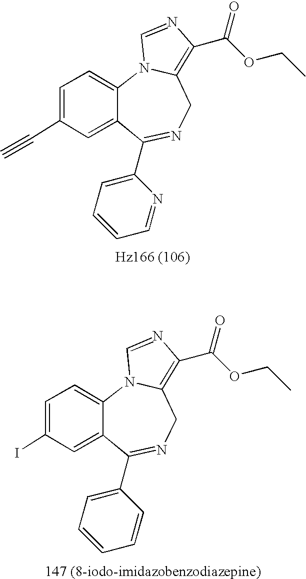

To assess a compound's undesirable side effects (toxicity), animals are monitored for overt signs of impaired neurological or muscular function. In mice, the rotorod procedure (Dunham, M. S.; Miya, T. A., A note on a simple apparatus for detecting neurological deficit in rats and mice. J. Amer. Pharm. Ass. Sci. Ed. 1957, 46, 208-209) is used to disclose minimal muscular or neurological impairment. When a mouse is placed on a rod that rotates at a speed of 6 rpm, the animal can maintain its equilibrium for long periods of time. The animal is considered toxic if it falls off this rotating rod three times during a 1-min period. In rats, minimal motor deficit is indicated by ataxia, which is manifested by an abnormal, uncoordinated gait. Rats used for evaluating toxicity are examined before the test drug is administered, since individual animals may have peculiarities in gait, equilibrium, placing response, etc., which might be attributed erroneously to the test substance. In addition to MMI, animals may exhibit a circular or zigzag gait, abnormal body posture and spread of the legs, tremors, hyperactivity, lack of exploratory behavior, somnolence, stupor, catalepsy, loss of placing response and changes in muscle tone.

Based on the subtype selective efficacy at α3 over α1 and α5 found in the Xenopus oocyte studies, ligands Hz166 106, JY-XHe-053 15, and XLi-JY-DMH (23) were expected to demonstrate the best anticonvulsant profile. Examination of the preliminary anticonvulsant screen (Table 4) to assess locomotor activity (loss of) at the National Institutes of Neurological Disorders and Stroke (NINDS) under the Anticonvulsant Screening Program (ASP) indicated that the 8-acetyleno-T-pyridoimidazobenzodiazepine Hz166 (106) had the best anti-seizure profile in mice when administrated i.p. It raised the seizure threshold level induced by subcutaneous

TABLE 4
Assessment of Antiseizure Activity at 100 mg/kg Dose IP
After 0.5 hr and 4.0 hr (Preliminary Screen)

| | | Mice IP | | | Rat IP | | |
|---|---|---|---|---|---|---|---|
| | | | | TOX | | | TOX |
| | Time | | | (Rotorod | | | (Rotorod |
| Compound | (hr) | MES | scMet | Test) | MES | scMet | Test) |
| XHe-II-053 (7) | 0.5 | 0/3 | 0/1 | 0/8 | | 1/4 | 2/4 |
| | 4.0 | 0/3 | 0/1 | 0/4 | | 0/4 | 0/4 |
| JC 221 (76) | 0.5 | 0/3 | 0/1 | 0/8 | | | |
| | 4.0 | 0/3 | 0/1 | 0/4 | | | |
| Hz166 (106) | 0.5 | 0/3 | 3/5 | 0/8 | | 7/8 | 0/8 |
| (XHe-II-053 2′N) | 4.0 | 0/3 | 0/1 | 0/4 | | 5/8 | 0/8 |
| JY-XHe-053 (15) | 0.5 | 0/3 | 5/5 | 6/8 | | 3/4 | 0/4 |
| (XHe-II-053 2′F) | 4.0 | 0/3 | 0/1 | 0/4 | | 3/4 | 0/4 |
| XLi-JY-DMH (23) | 0.5 | 3/3 | 1/1 | 6/8 | | | |
| (triazol analog) | 4.0 | 3/3 | 1/1 | 2/4 | | | |
| 147 (8-iodo-imidazo- | 0.5 | 0/3 | 0/1 | 0/8 | | 1/4 | 0/4 |
| benzodiazepine) | 4.0 | 0/3 | 0/1 | 0/4 | | 2/4 | 0/4 |

metrazole (scMet) in 60% of mice (3/5) with no motor impairment as indicated by the rotorod paradigm test (Tox). Ligand Hz166 (106) also appeared to have a relatively rapid onset and short duration of action because the antiseizure protection was absent after 4.0 hours. Toxicity in this report is based on motor impairment (locomotor, rotorod). The triazobenzodiazepine XLi-JY-DMH (23) showed increased antiseizure activity against scMet induced seizures with 100% protection and had a longer duration of action. This is not surprising since it is devoid of the 3-ethyl ester moiety. It was also the only benzodiazepine of this series that showed significant protection against the spread of seizure discharge through neural tissue (MES) in mice. Ligand XLi-JY-DMH (23) induced significant minimal motor impairment and sedative effects in mice (ip).

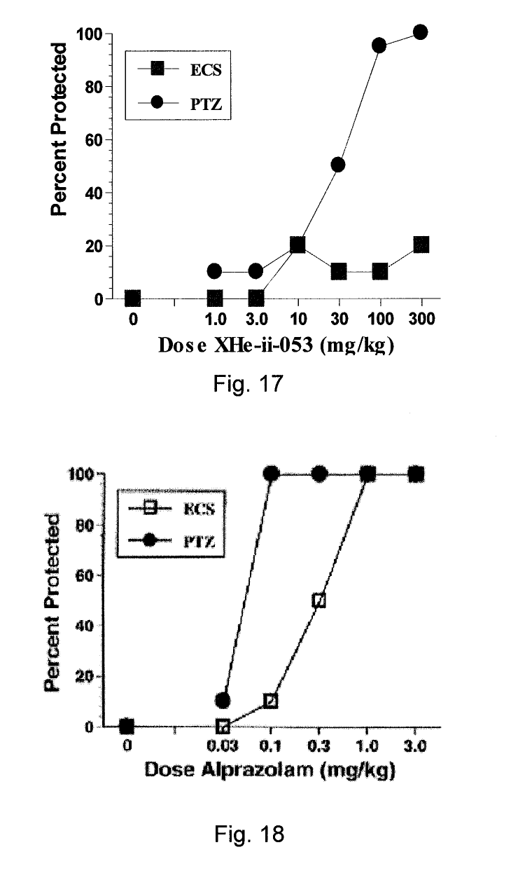

FIG. 17 shows the in vivo anticonvulsant activity of compound XHe-II-053 in the maximum electroconvulsive shock (ECS, ▪) and subcutaneous metrazole seizure (PTZ, ) models of epilepsy.

FIG. 18 shows the in vivo anticonvulsant activity of alprazolam in the maximum electroconvulsive shock (ECS, ▪) and subcutaneous metrazole seizure (PTZ, ) models of epilepsy.

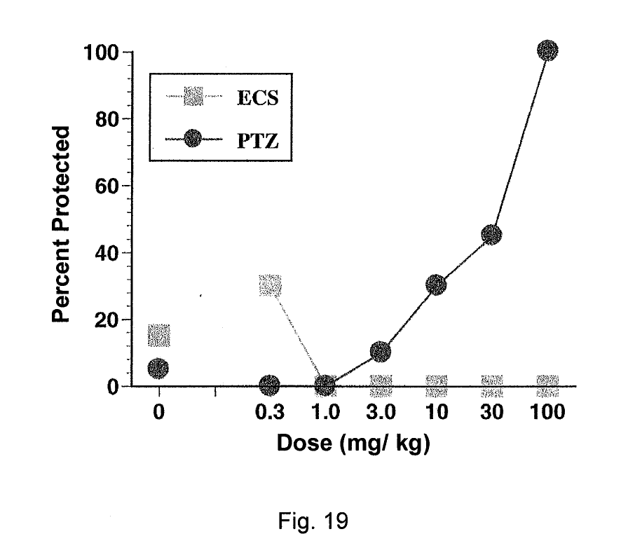

FIG. 19 shows the in vivo anticonvulsant activity of compound XLi—XHe-II-048 in the maximum electroconvulsive shock (ECS, ▪) and subcutaneous metrazole seizure (PTZ, ) models of epilepsy.

TABLE 5
Assessment of Antiseizure Activity in Mice at 50 mg/kg Dose PO
After 0.5 hr and 4.0 hr (Preliminary Screen)

| | | Mice IP | | |
|---|---|---|---|---|
| Compound | Time (hr) | MES | scMet | TOX (Rotorod Test) |
| XHe-II-053 (7) | 0.5 | | 1/4 | 2/4 |
| | 4.0 | | 0/4 | 0/4 |
| JC 221 (76) | 0.5 | | | |
| | 4.0 | | | |
| Hz166 (106) | 0.5 | 1/4 | 4/4 | 0/4 |
| (XHe-II-053 2′N) | 4.0 | 0/4 | 0/4 | 0/4 |
| JY-XHe-053 (15) | 0.5 | 1/4 | 3/4 | 0/4 |
| (XHe-II-053 2′F) | 4.0 | 0/4 | 4/4 | 0/4 |
| XLi-JY-DMH (23) | 0.5 | 0/4 | 3/4 | 0/4 |
| (triazol analog) | 4.0 | 0/4 | 2/4 | 0/4 |
| 147 (8-iodo-imidazo- | 0.5 | | 4/6 | 0/6 |
| benzodiazepine) | 4.0 | | 5/6 | 0/6 |

TABLE 6
Assessment of Antiseizure Activity of Imidazobenzodiazepines
in Rat via PO Administration.

| | | Rat PO | | | | |
|---|---|---|---|---|---|---|
| Compound | Time (h) | MES^{a} | scMet^{b} | | Tox_{b} | |
| Hz166 (106) | 0.25 | 0/4 | 0/4 | | 0/4 | |
| | 0.5 | 1/4 | 4/4 | | 0/4 | |
| | 1.0 | 0/4 | 2/4 | | 0/4 | |
| | 2.0 | 0/4 | 1/4 | | 0/4 | |
| | 4.0 | 0/4 | 0/4 | | 0/4 | |
| JY-XHe-053 (15) | 0.25 | nt^{d} | 1/4 | | 0/4 | |
| | 0.5 | 0/4 | 3/4 | | 0/4 | |
| | 1.0 | 0/4 | 3/4 | | 0/4 | |
| | 2.0 | 0/4 | 1/4 | | 0/4 | |
| | 4.0 | 1/4 | 4/4 | 2/4^{e} | 0/4 | 0/4^{e} |
| | 6.0 | 0/4 | nt^{d} | 2/4^{e} | nt^{d} | 0/4^{e} |

| | | | | | |
|---|---|---|---|---|---|
| XLi-JY-DMH (23) | 0.25 | 0/4 | 4/4 | 3/4^{c} | 0/4 |
| | 0.5 | 0/4 | 4/4 | 3/4^{c} | 0/4 |
| | 1.0 | 0/4 | 4/4 | 2/4^{c} | 0/4 |
| | 2.0 | 0/4 | 4/4 | 4/4^{c} | 0/4 |
| | 4.0 | 0/4 | 3/4 | 2/4^{c} | 0/4 |
| 147 (8-iodo-imidazo- | 0.25 | nt^{d} | | | |

| | | | | |
|---|---|---|---|---|
| benzodiazepine) | 0.5 | nt^{d} | 2/6 | 0/6^{f} |
| | 1.0 | | 5/6 | 0/6^{b} |

^{a}Dose of 30 mg/kg.
^{b}Dose of 50 mg/kg.
^{c}Dose of 15 mg/kg.
^{d}nt = not tested.
^{e}Dosed at 40 mg/kg.
^{f}observed some hyperactivity.

TABLE 7
Quantification of Antiseizure Activity (ED₅₀ MES, ED₅₀ scMet), TD₅₀TOX,
& Therapeutic Index (TI) via IP and PO routes.

| | | | | | | | TI | TI |
|---|---|---|---|---|---|---|---|---|
| | Mice IP | | | Rat PO | | | Mice IP | Rat PO |
| | ED₅₀ | ED₅₀ | TD₅₀ | ED₅₀ | ED₅₀ | TD₅₀ | TD₅₀/ED₅₀ | TD₅₀/ED₅₀ |
| Compound | MES | scMet | TOX | MES | scMet | TOX | (scMet) | (scMet) |
| Hz166 (106) | >300 | 16.28 | >500 | >250 | 98.5 | >500 | >30 | >5 |
| JY-XHe-053 (15) | >200 | 8.87 | >400 | >250 | 23.72 | >500 | >44 | >21 |
| XLi-JY-DMH (23) | >6 | 1.027 | 2.875 | >150 | 1.58 | 166.25 | 2.9 | >105 |
| Carbamazepine^{a} | 7.81 | >50 | 45.4 | 5.35 | >250 | 364 | <0.9 | 1.5 |
| Clonazepan^{a} | 25.6 | 0.02 | 0.26 | 7.86 | 0.61 | 2.38 | 13 | 3.9 |
| Phenytoin^{a} | 5.64 | >50 | 41.0 | 28.1 | >500 | >1000 | <0.82 | 2.0 |

MES maximal electroshock induced seizures; scMet sub-cutaneous pentyleletetrazole induced seizures;
TOX observed minimal muscular or neurological impairment as indicated by rotorod paradigm (mice) or abnormal, uncoordinated gait (rats);
TI therapeutic index;
IP intraperitoneal;
PO orally.
^{a}From “Discovery and Preclinical Development of Antiepileptic Drugs” in Antiepileptic Drugs, 5^{th} ed., Levy et al., editors, 2002. ^{b}

TABLE 8
Quantitative Assessment of Antiseizure Activity on
Imidazobenzodiazepines (IBZD) in Rat via IP Administration.

| | Rat IP | | |
|---|---|---|---|
| Compound | ED₅₀ MES | ED₅₀ scMet | TD₅₀ TOX |
| Hz166 (106) | nt | 35.5 | 95.34 |
| JY-XHe-053 (15) | >30 | 18.2 | 69.61 |
| XLi-JY-DMH (23) | nt | 4.2 | 2.08 |
| 8-iodo-imidazobenzodiazepine | nt | <50^{a} | >50^{b} |

Tested at 50 m/k at ¼, ½, 1, 2, 4 hr with results = 4/4, 1/4, 0/4, 2/4 and 2/4 protected;
^{b}tested at 50 m/k at 0.25, 0.5,, 1, 2, and 4 hrs with results = 0/4 toxic at all time points.

The antiseizure activity in rat animal models for MES, scMet and toxicity showed that ligands Hz166 (106), JY-XHe-053 (15), XLi-JY-DMH (23) and ligand 147 (8-iodo-imidazo-benzodiazepine) significantly increased the seizure threshold level of scMet protection in rats via both oral (po) and ip administration (Tables 4-6). In rats via the oral route the protection ranged from 1 to 98 mg/kg. Ligand XLi-JY-DMH (23) showed the most potency but via the i.p. route, the toxicity was greater than the efficacy. No motor impairment was observed in rats via oral administration for Hz166 (106) and JY-XHe-053 (15) up to 500 mg/kg. Ligand XLi-JY-DMH (23) had a TD₅₀ but showed a very large protective index. Ligand 147 (8-iodo-imidazo-benzodiazepine) was not quantified but activity was evident (5/6) at 50 mg/kg with no observed toxicity at that dose.

The quantitative antiseizure effects of benzodiazepines Hz166 (106), JY-XHe-053 (15), and XLi-JY-DMH (23) are shown in Table 7. Imidazobenzo-diazepine Hz166 (106) was much more active in the scMet seizure model than in MES, which suggested that it has potential use for the treatment of absence and myoclonic seizures. The ED₅₀ scMet for ligand Hz166 (106) was better than that of carbamazepine and phenyloin. Moreover, the TD₅₀ for Hz166 (106) of (>500 mg/kg) in mice ip provided a calculated therapeutic index (TI) greater than 30 in mice (ip). Similarly, JY-XHe-053 (15) showed better activity on scMet than MES in mice ip and rat po with ED₅₀s smaller than those reported for carbamazepine and phenyloin (Table 7). However in the MES, both carbamazepine and phenyloin have better ED₅₀s than ligand JY-XHe-053 (15). The TD₅₀ of JY-XHe-053 (15) was >400 mg/kg in both tests which provided a calculated TI of 44 in mice IP (Table 7). Triazobenzodiazepine XLi-JY-DMH (23) showed the highest activity of the ligands tested for scMet in mice and rats. However, only in rats via oral administration was a significant separation of protective effects and motor impairment found, and a TI>105.

To further characterize the anticonvulsant activity of some of these novel benzodiazepines, a hippocampus kindling screen was performed on Hz166 (106), JY-XHe-053 (15) and XLi-JY-DMH (23). The hippocampus kindling screen is a useful adjunct to the traditional MES and scMet tests for identification of a substance potential utility for treating complex partial seizures. Benzodiazepines Hz166 (106), JY-XHe-053 (15) and XLi-JY-DMH (23) appeared to block the kindled motor seizure as shown by the reduction of the seizure score from 4-5 to 3 (Table 9). No toxic effects were observed as indicated by the lack of motor impairment on the rats tested.

TABLE 9
Preliminary Hippocampus Kindling Screen-Rats IP

| | | | After- | | |
|---|---|---|---|---|---|
| | Seizure | Score | discharge | Duration (s) | |
| | Pre-Drug | Drug | Pre-Drug | Drug | |
| Compound | Low-High | Low-High | Low-High | Low-High | Tox |
| Hz166 (106) | 4-5 | 3- | 47-61 | 59- | 0/8^{a} |
| JY-XHe-053 | 5- | 3- | 30-41 | 38- | 0/2^{b} |
| (15) | | | | | |
| XLi-JY-DMH | 5- | 3- | 29-41 | 29- | 0/2^{c} |
| (23) | | | | | |

^{a}Dose of 50 mg/kg after 1.0 h.
^{b}Dose of 30 mg/kg after 1.0 h.
^{c}Dose of 3 mg/kg after 4 h

The short duration of action of Hz166 (106), JY-XHe-053 (15) and 8-iodo-imidazo benzodiazepine in mice IP (Table 4) is in agreement with esterase enzyme activity in these rodents, while administration in rats po (Table 6) would be expected to give slower absorption and less locomotor activity. Because the half-lives of such esters in primates and humans would be much longer, ligands Hz166 (106), JY-XHe-053 (15) and XLi-JY-DMH (23) represent potential anticonvulsant agents with little or no side effects. Certainly the efficacy profiles of Hz166 (106) and JY-XHe-053 (15) are consistent with this finding.

These subunit selective benzodiazepine derivatives demonstrate significant antiseizure activity in the scMet test in mice and rats and showed minimal or no motor impairment. Their subtype selective efficacy for the α2- and α3-GABA_{A} receptor subtypes over α1-GABA_{A} receptor subtype in oocytes was much greater than that of diazepam, consequently, ligands Hz166 (106) and JY-XHe-053 (15) displayed reduced or no motor impairment and no sedation in vivo. Therefore, ligands Hz166 (106) and JY-XHe-053 (15) appear to provide antiseizure activity, with minimal or no motor impairment by maintaining a good selectivity between α2/α3 versus α1 and an efficacy at α1 that is lower than that displayed by diazepam. The efficacy level at α1 appears to be of critical importance to avoid motor impairment in mice and rats. This is demonstrated by the fact that a slightly higher efficacy at α1 (289%) appears to result in some minimal motor impairment for ligand JY-XHe-053 (15) while ligand Hz166 (106) (255%) has no motor impairment. Ligand Hz166 (106) appears to have high enough efficacy at α2 and α3 to provide significant antiseizure activity with no toxicity in vivo (mice and rats) due to its lower efficacy at α1 subtypes compared to diazepam. Because of its simultaneous reduced efficacy at α1- and α5-GABA_{A} receptors, ligand Hz166 (106) represents an important potential anticonvulsant agent, which didn't develop tolerance when dosed for a five day period.

Since some clinically useful anti-epileptic drugs are ineffective at non-toxic doses in the standard MES and scMet tests, but still have anticonvulsant activities in vivo, the efficacy of Hz166 (106), JY-XHe-053 (15) and XLi-JY-DMH (23) was tested in the 6 Hz or ‘psychomotor’ test (Barton, M. E.; et al., Pharmacological characterization of the 6 Hz psychomotor seizure model of partial epilepsy. Epilepsy Res. 2001, 47, 217-27). Like the maximal electroshock (MES) test, the minimal clonic seizure (6 Hz) test is used to assess a compound's efficacy against electrically-induced seizures but uses a lower frequency (6 Hz) and longer duration of stimulation (3 s). Test compounds are pre-administered to mice via ip injection. At varying times, individual mice (four per time point) are challenged with sufficient current delivered through corneal electrodes to elicit a psychomotor seizure in 97% of animals (32 mA for 3s) (Toman, J. E.; et al., The search for new drugs against epilepsy. Tex Rep Biol Med. 1952, 10, 96-104). Untreated mice will display seizures characterized by a minimal clonic phase followed by stereotyped, automatistic behaviors described originally as being similar to the aura of human subjects with partial seizures. Animals not displaying this behavior are considered protected (Table 10).

The data from this screen indicates that all three compounds, (Hz166 (106), JY-XHe-053 (15) and XLi-JY-DMH (23)) were significantly protective in this model at doses significantly lower than where i.p. mouse toxicity becomes evident. It also demonstrates that XLi-JY-DMH (23) is the most highly potent of the three (Table 10).

TABLE 10
Minimal Clonic Seizure Model (6 Hz) Mice (IP)

| | Mice IP | | Mice IP (6 Hz) DSE (100 m/k) | | | | |
|---|---|---|---|---|---|---|---|
| | (6 Hz test) (mg/kg) | | (44 mA) | | | | |
| | ED₅₀ 32 mA | ED₅₀ 44 mA | 0.25 hr | 0.5 hr | 1.0 hr | 2.0 hr | 4 hr |
| Hz166 (106) | 25.84 | 115.22 | 4/4 | 1/4 | 1/4 | 0/4 | 0/4 |
| JY-XHe-053 (15) | 39.22 | Not tested | 2/4 | 4/4 | 2/4 | 2/4 | 0/4 |
| XLi-JY-DMH (23) | 0.17 | 0.40 | nt | nt | nt | nt | nt |
| Carbamazepine (CBZ)^{a} | >40^{a} | | | | | | |
| Clonazepan | 0.04 | | | | | | |
| Phenytoin (PHT)^{b} | >40^{b} | | | | | | |

^{ab}For the IP route in mice the TD₅₀ for CBZ is 41 mg/kg the TD₅₀ for PHT is 51 mg/kg so there is little of no separation of efficacy and toxicity.

### Tolerance Studies:

Benzodiazepines can be highly effective drugs in certain treatment paradigms. They are routinely employed for emergency situations such as status epilepticus and other acute conditions. But their use in chronic convulsant diseases has been limited due to side effects such as sedation and with high doses respiratory depression, hypotension and other effects. Further it has long been purported that chronic administration of this class of drugs can lead to tolerance to the anticonvulsant effects. This has limited their utility as first line treatment for chronic anticonvulsant conditions. Discovery of a potent BDZ with a decreased side effect profile and efficacy over extended treatment periods would be highly desirable.

In order to assess the effects of tolerance of the ligand Hz166 (106), whether tolerance could be detected using a chronic (5 day) dose of the candidate drug was studied. With typical benzodiazepines (for example diazepam), tolerance to the anticonvulsant effects of the drug are evident before 5 days have passed (J. Stables, private communication, NINDS), consequently studies were done for only 5 days. The dose utilized was the predetermined ED₅₀ against the scMet seizure model. In this model a subcutaneous injection of the convulsant Metrazol is used to produce highly reproducible clonic seizures in laboratory animals. The scMet test detects the ability of a test compound to raise the seizure threshold of an animal and thus protect it from exhibiting a clonic seizure. Animals are pretreated with various doses of the test compound. At the time of peak effect (TPE) of the test compound, a dose of Metrazol that induce convulsions in 97% of animals (CD₉₇: 85 mg/kg mice) is injected into a loose fold of skin in the midline of the neck. The animals are placed in isolation cages to minimize stress (Swinyard et al., 1961) and observed for the next 30 minutes for the presence or absence of a seizure. An episode of clonic spasms, approximately 3-5 seconds, of the fore and/or hindlimbs, jaws, or vibrissae is taken as the endpoint. Animals which do not meet this criterion are considered protected.

In this study, Sprague Dawley rats were used (average weight 150 gm). Three groups of 8 rats each were treated with ligand Hz166 (106) in the following manner: Group 1 received chronic 5-day dosing of the candidate drug. The scMet ED₅₀ of ligand Hz166 (106) (35.5 mg/kg) was administered via the ip route once daily for 5 consecutive days. It should be noted that the ED₅₀ calculation for the scMet protection using ligand Hz166 (106) produced a confidence limit between 22.67 and 48.32 mg/kg. In Group 2 identified as the acute dosing group methylcellulose was administered ip as a 4-day solvent control and a single dose of ligand Hz166 (106) was acutely administered ip on day 5. Finally, Group 3 was the solvent control group. These animals were administered the test solvent (methylcellulose 0.5%) as a control. Here methylcellulose was administered i.p. for all 5 days.

On day 5, all animals from each group were tested in the scMet model using the previously determined time of peak effect (TPE) of 15 minutes. The results are reported below. Immediately after testing, blood was collected via cardiac puncture. Samples were spun down, plasma was collected and acetonitrile added. Samples were frozen at −80° C. and shipped to a commercial laboratory (Enthalpy) for analysis.

TABLE 11

| | |
|---|---|
| Group 1 | 3/8 protected (chronic dosing) |
| Group 2 | 1/8 (acute dosing) |
| Group 3 | 0/8 (control, no drug) |

Results: Responses are noted as a ratio of the number of responders (animal protected) over the number of animals tested at the TPE (15 min)

In these studies, the chronic treated group (Group 1) showed approximately 40% protection (3/8) against scMet induced seizures using 35 mg/kg of ligand Hz166 (106) on day 5 (drug administered daily for 5 days). The acute treatment (Group 2) showed only 1/8 animals protected and the control group showed no protection (0/8). From this experiment it is concluded that under the conditions tested, there was no tolerance to the anticonvulsant effects of treatment of ligand Hz166 (106) for 5 days using the calculated ED₅₀. In previous studies with ligand Hz166 (106) versus scMet (pentylene tetrazole), oftentimes 4/4 animals were protected. This varied to 2/4 and also to 1/4. In this study in the acute dosing (1 day) only 1 out of 8 animals were protected, consequently, the dose should have been slightly higher in this strain of the rats than the ED₅₀. In the original preliminary study (different rats), 5/8 animals were protected, in the chronically treated group and 5/8 were protected in the acutely treated group, however, in that study the control rats (diazepam) showed no protection. The important result here is that after dosing for 5 days (chronic dosing) 3/8 animals were protected. Since the plasma levels (ligand Hz166 (106) and its carboxylic acid) of drug and metabolite were the same in acute and chronic dosing, this increase in protection in Group 1 (chronic dosing) was not due to increased plasma levels. The reasons for this require further study, however, it is clear ligand Hz166 (106) did not develop tolerance in these studies, in agreement with the low oocyte efficacy of ligand Hz166 (106) at α1 and α5 subtypes. Ligand Hz166 (106) did not develop tolerance and it is conceivable the related ligand 5 will not either. It must be pointed out that these esters are labile in rodents (esterase activity), but are much longer lived in dogs and primates.

Eight subjects in Group 1 were dosed with 35.5 mg/kg given for five days, and eight subjects in Group 2 were dosed with 35.5 mg/kg given on day 5 only. Group 3 (8 subjects) was dosed with vehicle only. The plasma samples obtained from these subjects were analyzed for HZ-166 and HZ-166 acid metabolite by LC-MS/MS and the concentration of each averaged between the subjects within the group, excluding outliers (Table 13). The subjects dosed with vehicle only contained no measurable amounts of HZ-166 or HZ-166 acid (lower limit of quantitation=1 ng/mL for each analyte). The average concentration of Hz-166 in the plasma was approximately 5% that of the acid metabolite for the dosed subjects. The averaged sums of Hz-166 and acid metabolite in Group 1 and Group 2 were 9.36 nmol/mL and 9.26 nmol/mL respectively, giving a percent difference between the groups of 1% between the two groups (Table 13).

In these studies, doses higher than 500 mg/kg were not tested, since the dose of 500 mg/kg was clearly not sedating. These compounds showed no sedative or locomotor effects in rats orally. Ligand 147 (8-Iodo-imidazobenzodiazepine) demonstrated protective effects in rats orally with no motor impairment. Ligand 147 (8-Iodo-imidazobenzodiazepine) showed little activity in mice ip, but demonstrated significant protection in rats orally (Table 7). The onset of the action of ligand 147 (8-Iodo-imidazobenzodiazepine) was very fast; after 15 minutes 100% of the rats were protected from scMet. At a lower dose of 30 mg/kg. Ligand 147 (8-Iodo-imidazobenzodiazepine) showed 80% protection (4/5) after 10 minutes with no motor impairment. Since the calculated logP for ligand 147 (8-Iodo-imidazo-benzodiazepine) (4.59) was significantly greater than Hz166 (106) (2.48), it is possible that ligand 147 crosses the blood brain barrier more rapidly than Hz166 (106), reaches a maximum effective concentration more rapidly and is consequently metabolized more rapidly when administered IP.

TABLE 12
Results, HZ-166 and HZ-166 acid.

| | Conc HZ-166 | Conc HZ-166 | |
|---|---|---|---|
| Subject No. | (ng/mL) | acid (ng/mL) | Comment |
| G1-1 | BLQ < (1.00) | BLQ < (1.00) | * |
| G1-2 | 118 | 3780 | |
| G1-3 | 198 | 3310 | |
| G1-4 | 30.5 | 1900 | |
| G1-5 | 94.3 | 2910 | |
| G1-6 | 85.4 | 2570 | |
| G1-7 | 168 | 3430 | |
| G1-8 | 138 | 2830 | |
| G2-1 | 202 | 3270 | |
| G2-2 | 165 | 2180 | |
| G2-3 | 8.67 | 167 | * |
| G2-4 | 209 | 3120 | |
| G2-5 | 273 | 3100 | |
| G2-6 | 210 | 3690 | |
| G2-7 | 93.3 | 2590 | |
| G2-8 | 176 | 2080 | |
| G3-1 | BLQ < (1.00) | BLQ < (1.00) | |
| G3-2 | 12.1 | 2940 | * |
| G3-3 | BLQ < (1.00) | BLQ < (1.00) | |
| G3-4 | BLQ < (1.00) | BLQ < (1.00) | |
| G3-5 | BLQ < (1.00) | BLQ < (1.00) | |
| G3-6 | BLQ < (1.00) | BLQ < (1.00) | |
| G3-7 | BLQ < (1.00) | BLQ < (1.00) | |
| G3-8 | BLQ < (1.00) | BLQ < (1.00) | |

* = Outlier within group, data not included in the calculated averages.
Data were converted from ng/mL to nmol/mL before taking averages.

TABLE 13
Averages with outliers excluded

| | HZ-166 acid | HZ-166 | Total HZ-166 + acid |
|---|---|---|---|
| Group | (nmol/mL) | (nmol/mL) | (nmol/mL) |
| 1 | 9.03 | 0.334 | 9.36 |
| 2 | 8.72 | 0.533 | 9.26 |
| 3 | 0 | 0 | 0 |

The anticonvulsant actions of ligands SH-053-2′-FS—CH₃ (120), SH-TS-CH₃ (143), and SH-TR-CH₃ (148) were similarly tested in the rat and mouse models. The results are summarized in Tables 14-20, below.

TABLE 14
Assessment of Anticonvulsant Activity in Mice
After 0.5 Hr and 4.0 Hr (Preliminary Screen)

| | | Mice IP | | |
|---|---|---|---|---|
| | | | | TOX |
| Compound | Time (Hr) | MES | scMet | (Rotorod Test) |
| SH-053-2′-FS-CH₃ (120) | 0.5 | 0/1 | 0/1 | 0/4 |
| 30 mg/kg | 4.0 | 0/1 | 0/1 | 0/2 |
| SH-053-2′-FS-CH₃ (120) | 0.5 | 0/3 | 2/5 | 1/8 |
| 100 mg/kg | 4.0 | 0/3 | 0/1 | 0/4 |
| SH-053-2′-FS-CH₃ (120) | 0.5 | 0/1 | 1/1 | 2/4 |
| 300 mg/kg | 4.0 | 0/1 | 0/1 | 0/2 |
| SH-TS-CH₃ (143) | 0.5 | 0/4 | 2/4 | 0/8 |
| 3 mg/kg | 4.0 | —/— | —/— | —/— |
| SH-TS-CH₃ (143) | 0.5 | 0/4 | 4/4 | 5/8¹⁴ |
| 10 mg/kg | 4.0 | —/— | —/— | —/— |
| SH-TS-CH₃ (143) | 0.5 | 1/1 | 1/1 | 4/4¹⁴ |
| 30 mg/kg | 4.0 | 0/1 | 0/1 | 0/2 |
| SH-TS-CH₃ (143) | 0.5 | 3/3 | 1/1 | 8/8¹⁴ |
| 100 mg/kg | 4.0 | 0/3 | 1/1 | 0/4 |
| SH-TS-CH₃ (143) | 0.5 | 1/1 | 1/1 | 4/4¹³ |
| 300 mg/kg | 4.0 | 1/1 | 1/1 | 2/2¹⁴ |
| SH-TR-CH₃ (146) | 0.5 | 0/1 | 0/1 | 0/4 |
| 30 mg/kg | 4.0 | 0/1 | 0/1 | 0/2 |
| SH-TR-CH₃ (146) | 0.5 | 0/3 | 0/1 | 1/8 |
| 100 mg/kg | 4.0 | 0/3 | 0/1 | 0/4 |
| SH-TR-CH₃ (146) | 0.5 | 0/1 | 1/1 | 1/4 |
| 300 mg/kg | 4.0 | 0/1 | 0/1 | 0/2 |

Notes:
¹⁴Unable to grasp rotorod
¹³Loss of righting reflex

TABLE 15
Assessment of Anticonvulsant Activity of Imidazobenzodiazepines
in Rat via PO Administration.

| | | Rat PO | | |
|---|---|---|---|---|
| Compound | Time (h) | MES | scMet | Tox |
| SH-053-2′-FS-CH₃ | 0.25 | | 1/4 | 0/4 |
| (120) | 0.5 | | 0/4 | 0/4 |
| 50 mg/kg | 1.0 | | 0/4 | 0/4 |
| | 2.0 | | 1/4 | 0/4 |
| | 4.0 | | 0/4 | 0/4 |
| SH-TS-CH₃ (143) | 0.25 | 0/4 | 0/4⁵ | 0/4 |
| 50 mg/kg | 0.5 | 0/4 | 1/4 | 0/4 |
| | 1.0 | 0/4 | 0/4⁵ | 0/4 |
| | 2.0 | 0/4 | 2/4²⁵ | 0/4 |
| | 4.0 | 0/4 | 0/4⁵ | 0/4 |
| SH-TR-CH₃ (146) | 0.25 | 0/4 | | 0/4 |
| 50 mg/kg | 0.5 | 0/4 | | 0/4 |
| | 1.0 | 0/4 | | 1/4 |
| | 2.0 | 0/4 | | 1/4 |
| | 4.0 | 0/4 | | 0/4 |

Notes:
⁵Death following clonic seizure
²⁵Myoclonic jerks

TABLE 16
Anticonvulsant Evaluation (6 Hz) Mice (IP)
ED₅₀

| | | | | STD | | |
|---|---|---|---|---|---|---|
| | ED₅₀ | (95% C.I.) | Slope | ERR | I (mA) | Hr |
| SH-053-2′-FS-CH₃ | 14.15 | 5.85-23.77 | 2.26 | 0.72 | 22 | 1 |
| (120) | | | | | | |
| SH-053-2′-FS-CH₃ | 71.2 | 35.49-104.68 | 3.87 | 1.44 | 32 | 1 |
| (120) | | | | | | |
| SH-TS-CH₃ (143) | 3.98 | 2.96-5.56 | 4.27 | 1.06 | 44 | 0.5 |
| SH-TR-CH₃ (146) | 2.08 | 1.2-3.21 | 3.04 | 0.91 | 32 | 0.25 |
| SH-TR-CH₃ (146) | 4.55 | 3.76-6.54 | 9.09 | 3.56 | 44 | 0.25 |

TABLE 17
Anticonvulsant Evaluation (6 Hz) Mice (IP)
ED₅₀ Biological Response

| | Dose (mg/kg) | | | | | | | | | | | | | | | | | |
|---|---|---|---|---|---|---|---|---|---|---|---|---|---|---|---|---|---|---|
| | 0.87 | 1.5 | 1.75 | 2.3 | 2.5 | 3 | 3.5 | 3.7 | 5 | 6 | 7 | 10 | 15 | 30 | 50 | 60 | 90 | 180 |
| SH-053-2′-FS-CH₃ | | | | | | | | | 2/8 | | | | 3/8 | 6/8 | | 8/8 | | |
| (22 mA, 1 Hr) | | | | | | | | | | | | | | | | | | |
| SH-053-2′-FS-CH₃ | | | | | | | | | | | | 0/8 | | | 3/8 | | 4/8 | 8/8 |
| (32 mA, 1 Hr) | | | | | | | | | | | | | | | | | | |
| SH-TS-CH₃ | | 0/8 | | 1/8 | | 4/8 | | | | 5/8 | | 8/8 | | | | | | |
| (44 mA, 0.5 Hr) | | | | | | | | | | | | | | | | | | |
| SH-TR-CH₃ | 1/8 | | 4/8 | | | | 5/8 | | | | 8/8 | | | | | | | |
| (32 mA, 0.25 Hr) | | | | | | | | | | | | | | | | | | |
| SH-TR-CH₃ | | | | | 0/8 | | | 2/8 | 5/8 | | | 8/8 | | | | | | |
| (44 mA, 0.25 Hr) | | | | | | | | | | | | | | | | | | |

\grave{ }18.
Anticonvulsant Evaluation (6 Hz) Mice (IP)
Time to Peak Effect (Hr)

| | Dose | I | | | | | |
|---|---|---|---|---|---|---|---|
| | (mg/kg) | (mA) | 0.25 | 0.5 | 1.0 | 2.0 | 4.0 |
| SH-053-2′-FS-CH₃ | 30 | 22 | | | 6/8 | 2/8 | |
| (120) | | | | | | | |
| SH-053-2′-FS-CH₃ | 50 | 22 | | 1/4 | 3/4 | 3/4 | |
| (120) | | | | | | | |
| SH-053-2′-FS-CH₃ | 100 | 22 | 2/4 | 4/4 | 4/4 | 4/4 | 2/4 |
| (120) | | | | | | | |
| SH-053-2′-FS-CH₃ | 100 | 32 | 2/4 | 1/4 | 4/4 | 2/4 | 2/4 |
| (120) | | | | | | | |
| SH-TR-CH₃ (146) | 3.5 | 32 | 4/4 | 1/4 | | | |
| SH-TR-CH₃ (146) | 7 | 32 | 4/4 | 4/4 | 2/4 | | |
| SH-TR-CH₃ (146) | 15 | 32 | 4/4 | 4/4¹⁴ | 4/4¹⁴ | 2/4 | 1/4 |
| SH-TR-CH₃ (146) | 50 | 32 | 4/4¹⁴ | 4/4¹⁴ | | | |
| SH-TR-CH₃ (146) | 100 | 32 | 4/4¹⁴ | 4/4¹⁴ | 4/4¹⁴ | 4/4¹⁴ | 4/4¹⁴ |

Notes:
¹⁴Unable to grasp rotorod

TABLE 19
Anticonvulsant Evaluation, Pilocarpine-induced Status, Rats
Time 0 Hrs, Time to Peak Effect (Hr)

| | Dose | | | | | |
|---|---|---|---|---|---|---|
| | (mg/kg) | 0.25 | 0.5 | 1.0 | 2.0 | 4.0 |
| SH-TR-CH₃ (146) | 100 | 0/2 | 0/2 | 0/2 | 0/2 | 0/2 |
| SH-TR-CH₃ (146) | 300 | 0/2 | 0/2 | 0/2 | 0/2 | 0/2 |
| SH-TR-CH₃ (146) | 500 | 0/2¹² | 0/2¹² | 0/2¹² | 0/2¹² | 0/2 |

Notes:
¹²Ataxia

TABLE 20
Pilocarpine-induced Status, Rats, Avg. Weight Change (g) ± SEM^{b}

| | Dose | Time^{a} | | Protected | Non-Protected |
|---|---|---|---|---|---|
| | (mg/kg) | (Hrs) | | Rats | Rats |
| SH-TR-CH₃ (146) | 600 | 0 | 4/7 | −5.0 ± 4.8 | −13.3 ± 1.0 |
| SH-TR-CH₃ (146) | 1,000 | 0.5 | 1/7^{c} | +5.0 ± 0.0 | −19.0 ± 0.8 |

Notes:
^{a}Post first Stage III seizure,
^{b}Weight change 24 hours Post first Stage III seizure,
^{c}death

1. A method for the treatment and prevention of seizures comprising administering to a subject in need of such treatment an effective amount of a compound selected from the group consisting of compounds according to Formulas A, B, C, D, I, III, or IV or a salt thereof, 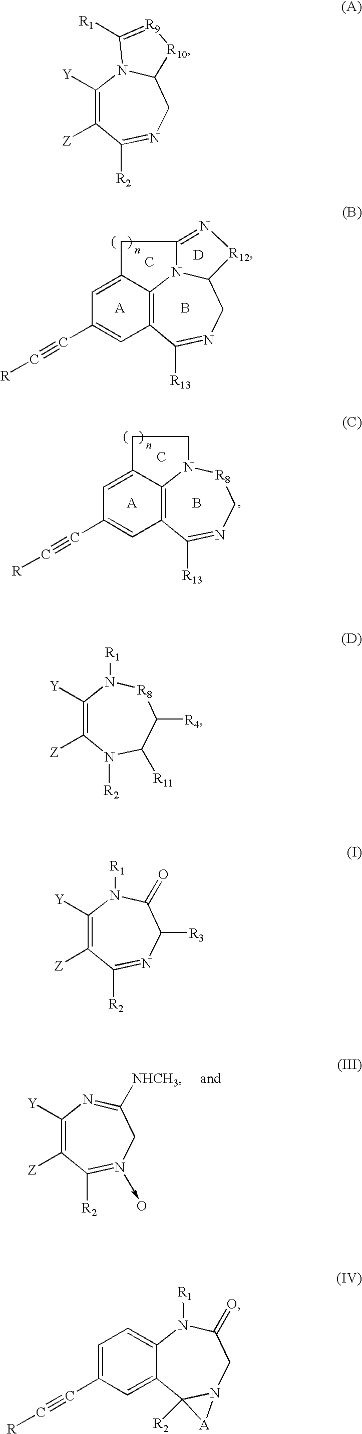
wherein Y and Z are taken together with the two intervening carbon atoms to form a ring selected from phenyl and thienyl, which ring is substituted at the C(8) position with at least the substituent —C≡C—R, where R is H, Si(CH₃)₃, isopropyl, methyl, or cycloalkyl;
R₂ is a substituted or unsubstituted at least partially unsaturated 5 or 6 membered cyclic or heterocyclic ring, wherein if substituted the substituent is one or more of F, Cl, Br, or NO₂ at the 2′-position; and R₁₀ is N, CH, C—CO₂—R₅, 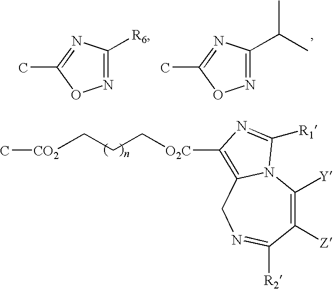
where n is 0 to 4 inclusive, 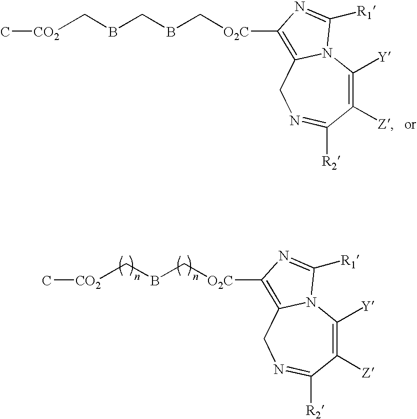
where n is 1 or 2 inclusive,
wherein Y′ and Z′ are taken together with the two intervening carbon atoms to form a ring selected from phenyl and thienyl, which ring is substituted at the C(8)′ position with at least the substituent —C≡C—R′, where R′ is H, Si(CH₃)₃, isopropyl, methyl, or cyclopropyl;
R₅ is a branched or straight chain C₁ to C₄ halogenated or unhalogenated alkyl or a methyl cyclopropyl;
R₆ is a branched or straight chain C₁ to C₄ alkyl or a methyl cyclopropyl;
R₁′ is one of H, CH₃, CF₃, CH₂CF₃, CH₂CH₃, or cyclopropyl;
R₂′ is a substituted or unsubstituted at least partially unsaturated 5 or 6 membered cyclic or heterocyclic ring, wherein if substituted the substituent is one or more of F, Cl, Br, or NO₂ at the 2′-position; and wherein when R₁₀ is C—CO₂—R₅, R₁ is one of H, CH₃, CF₃, CH₂CF₃, CH₂CH₃, CH₂C≡CH, or cyclopropyl;
otherwise, R₁ is one of H, CH₃, CF₃, CH₂CH₃, CH₂CF₃, or cyclopropyl; and wherein when R₁₀ is 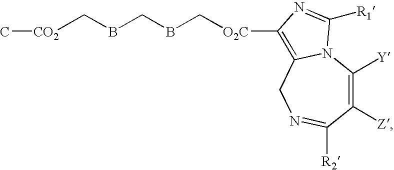
B is O or NH and wherein —BCH₂B— is optionally replaced with —N(R₇)—N(R₇)—, where R₇ is one of H, CH₃, alkyl, or cycloalkyl; and when R₁₀ is 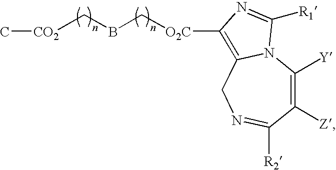
B is O or NH or —N(R₇)—N(R₇)—, where R₇ is one of H, CH₃, alkyl, or cycloalkyl;
and R₉ is N or 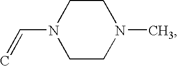
wherein when R₉ is 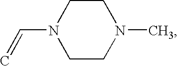
R₁ is ═O and R₁₀ is N; R₃ is one of —H, —OH, —OCON(CH₃)₂, —COOH, —COOCH₃, —COOC₂H₅, 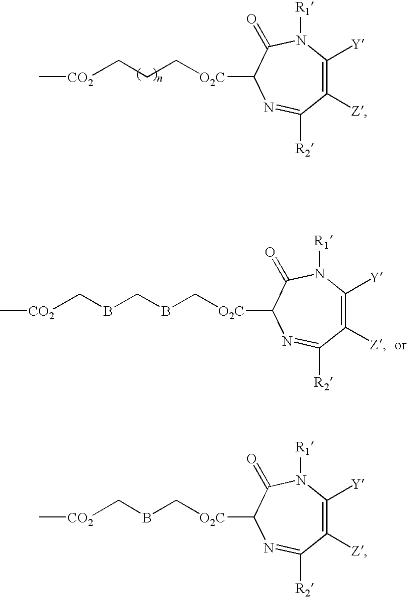
wherein if R₃ is 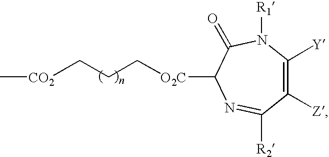
n is 0 to 4; Y′ and Z′ are taken together with the two intervening carbon atoms to form a ring selected from phenyl and thienyl, which ring is substituted at the C(7)′ position with at least the substituent —C≡C—R′, where R′ is H, Si(CH₃)₃, isopropyl, methyl, or cyclopropyl;
R₁ and R₁′ are independently one of H, CH₃, CF₃, CH₂CF₃, CH₂CH₃, or cyclopropyl;
R₂ and R₂′ are independently a substituted or unsubstituted at least partially unsaturated 5 or 6 membered cyclic or heterocyclic ring, wherein if substituted the substituent is at least one of F, Cl, Br, or NO₂ at the 2′-position, or
wherein if R₃ is 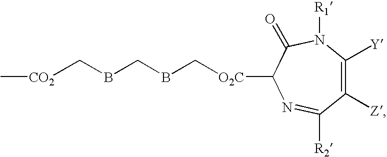
Y′ and Z′ are taken together with the two intervening carbon atoms to form a ring selected from phenyl and thienyl, which ring is substituted at the C(7)′ position with at least the substituent —C≡C—R′, where R′ is H, Si(CH₃)₃, t-butyl, isopropyl, methyl, or cyclopropyl; R₁ and R₁′ are independently one of H, CH₃, CF₃, CH₂CH₃, CH₂CF₃ or cyclopropyl; R₂ and R₂′ are independently a substituted or unsubstituted at least partially unsaturated 5 or 6 membered cyclic or heterocyclic ring, wherein if substituted the substituent is at least one of F, Cl, Br, or NO₂ at the 2′-position; B is O or NH and wherein —BCH₂B— is optionally replaced with —N(R₇)—N(R₇)—, where R₇ is one of H, branched or straight chain C₁ to C₄ alkyl, cycloalkyl,
wherein if R₃ is 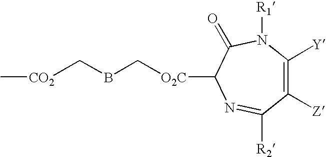
Y′ and Z′ are taken together with the two intervening carbon atoms to form a ring selected from phenyl and thienyl, which ring is substituted at the C(7)′ position with at least the substituent —C≡C—R′, where R′ is H, Si(CH₃)₃, t-butyl, isopropyl, methyl, or cyclopropyl;
R₁ and R₁′ are independently one of H, CH₃, CF₃, CH₂CH₃, CH₂CF₃ or cyclopropyl;
R₂ and R₂′ are independently a substituted or unsubstituted at least partially unsaturated 5 or 6 membered cyclic or heterocyclic ring, wherein if substituted the substituent is one or more of F, Cl, Br, or NO₂ at the 2′-position;
B is O, NH, or —N(R₇)—N(R₇)—, where R₇ is one of H, branched or straight chain C₁ to C₄ alkyl, or cycloalkyl, wherein R₂ is not phenyl when R₃ is H, R₁ is H or CH₃ and Y and Z taken together form a phenyl ring;
wherein R₈ is C═O, C═S, or C—NHCH₃;
R₁₁ is O or N and R₄ is H, O or C,
wherein when R₁₁ is O, R₄ is H, and R₈ is C═O;
when R₁₁ is N, R₄ is C and R₁₁ and R₄ are taken together with an intervening nitrogen atom to form a pyrazole ring;
when R₁₁ is N, R₄ is O and R₁₁ and R₄ are taken together with an intervening carbon atom to form an oxazole ring; or
when R₁₁ is N, R₄ is C and R₁₁ and R₄ are taken together with two intervening carbon atoms to form a pyridine ring;
and R₁₂ is N or CH, R₁₃ is phenyl, 2-thienyl, 3-thienyl, 2-pyridyl, or 2-pyridyl N—O, and if the compound is according to Formula B or C, n is an integer from 1 to 2 inclusive and ring C is saturated or unsaturated.

2. The method of claim 1 wherein the compound has the formula 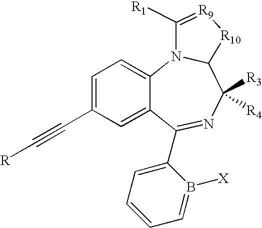
or a salt thereof, where R is H or Si(CH₃)₃,
R₁ is —H, —CH₃, ═O, or —CF₃, R₃ and R₄ are both H, or R₃ is H and R₄ is S(CH₃), or R₃ is R(CH₃) and R₄ is H, R₉ is N, R₁₀ is N, C—CO₂CH₂CH₃, C—CO₂CH₂CF₃, or C—CO₂C(CH₃)₃, and B is C or N, and X is absent, H, F, Cl, or Br.

3. The method of claim 1 wherein the compound is 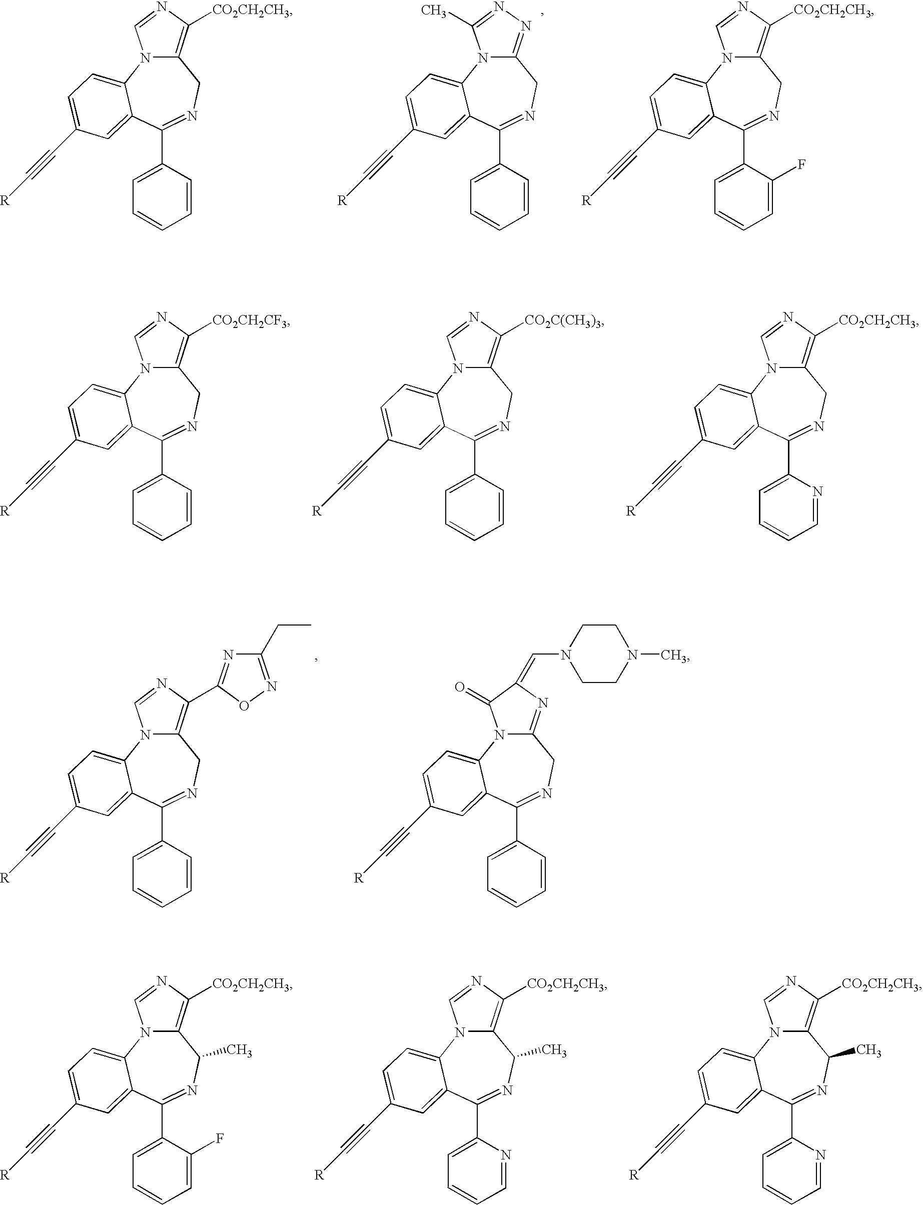 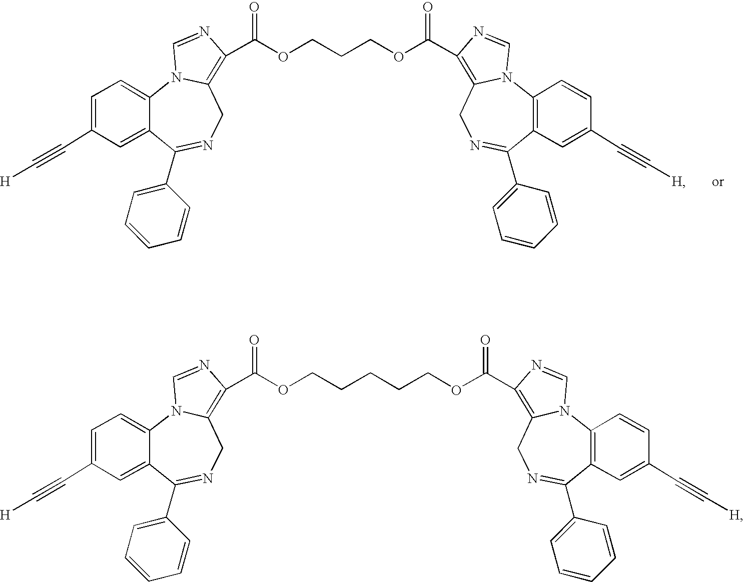
where R is H or Si(CH₃)₃.

4. The method of claim 2 wherein the compound is 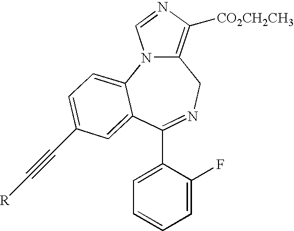
where R is H or Si(CH₃)₃.

5. The method of claim 2 wherein the compound is 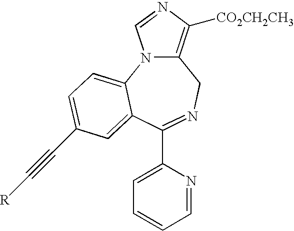
where R is H or Si(CH₃)₃.

6. The method of claim 2, wherein the compound is 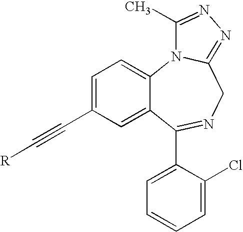
where R is H or Si(CH₃)₃.

7. The method of claim 1 wherein the compound is 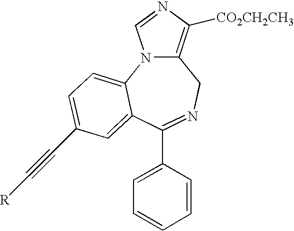
where R is H or Si(CH₃)₃.

8. A method for the treatment and prevention of seizures comprising administering to a subject in need of such treatment an effective amount of a compound of the formula 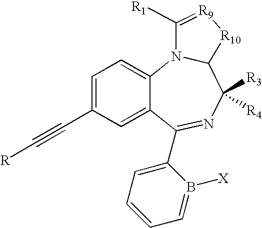
or a salt thereof, where R is H or Si(CH₃)₃,
R₁ is —H, —CH₃, —CF₃, or ═O, R₃ and R₄ are both H, or R₃ is H and R₄ is SCH₃, or R₃ is RCH₃ and R₄ is H,
R₉ is N, or 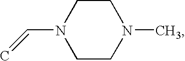
R₁₀ is N, C—CO₂CH₂CH₃, C—CO₂CH₂CF₃, C—CO₂C(CH₃)₃, C—R₁₅, C—R₁₆, or 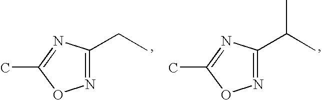
and B is C or N, and X is absent, H, F, Cl, or Br, and when R₁₀ is C—R₁₅, R₁₅ is 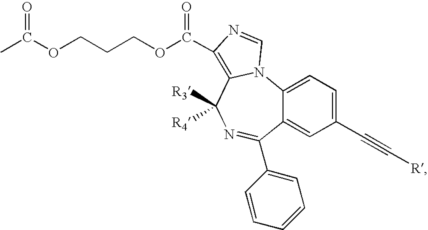
R₁ is H, R′ is H or Si(CH₃)₃, R′₃ and R′₄ are both H, or R′₃ is H and R′₄ is S(CH₃), or R′₃ is R(CH₃), and R′₄ is H, and when R₁₀ is C—R₁₅, R₁₅ is R₁₆ is 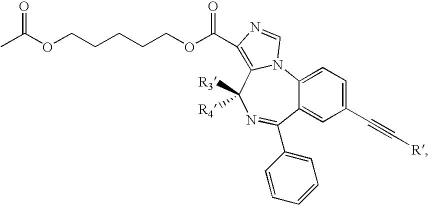
R₁ is H, R′ is H or Si(CH₃)₃, R′₃ and R′₄ are both H, or R′₃ is H and R′₄ is S(CH₃), or R′₃ is R(CH₃), and R′₄ is H.

9. The method of claim 8 wherein the compound has the formula 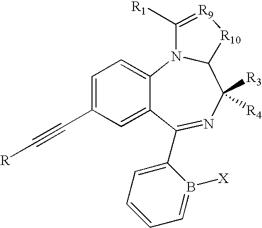
or a salt thereof, where R is H or Si(CH₃)₃,
R₁ is —H, —CH₃, ═O, or —CF₃, R₃ and R₄ are both H, or R₃ is H and R₄ is S(CH₃), or R₃ is R(CH₃) and R₄ is H, R₉ is N, R₁₀ is N, C—CO₂CH₂CH₃, C—CO₂CH₂CF₃, or C—CO₂C(CH₃)₃, and B is C or N, and X is absent, H, F, Cl, or Br.

10. The method of claim 8 wherein the compound is 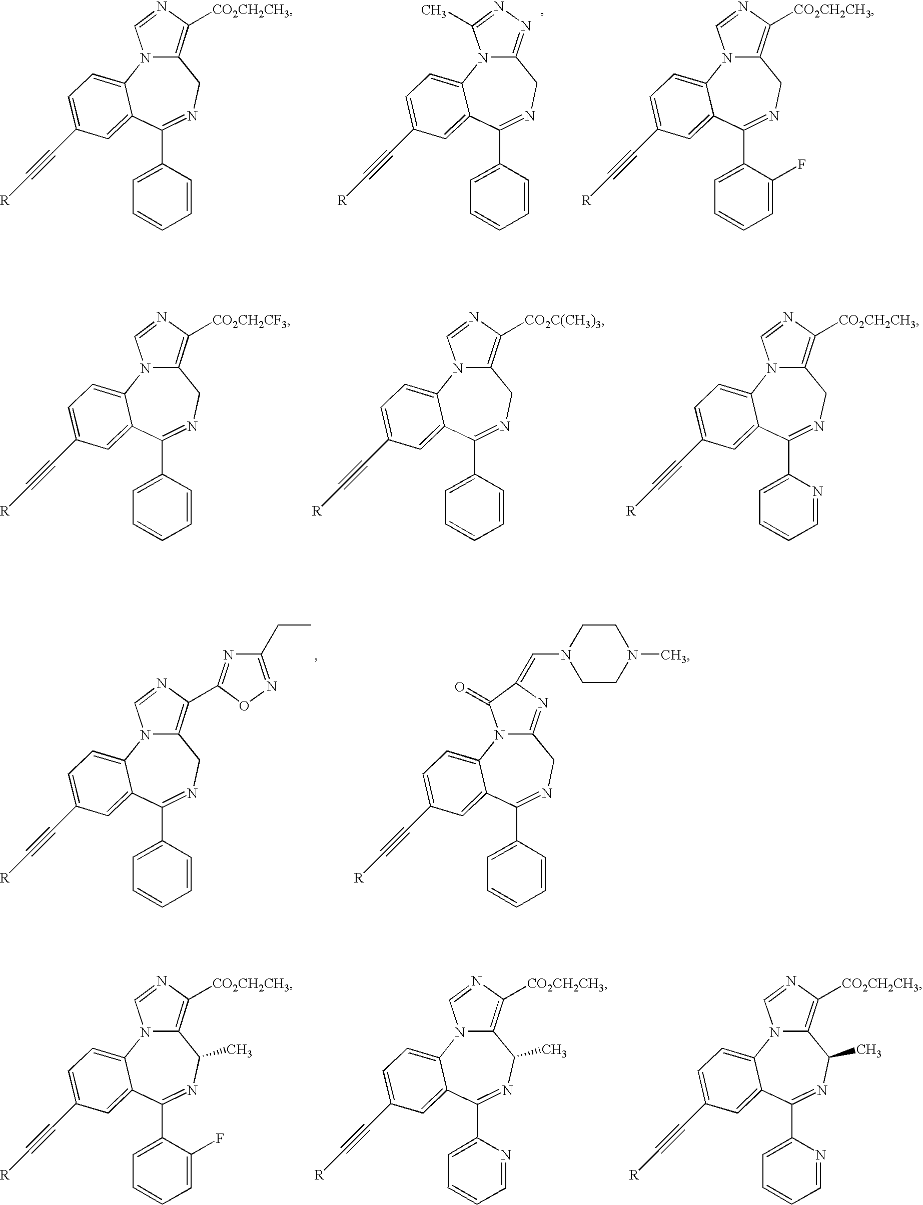 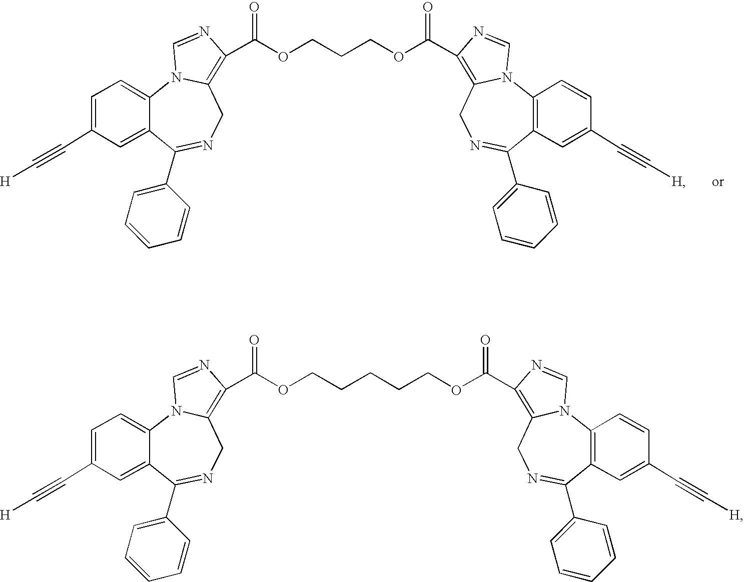
where R is H or Si(CH₃)₃.

11. The method of claim 8 wherein the compound is 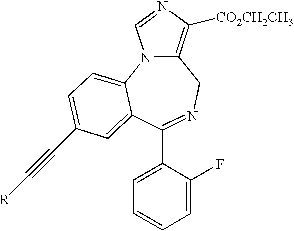
where R is H or Si(CH₃)₃.

12. The method of claim 8 wherein the compound is 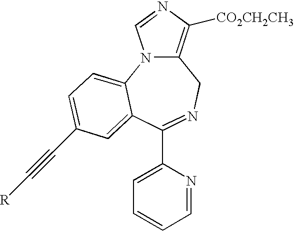
where R is H or Si(CH₃)₃.

13. The method of claim 8, wherein the compound is 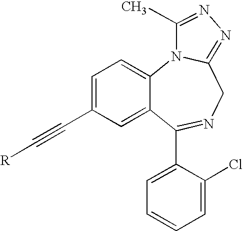
where R is H or Si(CH₃)₃.

14. The method of claim 8 wherein the compound is 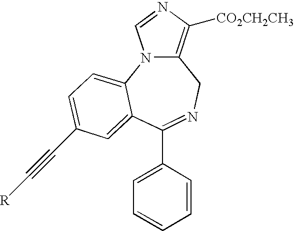
where R is H or Si(CH₃)₃.
